(19) United States
(12) Patent Application Publication
(10) Pub. No.: US 2019/0136230 A1
(43) Pub. Date: May 9, 2019

(54) GENETICALLY ENGINEERED CELLS AND METHODS OF MAKING THE SAME

(71) Applicants: Juno Therapeutics, Inc., Seattle, WA (US); Editas Medicine, Inc., Cambridge, MA (US)

(72) Inventors: Blythe SATHER, Seattle, WA (US); G. Grant WELSTEAD, Cambridge, MA (US); David A. BUMCROT, Cambridge, MA (US); Ari E. FRIEDLAND, Cambridge, MA (US); Jon JONES, Seattle, WA (US); Morgan L. MAEDER, Cambridge, MA (US); Chris NYE, Seattle, WA (US); Eugenio Marco RUBIO, Cambridge, MA (US); Ruth SALMON, Seattle, WA (US)

(73) Assignees: Juno Therapeutics, Inc., Seattle, WA (US); Editas Medicine, Inc., Cambridge, MA (US)

(21) Appl. No.: 16/098,845

(22) PCT Filed: May 6, 2017
(86) PCT No.: PCT/US2017/031464

§ 371 Date: Nov. 2, 2018

Related U.S. Application Data
(60) Provisional application No. 62/333,144, filed on May 6, 2016.
(60) Provisional application No. 62/332,657, filed on May 6, 2016.

(51) Int. Cl. C12N 15/11; C07K 14/725; C12N 5/0783; C07K 14/705; C12N 9/22; C07K 16/28; A61P 35/00; A61K 38/17; A61K 31/7088; A61K 38/46; A61K 39/395

(52) U.S. Cl. CPC C12N 15/11; A61K 2039/505; C12N 5/0636; C07K 14/70521; C07K 14/70578; C12N 9/22; C07K 16/2803; C07K 16/2878; A61P 35/00; A61K 38/1774; A61K 31/7088; A61K 38/177; A61K 38/465; A61K 39/39558; A61K 39/3955; C07K 2317/622; C07K 2319/30; C07K 2319/02; C12N 2800/80; C12N 2310/20; C12N 2501/2302; C12N 2501/2307; C12N 2501/2315; C12N 2501/998; C12N 2310/336; C12N 2310/317; C07K 2317/73; C07K 14/7051

(57) ABSTRACT

Provided are CRIS PR/CAS-related methods, compositions and components for editing a target nucleic acid sequence, or modulating expression of a target nucleic acid sequence, and applications thereof in connection with cancer immunotherapy comprising adoptive transfer of engineered T cells or T cell precursors.

### CROSS-REFERENCE TO RELATED APPLICATIONS

This application claims priority from U.S. provisional application No. 62/333,144 filed May 6, 2016, entitled “Genetically Engineered Cells And Methods Of Making The Same,” and U.S. provisional application No. 62/332,657 filed May 6, 2016, entitled “CRISPR-CAS-Related Methods, Compositions And Components For Cancer Immunotherapy,” the contents of each of which is incorporated by reference in its entirety.

### INCORPORATION BY REFERENCE OF SEQUENCE LISTING

The present application is being filed along with a Sequence Listing in electronic format. The Sequence Listing is provided as a file entitled 735042006440SEQLIST.TXT, created May 4, 2017, which is 12,031,926 bytes in size. The information in the electronic format of the Sequence Listing is incorporated by reference in its entirety.

### FIELD

The present disclosure relates to CRISPR/CAS-related methods, compositions and components for editing a target nucleic acid sequence, or modulating expression of a target nucleic acid sequence, and applications thereof in connection with cancer immunotherapy comprising adoptive transfer of engineered T cells or T cell precursors.

### BACKGROUND

Various strategies are available for producing and administering engineered cells for adoptive therapy. For example, strategies are available for engineering immune cells expressing genetically engineered antigen receptors, such as CARs, and for suppression or repression of gene expression in the cells. Improved strategies are needed to improve efficacy of the cells, for example, by avoiding suppression of effector functions and improving the activity and/or survival of the cells upon administration to subjects. Provided are methods, cells, compositions, kits, and systems that meet such needs.

### SUMMARY

Provided are compositions that include an engineered immune cell containing a recombinant receptor and an agent capable of inducing a genetic disruption of a PDCD1 gene or a genetic disruption of a PDCD1 gene encoding the PD-1 polypeptide, such as for use in adoptive cell therapy, for example, to treat diseases and/or conditions in the subjects. Also provided are methods for producing or generating such compositions or cells, cells, cell populations, compositions, and methods of using such compositions or cells. The compositions and cells generally include agents capable of inducing a genetic disruption or prevention or reduction of expression of a PDCD1 gene, or a genetic disruption of a PDCD1 gene. Also provided are methods for administering to subjects the provided compositions, cell populations or cells expressing genetically engineered (recombinant) cell surface receptors and contain a genetic disruption of a PDCD1 gene, such as produced by the methods, for example, for adoptive cell therapy to treat diseases and/or conditions in the subjects.

In some embodiments, provided are compositions containing (a) an engineered immune cell containing a recombinant receptor that specifically binds to an antigen; and (b) an agent capable of inducing a genetic disruption of a PDCD1 gene encoding a PD-1 polypeptide, wherein said agent is capable of inducing said genetic disruption in, and/or preventing or reducing PD-1 expression in, at least 70%, at least 75%, at least 80%, or at least or greater than 90% of the cells in the composition and/or at least 70%, at least 75%, at least 80%, or at least or greater than 90% of the cells in the composition that express the recombinant receptor.

In some embodiments, provided are compositions containing (a) an engineered immune cell containing a nucleic acid encoding a recombinant receptor that specifically binds to an antigen; and (b) an agent capable of inducing a genetic disruption of a PDCD1 gene encoding a PD-1 polypeptide, wherein said agent is capable of inducing said genetic disruption in, and/or preventing or reducing PD-1 expression in, at least 70%, at least 75%, at least 80%, or at least or greater than 90% of the cells in the composition and/or at least 70%, at least 75%, at least 80%, or at least or greater than 90%, of the cells in the composition that express the recombinant receptor.

In some embodiments provided herein, the composition includes engineered immune cells that express the recombinant receptor on its surface.

In some embodiments, provided are compositions containing a cell population that contains an engineered immune cell that contains (a) a recombinant receptor that specifically binds to an antigen; and (b) a genetic disruption of a PDCD1 gene encoding a PD-1 polypeptide, said genetic disruption preventing or reducing the expression of said PD-1 polypeptide, wherein at least about 70%, at least about 75%, or at least about 80% or at least or greater than about 90% of the cells in the composition contain the genetic disruption; do not express the endogenous PD-1 polypeptide; do not contain a contiguous PDCD1 gene, do not contain a PDCD1 gene, and/or do not contain a functional PDCD1 gene; and/or do not express a PD-1 polypeptide; and/or at least about 70%, at least about 75%, or at least about 80% or at least or greater than about 90% of the cells in the composition that express the recombinant receptor contain the genetic disruption, do not express the endogenous PD-1 polypeptide, and/or do not express a PD-1 polypeptide.

In some embodiments, provided are composition s containing a cell population that contains an engineered immune cell that contains (a) a recombinant receptor that specifically binds to an antigen, wherein the engineered immune cell is capable of inducing cytotoxicity, proliferating and/or secreting a cytokine upon binding of the recombinant receptor to said antigen; and (b) a genetic disruption of a PDCD1 gene encoding a PD-1 polypeptide, said genetic disruption capable of preventing or reducing the expression of said PD-1 polypeptide, optionally wherein said prevention or reduction is in at least at or about or greater than at or about 70%, 75%, 80%, 85%, or 90% of the cells in the composition and/or of the cells in the composition that express the recombinant receptor.

In some embodiments, provided are compositions containing a cell population that contains a population of engineered immune cells, each containing (a) a recombinant receptor that specifically binds to an antigen; and (b) a genetic disruption of a PDCD1 gene encoding a PD-1 polypeptide, wherein said genetic disruption is capable of preventing or reducing the expression of said PD-1 polypeptide, wherein: the engineered immune cells, on average, exhibit expression and/or surface expression of the receptor at a level that is the same, about the same or substantially the same, as compared to the average expression and/or surface expression level, respectively, of said recombinant receptor in other cells in the composition that contain the recombinant receptor and do not contain the genetic disruption, or the engineered immune cells do not express the PD-1 polypeptide and on average, exhibit expression and/or surface expression of the receptor at a level is the same, about the same, or substantially the same as compared to the average expression and/or surface level, respectively, in cells of the composition that contain the recombinant receptor and that express the PD-1 polypeptide.

In some embodiments, the recombinant receptor is capable, upon incubation with the antigen, a cell expressing the antigen, and/or an antigen-receptor activating substance, of specifically binding to the antigen, of activating or stimulating the engineered T cell, of inducing cytotoxicity, or of inducing proliferation, survival, and/or cytokine secretion by the immune cell, optionally as measured in an in vitro assay, optionally in an in vitro assay, which optionally contains incubation for 12, 24, 36, 48, or 60 hours, optionally in the presence of one or more cytokines. In some embodiments, the engineered immune cell is capable, upon incubation with the antigen, a cell expressing the antigen, and/or an antigen-receptor activating substance, of specifically binding to the antigen, of inducing cytotoxicity, proliferating, surviving, and/or secreting a cytokine, optionally as measured in an in vitro assay, which optionally contains incubation for 12, 24, 36, 48, or 60 hours, optionally in the presence of one or more cytokines and optionally does or does not contain exposing the immune cell to a PD-L1-expressing cell.

In some embodiments, the level or degree or extent or duration of the binding, cytotoxicity, proliferation, survival, or cytokine secretion is the same, about the same or substantially the same as compared to that detected or observed for an immune cell containing the recombinant receptor but not containing the genetic disruption of a PDCD1 gene, when assessed under the same conditions. In some embodiments, the binding, cytotoxicity, proliferation, survival, and/or cytokine secretion is as measured, optionally in an in vitro assay, following withdrawal and re-exposure to the antigen, antigen-expressing cell, and/or substance.

In some embodiments, the immune cell is a primary cell from a subject. In some embodiments, the immune cell is a human cell. In some embodiments, the immune cell is a white blood cell, such as an NK cell or a T cell. In some embodiments, the immune cell contains a plurality of T cells containing unfractionated T cells, contains isolated CD8+ cells or is enriched for CD8+ T cells, or contains isolated CD4+ T cells or is enriched for CD4+ cells, and/or is enriched for a subset thereof selected from the group consisting of naïve cells, effector memory cells, central memory cells, stem central memory cells, effector memory cells, and long-lived effector memory cells. In some embodiments, the percentage, of T cells, or T cells expressing the receptor, and containing the genetic disruption in the composition, that exhibit a non-activated, long-lived memory, or central memory phenotype, is the same or substantially the same as a population of cells the same or substantially the same as the composition but not containing the genetic disruption or but expressing the PD-1 polypeptide.

In some embodiments, the percentage of T cells in the composition exhibiting a non-activated, long-lived memory, or central memory phenotype is the same, about the same or substantially the same as compared to the percentage of T cells exhibiting the phenotype in a composition containing T cells, containing the recombinant receptor but not containing the genetic disruption of a PDCD1 gene encoding a PD-1 polypeptide when assessed under the same conditions, which optionally is compared in the absence or presence of contacting or exposing the immune cell to PD-L1. In some embodiments, the phenotype is as assessed following incubation of the composition at or about 37° C.±2° C. for at least 12 hours, 24 hours, 48 hours, 96 hours, 6 days, 12 days, 24 days, 36 days, 48 days or 60 days. In some embodiments, the incubation is in vitro. In some embodiments, at least a portion of the incubation is performed in the presence of a stimulating agent, which at least a portion is optionally for up to 1 hour, 6 hours, 24 hours, or 48 hours of the incubation. In some embodiments, the stimulating agent is an agent capable of inducing proliferation of T cells, CD4+ T cells and/or CD8+ T cells. In some embodiments, the stimulating agent is or contains an antibody specific for CD3 an antibody specific for CD28 and/or a cytokine. In some embodiments, the T cell containing the recombinant receptor contains one or more phenotypic markers selected from CCR7+, 4-1BB+(CD137+), TIM3+, CD27+, CD62L+, CD127+, CD45RA+, CD45RO−, t-bet1^{low}, IL-7Ra+, CD95+, IL-2Rβ+, CXCR3+ or LFA-1+.

In some embodiments, the recombinant receptor is a functional non-TCR antigen receptor or a transgenic TCR. In some embodiments, the recombinant receptor is a chimeric antigen receptor (CAR), such as a CAR containing an antigen-binding domain that is an antibody or an antibody fragment. In some embodiments, the antibody fragment contained in the recombinant receptor is a single chain fragment. In some embodiments, the antibody fragment contains antibody variable regions joined by a flexible immunoglobulin linker. In some embodiments, the fragment contains an scFv.

In some embodiments, the antigen is associated with a disease or disorder, such as an infectious disease or condition, an autoimmune disease, an inflammatory disease or a tumor or a cancer. In some embodiments, the recombinant receptor specifically binds to a tumor antigen. In some embodiments, the antigen that the recombinant receptor binds to is selected from ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acethycholine receptor, GD2, GD3, HMW-MAA, IL-22R-alpha, IL-13R-alpha2, kdr, kappa light chain, Lewis Y, L1-cell adhesion molecule (CD171), MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1), cyclin A1 (CCNA1) or interleukin 12.

In some embodiments, the recombinant receptor contains an intracellular signaling domain containing an ITAM. In some embodiments, the intracellular signaling domain contains an intracellular domain of a CD3-zeta (CD3ζ) chain. In some embodiments, the recombinant receptor further contains a costimulatory signaling region, such as a costimulatory signaling region containing a signaling domain of CD28 or 4-1BB.

In some embodiments, wherein the agent capable of inducing a genetic disruption of a PDCD1 gene contains at least one of (a) a least one guide RNA (gRNA) having a targeting domain that is complementary with a target domain of a PDCD1 gene or (b) at least one nucleic acid encoding the at least one gRNA. In some embodiments, the agent contains at least one complex of a Cas9 molecule and a gRNA having a targeting domain that is complementary with a target domain of a PDCD1 gene. In some embodiments, the guide RNA further contains a first complementarity domain, a second complementarity domain that is complementary to the first complementarity domain, a proximal domain and optionally a tail domain. In some embodiments, the first complementarity domain and second complementarity domain are joined by a linking domain. In some embodiments, the guide RNA contains a 3′ poly-A tail and a 5′ Anti-Reverse Cap Analog (ARCA) cap. In some embodiments, the Cas9 molecule is an enzymatically active Cas9.

In some embodiments, the at least one gRNA includes a targeting domain containing a sequence selected from the group consisting of GUCUGGGCGGUGCUACAACU (SEQ ID NO:508), GCCCUGGCCAGUCGUCU (SEQ ID NO: 514), CGUCUGGGCGGUGCUACAAC (SEQ ID NO:1533), UGUAGCACCGCCCAGACGAC (SEQ ID NO:579), CGACUGGCCAGGGCGCCUGU (SEQ ID NO:582) and CACCUACCUAAGAACCAUCC (SEQ ID NO:723). In some embodiments, the at least one gRNA includes a targeting domain containing the sequence CGACUGGCCAGGGCGCCUGU (SEQ ID NO:582).

In some embodiments, the Cas9 molecule is an S. aureus Cas9 molecule. In some embodiments, the Cas9 molecules is an S. pyogenes Cas9. In some compositions, the Cas9 molecule lacks an active RuvC domain or an active HNH domain. In some embodiments, the Cas9 molecule is an S. pyogenes Cas9 molecule containing a D10A mutation. In some embodiments, the Cas9 molecule is an S. pyogenes Cas9 molecule containing an N863A mutation.

In some of the embodiments provided herein, the genetic disruption contains creation of a double strand break which is repaired by non-homologous end joining (NHEJ) to effect insertions and deletions (indels) in the PDCD1 gene.

In some embodiments, at least about 70%, at least about 75%, or at least about 80% of the cells in the composition contain the genetic disruption; do not express the endogenous PD-1 polypeptide; do not contain a contiguous PDCD1 gene, a PDCD1 gene, and/or a functional PDCD1 gene; and/or do not express a PD-1 polypeptide; and/or at least about 70%, at least about 75%, or at least about 80% of the cells in the composition that express the recombinant receptor contain the genetic disruption, do not express the endogenous PD-1 polypeptide, or do not express a PD-1 polypeptide. In some embodiments, greater than 80%, 81%, 82%, 83%, 84%, 85%, 86%, 87%, 88%, 89%, 90%, 91%, 92%, 93%, 94%, or 95% of the cells in the composition contain the genetic disruption; do not express the endogenous PD-1 polypeptide; do not contain a contiguous PDCD1 gene, a PDCD1 gene, and/or a functional PDCD1 gene; and/or do not express a PD-1 polypeptide; and/or greater than 80%, 81%, 82%, 83%, 84%, 85%, 86%, 87%, 88%, 89%, 90%, 91%, 92%, 93%, 94%, or 95% of the cells in the composition that express the recombinant receptor contain the genetic disruption, do not express the endogenous PD-1 polypeptide, or do not express a PD-1 polypeptide.

In some embodiments, both alleles of the gene in the genome are disrupted.

In some embodiments, cells in the composition and/or the cells in the composition that express the recombinant receptor are not enriched or selected for cells that contain the genetic disruption; do not express the endogenous PD-1 polypeptide; do not contain a contiguous PDCD1 gene, a PDCD1 gene, and/or a functional PDCD1 gene; and/or do not express a PD-1 polypeptide.

In some embodiments, no more than 2, no more than 5 or no more than 10 other genes in each cell in the composition, or each cell in the composition that expresses the recombinant receptor, on average, are disrupted or are disrupted by the agent, such as no other genes in each cell in the composition or each cell in the composition that expresses the recombinant receptor are disrupted in the cell or are disrupted by the agent.

In some embodiments, any of the compositions provided herein further contains a pharmaceutically acceptable buffer.

Also provided herein are methods of producing a genetically engineered immune cell, including: (a) introducing into an immune cell a nucleic acid molecule encoding a recombinant receptor that specifically binds to an antigen; and (b) introducing into the immune cell an agent capable of inducing a genetic disruption of a PDCD1 gene encoding a PD-1 polypeptide including one of (i) at least one gRNA having a targeting domain that is complementary with a target domain of the PDCD1 gene or (ii) at least one nucleic acid encoding the at least one gRNA.

Also provided herein are methods of producing a genetically engineered immune cell, including introducing into an immune cell expressing a recombinant receptor that specifically binds to an antigen an agent capable of inducing a genetic disruption of a PDCD1 gene encoding a PD-1 polypeptide including one of (i) at least one gRNA having a targeting domain that is complementary with a target domain of the PDCD1 gene or (ii) at least one nucleic acid encoding the at least one gRNA.

In some embodiments, the agent includes at least one complex of a Cas9 molecule and a gRNA having a targeting domain that is complementary with a target domain of a PDCD1 gene.

In some embodiments, the guide RNA further includes a first complementarity domain, a second complementarity domain that is complementary to the first complementarity domain, a proximal domain and optionally a tail domain. In some embodiments, the first complementarity domain and second complementarity domain are joined by a linking domain. In some embodiments, the guide RNA includes a 3′ poly-A tail and a 5′ Anti-Reverse Cap Analog (ARCA) cap.

In some embodiments, introduction includes contacting the cells with the agent or a portion thereof, in vitro. In some embodiments, introduction of the agent includes electroporation. In some embodiments, the introduction further includes incubating the cells, in vitro prior to, during or subsequent to the contacting of the cells with the agent or prior to, during or subsequent to the electroporation. In some embodiments, the introduction in (a) includes transduction and the introduction further includes incubating the cells, in vitro, prior to, during or subsequent to the transduction. In some embodiments, at least a portion of the incubation is in the presence of (i) a cytokine selected from the group consisting of IL-2, IL-7, and IL-15, and/or (ii) a stimulating or activating agent or agents, optionally including anti-CD3 and/or anti-CD28 antibodies. In some embodiments, the introduction in (a) includes: prior to transduction, incubating the cells with IL-2 at a concentration of 20 U/mL to 200 U/mL, optionally about 100 U/mL; IL-7 at a concentration of 1 ng/mL to 50 ng/mL, optionally about 10 ng/mL and/or IL-15 at a concentration of 0.5 ng/mL to 20 ng/mL, optionally about 5 ng/mL; and subsequent to transduction, incubating the cells with IL-2 at a concentration of 10 U/mL to 200 U/mL, optionally about 50 U/mL; IL-7 at a concentration of 0.5 ng/mL to 20 ng/mL, optionally about 5 ng/mL and/or IL-15 at a concentration of 0.1 ng/mL to 10 ng/mL, optionally about 0.5 ng/mL.

In some embodiments, the incubation independently is carried out for up to or approximately 24, 36, 48 hours, 3, 4, 5, 6, 7, 8, 9, 10, 11, 12, 13, 14, 15, 16, 17, 18, 19, 20 or 21 days, such as for 24-48 hours or 36-48 hours.

In some embodiments, the cells are contacted with the agent at a ratio of approximately 1 microgram per 100,000, 200,000, 300,000, 400,000, or 500,000 cells.

In some embodiments, the incubation is at a temperature of 30° C.±2° C. to 39° C.±2° C.; or the incubation is at a temperature that is at least or about at least 30° C.±2° C., 32° C.±2° C., 34° C.±2° C. or 37° C.±2° C. In some embodiments, at least a portion of the incubation is at 30° C.±2° C. and at least a portion of the incubation is at 37° C.±2° C. In some embodiments, the method further includes resting the cells between the introducing in (a) and the introducing in (b).

In some of any such embodiments provided herein, the Cas9 molecule is an enzymatically active Cas9. In some embodiments, the at least one gRNA includes a targeting domain including a sequence selected from the group consisting of GUCUGGGCGGUGCUACAACU (SEQ ID NO:508), GCCCUGGCCAGUCGUCU (SEQ ID NO: 514), CGUCUGGGCGGUGCUACAAC (SEQ ID NO:1533), UGUAGCACCGCCCAGACGAC (SEQ ID NO:579), CGACUGGCCAGGGCGCCUGU (SEQ ID NO:582) and CACCUACCUAAGAACCAUCC (SEQ ID NO:723). In some embodiments, 59-78, the at least one gRNA includes a targeting domain including the sequence CGACUGGCCAGGGCGCCUGU (SEQ ID NO:582).

In some embodiments, the Cas9 molecule is an S. aureus Cas9 molecule. In some embodiments, the Cas9 molecules is an S. pyogenes Cas9. In some embodiments, the Cas9 molecule lacks an active RuvC domain or an active HNH domain. In some embodiments, the Cas9 molecule is an S. pyogenes Cas9 molecule including a D10A mutation. In some embodiments, the Cas9 molecule is an S. pyogenes Cas9 molecule including an N863A mutation.

In some embodiments, the genetic disruption includes creation of a double strand break which is repaired by non-homologous end joining (NHEJ) to effect insertions and deletions (indels) in the PDCD1 gene.

In some embodiments, the recombinant receptor is a functional non-TCR antigen receptor or a transgenic TCR. In some embodiments, the recombinant receptor is a chimeric antigen receptor (CAR). In some embodiments, the CAR includes an antigen-binding domain that is an antibody or an antibody fragment. In some embodiments, the antibody fragment is a single chain fragment. In some embodiments, the antibody fragment includes antibody variable regions joined by a flexible immunoglobulin linker. In some embodiments, the fragment includes an scFv. In some embodiments, the antigen is associated with a disease or disorder, such as an infectious disease or condition, an autoimmune disease, an inflammatory disease or a tumor or a cancer. In some embodiments, the recombinant receptor specifically binds to a tumor antigen.

In some embodiments, the recombinant receptor binds is selected from ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acethycholine e receptor, GD2, GD3, HMW-MAA, IL-22R-alpha, IL-13R-alpha2, kdr, kappa light chain, Lewis Y, L1-cell adhesion molecule (CD171), MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1), cyclin A1 (CCNA1) or interleukin 12.

In some embodiments, the recombinant receptor includes an intracellular signaling domain including an ITAM. In some embodiments, the intracellular signaling domain includes an intracellular domain of a CD3-zeta (CD3) chain. In some embodiments, the recombinant receptor further includes a costimulatory signaling region, such as the costimulatory signaling region including a signaling domain of CD28 or 4-1BB.

In some embodiments, the nucleic acid encoding the recombinant receptor is a viral vector, such as a retroviral vector. In some embodiments, the viral vector is a lentiviral vector or a gammaretroviral vector. In some embodiments, introduction of the nucleic acid encoding the recombinant vector is by transduction, which optionally is retroviral transduction.

In some embodiments, the immune cell is a primary cell from a subject. In some embodiments, the immune cell is a human cell. In some embodiments, the immune cell is a white blood cell, such as an NK cell or T cell. In some embodiments, the immune cell is a T cell that is an unfractionated T cell, isolated CD8+ T cell, or isolated CD4+ T cell. In some embodiments, any of the method provided herein is performed on a plurality of immune cells.

In some embodiments, subsequent to introducing the agent and introducing the recombinant receptor, cells are not enriched or selected for (a) cells including the genetic disruption or not expressing the endogenous PD-1 polypeptide, (b) cells expressing the recombinant receptor or both (a) and (b). In some embodiments, any of the methods further include enriching or selecting for (a) cells including the genetic disruption or not expressing the endogenous PD-1 polypeptide, (b) cells expressing the recombinant receptor or for both (a) and (b). In some embodiments, any of the methods further include incubating the cells at or at about 37° C.±2° C. In some embodiments, the incubation is carried out for a time between at or about 1 hour and at or about 96 hours, between at or about 4 hours and at or about 72 hours, between at or about 8 hours and at or about 48 hours, between at or about 12 hours and at or about 36 hours, between at or about 6 hours and at or about 24 hours, between at or about 36 hours and at or about 96 hours, inclusive. In some embodiments, the incubation or a portion of the incubation is performed in the presence of a stimulating agent. In some embodiments, stimulating agent is an agent capable of inducing proliferation of T cells, CD4+ T cells and/or CD8+ T cells. In some embodiments, the stimulating agent is or includes an antibody specific for CD3 an antibody specific for CD28 and/or a cytokine.

In some embodiments, any of the methods provided herein further includes formulating cells produced by the method in a pharmaceutically acceptable buffer.

In some embodiments, any of the methods provided herein produce a population of cells in which: at least about 70%, at least about 75%, or at least about 80% of the cells both 1) include the genetic disruption; do not express the endogenous PD-1 polypeptide; do not include a contiguous PDCD1 gene, a PDCD1 gene, and/or a functional PDCD1 gene; and/or do not express a PD-1 polypeptide; and 2) express the recombinant receptor; or at least about 70%, at least about 75%, or at least about 80% of the cells that express the recombinant receptor include the genetic disruption, do not express the endogenous PD-1 polypeptide, or do not express a PD-1 polypeptide.

In some embodiments, any of the methods provided herein produce a population of cells in which: greater than 80%, 81%, 82%, 83%, 84%, 85%, 86%, 87%, 88%, 89%, 90%, 91%, 92%, 93%, 94%, or 95% of the cells both 1) include the genetic disruption; do not express the endogenous PD-1 polypeptide; do not include a contiguous PDCD1 gene, a PDCD1 gene, and/or a functional PDCD1 gene; and/or do not express a PD-1 polypeptide and 2) express the recombinant receptor; and/or greater than 80%, 81%, 82%, 83%, 84%, 85%, 86%, 87%, 88%, 89%, 90%, 91%, 92%, 93%, 94%, or 95% of the cells that express the recombinant receptor include the genetic disruption, do not express the endogenous PD-1 polypeptide, or do not express a PD-1 polypeptide.

In some embodiments of any of the methods provided herein, both alleles of the gene in the genome are disrupted.

In some embodiments, also provided are a genetically engineered immune cell produced by any of the methods provided herein.

In some embodiments, also provided are a plurality of genetically engineered immune cells produced by any of the methods provided herein.

In some embodiments, provided are such genetically engineered immune cells wherein: at least about 70%, at least about 75%, or at least about 80% of the cells both 1) include the genetic disruption; do not express the endogenous PD-1 polypeptide; do not include a contiguous PDCD1 gene, a PDCD1 gene, and/or a functional PDCD1 gene; and/or do not express a PD-1 polypeptide; and 2) express the recombinant receptor; or at least about 70%, at least about 75%, or at least about 80% of the cells that express the recombinant receptor include the genetic disruption, do not express the endogenous PD-1 polypeptide, or do not express a PD-1 polypeptide.

In some embodiments, provided are the plurality of genetically engineered immune cells wherein: greater than 80%, 81%, 82%, 83%, 84%, 85%, 86%, 87%, 88%, 89%, 90%, 91%, 92%, 93%, 94%, or 95% of the cells both 1) include the genetic disruption; do not express the endogenous PD-1 polypeptide; do not include a contiguous PDCD1 gene, a PDCD1 gene, and/or a functional PDCD1 gene; and/or do not express a PD-1 polypeptide and 2) express the recombinant receptor; and/or greater than 80%, 81%, 82%, 83%, 84%, 85%, 86%, 87%, 88%, 89%, 90%, 91%, 92%, 93%, 94%, or 95% of the cells that express the recombinant receptor include the genetic disruption, do not express the endogenous PD-1 polypeptide, or do not express a PD-1 polypeptide.

In some embodiments, also provided are compositions including any of the genetically engineered immune cells provided herein or any of the plurality of genetically engineered immune cells provided herein, and optionally a pharmaceutically acceptable buffer.

In some embodiments, also provided are methods of treatment, including administering any of the compositions provided herein to a subject having a disease or condition.

In some embodiments, the recombinant receptor specifically binds to an antigen associated with the disease or condition, such as a cancer, a tumor, an autoimmune disease or disorder, or an infectious disease.

In some embodiments, also provided are any of the pharmaceutical compositions provided herein for use in treating a disease or condition in a subject.

In some embodiments, in any of the pharmaceutical composition for use, the recombinant receptor specifically binds to an antigen associated with the disease or condition, such as a cancer, a tumor, an autoimmune disease or disorder, or an infectious disease.

Provided herein is a method of altering a T cell including contacting the T cell with one or more Cas9 molecule/gRNA molecule complexes, wherein the gRNA molecule(s) in the one or more Cas9 molecule/gRNA molecule complexes contain a targeting domain which is complementary with a target domain from the PDCD1 gene. In some embodiments, the T cell is from a subject suffering from cancer. In some examples, the cancer is selected from the group consisting of: lymphoma, chronic lymphocytic leukemia (CLL), B cell acute lymphocytic leukemia (B-ALL), acute lymphoblastic leukemia, acute myeloid leukemia, non-Hodgkin's lymphoma (NHL), diffuse large cell lymphoma (DLCL), multiple myeloma, renal cell carcinoma (RCC), neuroblastoma, colorectal cancer, breast cancer, ovarian cancer, melanoma, sarcoma, prostate cancer, lung cancer, esophageal cancer, hepatocellular carcinoma, pancreatic cancer, astrocytoma, mesothelioma, head and neck cancer, and medulloblastoma.

In some of any such embodiments, the T cell is from a subject having cancer or which could otherwise benefit from a mutation at a T cell target position of the PDCD1 gene. In some of any such embodiments, the contacting is performed ex vivo. In some of any such embodiments, the altered T cell is returned to the subject's body after the step of contacting. In some of any such embodiments, the T cell is from a subject suffering from cancer, the contacting is performed ex vivo and the altered T cell is returned to the subject's body after the step of contacting.

In some of any such embodiments, the one or more Cas9 molecule/gRNA molecule complexes are formed prior to the contacting. In some of any such embodiments, the gRNA molecule(s) contain a targeting domain that is the same as, or differs by no more than 3 nucleotides from, a targeting domain from any of SEQ ID NOS: 481-555, 563-1516, 1517-3748, 14657-16670, and 16671-21037. In some embodiments, the gRNA molecule(s) contain a targeting domain that is selected from SEQ ID NOS: 563-1516. In some cases, the gRNA molecule(s) contain a targeting domain that is selected from SEQ ID NOS: 1517-3748. In some instances, the gRNA molecule(s) contain a targeting domain that is selected from SEQ ID NOS: 14657-16670. In some aspects, the gRNA molecule(s) contain a targeting domain that is selected from SEQ ID NOS: 16671-21037.

In some embodiments, the gRNA molecule(s) contain a targeting domain that is selected from SEQ ID NOS: 481-500 and 508-547. In some cases, the gRNA molecule(s) contain a targeting domain that is selected from SEQ ID NOS: 501-507 and 548-555. In some embodiments, the gRNA molecule(s) contain a targeting domain that is selected from SEQ ID NOS: 508, 514, 576, 579, 582, and 723. In some cases, the gRNA molecule(s) contain a targeting domain that is selected from SEQ ID NOS: 508, 510, 511, 512, 514, 576, 579, 581, 582, 766, and 723.

In some of any such embodiments, the gRNA molecule(s) are modified at their 5′ end or contain a 3′ polyA tail. In some of any such embodiments, the gRNA molecule(s) are modified at their 5′ end and contain a 3′ polyA tail. In some instances, the gRNA molecule(s) lack a 5′ triphosphate group.

In some aspects, the gRNA molecule(s) include a 5′ cap. In some cases, the 5′ cap contains a modified guanine nucleotide that is linked to the remainder of the gRNA molecule via a 5′-5′ triphosphate linkage. In some instances, the 5′ cap contains two optionally modified guanine nucleotides that are linked via an optionally modified 5′-5′ triphosphate linkage.

In some of any such embodiments, the 3′ polyA tail includes about 10 to about 30 adenine nucleotides. In some of any such embodiments, the 3′ polyA tail includes about 20 adenine nucleotides. In some embodiments, the gRNA molecule(s) including the 3′ polyA tail were prepared by in vitro transcription from a DNA template.

In some embodiments, the 5′ nucleotide of the targeting domain is a guanine nucleotide, the DNA template includes a T7 promoter sequence located immediately upstream of the sequence that corresponds to the targeting domain, and the 3′ nucleotide of the T7 promoter sequence is not a guanine nucleotide. In some cases, the 5′ nucleotide of the targeting domain is not a guanine nucleotide, the DNA template includes a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide. In some of any such embodiments, the one or more Cas9 molecule/gRNA molecule complexes are delivered into the T cell via electroporation.

In some of any such embodiments, the gRNA molecule(s) contain a targeting domain which is complementary with a target domain from the PDCD1 gene and wherein the gRNA molecule(s) guide the Cas9 molecule to cleave the target domain with an efficiency of cleavage of at least 40%. In some instances, the efficiency of cleavage is determined using a labeled anti-PDCD1 antibody and a flow cytometry assay.

In some embodiments, the Cas9 molecule is guided by a single gRNA molecule and cleaves the target domain with a single double stranded break. In some examples, the Cas9 molecule is a S. pyogenes Cas9 molecule.

In some embodiments, the single gRNA molecule includes a targeting domain selected from the following targeting domains: GUCUGGGCGGUGCUACAACU (SEQ ID NO:508); GCCCUGGCCAGUCGUCU (SEQ ID NO:514); CGUCUGGGCGGUGCUACAAC (SEQ ID NO:576); UGUAGCACCGCCCAGACGAC (SEQ ID NO:579); CGACUGGCCAGGGCGCCUGU (SEQ ID NO:582); or CACCUACCUAAGAACCAUCC (SEQ ID NO:723).

In some embodiments, the Cas9 molecule is a nickase and two Cas9 molecule/gRNA molecule complexes are guided by two different gRNA molecules to cleave the target domain with two single stranded breaks on opposing strands of the target domain. In some cases, the Cas9 molecule is a S. pyogenes Cas9 molecule. In some instances, the S. pyogenes Cas9 molecule has a D10A mutation.

In some embodiments, the two gRNA molecules include targeting domains that are selected from the following pairs of targeting domains: CGACUGGCCAGGGCGCCUGU (SEQ ID NO:582) and GUCUGGGCGGUGCUACAACU (SEQ ID NO:508); CGACUGGCCAGGGCGCCUGU (SEQ ID NO:582) and GGGCGGUGCUACAACUGGGC (SEQ ID NO:510); CGACUGGCCAGGGCGCCUGU (SEQ ID NO:582) and GGCCAGGAUGGUUCUUAGGU (SEQ ID NO:511); CGACUGGCCAGGGCGCCUGU (SEQ ID NO:582) and GGAUGGUUCUUAGGUAGGUG (SEQ ID NO:512); CGACUGGCCAGGGCGCCUGU (SEQ ID NO:582) and CGUCUGGGCGGUGCUACAAC (SEQ ID NO:576); CGACUGGCCAGGGCGCCUGU (SEQ ID NO:582) and CUACAACUGGGCUGGCGGCC (SEQ ID NO:766); UGUAGCACCGCCCAGACGAC (SEQ ID NO:579) and GGCCAGGAUGGUUCUUAGGU (SEQ ID NO:511); UGUAGCACCGCCCAGACGAC (SEQ ID NO:579) and GGAUGGUUCUUAGGUAGGUG (SEQ ID NO:512); or ACCGCCCAGACGACUGGCCA (SEQ ID NO:581) and GGCCAGGAUGGUUCUUAGGU (SEQ ID NO:511). In some cases, the S. pyogenes Cas9 molecule has a N863A mutation.

In some embodiments, the two gRNA molecules include targeting domains that are selected from the following pairs of targeting domains: CGACUGGCCAGGGCGCCUGU (SEQ ID NO:582) and GUCUGGGCGGUGCUACAACU (SEQ ID NO:508); CGACUGGCCAGGGCGCCUGU (SEQ ID NO:582) and GGGCGGUGCUACAACUGGGC (SEQ I GGCCAGGAUGGUUCUUAGGU (SEQ ID NO:511).D NO:510); or CGACUGGCCAGGGCGCCUGU (SEQ ID NO:582) and

In some of any such embodiments, the gRNA molecule(s) are modular gRNA molecule(s). In some of any such embodiments, the gRNA molecule(s) are chimeric gRNA molecule(s).

In some embodiments, the gRNA molecule(s) includes from 5′ to 3′: a targeting domain; a first complementarity domain; a linking domain; a second complementarity domain; a proximal domain; and a tail domain. In some cases, the gRNA molecule(s) contain a linking domain of no more than 25 nucleotides in length and a proximal and tail domain, that taken together, are at least 20 nucleotides in length.

In some of any such embodiments, the gRNA molecule(s) guide the Cas9 molecule to cleave the target domain with an efficiency of cleavage of at least 60%. In some of any such embodiments, the gRNA molecule(s) guide the Cas9 molecule to cleave the target domain with an efficiency of cleavage of at least 80%. In some of any such embodiments, the gRNA molecule(s) guide the Cas9 molecule to cleave the target domain with an efficiency of cleavage of at least 90%.

In some of any such embodiments, the one or more Cas9 molecule/gRNA molecule complexes produce fewer than 5 off-targets. In some of any such embodiments, the one or more Cas9 molecule/gRNA molecule complexes produce fewer than 2 exonic off-targets. In some aspects, off-targets are identified by GUIDE-seq. In some instances, off-targets are identified by Amp-seq.

Provided herein is a Cas9 molecule/gRNA molecule complex, wherein the gRNA molecule contains a targeting domain which is complementary with a target domain from the PDCD1 gene, and the gRNA molecule is modified at its 5′ end and/or contains a 3′ polyA tail. In some embodiments, the gRNA molecule contains a targeting domain that is the same as, or differs by no more than 3 nucleotides from, a targeting domain from SEQ ID NOS: 481-555, 563-1516, 1517-3748, 14657-16670, and 16671-21037. In some aspects, the gRNA molecule contains a targeting domain that is selected from SEQ ID NOS: 563-1516. In some instances, the gRNA molecule contains a targeting domain that is selected from SEQ ID NOS: 1517-3748. In some cases, the gRNA molecule contains a targeting domain that is selected from SEQ ID NOS: 14657-16670. In some cases, the gRNA molecule contains a targeting domain that is selected from SEQ ID NOS: 16671-21037.

In some embodiments, the gRNA molecule contains a targeting domain that is selected from SEQ ID NOS: 481-500 and 508-547. In some instances, the gRNA molecule contains a targeting domain that is selected from SEQ ID NOS: 501-507 and 548-555. In some aspects, the gRNA molecule contains a targeting domain that is selected from SEQ ID NOS: 508, 514, 576, 579, 582, and 723. In some cases, the gRNA molecule contains a targeting domain that is selected from SEQ ID NOS: 508, 510, 511, 512, 514, 576, 579, 581, 582, 766, and 723.

In some of any such embodiments, the gRNA molecule is modified at its 5′ end. In some cases, the gRNA molecule lacks a 5′ triphosphate group. In some instances, the gRNA molecule includes a 5′ cap. In some embodiments, the 5′ cap contains a modified guanine nucleotide that is linked to the remainder of the gRNA molecule via a 5′-5′ triphosphate linkage. In some cases, the 5′ cap contains two optionally modified guanine nucleotides that are linked via an optionally modified 5′-5′ triphosphate linkage.

In some of any such embodiments, the 3′ polyA tail is includes about 10 to about 30 adenine nucleotides. In some of any such embodiments, the 3′ polyA tail includes about 20 adenine nucleotides. In some aspects, the gRNA molecule including the 3′ polyA tail was prepared by in vitro transcription from a DNA template. In some embodiments, the 5′ nucleotide of the targeting domain is a guanine nucleotide, the DNA template contains a T7 promoter sequence located immediately upstream of the sequence that corresponds to the targeting domain, and the 3′ nucleotide of the T7 promoter sequence is not a guanine nucleotide. In some instances, the 5′ nucleotide of the targeting domain is not a guanine nucleotide, the DNA template includes a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

In some of any such embodiments, the Cas9 molecule cleaves a target domain with a double stranded break. In some examples, the Cas9 molecule is a S. pyogenes Cas9 molecule. In some cases, the targeting domain is selected from the following group of targeting domains:

(SEQ ID NO: 508)

GUCUGGGCGGUGCUACAACU;

(SEQ ID NO: 514)

GCCCUGGCCAGUCGUCU;

(SEQ ID NO: 576)

CGUCUGGGCGGUGCUACAAC;

(SEQ ID NO: 579)

UGUAGCACCGCCCAGACGAC;

(SEQ ID NO: 582)

CGACUGGCCAGGGCGCCUGU;
or

(SEQ ID NO: 723)

CACCUACCUAAGAACCAUCC.

In some of any such embodiments, the Cas9 molecule cleaves a target domain with a single stranded break. In some cases, the Cas9 molecule is a S. pyogenes Cas9 molecule. In some examples, the S. pyogenes Cas9 molecule has a D10A mutation. In some cases, the targeting domain is selected from the following group of targeting domains: CGACUGGCCAGGGCGCCUGU (SEQ ID NO:582) and GUCUGGGCGGUGCUACAACU (SEQ ID NO:508); CGACUGGCCAGGGCGCCUGU (SEQ ID NO:582) and GGGCGGUGCUACAACUGGGC (SEQ ID NO:510); CGACUGGCCAGGGCGCCUGU (SEQ ID NO:582) and GGCCAGGAUGGUUCUUAGGU (SEQ ID NO:511); CGACUGGCCAGGGCGCCUGU (SEQ ID NO:582) and GGAUGGUUCUUAGGUAGGUG (SEQ ID NO:512); CGACUGGCCAGGGCGCCUGU (SEQ ID NO:582) and CGUCUGGGCGGUGCUACAAC (SEQ ID NO:576); CGACUGGCCAGGGCGCCUGU (SEQ ID NO:582) and CUACAACUGGGCUGGCGGCC (SEQ ID NO:766); UGUAGCACCGCCCAGACGAC (SEQ ID NO:579) and GGCCAGGAUGGUUCUUAGGU (SEQ ID NO:511); UGUAGCACCGCCCAGACGAC (SEQ ID NO:579) and GGAUGGUUCUUAGGUAGGUG (SEQ ID NO:512); or ACCGCCCAGACGACUGGCCA (SEQ ID NO:581) and GGCCAGGAUGGUUCUUAGGU (SEQ ID NO:511). In some instances, the S. pyogenes Cas9 molecule has a N863A mutation.

In some embodiments, the targeting domain is selected from the following group of targeting domains: CGACUGGCCAGGGCGCCUGU (SEQ ID NO:582) and GUCUGGGCGGUGCUACAACU (SEQ ID NO:508); CGACUGGCCAGGGCGCCUGU (SEQ ID NO:582) and GGGCGGUGCUACAACUGGGC (SEQ ID NO:510); or CGACUGGCCAGGGCGCCUGU (SEQ ID NO:582) and GGCCAGGAUGGUUCUUAGGU (SEQ ID NO:511).

In some of any such embodiments, the gRNA molecule is a modular gRNA molecule. In some of any such embodiments, the gRNA molecule is a chimeric gRNA molecule.

In some embodiments, the gRNA molecule includes from 5′ to 3′: a targeting domain; a first complementarity domain; a linking domain; a second complementarity domain; a proximal domain; and a tail domain. In some aspects, the gRNA molecule contains a linking domain of no more than 25 nucleotides in length and a proximal and tail domain, that taken together, are at least 20 nucleotides in length.

Provided herein is a gRNA molecule that contains a targeting domain which is complementary with a target domain from the PDCD1 gene, wherein the gRNA molecule is modified at its 5′ end and/or contains a 3′ polyA tail. In some embodiments, the gRNA molecule contains a targeting domain that is the same as, or differs by no more than 3 nucleotides from, a targeting domain from any of SEQ ID NOS: 481-555, 563-1516, 1517-3748, 14657-16670, and 16671-21037. In some cases, the gRNA molecule contains a targeting domain that is selected from SEQ ID NOS: 563-1516. In some cases, the gRNA molecule contains a targeting domain that is selected from SEQ ID NOS: 1517-3748. In some examples, the gRNA molecule contains a targeting domain that is selected from SEQ ID NOS: 14657-16670. In some aspects, the gRNA molecule contains a targeting domain that is selected from SEQ ID NOS: 16671-21037.

In some embodiments, the gRNA molecule contains a targeting domain that is selected from SEQ ID NOS: 481-500 and 508-547. In some cases, the gRNA molecule contains a targeting domain that is selected from SEQ ID NOS: 501-507 and 548-555. In some instances, the gRNA molecule contains a targeting domain that is selected from SEQ ID NOS: 508, 514, 576, 579, 582, and 723. In some embodiments, the gRNA molecule contains a targeting domain that is selected from SEQ ID NOS: 508, 510, 511, 512, 514, 576, 579, 581, 582, 766, and 723.

In some of any such embodiments, the gRNA molecule is modified at its 5′ end. In some cases, the gRNA molecule lacks a 5′ triphosphate group. In some aspects, the gRNA molecule includes a 5′ cap. In some examples, the 5′ cap contains a modified guanine nucleotide that is linked to the remainder of the gRNA molecule via a 5′-5′ triphosphate linkage. In some embodiments, the 5′ cap contains two optionally modified guanine nucleotides that are linked via an optionally modified 5′-5′ triphosphate linkage.

In some of any such embodiments, the gRNA molecule includes a 3′ polyA tail containing about 10 to about 30 adenine nucleotides. In some of any such embodiments, the gRNA molecule contains a 3′ polyA tail which contains about 20 adenine nucleotides.

In some embodiments, the gRNA molecule including the 3′ polyA tail was prepared by in vitro transcription from a DNA template. In some instances, the 5′ nucleotide of the targeting domain is a guanine nucleotide, the DNA template contains a T7 promoter sequence located immediately upstream of the sequence that corresponds to the targeting domain, and the 3′ nucleotide of the T7 promoter sequence is not a guanine nucleotide. In some cases, the 5′ nucleotide of the targeting domain is not a guanine nucleotide, the DNA template includes a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

In some of any such embodiments, the gRNA molecule is a S. pyogenes gRNA molecule. In some embodiments, the targeting domain is selected from the following group of targeting domains: GUCUGGGCGGUGCUACAACU (SEQ ID NO:508); GCCCUGGCCAGUCGUCU (SEQ ID NO:514); CGUCUGGGCGGUGCUACAAC (SEQ ID NO:576); UGUAGCACCGCCCAGACGAC (SEQ ID NO:579); CGACUGGCCAGGGCGCCUGU (SEQ ID NO:582); or CACCUACCUAAGAACCAUCC (SEQ ID NO:723). In some cases, the targeting domain is selected from the following group of targeting domains: GCCCUGGCCAGUCGUCU (SEQ ID NO:514); or CACCUACCUAAGAACCAUCC (SEQ ID NO:723). In some instances, the targeting domain is selected from the following group of targeting domains: GGGCGGUGCUACAACUGGGC (SEQ ID NO:510); GGCCAGGAUGGUUCUUAGGU (SEQ ID NO:511); GGAUGGUUCUUAGGUAGGUG (SEQ ID NO:512); ACCGCCCAGACGACUGGCCA (SEQ ID NO:581) and CUACAACUGGGCUGGCGGCC (SEQ ID NO:766). In some instances, the targeting domain is selected from the following group of targeting domains:

(SEQ ID NO: 511)

GGCCAGGAUGGUUCUUAGGU;

(SEQ ID NO: 512)

GGAUGGUUCUUAGGUAGGUG;

(SEQ ID NO: 766)

CUACAACUGGGCUGGCGGCC.

In some of any such embodiments, the gRNA molecule is a modular gRNA molecule. In some of any such embodiments, the gRNA molecule is a chimeric gRNA molecule. In some embodiments, the gRNA molecule contains from 5′ to 3′: a targeting domain; a first complementarity domain; a linking domain; a second complementarity domain; a proximal domain; and a tail domain. In some embodiments, the gRNA molecule contains a linking domain of no more than 25 nucleotides in length and a proximal and tail domain, that taken together, are at least 20 nucleotides in length.

Provided herein is a method of making a cell for implantation, including contacting the cell with one or more Cas9 molecule/gRNA molecule complexes, wherein the gRNA molecule(s) in the one or more Cas9 molecule/gRNA molecule complexes contain a targeting domain which is complementary with a target domain from the PDCD1 gene. In some cases, the gRNA molecule(s) contain a targeting domain which is complementary with a target domain from the PDCD1 gene and wherein the gRNA molecule(s) guide the Cas9 molecule to cleave the target domain with an efficiency of cleavage of at least 40%. In some aspects, the efficiency of cleavage is determined using a labeled anti-PDCD1 antibody and a flow cytometry assay.

In some of any such embodiments, the gRNA molecule(s) are modified at their 5′ end or include a 3′ polyA tail. In some of any such embodiments, the gRNA molecule(s) are modified at their 5′ end and include a 3′ polyA tail. In some embodiments, the gRNA molecule(s) lack a 5′ triphosphate group. In some examples, the gRNA molecule(s) include a 5′ cap. In some cases, the 5′ cap contains a modified guanine nucleotide that is linked to the remainder of the gRNA molecule via a 5′-5′ triphosphate linkage. In some embodiments, the 5′ cap contains two optionally modified guanine nucleotides that are linked via an optionally modified 5′-5′ triphosphate linkage.

In some of any such embodiments, the 3′ polyA tail contains about 10 to about 30 adenine nucleotides. In some of any such embodiments, the 3′ polyA tail contains about 20 adenine nucleotides. In some cases, the gRNA molecule(s) including the 3′ polyA tail were prepared by in vitro transcription from a DNA template. In some embodiments, the 5′ nucleotide of the targeting domain is a guanine nucleotide, the DNA template includes a T7 promoter sequence located immediately upstream of the sequence that corresponds to the targeting domain, and the 3′ nucleotide of the T7 promoter sequence is not a guanine nucleotide. In some cases, the 5′ nucleotide of the targeting domain is not a guanine nucleotide, the DNA template includes a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

In some of any such embodiments, the one or more Cas9 molecule/gRNA molecule complexes are delivered into the cell via electroporation. In some of any such embodiments, the Cas9 molecule is guided by a single gRNA molecule and cleaves the target domain with a single double stranded break. In some embodiments, the Cas9 molecule is a S. pyogenes Cas9 molecule.

In some embodiments, the single gRNA molecule contains a targeting domain selected from the following targeting domains: GUCUGGGCGGUGCUACAACU (SEQ ID NO:508); GCCCUGGCCAGUCGUCU (SEQ ID NO:514); CGUCUGGGCGGUGCUACAAC (SEQ ID NO:576); UGUAGCACCGCCCAGACGAC (SEQ ID NO:579); CGACUGGCCAGGGCGCCUGU (SEQ ID NO:582); or CACCUACCUAAGAACCAUCC (SEQ ID NO:723).

In some of any such embodiments, the Cas9 molecule is a nickase and two Cas9 molecule/gRNA molecule complexes are guided by two different gRNA molecules to cleave the target domain with two single stranded breaks on opposing strands of the target domain.

In some embodiments, the Cas9 molecule is a S. pyogenes Cas9 molecule having a D10A mutation. In some examples, the two gRNA molecules include targeting domains that are selected from the following pairs of targeting domains: CGACUGGCCAGGGCGCCUGU (SEQ ID NO:582) and GUCUGGGCGGUGCUACAACU (SEQ ID NO:508); CGACUGGCCAGGGCGCCUGU (SEQ ID NO:582) and GGGCGGUGCUACAACUGGGC (SEQ ID NO:510); CGACUGGCCAGGGCGCCUGU (SEQ ID NO:582) and GGCCAGGAUGGUUCUUAGGU (SEQ ID NO:511); CGACUGGCCAGGGCGCCUGU (SEQ ID NO:582) and GGAUGGUUCUUAGGUAGGUG (SEQ ID NO:512); CGACUGGCCAGGGCGCCUGU (SEQ ID NO:582) and CGUCUGGGCGGUGCUACAAC (SEQ ID NO:576); CGACUGGCCAGGGCGCCUGU (SEQ ID NO:582) and CUACAACUGGGCUGGCGGCC (SEQ ID NO:766); UGUAGCACCGCCCAGACGAC (SEQ ID NO:579) and GGCCAGGAUGGUUCUUAGGU (SEQ ID NO:511); UGUAGCACCGCCCAGACGAC (SEQ ID NO:579) and GGAUGGUUCUUAGGUAGGUG (SEQ ID NO:512); or ACCGCCCAGACGACUGGCCA (SEQ ID NO:581) and GGCCAGGAUGGUUCUUAGGU (SEQ ID NO:511).

In some instances, the S. pyogenes Cas9 molecule has a N863A mutation. In some embodiments, the two gRNA molecules include targeting domains that are selected from the following pairs of targeting domains: CGACUGGCCAGGGCGCCUGU (SEQ ID NO:582) and GUCUGGGCGGUGCUACAACU (SEQ ID NO:508); CGACUGGCCAGGGCGCCUGU (SEQ ID NO:582) and GGGCGGUGCUACAACUGGGC (SEQ ID NO:510); or CGACUGGCCAGGGCGCCUGU (SEQ ID NO:582) and GGCCAGGAUGGUUCUUAGGU (SEQ ID NO:511).

In some of any such embodiments, the gRNA molecule(s) are modular gRNA molecule(s). In some of any such embodiments, the gRNA molecule(s) are chimeric gRNA molecule(s). In some examples, the gRNA molecule(s) contains from 5′ to 3′: a targeting domain; a first complementarity domain; a linking domain; a second complementarity domain; a proximal domain; and a tail domain. In some instances, the gRNA molecule(s) contain a linking domain of no more than 25 nucleotides in length and a proximal and tail domain, that taken together, are at least 20 nucleotides in length.

In some of any such embodiments, the gRNA molecule(s) guide the Cas9 molecule to cleave the target domain with an efficiency of cleavage of at least 60%. In some of any such embodiments, the gRNA molecule(s) guide the Cas9 molecule to cleave the target domain with an efficiency of cleavage of at least 80%. In some of any such embodiments, the gRNA molecule(s) guide the Cas9 molecule to cleave the target domain with an efficiency of cleavage of at least 90%.

In some of any such embodiments, the one or more Cas9 molecule/gRNA molecule complexes produce fewer than 5 off-targets. In some of any such embodiments, the one or more Cas9 molecule/gRNA molecule complexes produce fewer than 2 exonic off-targets. In some aspects, off-targets are identified by GUIDE-seq. In some examples, off-targets are identified by Amp-seq.

### BRIEF DESCRIPTION OF THE DRAWINGS

The drawings are first briefly described.

FIG. 1A-1G are representations of several exemplary gRNAs.

FIG. 1A depicts a modular gRNA molecule derived in part (or modeled on a sequence in part) from Streptococcus pyogenes (S. pyogenes) as a duplexed structure (SEQ ID NO:42 and 43, respectively, in order of appearance);

FIG. 1B depicts a unimolecular (or chimeric) gRNA molecule derived in part from S. pyogenes as a duplexed structure (SEQ ID NO:44);

FIG. 1C depicts a unimolecular gRNA molecule derived in part from S. pyogenes as a duplexed structure (SEQ ID NO:45);

FIG. 1D depicts a unimolecular gRNA molecule derived in part from S. pyogenes as a duplexed structure (SEQ ID NO:46);

FIG. 1E depicts a unimolecular gRNA molecule derived in part from S. pyogenes as a duplexed structure (SEQ ID NO:47);

FIG. 1F depicts a modular gRNA molecule derived in part from Streptococcus thermophilus (S. thermophilus) as a duplexed structure (SEQ ID NO:48 and 49, respectively, in order of appearance);

FIG. 1G depicts an alignment of modular gRNA molecules of S. pyogenes and S. thermophilus (SEQ ID NO:50-53, respectively, in order of appearance).

FIG. 2A-2G depict an alignment of Cas9 sequences from Chylinski et al. (RNA Biol. 2013; 10(5): 726-737). The N-terminal RuvC-like domain is boxed and indicated with a “y”. The other two RuvC-like domains are boxed and indicated with a “b”. The HNH-like domain is boxed and indicated by a “g”. Sm: S. nutans (SEQ ID NO:1); Sp: S. pyogenes (SEQ ID NO:2); St: S. thermophilus (SEQ ID NO:3); Li: L. innocua (SEQ ID NO:4). Motif: this is a motif based on the four sequences: residues conserved in all four sequences are indicated by single letter amino acid abbreviation; “*” indicates any amino acid found in the corresponding position of any of the four sequences; and “-” indicates any amino acid, e.g., any of the 20 naturally occurring amino acids.

FIG. 3A-3B show an alignment of the N-terminal RuvC-like domain from the Cas9 molecules disclosed in Chylinski et al (SEQ ID NO:54-103, respectively, in order of appearance). The last line of FIG. 3B identifies 4 highly conserved residues.

FIG. 4A-4B show an alignment of the N-terminal RuvC-like domain from the Cas9 molecules disclosed in Chylinski et al. with sequence outliers removed (SEQ ID NO:104-177, respectively, in order of appearance). The last line of FIG. 4B identifies 3 highly conserved residues.

FIG. 5A-5C show an alignment of the HNH-like domain from the Cas9 molecules disclosed in Chylinski et al (SEQ ID NO:178-252, respectively, in order of appearance). The last line of FIG. 5C identifies conserved residues.

FIG. 6A-6B show an alignment of the HNH-like domain from the Cas9 molecules disclosed in Chylinski et al. with sequence outliers removed (SEQ ID NO:253-302, respectively, in order of appearance). The last line of FIG. 6B identifies 3 highly conserved residues.

FIG. 7A-7B depict an alignment of Cas9 sequences from S. pyogenes and Neisseria meningitidis (N. meningitidis). The N-terminal RuvC-like domain is boxed and indicated with a “Y”. The other two RuvC-like domains are boxed and indicated with a “B”. The HNH-like domain is boxed and indicated with a “G”. Sp: S. pyogenes; Nm: N. meningitidis. Motif: this is a motif based on the two sequences: residues conserved in both sequences are indicated by a single amino acid designation; “*” indicates any amino acid found in the corresponding position of any of the two sequences; “-” indicates any amino acid, e.g., any of the 20 naturally occurring amino acids, and “-” indicates any amino acid, e.g., any of the 20 naturally occurring amino acids, or absent.

FIG. 8 shows a nucleic acid sequence encoding Cas9 of N. meningitidis (SEQ ID NO:303). Sequence indicated by an “R” is an SV40 NLS; sequence indicated as “G” is an HA tag; and sequence indicated by an “O” is a synthetic NLS sequence; the remaining (unmarked) sequence is the open reading frame (ORF).

FIG. 9A shows schematic representations of the domain organization of S. pyogenes Cas9 and the organization of the Cas9 domains, including amino acid positions, in reference to the two lobes of Cas9 (recognition (REC) and nuclease (NUC) lobes).

FIG. 9B shows schematic representations of the domain organization of S. pyogenes Cas9 and the percent homology of each domain across 83 Cas9 orthologs.

FIG. 10A shows an exemplary structure of a unimolecular gRNA molecule derived in part from S. pyogenes as a duplexed structure (SEQ ID NO:40).

FIG. 10B shows an exemplary structure of a unimolecular gRNA molecule derived in part from S. aureus as a duplexed structure (SEQ ID NO:41).

FIG. 11 shows results from an experiment assessing the activity of gRNAs directed against TRBC gene in 293 cells using S. aureus Cas9. 293s were transfected with two plasmids—one encoding S. aureus Cas9 and the other encoding the listed gRNA. The graph summarizes the average % NHEJ observed at the TRBC2 locus for each gRNA, which was calculated from a T7E1 assay performed on genomic DNA isolated from duplicate samples.

FIG. 12 shows results from an experiment assessing the activity of gRNAs directed against TRBC gene in 293 cells using S. pyrogenes Cas9. 293 cells were transfected with two plasmids—one encoding S. pyogenes Cas9 and the other encoding the listed gRNA. The graph shows the average % NHEJ observed at both the TRBC1 and TRBC2 loci for each gRNA, which was calculated from a T7E1 assay performed on genomic DNA isolated from duplicate samples.

FIG. 13 shows results from an experiment assessing the activity of gRNAs directed against TRAC gene in 293 cells using S. aureus Cas9. 293 cells were transfected with two plasmids—one encoding S. aureus Cas9 and the other encoding the listed gRNA. The graph shows the average % NHEJ observed at the TRAC locus for each gRNA, which was calculated from a T7E1 assay performed on genomic DNA isolated from duplicate samples.

FIG. 14 shows results from an experiment assessing the activity of gRNAs directed against TRAC gene in 293 cells using S. pyogenes Cas9. 293 cells were transfected with two plasmids—one encoding S. pyogenes Cas9 and the other encoding the listed gRNA. The graph shows the average % NHEJ observed at the TRAC locus for each gRNA, which was calculated from a T7E1 assay performed on genomic DNA isolated from duplicate samples.

FIG. 15 shows results from an experiment assessing the activity of gRNAs directed against PDCD1 gene in 293 cells using S. aureus Cas9. 293 cells were transfected with two plasmids—one encoding S. aureus Cas9 and the other encoding the listed gRNA. The graph shows the average % NHEJ observed at the PDCD1 locus for each gRNA, which was calculated from a T7E1 assay performed on genomic DNA isolated from duplicate samples.

FIG. 16 shows results from an experiment assessing the activity of gRNAs directed against PDCD1 gene in 293 cells using S. pyogenes Cas9. 293 cells were transfected with two plasmids—one encoding S. pyogenes Cas9 and the other encoding the listed gRNA. The graph shows the average % NHEJ observed at the PDCD1 locus for each gRNA, which was calculated from a T7E1 assay performed on genomic DNA isolated from duplicate samples.

FIG. 17A-C depict results showing a loss of CD3 expression in CD4+ T cells due to delivery of S. pyogenes Cas9 mRNA and TRBC and TRAC gene specific gRNAs

FIG. 17A shows CD4+ T cells electroporated with S. pyogenes Cas9 mRNA and the gRNA indicated (TRBC-210 (GCGCUGACGAUCUGGGUGAC) (SEQ ID NO:413), TRAC-4 (GCUGGUACACGGCAGGGUCA) (SEQ ID NO:453) or AAVS1 (GUCCCCUCCACCCCACAGUG) (SEQ ID NO:51201)) and stained with an APC-CD3 antibody and analyzed by FACS. The cells were analyzed on day 2 and day 3 after the electroporation.

FIG. 17B shows quantification of the CD3 negative population from the plots in (A).

FIG. 17C shows % NHEJ results from the T7E1 assay performed on TRBC2 and TRAC loci.

FIG. 18A-C depict results showing a loss of CD3 expression in Jurkat T cells due to delivery of S. aureus Cas9/gRNA RNP targeting TRAC gene

FIG. 18A shows Jurkat T cells electroporated with S. aureus Cas9/gRNA TRAC-233 (GUGAAUAGGCAGACAGACUUGUCA) (SEQ ID NO:474) RNPs targeting TRAC gene and stained with an APC-CD3 antibody and analyzed by FACS. The cells were analyzed on day 1, day 2 and day 3 after the electroporation.

FIG. 18B shows quantification of the CD3 negative population from the plots in (A).

FIG. 18C shows % NHEJ results from the T7E1 assay performed on the TRAC locus.

FIG. 19 shows the structure of the 5′ ARCA cap.

FIG. 20 depicts results from the quantification of live Jurkat T cells post electroporation with Cas9 mRNA and AAVS1 gRNAs. Jurkat T cells were electroporated with S. pyogenes Cas9 mRNA and the respective modified gRNA. 24 hours after electroporation, 1×10⁵ cells were stained with FITC-conjugated Annexin-V specific antibody for 15 minutes at room temperature followed by staining with propidium iodide immediately before analysis by flow cytometry. The percentage of cells that did not stain for either Annexin-V or PI is presented in the bar graph.

FIG. 21A-C depict loss of CD3 expression in Naive CD3+ T cells due to delivery of S. aureus Cas9/gRNA RNP targeting TRAC.

FIG. 21A depicts naïve CD3+ T cells electroporated with S. aureus Cas9/gRNA (with targeting domain GUGAAUAGGCAGACAGACUUGUCA (SEQ ID NO:474) RNPs targeting TRAC were stained with an APC-CD3 antibody and analyzed by FACS. The cells were analyzed on day 4 after the electroporation. The negative control are cells with the gRNA with the targeting domain GUGAAUAGGCAGACAGACUUGUCA (SEQ ID NO:474) without a functional Cas9.

FIG. 21B depicts quantification of the CD3 negative population from the plots in FIG. 21A.

FIG. 21C depicts % NHEJ results from the T7E1 assay performed on the TRAC locus.

FIG. 22 depicts genomic editing at the PDCD1 locus in Jurkat T cells after delivery of S. pyogenes Cas9 mRNA and PDCD1 gRNA (with a targeting domain GUCUGGGCGGUGCUACAACU (SEQ ID NO:508)) or S. pyogenes Cas9/gRNA (with a targeting domain GUCUGGGCGGUGCUACAACU (SEQ ID NO:508)) RNP targeting PDCD1. Quantification of % NHEJ results from the T7E1 assay performed on the PDCD1 locus at 24, 48, and 72 hours. Higher levels of % NHEJ were detected with RNP vs mRNA delivery using the exemplary target gRNA (SEQ ID NO:508) claimed).

FIG. 23 depicts percentage of cells negative for PD-1 surface expression following electroporation of primary T cells with Cas9/gRNA RNP comprising different labeled gRNAs targeting the PDCD1 locus.

FIG. 24A depicts genomic editing at the PDCD1 locus in activated primary T cells after delivery of an S. pyogenes Cas9/gRNA RNP targeting PDCD1. Primary CD4 T cells isolated from multiple healthy donors were treated with the same RNP and PDCD1 expression was assessed by flow cytometry after reactivation. The average of the percentage of PDCD1 negative cells from multiple experiments is plotted and the standard deviation is depicted by the error bars.

FIG. 24B depicts surface expression of CD4 and PD-1 in primary CD4+ T cells following electroporation with Cas9/gRNA RNP comprising different labeled gRNAs targeting the PDCD1 locus or control AAVS1 locus.

FIG. 25 depicts surface expression of CD45RA and CD62L in primary CD8+ T cells following electroporation with Cas9/gRNA RNP comprising different labeled gRNAs targeting the PDCD1 locus or control AAVS1 locus.

FIG. 126 depicts surface expression of PD-1 and a surrogate marker (EGFRt) for anti-CD19 chimeric antigen receptor (CAR) expression on CD8+ or CD4+ T cells transduced with anti-CD19 CAR or mock transduction control (mock), following electroporation with Cas9/gRNA RNP targeting PDCD1 locus (PD-1KO), Cas9/gRNA RNP targeting AAVS1 control (AAVS1-KO) or untreated control.

FIGS. 27A and 27B show mean fluorescence intensity (MFI) of T cell surface marker expression of CD8+(FIG. 27A) or CD4+(FIG. 27B) T cells transduced with anti-CD19 CAR (CAR) or mock transduction control (mock) following electroporation with Cas9/gRNA RNP targeting PDCD1 locus (PD-1KO) or Cas9/gRNA RNP targeting AAVS1 control (AAVS1-KO). The MFI of surface markers CD45RA, CD69, 41BB, CCR7, CD27, CD25, CD62L, TIM3 are and CD45RO are depicted.

FIG. 28A depicts the percentage of cells containing an indel at the PDCD1 locus in T cells transduced with anti-CD19 CAR (CAR+) or mock transduction control (mock) following electroporation with Cas9/gRNA RNP targeting PDCD1 locus (PD-1KO) or Cas9/gRNA RNP targeting AAVS1 control (AAVS1-KO). FIG. 28B depicts the relative number of reads from MiSeq sequencing analysis that contained a deletion or an insertion at each position relative to the PDCD1 gRNA used. The position of the guide RNA is depicted as a thick vertical line around position 60 on the x-axis.

FIG. 29 shows T cell proliferation of primary CD8+ and CD4+ T cells that were transduced with anti-CD19 CAR (CAR+) or mock transduction control (mock) and electroporated with Cas9/gRNA RNP targeting PDCD1 locus or Cas9/gRNA RNP targeting AAVS1 control. T cell proliferation was assessed after co-culture with CD19-expressing cells or ROR-1-expressing control cells as measured using CellTrace™ Violet.

FIG. 30A-C depicts cytokine secretion in cell supernatants of primary T-cells transduced with anti-CD19 CAR (CAR+) or mock transduction control (mock) and electroporated with Cas9/gRNA RNP targeting PDCD1 locus or Cas9/gRNA RNP targeting AAVS1 control following co-culture with CD19-expressing cells or ROR-1-expressing control cells. FIG. 30A depicts IFN-γ in cell supernatants. FIG. 30B depicts interleukin-2 (IL-2) secretion in cell supernatants. FIG. 30C depicts tumor necrosis factor alpha (TNF-α) secretion in cell supernatants.

FIG. 31 depicts activated CD4 T cells treated with pairs of either S. pyogenes D10A or N863A nickase RNPs. After restimulation with PMA/IO, the expression of PDCD1 was assessed by flow cytometry using a PE-conjugated anti-PDCD1 antibody. The percentage of PDCD1 negative cells is graphed with the error bars referring to the standard deviation of duplicate samples. Samples 25 and 26 are D10A and N863A with a single gRNA which served as negative controls while sample 27 is wild type Cas9 with a single gRNA which served as a positive control.

### DETAILED DESCRIPTION

### I. Targeting Pd-1 Knockout in Cells Expressing a Recombinant Receptor

Provided are cells and cell compositions, including immune cells such as T cells and NK cells, that express a recombinant receptor, such as a transgenic or engineered T cell receptor (TCR) and/or a chimeric antigen receptor (CAR). The cells generally are engineered by introducing one or more nucleic acid molecules encoding such recombinant receptors or product thereof. Among such recombinant receptors are genetically engineered antigen receptors, including engineered TCRs and functional non-TCR antigen receptors, such as chimeric antigen receptors (CARs), including activating, stimulatory, and costimulatory CARs, and combinations thereof. The provided cells also have a genetic disruption of a PDCD1 gene encoding a programmed death-1(PD-1) polypeptide. Also provided are methods of producing such genetically engineered cells. In some embodiments, the cells and compositions can be used in adoptive cell therapy, e.g. adoptive immunotherapy.

In some embodiments, the provided cells, compositions and methods alter or reduce the effects of T cell inhibitory pathways or signals involving the inhibitory interactions between programmed death-1 (PD-1) and its ligand PD-L1. In some embodiments, the upregulation and/or expression of either one or both of a costimulatory inhibitory receptor or its ligand can negatively control T cell activation and T cell function. PD-1 (an exemplary amino acid and encoding nucleic acid sequence set forth in SEQ ID NO:51207 and 51208, respectively) is an immune inhibitory receptor that belongs to the B7:CD28 costimulatory molecular family and reacts with its ligands PD-L1 and PD-L2 to inhibit T cell function. PD-L1 (an exemplary amino acid and encoding nucleic acid sequence set forth in SEQ ID NO: 51209 and 51210, respectively; see also GenBank Acc. No. AF233516) is primarily reported to be expressed on antigen presenting cells or cancer cells where it interacts with T-cell expressed PD-1 to inhibit the activation of the T cell. In some cases, PD-L1 also has been reported to be expressed on T cells. In some cases, interaction of PD-1 and PD-L1 suppresses activity of cytotoxic T cells and, in some aspects, can inhibit tumor immunity to provide an immune escape for tumor cells. In some embodiments, expression of PD-1 and PD-L1 on T cells and/or in the tumor microenvironment can reduce the potency and efficacy of adoptive T cell therapy.

Thus, in some embodiments, such inhibitory pathways may otherwise impair certain desirable effector functions in the context of adoptive cell therapy. Tumor cells and/or cells in the tumor microenvironment often upregulate ligands for PD-1 (such as PD-L1 and PD-L2), which in turn leads to ligation of PD-1 on tumor-specific T cells expressing PD-1, delivering an inhibitory signal. PD-1 also often is upregulated on T cells in the tumor microenvironment, e.g., on tumor-infiltrating T cells, which can occur following signaling through the antigen receptor or certain other activating signals.

In some cases, such events may contribute to genetically engineered (e.g., CAR+) T cells acquiring an exhausted phenotype, such as when present in proximity with other cells that express PD-L1, which in turn can lead to reduced functionality. Exhaustion of T cells may lead to a progressive loss of T cell functions and/or in depletion of the cells (Yi et al. (2010) Immunology, 129:474-481). T cell exhaustion and/or the lack of T cell persistence is a barrier to the efficacy and therapeutic outcomes of adoptive cell therapy; clinical trials have revealed a correlation between greater and/or longer degree of exposure to the antigen receptor (e.g. CAR)-expressing cells and treatment outcomes.

Certain methods have been aimed at blocking PD-1 signaling or disrupting PD-1 expression in T cells, including in the context of cancer therapy. Such blockade or disruption may be through the administration of blocking antibodies, small molecules, or inhibitory peptides, or through the knockout or reduction of expression of PD-1 in T cells, e.g., in adoptively transferred T cells. The disruption of PD-1 in transferred T cells, however, may not be entirely satisfactory. In some cases, the disruption of the gene encoding PD-1 may not be permanent such that elimination of PD-1 expression on the surface of the cell may be only temporary. In other aspects, the efficiency of genetic disruption in cells is not sufficiently high such that a relatively high number of cells targeted for disruption retain expression of a targeted gene. In some cases, certain disruption methods, such as using CRISPR/Cas9 can lead to off-target effects due to limited cleavage specificity that may lead to non-specific disruption of a non-target gene or genes. In some cases, such problems can limit the efficacy of engineered cells into which disruption of a gene (e.g. PD-1) is desired.

In some embodiments, the provided cells, compositions and methods result in the reduction, deletion, elimination, knockout or disruption in expression of PDCD1 in immune cells (e.g. T cells). In some aspects, the disruption is carried out by gene editing, such as using an RNA-guided nuclease such as a clustered regularly interspersed short palindromic nucleic acid (CRISPR)-Cas system, such as CRISPR-Cas9 system, specific for the PD-1 gene (PDCD1) being disrupted. In some embodiments, an agent containing a Cas9 and a guide RNA (gRNA) containing a targeting domain, which targets a region of the PDCD1 locus, is introduced into the cell. In some embodiments, the agent is or comprises a ribonucleoprotein (RNP) complex of Cas9 and gRNA containing the PDCD1-targeted targeting domain (Cas9/gRNA RNP). In some embodiment, the introduction includes contacting the agent or portion thereof with the cells, in vitro, which can include cultivating or incubating the cell and agent for up to 24, 36 or 48 hours or 3, 4, 5, 6, 7, or 8 days. In some embodiments, the introduction further can include effecting delivery of the agent into the cells. In various embodiments, the methods, compositions and cells according to the present disclosure utilize direct delivery of ribonucleoprotein (RNP) complexes of Cas9 and gRNA to cells, for example by electroporation. In some embodiments, the RNP complexes include a gRNA that has been modified to include a 3′ poly-A tail and a 5′ Anti-Reverse Cap Analog (ARCA) cap. In some cases, electroporation of the cells to be modified includes cold-shocking the cells, e.g. at 32° C. following electroporation of the cells and prior to plating.

In some embodiments, prior to, during or subsequent to contacting the agent with the cells and/or prior to, during or subsequent to effecting delivery (e.g. electroporation), the provided methods include incubating the cells in the presence of a cytokine, a stimulating agent and/or an agent that is capable of inducing proliferation of the immune cells (e.g. T cells). In some embodiments, at least a portion of the incubation is in the presence of a stimulating agent that is or comprises an antibody specific for CD3 an antibody specific for CD28 and/or a cytokine. In some embodiments, at least a portion of the incubation is in the presence of a cytokine, such as one or more of IL-2, IL-7 and IL-15. In some embodiments, the incubation is for up to 8 days hours before or after the electroporation, such as up to 24 hours, 36 hours or 48 hours or 3, 4, 5, 6, 7 or 8 days. In some embodiments, the incubation in the presence of a stimulating agent (e.g. anti-CD3/anti-CD28) and/or a cytokine (e.g. IL-2, IL-7 and/or IL-15) is for up to 24 hours, 25 hours or 48 hours prior to the electroporation.

In some aspects, the provided compositions and methods include those in which at least or greater than about 50%, 60%, 65%, 70%. 75%, 80%, 85%, 90% or 95% of cells in a composition of cells into which an agent (e.g. gRNA/Cas9) for knockout or genetic disruption of a PDCD1 gene was introduced contain the genetic disruption; do not express the endogenous PD-1 polypeptide; do not contain a contiguous PDCD1 gene, a PDCD1 gene, and/or a functional PDCD1 gene. In some embodiments, the methods, compositions and cells according to the present disclosure include those in which at least or greater than about 50%, 60%, 65%, 70%. 75%, 80%, 85%, 90% or 95% of cells in a composition of cells into which an agent (e.g. gRNA/Cas9) for knockout or genetic disruption of a PDCD1 gene was introduced do not express a PD-1 polypeptide, such as on the surface of the cells. In some embodiments, at least or greater than about 50%, 60%, 65%, 70%. 75%, 80%, 85%, 90% or 95% of cells in a composition of cells into which an agent (e.g. gRNA/Cas9) for knockout or genetic disruption of a PDCD1 gene was introduced are knocked out in both alleles, i.e. comprise a biallelic deletion, in such percentage of cells.

In some embodiments, provided are compositions and methods in which the Cas9-mediated cleavage efficiency (% indel) in or near the PDCD1 gene (e.g. within or about within 100 base pairs, within or about within 50 base pairs, or within or about within 25 base pairs or within or about within 10 base pairs upstream or downstream of the cut site) is at least or greater than about 50%, 60%, 65%, 70%, 75%, 80%, 85%, 90% or 95% in cells of a composition of cells into which an agent (e.g. gRNA/Cas9) for knockout or genetic disruption of a PDCD1 gene has been introduced. In some embodiments, the provided cells, compositions and methods results in a reduction or disruption of signals delivered via the immune checkpoint molecule PD-1 in at least or greater than about 50%, 60%, 65%, 70%, 75%, 80%, 85%, 90% or 95% of cells in a composition of cells into which an agent (e.g. gRNA/Cas9) for knockout or genetic disruption of a PDCD1 gene was introduced.

In some embodiments, compositions according to the provided disclosure that comprise cells engineered with a recombinant receptor and comprise the reduction, deletion, elimination, knockout or disruption in expression of PD-1 (e.g. genetic disruption of a PDCD1 gene) retain the functional property or activities of the recombinant receptor (e.g. CAR) compared to the recombinant receptor expressed in engineered cells of a corresponding or reference composition in which such are engineered with the recombinant receptor but do not comprise the genetic disruption of a PDCD1 gene or express the PD-1 polypeptide when assessed under the same conditions. In some embodiments, the recombinant receptor (e.g. CAR) retains specific binding to the antigen. In some embodiments, the recombinant receptor (e.g. CAR) retains activating or stimulating activity, upon antigen binding, to induce cytotoxicity, proliferation, survival or cytokine secretion in cells. In some embodiments, the engineered cells of the provided compositions retain a functional property or activity compared to a corresponding or reference composition comprising engineered cells in which such are engineered with the recombinant receptor but do not comprise the genetic disruption of a PDCD1 gene or express the PD-1 polypeptide when assessed under the same conditions. In some embodiments, the cells retain cytotoxicity, proliferation, survival or cytokine secretion compared to such a corresponding or reference composition.

In some embodiments, the cells in the composition retain a phenotype of the immune cell or cells compared to the phenotype of cells in a corresponding or reference composition when assessed under the same conditions. In some embodiments, cells in the composition include naïve cells, effector memory cells, central memory cells, stem central memory cells, effector memory cells, and long-lived effector memory cells. In some embodiments, the percentage of T cells, or T cells expressing the recombinant receptor (e.g. CAR), and comprising the genetic disruption of a PDCD1 gene exhibit a non-activated, long-lived memory or central memory phenotype that is the same or substantially the same as a corresponding or reference population or composition of cells engineered with the recombinant receptor but not containing the genetic disruption or expressing the PD-1 polypeptide. In some embodiments, the provided composition comprises T cells comprising the recombinant receptor (e.g. CAR) and one or more phenotypic markers selected from CCR7+, 4-1BB+(CD137+), TIM3+, CD27+, CD62L+, CD127+, CD45RA+, CD45RO−, t-bet1^{low}, IL-7Ra+, CD95+, IL-2Rβ+, CXCR3+ or LFA-1+.

In some embodiments, such property, activity or phenotype can be measured in an in vitro assay, such as by incubation of the cells in the presence of the antigen, a cell expressing the antigen and/or an antigen-receptor activating substance. In some embodiments, the incubation is at or about 37° C.±2° C. In some embodiments, the incubation can be for up to or up to about 12, 24, 36, 48 or 60 hours, and optionally can be in the presence of one or more cytokines (e.g. IL-2, IL-15 and/or IL-17). In some embodiments, any of the assessed activities, properties or phenotypes can be assessed at various days following electroporation or other introduction of the agent, such as after or up to3, 4, 5, 6, 7 days. In some embodiments, such activity, property or phenotype is retained by at least 80%, 85%, 90%, 95% or 100% of the cells in the composition compared to the activity of a corresponding composition containing cells engineered with the recombinant receptor but not comprising the genetic disruption of a PDCD1 gene when assessed under the same conditions.

As used herein, reference to a “corresponding composition” or a “corresponding population of cells” (also called a “reference composition” or a “reference population of cells”) refers to T cells or cells obtained, isolated, generated, produced and/or incubated under the same or substantially the same conditions, except that the T cells or population of T cells were not introduced with the agent. In some aspects, except for not containing introduction of the agent, such cells or T cells are treated identically or substantially identically as T cells or cells that have been introduced with the agent, such that any one or more conditions that can influence the activity or properties of the cell, including the upregulation or expression of the inhibitory molecule, is not varied or not substantially varied between the cells other than the introduction of the agent. For example, for purposes of assessing reduction in expression and/or inhibition of upregulation of one or more inhibitory molecules (e.g. PD-1), T cells containing introduction of the agent and T cells not containing introduction of the agent are incubated under the same conditions known to lead to expression and or upregulation of the one or more inhibitory molecule in T cells.

Methods and techniques for assessing the expression and/or levels of T cell markers, including inhibitory molecules, such as PD-1, are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods. In some embodiments, antigen receptor (e.g. CAR)-expressing cells can be detected by flow cytometry or other immunoaffinity based method for expression of a marker unique to such cells, and then such cells can be co-stained for another T cell surface marker or markers, such as an inhibitory molecule (e.g. PD-1). In some embodiments, T cells expressing an antigen receptor (e.g. CAR) can be generated to contain a truncated EGFR (EGFRt) as a non-immunogenic selection epitope, which then can be used as a marker to detect the such cells (see e.g. U.S. Pat. No. 8,802,374).

In some embodiments, the cells, compositions and methods provide for the deletion, knockout, disruption, or reduction in expression of PD-1 in immune cells (e.g. T cells) to be adoptively transferred (such as cells engineered to express a CAR or transgenic TCR). In some embodiments, the methods are performed ex vivo on primary cells, such as primary immune cells (e.g. T cells) from a subject. In some aspects, methods of producing or generating such genetically engineered T cells include introducing into a population of cells containing immune cells (e.g. T cells) one or more nucleic acid encoding a recombinant receptor (e.g. CAR) and an agent or agents that is capable of disrupting, a gene that encode the immune inhibitory molecule PD-1.

As used herein, the term “introducing” encompasses a variety of methods of introducing DNA into a cell, either in vitro or in vivo, such methods including transformation, transduction, transfection (e.g. electroporation), and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors.

The population of cells containing T cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, T cells can be separated or selected to enrich T cells in the population using positive or negative selection and enrichment methods. In some embodiments, the population contains CD4+, CD8+ or CD4+ and CD8+ T cells. In some embodiments, the step of introducing the nucleic acid encoding a genetically engineered antigen receptor and the step of introducing the agent (e.g. Cas9/gRNA RNP) can occur simultaneously or sequentially in any order. In some embodiments, subsequent to introduction of the genetically engineered antigen receptor (e.g. CAR) and one or more agents (e.g. Cas9/gRNA RNP), the cells are cultured or incubated under conditions to stimulate expansion and/or proliferation of cells.

Thus, provided are cells, compositions and methods that enhance immune cell, such as T cell, function in adoptive cell therapy, including those offering improved efficacy, such as by increasing activity and potency of administered genetically engineered (e.g. CAR+) cells, while maintaining persistence or exposure to the transferred cells over time. In some embodiments, the genetically engineered cells, such as CAR-expressing T cells, exhibit increased expansion and/or persistence when administered in vivo to a subject, as compared to certain available methods.

In some embodiments, the provided compositions containing recombinant receptor-expressing cells, such as CAR-expressing cells, exhibit increased persistence when administered in vivo to a subject. In some embodiments, the persistence of genetically engineered cells, such as CAR-expressing T cells, in the subject upon administration is greater as compared to that which would be achieved by alternative methods, such as those involving administration of cells genetically engineered by methods in which T cells were not introduced with an agent that reduces expression of or disrupts a gene encoding PD-1. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the recombinant receptor (e.g., CAR-expressing cells) in the blood or serum or organ or tissue (e.g., disease site) of the subject. In some aspects, persistence is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the recombinant receptor (e.g. CAR-expressing cells) can be used to distinguish the administered cells from endogenous cells in a subject.

Also provided are methods and uses of the cells, such as in adoptive therapy in the treatment of cancers. Also provided are methods for engineering, preparing, and producing the cells, compositions containing the cells, and kits and devices containing and for using, producing and administering the cells. Also provided are methods, compounds, and compositions for producing the engineered cells. Provided are methods for cell isolation, genetic engineering and gene disruption. Provided are nucleic acids, such as constructs, e.g., viral vectors encoding the genetically engineered antigen receptors and/or encoding an agent for effecting disruption, and methods for introducing such nucleic acids into the cells, such as by transduction. Also provided are compositions containing the engineered cells, and methods, kits, and devices for administering the cells and compositions to subjects, such as for adoptive cell therapy. In some aspects, the cells are isolated from a subject, engineered, and administered to the same subject. In other aspects, they are isolated from one subject, engineered, and administered to another subject.

### II. Genetically Engineered Cells and Methods of Producing Cells Expressing a Recombinant Receptor

Provided are cells for adoptive cell therapy, e.g., adoptive immunotherapy, and method for producing or generating the cells. The cells include immune cells such as T cells. The cells generally are engineered by introducing one or more genetically engineered nucleic acid or product thereof. Among such products are genetically engineered antigen receptors, including engineered T cell receptors (TCRs) and functional non-TCR antigen receptors, such as chimeric antigen receptors (CARs), including activating, stimulatory, and costimulatory CARs, and combinations thereof. In some embodiments, the cells also are introduced, either simultaneously or sequentially with the nucleic acid encoding the genetically engineered antigen receptor, with an agent (e.g. Cas9/gRNA RNP) that is capable of disrupting a gene encoding the immune inhibitory molecule PD-1.

In some embodiments, the cells (e.g. T cells) can be incubated or cultivated prior to, during and/or subsequent to introducing the nucleic acid molecule encoding the recombinant receptor and/or the agent (e.g. Cas9/gRNA RNP). In some embodiments, the cells (e.g. T cells) can be incubated or cultivated prior to, during or subsequent to the introduction of the nucleic acid molecule encoding the recombinant receptor, such as prior to, during or subsequent to the transduction of the cells with a viral vector (e.g. lentiviral vector) encoding the recombinant receptor. In some embodiments, the cells (e.g. T cells) can be incubated or cultivated prior to, during or subsequent to the introduction of the agent (e.g. Cas9/gRNA RNP), such as prior to, during or subsequent to contacting the cells with the agent or prior to, during or subsequent to delivering the agent into the cells, e.g. via electroporation. In some embodiments, the incubation can be both in the context of introducing the nucleic acid molecule encoding the recombinant receptor and introducing the agent, e.g. Cas9/gRNA RNP. In some embodiments, the incubation can be in the presence of a cytokine, such as IL-2, IL-7 or IL-15, or in the presence of a stimulating or activating agents that induces the proliferation or activation of cells, such as an anti-CD3/anti-CD28 antibodies.

In some embodiments, the method includes activating or stimulating cells with a stimulating or activating agent (e.g. anti-CD3/anti-CD28 antibodies) prior to introducing the nucleic acid molecule encoding the recombinant receptor and the agent, e.g. Cas9/gRNA RNP. In some embodiments, incubation also can be performed in the presence of a cytokine, such as IL-2 (e.g. 1 U/ML to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g. 0.1 ng/mL to 50 ng/mL, such as 0.5 ng/mL to 25 ng/mL, for example, at least or about 1 ng/mL or 5 ng/mL). In some embodiment, the cells are incubated for 6 hours to 96 hours, such as 24-48 hours or 24-36 hours prior to introducing the nucleic acid molecule encoding the recombinant receptor (e.g. via transduction).

In some embodiments, the introducing the agent, e.g. Cas9/gRNA RNP, is after introducing the nucleic acid molecule encoding the recombinant receptor. In some embodiments, prior to the introducing of the agent, the cells are rested, e.g. by removal of any stimulating or activating agent. In some embodiments, prior to introducing the agent, the stimulating or activating agent and/or cytokines are not removed.

In some embodiments, subsequent to the introduction of the nucleic acid molecule and/or the introducing of the agent, e.g. Cas9/gRNA, the cells are incubated, cultivated or cultured in the presence of a cytokine, such as IL-2 (e.g. 1 U/ML to 500 U/mL, such as 1 U/mL to 100 U/mL, for example at least or about 25 U/mL or 50 U/mL), IL-7 (e.g. 0.5 ng/mL to 50 ng/mL, such as 1 ng/mL to 20 ng/mL, for example, at least or about 1 ng/mL or 5 ng/mL) or IL-15 (e.g. 0.1 ng/mL to 50 ng/mL, such as 0.1 ng/mL to 10 ng/mL, for example, at least or about 0.1 ng/mL, 0.5 ng/mL or 1 ng/mL).

In some embodiments, the incubation during any portion of the process or all of the process can be at a temperature of 30° C.±2° C. to 39° C.±2° C., such as at least or about at least 30° C.±2° C., 32° C.±2° C., 34° C.±2° C. or 37° C.±2° C. In some embodiments, at least a portion of the incubation is at 30° C.±2° C. and at least a portion of the incubation is at 37° C.±2° C.

A. Cells and Preparation of Cells for Genetic Engineering

Recombinant receptors that bind to a specific antigen and agents ((e.g. Cas9/gRNA RNP) for gene editing of a PDCD1 gene encoding a PD-1 polypeptide can be introduced into a wide variety of cells. In some embodiments, a recombinant receptor is engineered and/or the PDCD1 target gene is manipulated ex vivo and the resulting genetically engineered cells are administered to a subject. Sources of target cells for ex vivo manipulation may include, e.g., the subject's blood, the subject's cord blood, or the subject's bone marrow. Sources of target cells for ex vivo manipulation may also include, e.g., heterologous donor blood, cord blood, or bone marrow.

In some embodiments, the cells, e.g., engineered cells, are eukaryotic cells, such as mammalian cells, e.g., human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In some embodiments, the target cell is a T cell, e.g., a CD8+ T cell (e.g., a CD8+ naïve T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naïve T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the methods include isolating cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker−) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Rα (CD127). In some examples, CD8+ T cells are enriched for cells positive for CD45RO (or negative for CD45RA) and for CD62L.

For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining TCM-enriched CD8+ T cells and CD4+ T cells further enhances efficacy.

In embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells are sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L- and CD45RO.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the methods include re-introducing the engineered cells into the same patient, before or after cryopreservation.

B. Recombinant Receptors

In some embodiments, the cells comprise one or more nucleic acids encoding a recombinant receptor introduced via genetic engineering, and genetically engineered products of such nucleic acids. In some embodiments, the cells can be produced or generated by introducing into a cell (e.g. via transduction of a viral vector, such as a retroviral or lentiviral vector) a nucleic acid molecule encoding the recombinant receptor. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, the target cell has been altered to bind to one or more target antigen, such as one or more tumor antigen. In some embodiments, the target antigen is selected from ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag. In some embodiments, the target cell has been altered to bind one or more of the following tumor antigens, e.g., by a TCR or a CAR. Tumor antigens may include, but are not limited to, AD034, AKT1, BRAP, CAGE, CDX2, CLP, CT-7, CT8/HOM-TES-85, cTAGE-1, Fibulin-1, HAGE, HCA587/MAGE-C2, hCAP-G, HCE661, HER2/neu, HLA-Cw, HOM-HD-21/Galectin9, HOM-MEEL-40/SSX2, HOM-RCC-3.1.3/CAXII, HOXA7, HOXB6, Hu, HUB1, KM-HN-3, KM-KN-1, KOC1, KOC2, KOC3, KOC3, LAGE-1, MAGE-1, MAGE-4a, MPP11, MSLN, NNP-1, NY-BR-1, NY-BR-62, NY-BR-85, NY-CO-37, NY-CO-38, NY-ESO-1, NY-ESO-5, NY-LU-12, NY-REN-10, NY-REN-19/LKB/STK11, NY-REN-21, NY-REN-26/BCR, NY-REN-3/NY-CO-38, NY-REN-33/SNC6, NY-REN-43, NY-REN-65, NY-REN-9, NY-SAR-35, OGFr, PLU-1, Rab38, RBPJkappa, RHAMM, SCP1, SCP-1, SSX3, SSX4, SSX5, TOP2A, TOP2B, or Tyrosinase.

I. Antigen Receptors

a) Chimeric Antigen Receptors (CARs)

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens that may be targeted by the receptors include, but are not limited to, αvβ6 integrin (avb6 integrin), B cell maturation antigen (BCMA), B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, fetal acetylcholine receptor, ganglioside GD2, O-acetylated GD2 (OGD2), ganglioside GD3, glycoprotein 100 (gp100), Her2/neu (receptor tyrosine kinase erbB2), Her3 (erb-B3), Her4 (erb-B4), erbB dimers, human high molecular weight-melanoma-associated antigen (HMW-MAA), hepatitis B surface antigen, Human leukocyte antigen A1 (HLA-AI), human leukocyte antigen A2 (HLA-A2), IL-22 receptor alpha(IL-22Ra), IL-13 receptor alpha 2 (IL-13Ra2), kinase insert domain receptor (kdr), kappa light chain, L1 cell adhesion molecule (L1CAM), CE7 epitope of L1-CAM, Leucine Rich Repeat Containing 8 Family Member A (LRRC8A), Lewis Y, melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, preferentially expressed antigen of melanoma (PRAME), progesterone receptor, a prostate specific antigen, prostate stem cell antigen (PSCA), prostate specific membrane antigen (PSMA), receptor tyrosine kinase like orphan receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms tumor 1 (WT-1), and a pathogen-specific antigen.

In some embodiments, antigens targeted by the receptors in some embodiments include orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, OEPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, and MAGE A3, CE7, Wilms Tumor 1 (WT-1), a cyclin, such as cyclin A1 (CCNA1), and/or biotinylated molecules, and/or molecules expressed by HIV, HCV, HBV or other pathogens.

In some embodiments, the CAR has binding specificity for a tumor associated antigen, e.g., CD19, CD20, carbonic anhydrase IX (CAIX), CD171, CEA, ERBB2, GD2, alpha-folate receptor, Lewis Y antigen, prostate specific membrane antigen (PSMA) or tumor associated glycoprotein 72 (TAG72).

In some embodiments, the CAR binds a pathogen-specific antigen. In some embodiments, the CAR is specific for viral antigens (such as HIV, HCV, HBV, etc.), bacterial antigens, and/or parasitic antigens.

Among the chimeric receptors are chimeric antigen receptors (CARs). The chimeric receptors, such as CARs, generally include an extracellular antigen binding domain, such as a portion of an antibody molecule, generally a variable heavy (V_{H}) chain region and/or variable light (V_{L}) chain region of the antibody, e.g., an scFv antibody fragment.

In some embodiments, the antibody portion of the recombinant receptor, e.g., CAR, further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers, e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain.

Exemplary spacers include, but are not limited to, those described in Hudecek et al. (2013) Clin. Cancer Res., 19:3153 or international patent application publication number WO2014031687. In some embodiments, the spacer has the sequence set forth in SEQ ID NO: 51213, and is encoded by the sequence set forth in SEQ ID NO: 51212. In some embodiments, the spacer has the sequence set forth in SEQ ID NO: 51214. In some embodiments, the spacer has the sequence set forth in SEQ ID NO: 51215. In some embodiments, the constant region or portion is of IgD. In some embodiments, the spacer has the sequence set forth in SEQ ID NO:51216. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 51213, 51214, 51215 or 51216.

This antigen recognition domain generally is linked to one or more intracellular signaling components, such as signaling components that mimic activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD64, CD80, CD86, CD 134, CD137, CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from the CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published application No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 51218 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 51218. An exemplary T2A linker sequence comprises the sequence of amino acids set forth in SEQ ID NO: 51217 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 51217.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 51219 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:51219; in some embodiments, the transmembrane-domain containing portion of the recombinant receptor comprises the sequence of amino acids set forth in SEQ ID NO: 51220 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the chimeric antigen receptor contains an intracellular domain of a T cell costimulatory molecule. In some aspects, the T cell costimulatory molecule is CD28 or 41BB.

In some embodiments, the intracellular signaling domain comprises an intracellular costimulatory signaling domain of human CD28 or functional variant or portion thereof thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 51221 or 51222 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 51221 or 51222. In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 51223 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 51223.

In some embodiments, the intracellular signaling domain comprises a human CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No. 7,446,190 or 8,911,993. In some embodiments, the intracellular signaling domain comprises the sequence of amino acids set forth in SEQ ID NO: 51224, 51225 or 51226 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 51224, 51225 or 51226.

In some aspects, the spacer contains only a hinge region of an IgG, such as only a hinge of IgG4 or IgG1, such as the hinge only spacer set forth in SEQ ID NO:51213. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains, such as set forth in SEQ ID NO:396. In some embodiments, the spacer is an Ig hinge, e.g., an IgG4 hinge, linked to a CH3 domain only, such as set forth in SEQ ID NO:51214. In some embodiments, the spacer is or comprises a glycine-serine rich sequence or other flexible linker such as known flexible linkers.

For example, in some embodiments, the CAR includes an antibody or fragment that specifically binds an antigen, a spacer such as any of the Ig-hinge containing spacers, a CD28 transmembrane domain, a CD28 intracellular signaling domain, and a CD3 zeta signaling domain. In some embodiments, the CAR includes the an antibody or fragment that specifically binds an antigen, a spacer such as any of the Ig-hinge containing spacers, a CD28 transmembrane domain, a CD28 intracellular signaling domain, and a CD3 zeta signaling domain. In some embodiments, such CAR constructs further includes a T2A ribosomal skip element and/or a tEGFR sequence, e.g., downstream of the CAR.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

b) T Cell Receptors

In some embodiments, the genetically engineered antigen receptors include recombinant T cell receptors (TCRs) and/or TCRs cloned from naturally occurring T cells. Thus, in some embodiments, the target cell has been altered to contain specific T cell receptor (TCR) genes (e.g., a TRAC and TRBC gene). TCRs or antigen-binding portions thereof include those that recognize a peptide epitope or T cell epitope of a target polypeptide, such as an antigen of a tumor, viral or autoimmune protein. In some embodiments, the TCR has binding specificity for a tumor associated antigen, e.g., carcinoembryonic antigen (CEA), GP100, melanoma antigen recognized by T cells 1 (MART1), melanoma antigen A3 (MAGEA3), NYESO1 or p53.

In some embodiments, a “T cell receptor” or “TCR” is a molecule that contains a variable α and β chains (also known as TCRα and TCRβ, respectively) or a variable γ and δ chains (also known as TCRγ and TCRδ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. Generally, a TCR is or can be expressed on the surface of T cells (or T lymphocytes) where it is generally responsible for recognizing antigens bound to major histocompatibility complex (MHC) molecules.

In some embodiments, thethe TCR is a full TCRs or an antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

In some embodiments, the variable domains of the TCR contain hypervariable loops, or CDRs, which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR contains a variable alpha domain (V_{α}) and/or a variable beta domain (V_{β}) or antigen-binding fragments thereof. In some embodiments, the α-chain and/or β-chain of a TCR also can contain a constant domain, a transmembrane domain and/or a short cytoplasmic tail (see, e.g., Janeway et al., Immunobiology: The Immune System in Health and Disease, 3^{rd} Ed., Current Biology Publications, p. 4:33, 1997). In some embodiments, the α chain constant domain is encoded by the TRAC gene (IMGT nomenclature) or is a variant thereof. In some embodiments, the β chain constant region is encoded by TRBC1 or TRBC2 genes (IMGT nomenclature) or is a variant thereof. In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

It is within the level of a skilled artisan to determine or identify the various domains or regions of a TCR. In some aspects, residues of a TCR are known or can be identified according to the International Immunogenetics Information System (IMGT) numbering system (see e.g. www.imgt.org; see also, Lefranc et al. (2003) Developmental and Comparative Immunology, 2&; 55-77; and The T Cell Factsbook 2nd Edition, Lefranc and LeFranc Academic Press 2001). Using this system, the CDR1 sequences within a TCR Vα chains and/or Vβ chain correspond to the amino acids present between residue numbers 27-38, inclusive, the CDR2 sequences within a TCR Vα chain and/or Vβ chain correspond to the amino acids present between residue numbers 56-65, inclusive, and the CDR3 sequences within a TCR Vα chain and/or Vβ chain correspond to the amino acids present between residue numbers 105-117, inclusive.

In some embodiments, the TCR may be a heterodimer of two chains α and β (or optionally γ and δ) that are linked, such as by a disulfide bond or disulfide bonds. In some embodiments, the constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains. In some embodiments, each of the constant and variable domains contain disulfide bonds formed by cysteine residues.

In some embodiments, the TCR for engineering cells as described is one generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences. In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the T-cells can be a cultured T-cell hybridoma or clone. In some embodiments, the TCR or antigen-binding portion thereof can be synthetically generated from knowledge of the sequence of the TCR.

In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments as described, the TCR can contain an introduced disulfide bond or bonds. In some embodiments, the native disulfide bonds are not present. In some embodiments, the one or more of the native cysteines (e.g. in the constant domain of the α chain and β chain) that form a native interchain disulfide bond are substituted to another residue, such as to a serine or alanine. In some embodiments, an introduced disulfide bond can be formed by mutating non-cysteine residues on the alpha and beta chains, such as in the constant domain of the α chain and β chain, to cysteine. Exemplary non-native disulfide bonds of a TCR are described in published International PCT No. WO2006/000830 and WO2006037960. In some embodiments, cysteines can be introduced at residue Thr48 of the α chain and Ser57 of the β chain, at residue Thr45 of the α chain and Ser77 of the β chain, at residue Tyr10 of the α chain and Ser17 of the β chain, at residue Thr45 of the α chain and Asp59 of the β chain and/or at residue Ser15 of the α chain and Glu15 of the β chain. In some embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

In some embodiments, the TCR chains contain a transmembrane domain. In some embodiments, the transmembrane domain is positively charged. In some cases, the TCR chain contains a cytoplasmic tail. In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). A TCR may be cell-bound or in soluble form. In some embodiments, for purposes of the provided methods, the TCR is in cell-bound form expressed on the surface of a cell.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native interchain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR, which is a single amino acid strand containing an α chain and a β chain that is able to bind to MHC-peptide complexes. Typically, a scTCR can be generated using methods known to those of skill in the art, See e.g., International published PCT Nos. WO 96/13593, WO 96/18105, WO99/18129, WO04/033685, WO2006/037960, WO2011/044186; U.S. Pat. No. 7,569,664; and Schlueter, C. J. et al. J. Mol. Biol. 256, 859 (1996).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an amino acid sequence corresponding to a TCR β chain variable region, a second segment constituted by an amino acid sequence corresponding to a TCR α chain variable region sequence fused to the N terminus of an amino acid sequence corresponding to a TCR α chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, for the scTCR to bind an MHC-peptide complex, the α and β chains must be paired so that the variable region sequences thereof are orientated for such binding. Various methods of promoting pairing of an α and β in a scTCR are well known in the art. In some embodiments, a linker sequence is included that links the α and β chains to form the single polypeptide strand. In some embodiments, the linker should have sufficient length to span the distance between the C terminus of the α chain and the N terminus of the β chain, or vice versa, while also ensuring that the linker length is not so long so that it blocks or reduces bonding of the scTCR to the target peptide-MHC complex.

In some embodiments, the linker of a scTCRs that links the first and second TCR segments can be any linker capable of forming a single polypeptide strand, while retaining TCR binding specificity. In some embodiments, the linker sequence may, for example, have the formula —P-AA-P—, wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)₅-P— or -PGGG-(SGGGG)₆-P—, wherein P is proline, G is glycine and S is serine (SEQ ID NO:51227 or 51228). In some embodiments, the linker has the sequence GSADDAKKDAAKKDGKS (SEQ ID NO:51229).

In some embodiments, a scTCR contains a disulfide bond between residues of the single amino acid strand, which, in some cases, can promote stability of the pairing between the α and β regions of the single chain molecule (see e.g. U.S. Pat. No. 7,569,664). In some embodiments, the scTCR contains a covalent disulfide bond linking a residue of the immunoglobulin region of the constant domain of the α chain to a residue of the immunoglobulin region of the constant domain of the β chain of the single chain molecule. In some embodiments, the disulfide bond corresponds to the native disulfide bond present in a native dTCR. In some embodiments, the disulfide bond in a native TCR is not present. In some embodiments, the disulfide bond is an introduced non-native disulfide bond, for example, by incorporating one or more cysteines into the constant region extracellular sequences of the first and second chain regions of the scTCR polypeptide. Exemplary cysteine mutations include any as described above. In some cases, both a native and a non-native disulfide bond may be present.

In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, any of the TCRs, including a dTCR or scTCR, can be linked to signaling domains that yield an active TCR on the surface of a T cell. In some embodiments, the TCR is expressed on the surface of cells. In some embodiments, the TCR does contain a sequence corresponding to a transmembrane sequence. In some embodiments, the transmembrane domain can be a Cα or Cβ transmembrane domain. In some embodiments, the transmembrane domain can be from a non-TCR origin, for example, a transmembrane region from CD3z, CD28 or B7.1. In some embodiments, the TCR does contain a sequence corresponding to cytoplasmic sequences. In some embodiments, the TCR contains a CD3z signaling domain. In some embodiments, the TCR is capable of forming a TCR complex with CD3.

In some embodiments, the TCR or antigen-binding fragment thereof exhibits an affinity with an equilibrium binding constant for a target antigen of between or between about 10⁻⁵ and 10⁻¹² M and all individual values and ranges therein. In some embodiments, the target antigen is an MHC-peptide complex or ligand.

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. In some embodiments, to generate a vector encoding a TCR, the α and β chains can be PCR amplified from total cDNA isolated from a T cell clone expressing the TCR of interest and cloned into an expression vector. In some embodiments, the α and β chains can be synthetically generated.

In some embodiments, the TCR alpha and beta chains are isolated and cloned into a gene expression vector. n some embodiments, transcription units can be engineered as a bicistronic unit containing an IRES (internal ribosome entry site), which allows coexpression of gene products (e.g. encoding an α and β chains) by a message from a single promoter. Alternatively, in some cases, a single promoter may direct expression of an RNA that contains, in a single open reading frame (ORF), multiple genes (e.g. encoding an α and β chains) separated from one another by sequences encoding a self-cleavage peptide (e.g., T2A) or a protease recognition site (e.g., furin). The ORF thus encodes a single polyprotein, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream. Examples of 2A cleavage peptides, including those that can induce ribosome skipping, are T2A, P2A, E2A and F2A. In some embodiments, the α and β chains are cloned into different vectors. In some embodiments, the generated a and β chains are incorporated into a retroviral, e.g. lentiviral, vector.

In some embodiments, the TCR alpha and beta genes are linked via a picornavirus 2A ribosomal skip peptide so that both chains are coexpression. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; an Hackett et al. (2010) Molecular Therapy: The Journal of the American Society of Gene Therapy. 18:674-683.

2 Vectors and Methods of Engineering

The provided methods include expressing the recombinant receptors, including CARs or TCRs, for producing the genetically engineered cells expressing such binding molecules. The genetic engineering generally involves introduction of a nucleic acid encoding the recombinant or engineered component into the cell, such as by retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

Various methods for the introduction of genetically engineered components, e.g., antigen receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, nucleic acid encoding a recombinant receptor can be cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λ610, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the recombinant receptor. In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other promoters known to a skilled artisan also are contemplated.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

C. Gene Editing of PDCD1

In any of the embodiments provided herein, an engineered immune cell can be subject to gene alteration, or gene editing, that is targeted to a locus encoding a gene involved in immunomodulation. In some embodiments, the target locus for gene editing is the programmed cell death 1 (PDCD1) locus, which encodes the programmed cell death (PD-1) protein. In some embodiments, gene editing results in an insertion or a deletion at the targeted locus, or a “knockout” of the targeted locus and elimination of the expression of the encoded protein. In some embodiments, the gene editing is achieved by non-homologous end joining (NHEJ) using a CRISPR/Cas9 system. In some embodiments, one or more guide RNA (gRNA) molecule can be used with one or more Cas9 nuclease, Cas9 nickase, enzymatically inactive Cas9 or variants thereof. Exemplary features of the gRNA molecule(s) and the Cas9 molecule(s) are described below.

I. Guide RNA (gRNA) molecules

In some embodiments, the agent comprises a gRNA that targets a region of the PDCD1 locus. A “gRNA molecule” refers to a nucleic acid that promotes the specific targeting or homing of a gRNA molecule/Cas9 molecule complex to a target nucleic acid, such as a locus on the genomic DNA of a cell. gRNA molecules can be unimolecular (having a single RNA molecule), sometimes referred to herein as “chimeric” gRNAs, or modular (comprising more than one, and typically two, separate RNA molecules).

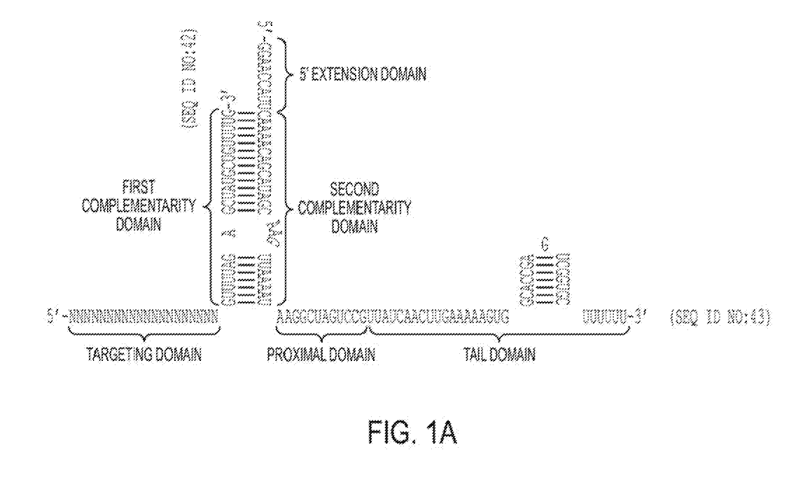

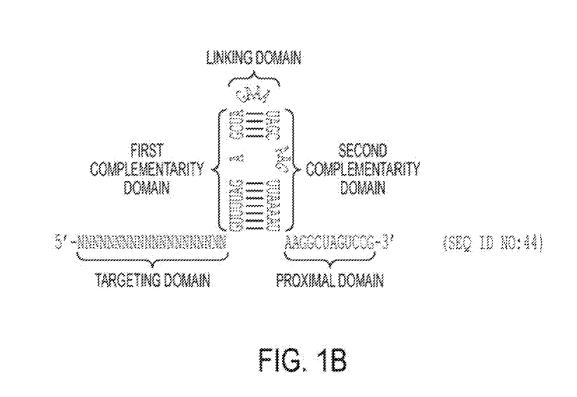

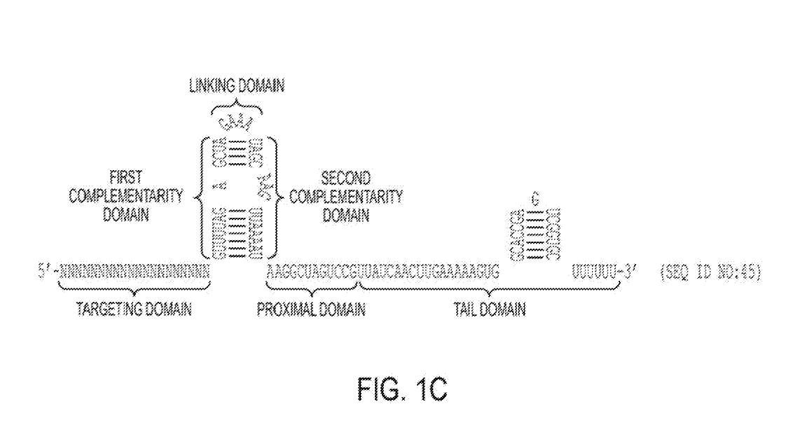

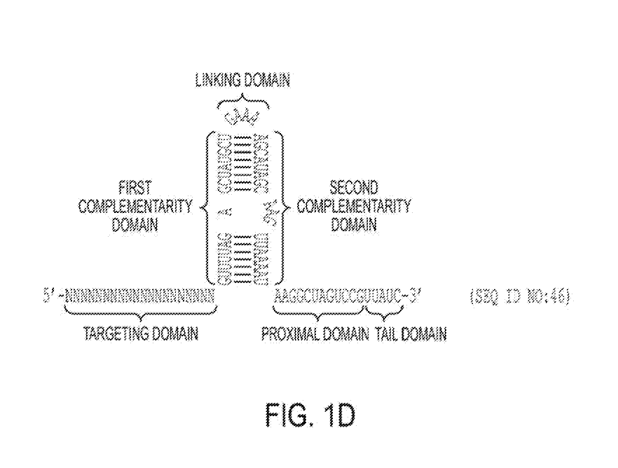

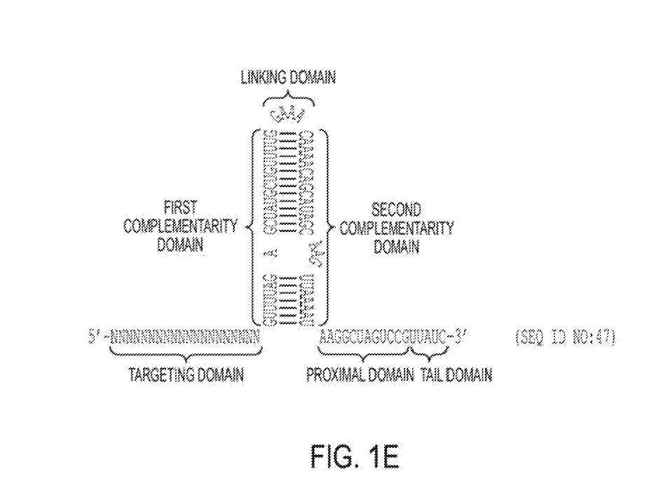

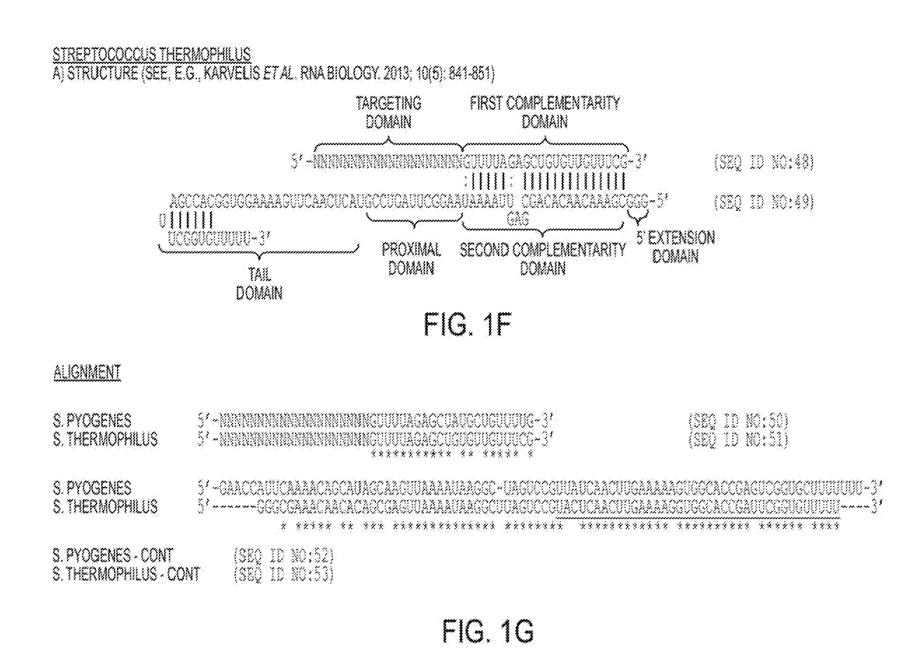

Several exemplary gRNA structures, with domains indicated thereon, are provided in FIG. 1. While not wishing to be bound by theory, with regard to the three dimensional form, or intra- or inter-strand interactions of an active form of a gRNA, regions of high complementarity are sometimes shown as duplexes in FIG. 1 and other depictions provided herein.

In some cases, the gRNA is a unimolecular or chimeric gRNA comprising, from 5′ to 3′:
a targeting domain which is complementary to a target nucleic acid, such as a sequence from the PDCD1 gene (coding sequence set forth in SEQ ID NO:51208); a first complementarity domain; a linking domain; a second complementarity domain (which is complementary to the first complementarity domain); a proximal domain; and optionally, a tail domain.

In other cases, the gRNA is a modular gRNA comprising first and second strands. In these cases, the first strand preferably includes, from 5′ to 3′: a targeting domain (which is complementary to a target nucleic acid, such as a sequence from the PDCD1 gene, coding sequence set forth in SEQ ID NO:51208) and a first complementarity domain. The second strand generally includes, from 5′ to 3′: optionally, a 5′ extension domain; a second complementarity domain; a proximal domain; and optionally, a tail domain.

These domains are discussed briefly below:

a) The Targeting Domain

FIG. 1 provides examples of the placement of targeting domains.

The targeting domain comprises a nucleotide sequence that is complementary, e.g., at least 80, 85, 90, 95, 98 or 99% complementary, e.g., fully complementary, to the target sequence on the target nucleic acid. The strand of the target nucleic acid comprising the target sequence is referred to herein as the “complementary strand” of the target nucleic acid. Guidance on the selection of targeting domains can be found, e.g., in Fu Y et al., Nat Biotechnol 2014 (doi: 10.1038/nbt.2808) and Sternberg S H et al., Nature 2014 (doi: 10.1038/nature13011).

The targeting domain is part of an RNA molecule and will therefore comprise the base uracil (U), while any DNA encoding the gRNA molecule will comprise the base thymine (T). While not wishing to be bound by theory, in an embodiment, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence. In an embodiment, the target domain itself comprises in the 5′ to 3′ direction, an optional secondary domain, and a core domain. In an embodiment, the core domain is fully complementary with the target sequence. In an embodiment, the targeting domain is 5 to 50 nucleotides in length. The strand of the target nucleic acid with which the targeting domain is complementary is referred to herein as the complementary strand. Some or all of the nucleotides of the domain can have a modification, e.g., to render it less susceptible to degradation, improve bio-compatibility, etc. By way of non-limiting example, the backbone of the target domain can be modified with a phosphorothioate, or other modification(s). In some cases, a nucleotide of the targeting domain can comprise a 2′ modification, e.g., a 2-acetylation, e.g., a 2′ methylation, or other modification(s).

In various embodiments, the targeting domain is 16-26 nucleotides in length (i.e. it is 16 nucleocides in length, or 17 nucleotides in length, or 18, 19, 20, 21, 22, 23, 24, 25 or 26 nucleotides in length.

Exemplary Targeting Domains

In some embodiments, the target sequence (target domain) is at or near the PDCD1 locus, such as any part of the PDCD1 coding sequence set forth in SEQ ID NO:51208. In some embodiments, the target nucleic acid complementary to the targeting domain is located at an early coding region of a gene of interest, such as PDCD1. Targeting of the early coding region can be used to knockout (i.e., eliminate expression of) the gene of interest. In some embodiments, the early coding region of a gene of interest includes sequence immediately following a start codon (e.g., ATG), or within 500 bp of the start codon (e.g., less than 500, 450, 400, 350, 300, 250, 200, 150, 100, 50 bp, 40 bp, 30 bp, 20 bp, or 10 bp). In particular examples, the target nucleic acid is within 200 bp, 150 bp, 100 bp, 50 bp, 40 bp, 30 bp, 20 bp or 10 bp of the start codon. In some examples, the targeting domain of the gRNA is complementary, e.g., at least 80, 85, 90, 95, 98 or 99% complementary, e.g., fully complementary, to the target sequence on the target nucleic acid, such as the target nucleic acid in the PDCD1 locus.

In some embodiments, the targeting domain for knockout or knockdown of PDCD1 is or comprises a sequence selected from any of SEQ ID NOS: 481-3748 or 14657-21037.

In some embodiments, the targeting domain is or comprises the sequence GUCUGGGCGGUGCUACAACU (SEQ ID NO:508), GCCCUGGCCAGUCGUCU (SEQ ID NO: 514), CGUCUGGGCGGUGCUACAAC (SEQ ID NO:1533), UGUAGCACCGCCCAGACGAC (SEQ ID NO:579), CGACUGGCCAGGGCGCCUGU (SEQ ID NO:582) and CACCUACCUAAGAACCAUCC (SEQ ID NO:723). In some embodiments, the targeting domain comprises the sequence GUCUGGGCGGUGCUACAACU (SEQ ID NO:508). In some embodiments, the targeting domain comprises the sequence GCCCUGGCCAGUCGUCU (SEQ ID NO: 514). In some embodiments, the targeting domain comprises the sequence CGUCUGGGCGGUGCUACAAC (SEQ ID NO:1533). In some embodiments, the targeting domain comprises the sequence UGUAGCACCGCCCAGACGAC (SEQ ID NO:579). In some embodiments, the targeting domain comprises the sequence CGACUGGCCAGGGCGCCUGU (SEQ ID NO:582) and CACCUACCUAAGAACCAUCC (SEQ ID NO:723).

In some embodiments, targeting domains include those for knocking out the PDCD1 gene using S. pyogenes Cas9 or using N. meningitidis Cas9.

In some embodiments, targeting domains include those for knocking out the PDCD1 gene using S. pyogenes Cas9. Any of the targeting domains can be used with a S. pyogenes Cas9 molecule that generates a double stranded break (Cas9 nuclease) or a single-stranded break (Cas9 nickase).

In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired with any gRNA comprising a plus strand targeting domain. In some embodiments, the two gRNAs are oriented on the DNA such that PAMs face outward and the distance between the 5′ ends of the gRNAs is 0-50 bp. In an embodiment, two gRNAs are used to target two Cas9 nucleases or two Cas9 nickases, for example, using a pair of Cas9 molecule/gRNA molecule complex guided by two different gRNA molecules to cleave the target domain with two single stranded breaks on opposing strands of the target domain. In some embodiments, the two Cas9 nickases can include a molecule having HNH activity, e.g., a Cas9 molecule having the RuvC activity inactivated, e.g., a Cas9 molecule having a mutation at D10, e.g., the D10A mutation, a molecule having RuvC activity, e.g., a Cas9 molecule having the HNH activity inactivated, e.g., a Cas9 molecule having a mutation at H840, e.g., a H840A, or a molecule having RuvC activity, e.g., a Cas9 molecule having the HNH activity inactivated, e.g., a Cas9 molecule having a mutation at N863, e.g., N863A. In some embodiments, each of the two gRNAs are complexed with a D10A Cas9 nickase

In some embodiments, the two targeting domains can include a gRNA with a targeting domain that is or comprises any of the sequences in Group A can be paired with a gRNA with any targeting domain from Group B (Table 1A). In some embodiments, a gRNA with a targeting domain from Group C can be paired with a gRNA with any targeting domain from Group D (Table 1A).

TABLE 1A

| Group A | Group B |
|---|---|
| UGACACGGAAGCGGCAGUCC | GCGUGACUUCCACAUGAGCG |
| (SEQ ID NO: 600); | (SEQ ID NO: 567); |
| CACGGAAGCGGCAGUCC | ACUUCCACAUGAGCGUGGUC |
| (SEQ ID NO: 601) | (SEQ ID NO: 590); |
| | UGACUUCCACAUGAGCG |
| | (SEQ ID NO: 592); |
| | UCCACAUGAGCGUGGUC |
| | (SEQ ID NO: 593) |
| Group C | Group D |
| CAUGUGGAAGUCACGCCCGU | AGGGCCCGGCGCAAUGACAG |
| (SEQ ID NO: 597); | (SEQ ID NO: 594); |
| AUGUGGAAGUCACGCCCGUU | GCCCGGCGCAAUGACAG |
| (SEQ ID NO: 598); | (SEQ ID NO: 568) |
| GUGGAAGUCACGCCCGU | |
| (SEQ ID NO: 571); | |
| UGGAAGUCACGCCCGUU | |
| (SED ID NO: 599) | |

In some embodiments, the two targeting domains can include a gRNA with a targeting domain that is or comprises any of the sequences in Group E can be paired with a gRNA with any targeting domain from Group F (Table 1B).

TABLE 1B

| Group E | Group F |
|---|---|
| CGACUGGCCAGGGCGCCUGU | GUCUGGGCGGUGCUACAACU (SEQ ID |
| (SEQ ID NO: 582) | NO: 508); |
| UGUAGCACCGCCCAGACGAC | GGGCGGUGCUACAACUGGGC (SEQ ID |
| (SEQ ID NO: 579) | NO: 510); |
| ACCGCCCAGACGACUGGCCA | GGCCAGGAUGGUUCUUAGGU (SEQ ID |
| (SEQ ID NO: 581) | NO: 511); |
| | GGAUGGUUCUUAGGUAGGUG (SEQ ID |
| | NO: 512); |
| | CGUCUGGGCGGUGCUACAAC (SEQ ID |
| | NO: 576); |
| | CUACAACUGGGCUGGCGGCC (SEQ ID |
| | NO: 766); |

In some embodiments, the two targeting domains can include a gRNA pairs from the following pairs in Table 1C. In some embodiments, the pair of Cas9 molecule/gRNA molecule complex include a gRNA pair from Table 1C, each complexed with a D10A Cas9 nickase. In some embodiments, the pair of Cas9 molecule/gRNA molecule complex include a gRNA pair from Table 1C, each complexed with N863A Cas9 nickase.

TABLE 1C

| | |
|---|---|
| CGACUGGCCAGGGCGCCUGU | GUCUGGGCGGUGCUACAACU; |
| (SEQ ID NO: 582) and | (SEQ ID NO: 508) |
| CGACUGGCCAGGGCGCCUGU | GGGCGGUGCUACAACUGGGC; |
| (SEQ ID NO: 582) and | (SEQ ID NO: 510) |
| CGACUGGCCAGGGCGCCUGU | GGCCAGGAUGGUUCUUAGGU; |
| (SEQ ID NO: 582) and | (SEQ ID NO: 511) |
| CGACUGGCCAGGGCGCCUGU | GGAUGGUUCUUAGGUAGGUG; |
| (SEQ ID NO: 582) and | (SEQ ID NO: 512) |
| CGACUGGCCAGGGCGCCUGU | CGUCUGGGCGGUGCUACAAC; |
| (SEQ ID NO: 582) and | (SEQ ID NO: 576) |
| CGACUGGCCAGGGCGCCUGU | CUACAACUGGGCUGGCGGCC; |
| (SEQ ID NO: 582) and | (SEQ ID NO: 766) |
| UGUAGCACCGCCCAGACGAC | GGCCAGGAUGGUUCUUAGGU; |
| (SEQ ID NO: 579) and | (SEQ ID NO: 511) |
| UGUAGCACCGCCCAGACGAC | GGAUGGUUCUUAGGUAGGUG; |
| (SEQ ID NO: 579) and | (SEQ ID NO: 512) or |
| ACCGCCCAGACGACUGGCCA | GGCCAGGAUGGUUCUUAGGU. |
| (SEQ ID NO: 581) and | (SEQ ID NO: 511) |

In some embodiments, an engineered immune cell can be subject to gene alteration, or gene editing, by additionally or alternatively targeting to a locus from one or more of FAS, BID, CTLA4, CBLB, PTPN6, TRAC and/or TRBC. In some embodiments, one or more of the FAS, BID, CTLA4, PDCD1, CBLB, PTPN6, TRAC and TRBC genes are targeted as a targeted knockout or knockdown, e.g., to affect T cell proliferation, survival and/or function. In an embodiment, said approach comprises knocking out or knocking down one T-cell expressed gene (e.g., FAS, BID, CTLA4, PDCD1, CBLB, PTPN6, TRAC or TRBC gene). In another embodiment, the approach comprises knocking out or knocking down two T-cell expressed genes, e.g., two of FAS, BID, CTLA4, PDCD1, CBLB, PTPN6, TRAC or TRBC genes. In another embodiment, the approach comprises knocking out or knocking down three T-cell expressed genes, e.g., three of FAS, BID, CTLA4, PDCD1, CBLB, PTPN6, TRAC or TRBC genes. In another embodiment, the approach comprises knocking out or knocking down four T-cell expressed genes, e.g., four of FAS, BID, CTLA4, PDCD1, CBLB, PTPN6, TRAC or TRBC genes. In another embodiment, the approach comprises knocking out or knocking down five T-cell expressed genes, e.g., five of FAS, BID, CTLA4, PDCD1, CBLB, PTPN6, TRAC or TRBC genes. In another embodiment, the approach comprises knocking out or knocking down six T-cell expressed genes, e.g., six of FAS, BID, CTLA4, PDCD1, CBLB, PTPN6, TRAC or TRBC genes. In another embodiment, the approach comprises knocking out or knocking down seven T-cell expressed genes, e.g., seven of FAS, BID, CTLA4, PDCD1, CBLB, PTPN6, TRAC or TRBC genes. In another embodiment, the approach comprises knocking out or knocking down eight T-cell expressed genes, e.g., each of FAS, BID, CTLA4, PDCD1, CBLB, PTPN6, TRAC and TRBC genes.

In some embodiments, the targeting domain for knockout or knockdown of FAS is or comprises a sequence selected from any of SEQ ID NOS: 8460-10759 or 27729-32635.

In some embodiments, the targeting domain for knockout or knockdown of BID is or comprises a sequence selected from any of SEQ ID NOS: 10760-13285 or 40252-45980.

In some embodiments, the targeting domain for knockout or knockdown of CTLA4 is or comprises a sequence selected from any of SEQ ID NOS: 13286-14656 or 45981-49273.

In some embodiments, the targeting domain for knockout or knockdown of CBLB is or comprises a sequence selected from any of SEQ ID NOS: 6119-8639 or 32636-40251.

In some embodiments, the targeting domain for knockout or knockdown of PTPN6 is or comprises a sequence selected from any of SEQ ID NOS: 3749-6118 or 21038-27728.

In some embodiments, the targeting domain for knockout or knockdown of TRAC is or comprises a sequence selected from any of SEQ ID NOS: 49274-49950.

In some embodiments, the targeting domain for knockout or knockdown of TRBC is or comprises a sequence selected from any of SEQ ID NOS: 49951-51200.

b) The First Complementarity Domain

FIGS. 1A-1G provide examples of first complementarity domains. The first complementarity domain is complementary with the second complementarity domain described below, and generally has sufficient complementarity to the second complementarity domain to form a duplexed region under at least some physiological conditions. The first complementarity domain is typically 5 to 30 nucleotides in length, and may be 5 to 25 nucleotides in length, 7 to 25 nucleotides in length, 7 to 22 nucleotides in length, 7 to 18 nucleotides in length, or 7 to 15 nucleotides in length. In various embodiments, the first complementary domain is 5, 6, 7, 8, 9, 10, 11, 12, 13, 14, 15, 16, 17, 18, 19, 20, 21, 22, 23, 24, or 25 nucleotides in length.

Typically, the first complementarity domain does not have exact complementarity with the second complementarity domain target. In some embodiments, the first complementarity domain can have 1, 2, 3, 4 or 5 nucleotides that are not complementary with the corresponding nucleotide of the second complementarity domain. For instance, a segment of 1, 2, 3, 4, 5 or 6, (e.g., 3) nucleotides of the first complementarity domain may not pair in the duplex, and may form a non-duplexed or looped-out region. In some instances, an unpaired, or loop-out, region, e.g., a loop-out of 3 nucleotides, is present on the second complementarity domain. This unpaired region optionally begins 1, 2, 3, 4, 5, or 6, e.g., 4, nucleotides from the 5′ end of the second complementarity domain.

The first complementarity domain can include 3 subdomains, which, in the 5′ to 3′ direction are: a 5′ subdomain, a central subdomain, and a 3′ subdomain. In an embodiment, the 5′ subdomain is 4-9, e.g., 4, 5, 6, 7, 8 or 9 nucleotides in length. In an embodiment, the central subdomain is 1, 2, or 3, e.g., 1, nucleotide in length. In an embodiment, the 3′ subdomain is 3 to 25, e.g., 4-22, 4-18, or 4 to 10, or 3, 4, 5, 6, 7, 8, 9, 10, 11, 12, 13, 14, 15, 16, 17, 18, 19, 20, 21, 22, 23, 24, or 25, nucleotides in length.

In some embodiments, the first and second complementarity domains, when duplexed, comprise 11 paired nucleotides, for example, in the gRNA sequence (one paired strand underlined, one bolded):

(SEQ ID NO: 5)

NNNNNNNNNNNNNNNNNNNNGUUUUAGAGCUAGAAAUAGCAAGUUAAAAU
AAGGCUAGUCCGUUAUCAACUUGAAAAAGUGGCACCGAGUCGGUGC.

In some embodiments, the first and second complementarity domains, when duplexed, comprise 15 paired nucleotides, for example in the gRNA sequence (one paired strand underlined, one bolded):

(SEQ ID NO: 27)

NNNNNNNNNNNNNNNNNNNNGUUUUAGAGCUAUGCUGAAAAGCAUAGCAA
GUUAAAAUAAGGCUAGUCCGUUAUCAACUUGAAAAAGUGGCACCGAGUCG
GUGC.

In some embodiments the first and second complementarity domains, when duplexed, comprise 16 paired nucleotides, for example in the gRNA sequence (one paired strand underlined, one bolded):

(SEQ ID NO: 28)

NNNNNNNNNNNNNNNNNNNNGUUUUAGAGCUAUGCUGGAAACAGCAUAGC
AAGUUAAAAUAAGGCUAGUCCGUUAUCAACUUGAAAAAGUGGCACCGAGU
CGGUGC.

In some embodiments the first and second complementarity domains, when duplexed, comprise 21 paired nucleotides, for example in the gRNA sequence (one paired strand underlined, one bolded):

(SEQ ID NO: 29)

NNNNNNNNNNNNNNNNNNNNGUUUUAGAGCUAUGCUGUUUUGGAAACAAA
ACAGCAUAGCAAGUUAAAAUAAGGCUAGUCCGUUAUCAACUUGAAAAAGU
GGCACCGAGUCGGUGC.

In some embodiments, nucleotides are exchanged to remove poly-U tracts, for example in the gRNA sequences (exchanged nucleotides underlined):

(SEQ ID NO: 30)

NNNNNNNNNNNNNNNNNNNNGUAUUAGAGCUAGAAAUAGCAAGUUAAUAU
AAGGCUAGUCCGUUAUCAACUUGAAAAAGUGGCACCGAGUCGGUGC;

(SEQ ID NO: 31)

NNNNNNNNNNNNNNNNNNNNGUUUAAGAGCUAGAAAUAGCAAGUUUAAAU
AAGGCUAGUCCGUUAUCAACUUGAAAAAGUGGCACCGAGUCGGUGC;
and

(SEQ ID NO: 32)

NNNNNNNNNNNNNNNNNNNNGUAUUAGAGCUAUGCUGUAUUGGAAACAAU
ACAGCAUAGCAAGUUAAUAUAAGGCUAGUCCGUUAUCAACUUGAAAAAGU
GGCACCGAGUCGGUGC.

The first complementarity domain can share homology with, or be derived from, a naturally occurring first complementarity domain. In an embodiment, it has at least 50% homology with a first complementarity domain disclosed herein, e.g., an S. pyogenes, S. aureus, N. meningtidis, or S. thermophilus, first complementarity domain.

It should be noted that one or more, or even all of the nucleotides of the first complementarity domain, can have a modification along the lines discussed above for the targeting domain.

c) The Linking Domain

FIGS. 1A-1G provide examples of linking domains.

In a unimolecular or chimeric gRNA, the linking domain serves to link the first complementarity domain with the second complementarity domain of a unimolecular gRNA. The linking domain can link the first and second complementarity domains covalently or non-covalently. In an embodiment, the linkage is covalent. In an embodiment, the linking domain covalently couples the first and second complementarity domains, see, e.g., FIGS. 1B-1E. In an embodiment, the linking domain is, or comprises, a covalent bond interposed between the first complementarity domain and the second complementarity domain. Typically the linking domain comprises one or more, e.g., 2, 3, 4, 5, 6, 7, 8, 9, or 10 nucleotides, but in various embodiments the linker can be 20, 30, 40, 50 or even 100 nucleotides in length.

In modular gRNA molecules, the two molecules are associated by virtue of the hybridization of the complementarity domains and a linking domain may not be present. See e.g., FIG. 1A.

A wide variety of linking domains are suitable for use in unimolecular gRNA molecules. Linking domains can consist of a covalent bond, or be as short as one or a few nucleotides, e.g., 1, 2, 3, 4, or 5 nucleotides in length. In an embodiment, a linking domain is 2, 3, 4, 5, 6, 7, 8, 9, 10, 15, 20, or 25 or more nucleotides in length. In an embodiment, a linking domain is 2 to 50, 2 to 40, 2 to 30, 2 to 20, 2 to 10, or 2 to 5 nucleotides in length. In an embodiment, a linking domain shares homology with, or is derived from, a naturally occurring sequence, e.g., the sequence of a tracrRNA that is 5′ to the second complementarity domain. In an embodiment, the linking domain has at least 50% homology with a linking domain disclosed herein.

As discussed above in connection with the first complementarity domain, some or all of the nucleotides of the linking domain can include a modification.

d) The 5′ Extension Domain

In some cases, a modular gRNA can comprise additional sequence, 5′ to the second complementarity domain, referred to herein as the 5′ extension domain, see, e.g., FIG. 1A. In an embodiment, the 5′ extension domain is, 2-10, 2-9, 2-8, 2-7, 2-6, 2-5, or 2-4 nucleotides in length. In an embodiment, the 5′ extension domain is 2, 3, 4, 5, 6, 7, 8, 9, or 10 or more nucleotides in length.

e) The Second Complementarity Domain

FIG. 1A-1G provide examples of second complementarity domains. The second complementarity domain is complementary with the first complementarity domain, and generally has sufficient complementarity to the second complementarity domain to form a duplexed region under at least some physiological conditions. In some cases, e.g., as shown in FIG. 1A-1B, the second complementarity domain can include sequence that lacks complementarity with the first complementarity domain, e.g., sequence that loops out from the duplexed region.

The second complementarity domain may be 5 to 27 nucleotides in length, and in some cases may be longer than the first complementarity region. For instance, the second complementary domain can be 7 to 27 nucleotides in length, 7 to 25 nucleotides in length, 7 to 20 nucleotides in length, or 7 to 17 nucleotides in length. More generally, the complementary domain may be 5, 6, 7, 8, 9, 10, 11, 12, 13, 14, 15, 16, 17, 18, 19, 20, 21, 22, 23, 24, 25, or 26 nucleotides in length.

In an embodiment, the second complementarity domain comprises 3 subdomains, which, in the 5′ to 3′ direction are: a 5′ subdomain, a central subdomain, and a 3′ subdomain. In an embodiment, the 5′ subdomain is 3 to 25, e.g., 4 to 22, 4 to18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In an embodiment, the central subdomain is 1, 2, 3, 4 or 5, e.g., 3, nucleotides in length. In an embodiment, the 3′ subdomain is 4 to 9, e.g., 4, 5, 6, 7, 8 or 9 nucleotides in length.

In an embodiment, the 5′ subdomain and the 3′ subdomain of the first complementarity domain, are respectively, complementary, e.g., fully complementary, with the 3′ subdomain and the 5′ subdomain of the second complementarity domain.

The second complementarity domain can share homology with or be derived from a naturally occurring second complementarity domain. In an embodiment, it has at least 50% homology with a second complementarity domain disclosed herein, e.g., an S. pyogenes, S. aureus, N. meningtidis, or S. thermophilus, first complementarity domain.

Some or all of the nucleotides of the second complementarity domain can have a modification, e.g., a modification found in Section VIII herein.

f) The Proximal Domain

FIGS. 1A-1G provide examples of proximal domains.

In an embodiment, the proximal domain is 5 to 20 nucleotides in length. In an embodiment, the proximal domain can share homology with or be derived from a naturally occurring proximal domain. In an embodiment, it has at least 50% homology with a proximal domain disclosed herein, e.g., an S. pyogenes, S. aureus, N. meningtidis, or S. thermophilus, proximal domain.

Some or all of the nucleotides of the proximal domain can have a modification along the lines described above.

g) The Tail Domain

FIGS. 1A-1G provide examples of tail domains.

As can be seen by inspection of the tail domains in FIG. 1A and FIGS. 1B-1F, a broad spectrum of tail domains are suitable for use in gRNA molecules. In various embodiments, the tail domain is 0 (absent), 1, 2, 3, 4, 5, 6, 7, 8, 9, or 10 nucleotides in length. In certain embodiments, the tail domain nucleotides are from or share homology with sequence from the 5′ end of a naturally occurring tail domain, see e.g., FIG. 1D or 1E. The tail domain also optionally includes sequences that are complementary to each other and which, under at least some physiological conditions, form a duplexed region.

Tail domains can share homology with or be derived from naturally occurring proximal tail domains. By way of non-limiting example, a given tail domain according to various embodiments of the present disclosure may share at least 50% homology with a naturally occurring tail domain disclosed herein, e.g., an S. pyogenes, S. aureus, N. meningtidis, or S. thermophilus, tail domain.

In certain cases, the tail domain includes nucleotides at the 3′ end that are related to the method of in vitro or in vivo transcription. When a T7 promoter is used for in vitro transcription of the gRNA, these nucleotides may be any nucleotides present before the 3′ end of the DNA template. When a U6 promoter is used for in vivo transcription, these nucleotides may be the sequence UUUUUU. When alternate pol-III promoters are used, these nucleotides may be various numbers or uracil bases or may include alternate bases.

As a non-limiting example, in various embodiments the proximal and tail domain, taken together comprise the following sequences:

(SEQ ID NO: 33)

AAGGCUAGUCCGUUAUCAACUUGAAAAAGUGGCACCGAGUCGGUGCU,

(SEQ ID NO: 34)

AAGGCUAGUCCGUUAUCAACUUGAAAAAGUGGCACCGAGUCGGUGGUGC,

(SEQ ID NO: 35)

AAGGCUAGUCCGUUAUCAACUUGAAAAAGUGGCACCGAGUCGGUGCGG
AUC,

(SEQ ID NO: 36)

AAGGCUAGUCCGUUAUCAACUUGAAAAAGUG,

(SEQ ID NO: 37)

AAGGCUAGUCCGUUAUCA,
or

(SEQ ID NO: 38)

AAGGCUAGUCCG.

In an embodiment, the tail domain comprises the 3′ sequence UUUUUU, e.g., if a U6 promoter is used for transcription.

In an embodiment, the tail domain comprises the 3′ sequence UUUU, e.g., if an H1 promoter is used for transcription.

In an embodiment, tail domain comprises variable numbers of 3′ Us depending, e.g., on the termination signal of the pol-III promoter used.

In an embodiment, the tail domain comprises variable 3′ sequence derived from the DNA template if a T7 promoter is used.

In an embodiment, the tail domain comprises variable 3′ sequence derived from the DNA template, e.g., if in vitro transcription is used to generate the RNA molecule.

In an embodiment, the tail domain comprises variable 3′ sequence derived from the DNA template, e.g., if a pol-II promoter is used to drive transcription.

In an embodiment a gRNA has the following structure:

5′ [targeting domain]-[first complementarity domain]-[linking domain]-[second complementarity domain]-[proximal domain]-[tail domain]-3′

wherein, the targeting domain comprises a core domain and optionally a secondary domain, and is 10 to 50 nucleotides in length;

the first complementarity domain is 5 to 25 nucleotides in length and, In an embodiment has at least 50, 60, 70, 80, 85, 90, 95, 98 or 99% homology with a reference first complementarity domain disclosed herein;

the linking domain is 1 to 5 nucleotides in length;

the proximal domain is 5 to 20 nucleotides in length and, in an embodiment has at least 50, 60, 70, 80, 85, 90, 95, 98 or 99% homology with a reference proximal domain disclosed herein; and

the tail domain is absent or a nucleotide sequence is 1 to 50 nucleotides in length and, in an embodiment has at least 50, 60, 70, 80, 85, 90, 95, 98 or 99% homology with a reference tail domain disclosed herein.

h) Exemplary Chimeric gRNAs

In an embodiment, a unimolecular, or chimeric, gRNA comprises, preferably from 5′ to 3′: a targeting domain, e.g., comprising 15, 16, 17, 18, 19, 20, 21, 22, 23, 24, 25, or 26 nucleotides (which is complementary to a target nucleic acid); a first complementarity domain; a linking domain; a second complementarity domain (which is complementary to the first complementarity domain); a proximal domain; and a tail domain, wherein, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the sequence from (a), (b), or (c), has at least 60, 75, 80, 85, 90, 95, or 99% homology with the corresponding sequence of a naturally occurring gRNA, or with a gRNA described herein. In an embodiment, the proximal and tail domain, when taken together, comprise at least 15, 18, 20, 25, 30, 31, 35, 40, 45, 49, 50, or 53 nucleotides. In an embodiment, there are at least 15, 18, 20, 25, 30, 31, 35, 40, 45, 49, 50, or 53 nucleotides 3′ to the last nucleotide of the second complementarity domain. In an embodiment, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain. In an embodiment, the targeting domain comprises, has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

In an embodiment, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number: NNNNNNNNNNNNNNNNNNNNGUUUUAGAGCUAGAAAUAGCAAGUUAAAAUAAGGCUAG UCCGUUAUCAACUUGAAAAAGUGGCACCGAGUCGGUGCUUUUUU (SEQ ID NO:40). In an embodiment, the unimolecular, or chimeric, gRNA molecule is a S. pyogenes gRNA molecule.

In some embodiments, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number: NNNNNNNNNNNNNNNNNNNNGUUUUAGUACUCUGGAAACAGAAUCUACUAAAAC AAGGCAAAAUGCCGUGUUUAUCUCGUCAACUUGUUGGCGAGAUUUUUU (SEQ ID NO:41). In an embodiment, the unimolecular, or chimeric, gRNA molecule is a S. aureus gRNA molecule.

In some embodiments, the targeting domain in the exemplary chimeric gRNA is or comprises a sequence selected from any of SEQ ID NOS: 481-3748.

In some embodiments, the targeting domain in the exemplary chimeric gRNA is or comprises a sequence selected from any of GUCUGGGCGGUGCUACAACU (SEQ ID NO:508), GCCCUGGCCAGUCGUCU (SEQ ID NO: 514), CGUCUGGGCGGUGCUACAAC (SEQ ID NO:1533), UGUAGCACCGCCCAGACGAC (SEQ ID NO:579), CGACUGGCCAGGGCGCCUGU (SEQ ID NO:582) and CACCUACCUAAGAACCAUCC (SEQ ID NO:723). In some embodiments, the targeting domain is or comprises the sequence GUCUGGGCGGUGCUACAACU (SEQ ID NO:508). In some embodiments, the targeting domain is or comprises the sequence GCCCUGGCCAGUCGUCU (SEQ ID NO: 514). In some embodiments, the targeting domain is or comprises the sequence CGUCUGGGCGGUGCUACAAC (SEQ ID NO:1533). In some embodiments, the targeting domain is or comprises the sequence UGUAGCACCGCCCAGACGAC (SEQ ID NO:579). In some embodiments, the targeting domain is or comprises the sequence CGACUGGCCAGGGCGCCUGU (SEQ ID NO:582) and CACCUACCUAAGAACCAUCC (SEQ ID NO:723).

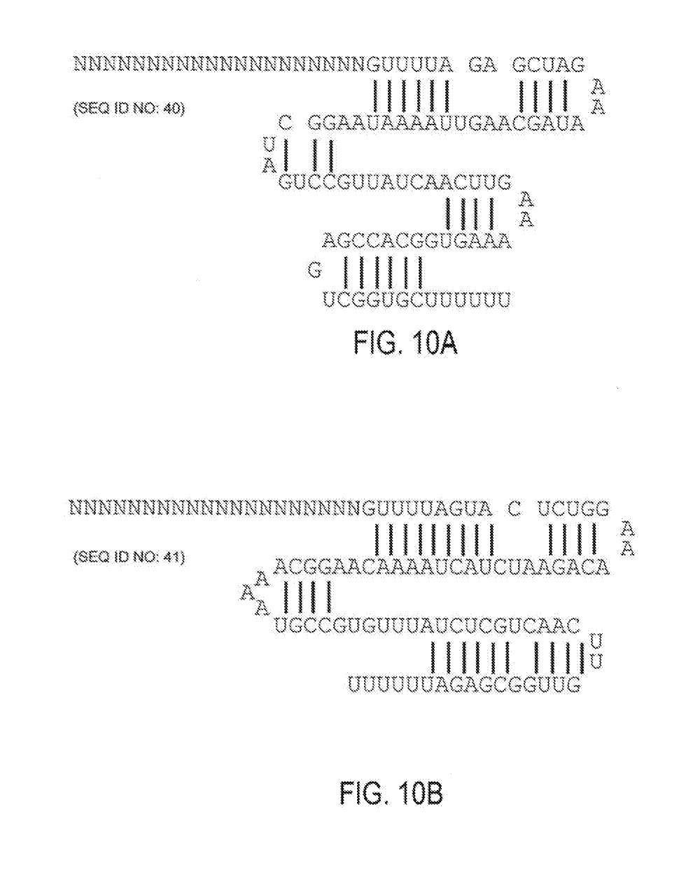

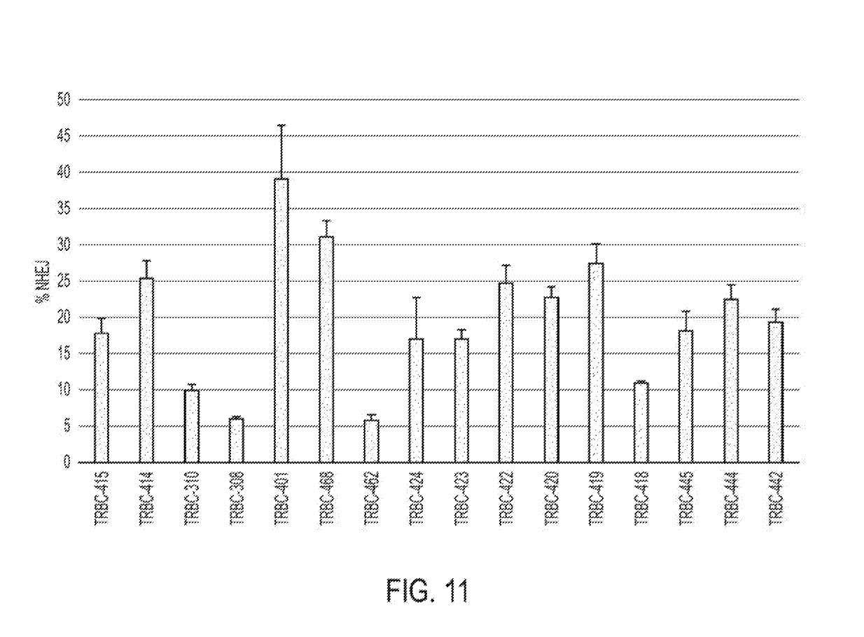

The sequences and structures of exemplary chimeric gRNAs are also shown in FIGS. 10A-10B.

i) Exemplary Modular gRNAs

In an embodiment, a modular gRNA comprises first and second strands. The first strand comprises, preferably from 5′ to 3′; a targeting domain, e.g., comprising 15, 16, 17, 18, 19, 20, 21, 22, 23, 24, 25, or 26 nucleotides; a first complementarity domain. The second strand comprises, preferably from 5′ to 3′: optionally a 5′ extension domain; a second complementarity domain; a proximal domain; and a tail domain, wherein: (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the sequence from (a), (b), or (c), has at least 60, 75, 80, 85, 90, 95, or 99% homology with the corresponding sequence of a naturally occurring gRNA, or with a gRNA described herein. In an embodiment, the proximal and tail domain, when taken together, comprise at least 15, 18, 20, 25, 30, 31, 35, 40, 45, 49, 50, or 53 nucleotides. In an embodiment there are at least 15, 18, 20, 25, 30, 31, 35, 40, 45, 49, 50, or 53 nucleotides 3′ to the last nucleotide of the second complementarity domain.

In an embodiment, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

In some embodiments, the targeting domain in the exemplary modular gRNA is or comprises a sequence selected from any of SEQ ID NOS: 481-3748.

In some embodiments, the targeting domain in the exemplary modular gRNA is or comprises a sequence selected from any of GUCUGGGCGGUGCUACAACU (SEQ ID NO:508), GCCCUGGCCAGUCGUCU (SEQ ID NO: 514), CGUCUGGGCGGUGCUACAAC (SEQ ID NO:1533), UGUAGCACCGCCCAGACGAC (SEQ ID NO:579), CGACUGGCCAGGGCGCCUGU (SEQ ID NO:582) and CACCUACCUAAGAACCAUCC (SEQ ID NO:723). In some embodiments, the targeting domain is or comprises the sequence GUCUGGGCGGUGCUACAACU (SEQ ID NO:508). In some embodiments, the targeting domain is or comprises the sequence GCCCUGGCCAGUCGUCU (SEQ ID NO: 514). In some embodiments, the targeting domain is or comprises the sequence CGUCUGGGCGGUGCUACAAC (SEQ ID NO:1533). In some embodiments, the targeting domain is or comprises the sequence UGUAGCACCGCCCAGACGAC (SEQ ID NO:579). In some embodiments, the targeting domain is or comprises the sequence CGACUGGCCAGGGCGCCUGU (SEQ ID NO:582) and CACCUACCUAAGAACCAUCC (SEQ ID NO:723).

2 Methods for Designing gRNAs

Methods for designing gRNAs are described herein, including methods for selecting, designing and validating targeting domains. Exemplary targeting domains are also provided herein. Targeting domains discussed herein can be incorporated into the gRNAs described herein.

Methods for selection and validation of target sequences as well as off-target analyses are described, e.g., in Mali et al., 2013 SCIENCE 339(6121): 823-826; Hsu et al. NAT BIOTECHNOL, 31(9): 827-32; Fu et al., 2014 NAT BIOTECHNOL, doi: 10.1038/nbt.2808. PubMed PMID: 24463574; Heigwer et al., 2014 NAT METHODS 11(2):122-3. doi: 10.1038/nmeth.2812. PubMed PMID: 24481216; Bae et al., 2014 BIOINFORMATICS PubMed PMID: 24463181; Xiao A et al., 2014 BIOINFORMATICS PubMed PMID: 24389662.

In some embodiments, a software tool can be used to optimize the choice of gRNA within a user's target sequence, e.g., to minimize total off-target activity across the genome. Off target activity may be other than cleavage. For example, for each possible gRNA choice using S. pyogenes Cas9, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the least off-target cleavage. Other functions, e.g., automated reagent design for gRNA vector construction, primer design for the on-target Surveyor assay, and primer design for high-throughput detection and quantification of off-target cleavage via next-generation sequencing, can also be included in the tool. Candidate gRNA molecules can be evaluated by art-known methods or as described herein.

In some embodiments, gRNAs for use with S. pyogenes, S. aureus, and N. meningitidis Cas9s are identified using a DNA sequence searching algorithm, e.g., using a custom gRNA design software based on the public tool cas-offinder (Bae et al. Bioinformatics. 2014; 30(10): 1473-1475). The custom gRNA design software scores guides after calculating their genome-wide off-target propensity. Typically matches ranging from perfect matches to 7 mismatches are considered for guides ranging in length from 17 to 24. In some aspects, once the off-target sites are computationally determined, an aggregate score is calculated for each guide and summarized in a tabular output using a web-interface. In addition to identifying potential gRNA sites adjacent to PAM sequences, the software also can identify all PAM adjacent sequences that differ by 1, 2, 3 or more nucleotides from the selected gRNA sites. In some embodiments, gGenomic DNA sequences for each gene are obtained from the UCSC Genome browser and sequences can be screened for repeat elements using the publicly available RepeatMasker program. RepeatMasker searches input DNA sequences for repeated elements and regions of low complexity. The output is a detailed annotation of the repeats present in a given query sequence.

Following identification, gRNAs can be ranked into tiers based on one or more of their distance to the target site, their orthogonality and presence of a 5′ G (based on identification of close matches in the human genome containing a relevant PAM, e.g., in the case of S. pyogenes, a NGG PAM, in the case of S. aureus, NNGRR (e.g, a NNGRRT or NNGRRV) PAM, and in the case of N. meningtidis, a NNNNGATT or NNNNGCTT PAM). Orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality are selected to minimize off-target DNA cleavage. It is to be understood that this is a non-limiting example and that a variety of strategies could be utilized to identify gRNAs for use with S. pyogenes, S. aureus and N. meningitidis or other Cas9 enzymes.

In some embodiments, gRNAs for use with the S. pyogenes Cas9 can be identified using the publicly available web-based ZiFiT server (Fu et al., Improving CRISPR-Cas nuclease specificity using truncated guide RNAs. Nat Biotechnol. 2014 Jan. 26. doi: 10.1038/nbt.2808. PubMed PMID: 24463574, for the original references see Sander et al., 2007, NAR 35:W599-605; Sander et al., 2010, NAR 38: W462-8). In addition to identifying potential gRNA sites adjacent to PAM sequences, the software also identifies all PAM adjacent sequences that differ by 1, 2, 3 or more nucleotides from the selected gRNA sites. In some aspects, genomic DNA sequences for each gene can be obtained from the UCSC Genome browser and sequences can be screened for repeat elements using the publicly available Repeat-Masker program. RepeatMasker searches input DNA sequences for repeated elements and regions of low complexity. The output is a detailed annotation of the repeats present in a given query sequence.

Following identification, gRNAs for use with a S. pyogenes Cas9 can be ranked into tiers, e.g. into 5 tiers. In some embodiments, the targeting domains for first tier gRNA molecules are selected based on their distance to the target site, their orthogonality and presence of a 5′ G (based on the ZiFiT identification of close matches in the human genome containing an NGG PAM). In some embodiments, both 17-mer and 20-mer gRNAs are designed for targets. In some aspects, gRNAs are also selected both for single-gRNA nuclease cutting and for the dual gRNA nickase strategy. Criteria for selecting gRNAs and the determination for which gRNAs can be used for which strategy can be based on several considerations. In some embodiments, gRNAs for both single-gRNA nuclease cleavage and for a dual-gRNA paired “nickase” strategy are identified. In some embodiments for selecting gRNAs, including the determination for which gRNAs can be used for the dual-gRNA paired “nickase” strategy, gRNA pairs should be oriented on the DNA such that PAMs are facing out and cutting with the D10A Cas9 nickase will result in 5′ overhangs. In some aspects, it can be assumed that cleaving with dual nickase pairs will result in deletion of the entire intervening sequence at a reasonable frequency. However, cleaving with dual nickase pairs can also often result in indel mutations at the site of only one of the gRNAs. Candidate pair members can be tested for how efficiently they remove the entire sequence versus just causing indel mutations at the site of one gRNA.

In some embodiments, the targeting domains for first tier gRNA molecules can be selected based on (1) a reasonable distance to the target position, e.g., within the first 500 bp of coding sequence downstream of start codon, (2) a high level of orthogonality, and (3) the presence of a 5′ G. In some embodiments, for selection of second tier gRNAs, the requirement for a 5′G can be removed, but the distance restriction is required and a high level of orthogonality was required. In some embodiments, third tier selection uses the same distance restriction and the requirement for a 5′G, but removes the requirement of good orthogonality. In some embodiments, fourth tier selection uses the same distance restriction but removes the requirement of good orthogonality and start with a 5′G. In some embodiments, fifth tier selection removes the requirement of good orthogonality and a 5′G, and a longer sequence (e.g., the rest of the coding sequence, e.g., additional 500 bp upstream or downstream to the transcription target site) is scanned. In certain instances, no gRNA is identified based on the criteria of the particular tier.

In some emobdiments, gRNAs are identified for single-gRNA nuclease cleavage as well as for a dual-gRNA paired “nickase” strategy.

In some aspects, gRNAs for use with the N. meningitidis and S. aureus Cas9s can be identified manually by scanning genomic DNA sequence for the presence of PAM sequences. These gRNAs canbe separated into two tiers. In some embodiments, for first tier gRNAs, targeting domains are selected within the first 500 bp of coding sequence downstream of start codon. In some embodiments, for second tier gRNAs, targeting domains are selected within the remaining coding sequence (downstream of the first 500 bp). In certain instances, no gRNA is identified based on the criteria of the particular tier.

In some embodiments, another strategy for identifying guide RNAs (gRNAs) for use with S. pyogenes, S. aureus and N. meningtidis Cas9s can use a DNA sequence searching algorithm. In some aspects, guide RNA design is carried out using a custom guide RNA design software based on the public tool cas-offinder (reference:Cas-OFFinder: a fast and versatile algorithm that searches for potential off-target sites of Cas9 RNA-guided endonucleases, Bioinformatics. 2014 Feb. 17. Bae S, Park J, Kim J S. PMID:24463181). Said custom guide RNA design software scores guides after calculating their genomewide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential gRNA sites adjacent to PAM sequences, the software also identifies all PAM adjacent sequences that differ by 1, 2, 3 or more nucleotides from the selected gRNA sites. In some embodiments, genomic DNA sequence for each gene is obtained from the UCSC Genome browser and sequences are screened for repeat elements using the publically available RepeatMasker program. RepeatMasker searches input DNA sequences for repeated elements and regions of low complexity. The output is a detailed annotation of the repeats present in a given query sequence.

In some embodiments, following identification, gRNAs are ranked into tiers based on their distance to the target site or their orthogonality (based on identification of close matches in the human genome containing a relavant PAM, e.g., in the case of S. pyogenes, a NGG PAM, in the case of S. aureus, NNGRR (e.g, a NNGRRT or NNGRRV) PAM, and in the case of N. meningtidiss, a NNNNGATT or NNNNGCTT PAM. In some aspects, targeting domains with good orthogonality are selected to minimize off-target DNA cleavage.

As an example, for S. pyogenes and N. meningtidiss targets, 17-mer, or 20-mer gRNAs can be designed. As another example, for S. aureus targets, 18-mer, 19-mer, 20-mer, 21-mer, 22-mer, 23-mer and 24-mer gRNAs can be designed.

In some embodiments, gRNAs for both single-gRNA nuclease cleavage and for a dual-gRNA paired “nickase” strategy are identified. In some embodiments for selecting gRNAs, including the determination for which gRNAs can be used for the dual-gRNA paired “nickase” strategy, gRNA pairs should be oriented on the DNA such that PAMs are facing out and cutting with the D10A Cas9 nickase will result in 5′ overhangs. In some aspects, it can be assumed that cleaving with dual nickase pairs will result in deletion of the entire intervening sequence at a reasonable frequency. However, cleaving with dual nickase pairs can also often result in indel mutations at the site of only one of the gRNAs. Candidate pair members can be tested for how efficiently they remove the entire sequence versus just causing indel mutations at the site of one gRNA.

For designing knock out strategies, in some embodiments, the targeting domains for tier 1 gRNA molecules for S. pyogenes are selected based on their distance to the target site and their orthogonality (PAM is NGG). In some cases, the targeting domains for tier 1 gRNA molecules are selected based on (1) a reasonable distance to the target position, e.g., within the first 500 bp of coding sequence downstream of start codon and (2) a high level of orthogonality. In some aspects, for selection of tier 2 gRNAs, a high level of orthogonality is not required. In some cases, tier 3 gRNAs remove the requirement of good orthogonality and a longer sequence (e.g., the rest of the coding sequence) can be scanned. In certain instances, no gRNA is identified based on the criteria of the particular tier.

For designing knock out strategies, in some embodiments, the targeting domain for tier 1 gRNA molecules for N. meningtidis were selected within the first 500 bp of the coding sequence and had a high level of orthogonality. The targeting domain for tier 2 gRNA molecules for N. meningtidis were selected within the first 500 bp of the coding sequence and did not require high orthogonality. The targeting domain for tier 3 gRNA molecules for N. meningtidis were selected within a remainder of coding sequence downstream of the 500 bp. Note that tiers are non-inclusive (each gRNA is listed only once). In certain instances, no gRNA was identified based on the criteria of the particular tier.

For designing knock out strategies, in some embodiments, the targeting domain for tier 1 grNA molecules for S. aureus is selected within the first 500 bp of the coding sequence, has a high level of orthogonality, and contains a NNGRRT PAM. In some embodiments, the targeting domain for tier 2 grNA molecules for S. aureus is selected within the first 500 bp of the coding sequence, no level of orthogonality is required, and contains a NNGRRT PAM. In some embodiments, the targeting domain for tier 3 gRNA molecules for S. aureus are selected within the remainder of the coding sequence downstream and contain a NNGRRT PAM. In some embodiments, the targeting domain for tier 4 gRNA molecules for S. aureus are selected within the first 500 bp of the coding sequence and contain a NNGRRV PAM. In some embodiments, the targeting domain for tier 5 gRNA molecules for S. aureus are selected within the remainder of the coding sequence downstream and contain a NNGRRV PAM. In certain instances, no gRNA is identified based on the criteria of the particular tier.

For designing of gRNA molecules for knocking down strategies, in some embodiments, the targeting domain for tier 1 gRNA molecules for S. pyogenes are selected within the first 500 bp upstream and downstream of the transcription start site and have a high level of orthogonality. In some embodiments, the targeting domain for tier 2 gRNA molecules for S. pyogenes are selected within the first 500 bp upstream and downstream of the transcription start site and do not require high orthogonality. In some embodiments, the targeting domain for tier 3 gRNA molecules for S. pyogenes are selected within the additional 500 bp upstream and downstream of transcription start site (e.g., extending to 1 kb up and downstream of the transcription start site). In certain instances, no gRNA is identified based on the criteria of the particular tier.

For designing of gRNA molecules for knocking down strategies, in some embodiments, the targeting domain for tier 1 gRNA molecules for N. meningtidis are selected within the first 500 bp upstream and downstream of the transcription start site and have a high level of orthogonality. In some embodiments, the targeting domain for tier 2 gRNA molecules for N. meningtidis are selected within the first 500 bp upstream and downstream of the transcription start site and do not require high orthogonality. In some embodiments, the targeting domain for tier 3 gRNA molecules for N. meningtidis are selected within the additional 500 bp upstream and downstream of transcription start site (e.g., extending to 1 kb up and downstream of the transcription start site). In certain instances, no gRNA is identified based on the criteria of the particular tier.

For designing of gRNA molecules for knocking down strategies, in some embodiments, the targeting domain for tier 1 gRNA molecules for S. aureus are selected within 500 bp upstream and downstream of transcription start site, a high level of orthogonality and PAM is NNGRRT. In some embodiments, the targeting domain for tier 2 gRNA molecules for S. aureus are selected within 500 bp upstream and downstream of transcription start site, no orthogonality requirement and PAM is NNGRRT. In some embodiments, the targeting domain for tier 3 gRNA molecules for S. aureus are selected within the additional 500 bp upstream and downstream of transcription start site (e.g., extending to 1 kb up and downstream of the transcription start site) and PAM is NNGRRT. In some embodiments, the targeting domain for tier 4 gRNA molecules for S. aureus are selected within 500 bp upstream and downstream of transcription start site and PAM is NNGRRV. In some embodiments, the targeting domain for tier 5 gRNA molecules for S. aureus are selected within the additional 500 bp upstream and downstream of transcription start site (extending to 1 kb up and downstream of the transcription start site) and PAM is NNGRRV. In certain instances, no gRNA is identified based on the criteria of the particular tier.

3. Cas9

Cas9 molecules of a variety of species can be used in the methods and compositions described herein. While the S. pyogenes, S. aureus, N. meningitidis, and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes, S. aureus, N. meningitidis, and S. thermophilus Cas9 molecules, Cas9 molecules from the other species can replace them. Such species include: Acidovorax avenae, Actinobacillus pleuropneumoniae, Actinobacillus succinogenes, Actinobacillus suis, Actinomyces sp., Cycliphilusdenitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae.

A Cas9 molecule, or Cas9 polypeptide, as that term is used herein, refers to a molecule or polypeptide that can interact with a gRNA molecule and, in concert with the gRNA molecule, homes or localizes to a site which comprises a target domain and PAM sequence. Cas9 molecule and Cas9 polypeptide, as those terms are used herein, refer to naturally occurring Cas9 molecules and to engineered, altered, or modified Cas9 molecules or Cas9 polypeptides that differ, e.g., by at least one amino acid residue, from a reference sequence, e.g., the most similar naturally occurring Cas9 molecule or a sequence of Table 2A.

a) Cas9 Domains

Crystal structures have been determined for two different naturally occurring bacterial Cas9 molecules (Jinek et al., Science, 343(6176):1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/nature13579).

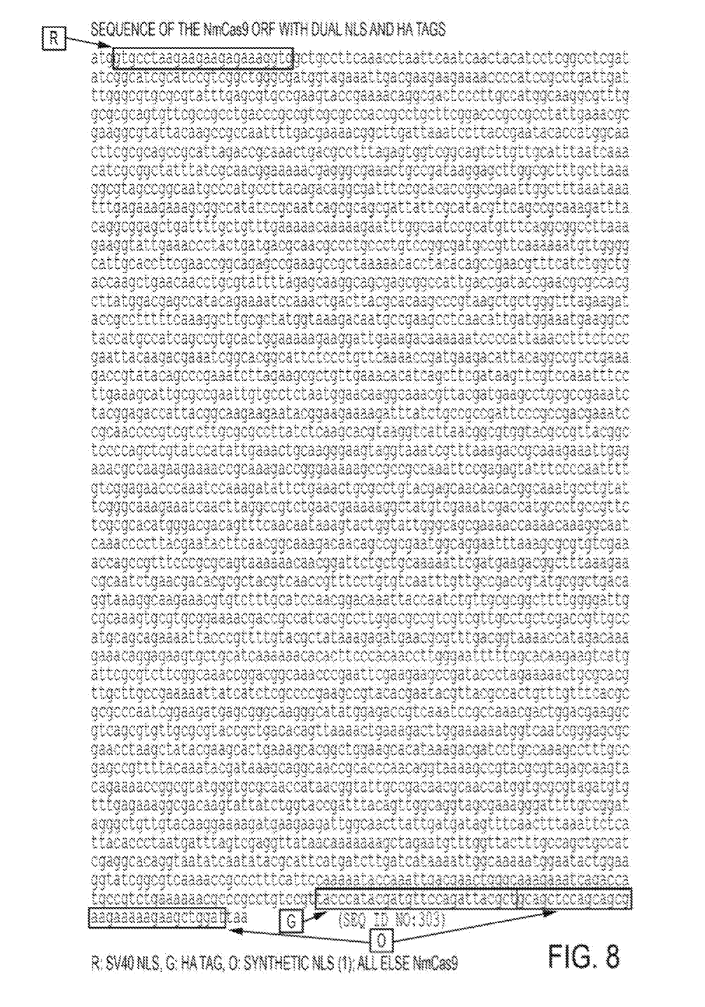

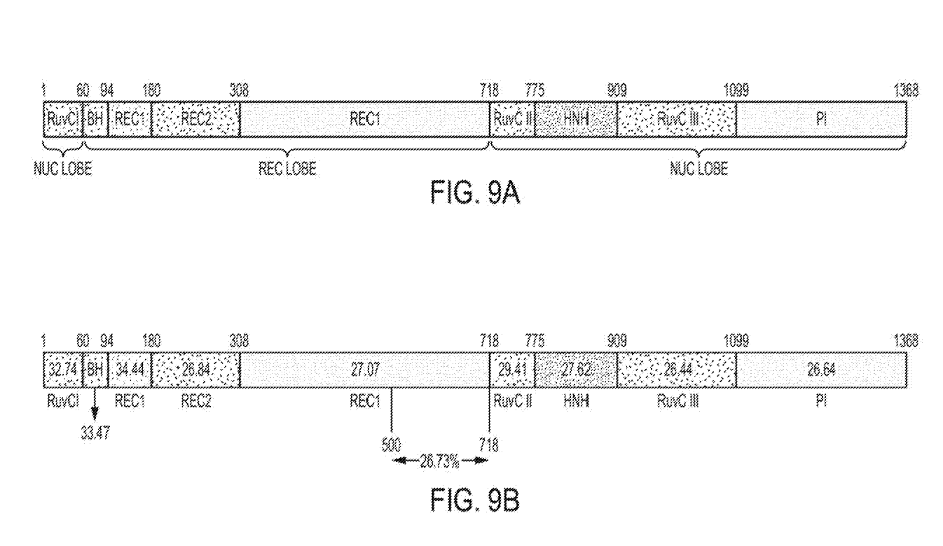

A naturally occurring Cas9 molecule comprises two lobes: a recognition (REC) lobe and a nuclease (NUC) lobe; each of which further comprises domains described herein. FIGS. 8A-8B provide a schematic of the organization of important Cas9 domains in the primary structure. The domain nomenclature and the numbering of the amino acid residues encompassed by each domain used throughout this disclosure is as described in Nishimasu et al. The numbering of the amino acid residues is with reference to Cas9 from S. pyogenes.

The REC lobe comprises the arginine-rich bridge helix (BH), the REC1 domain, and the REC2 domain. The REC lobe does not share structural similarity with other known proteins, indicating that it is a Cas9-specific functional domain. The BH domain is a long α-helix and arginine rich region and comprises amino acids 60-93 of the sequence of S. pyogenes Cas9. The REC1 domain is important for recognition of the repeat:anti-repeat duplex, e.g., of a gRNA or a tracrRNA, and is therefore critical for Cas9 activity by recognizing the target sequence. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat:anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule, and comprises amino acids 1099-1368 of the sequence of S. pyogenes Cas9.

(1) A RuvC-Like Domain and an HNH-Like Domain

In an embodiment, a Cas9 molecule or Cas9 polypeptide comprises an HNH-like domain and a RuvC-like domain. In an embodiment, cleavage activity is dependent on a RuvC-like domain and an HNH-like domain. A Cas9 molecule or Cas9 polypeptide, e.g., an eaCas9 molecule or eaCas9 polypeptide, can comprise one or more of the following domains: a RuvC-like domain and an HNH-like domain. In an embodiment, a Cas9 molecule or Cas9 polypeptide is an eaCas9 molecule or eaCas9 polypeptide and the eaCas9 molecule or eaCas9 polypeptide comprises a RuvC-like domain, e.g., a RuvC-like domain described below, and/or an HNH-like domain, e.g., an HNH-like domain described below.

(2) RuvC-Like Domains

In an embodiment, a RuvC-like domain cleaves, a single strand, e.g., the non-complementary strand of the target nucleic acid molecule. The Cas9 molecule or Cas9 polypeptide can include more than one RuvC-like domain (e.g., one, two, three or more RuvC-like domains). In an embodiment, a RuvC-like domain is at least 5, 6, 7, 8 amino acids in length but not more than 20, 19, 18, 17, 16 or 15 amino acids in length. In an embodiment, the Cas9 molecule or Cas9 polypeptide comprises an N-terminal RuvC-like domain of about 10 to 20 amino acids, e.g., about 15 amino acids in length.

(3) N-Terminal RuvC-Like Domains

Some naturally occurring Cas9 molecules comprise more than one RuvC-like domain with cleavage being dependent on the N-terminal RuvC-like domain. Accordingly, Cas9 molecules or Cas9 polypeptide can comprise an N-terminal RuvC-like domain. Exemplary N-terminal RuvC-like domains are described below.

In an embodiment, an eaCas9 molecule or eaCas9 polypeptide comprises an N-terminal RuvC-like domain comprising an amino acid sequence of formula I:

(SEQ ID NO: 8)

D-X1-G-X2-X3-X4-X5-G-X6-X7-X8-X9,

wherein,
X1 is selected from I, V, M, L and T (e.g., selected from I, V, and L);
X2 is selected from T, I, V, S, N, Y, E and L (e.g., selected from T, V, and I);
X3 is selected from N, S, G, A, D, T, R, M and F (e.g., A or N);
X4 is selected from S, Y, N and F (e.g., S);
X5 is selected from V, I, L, C, T and F (e.g., selected from V, I and L);
X6 is selected from W, F, V, Y, S and L (e.g., W);
X7 is selected from A, S, C, V and G (e.g., selected from A and S);
X8 is selected from V, I, L, A, M and H (e.g., selected from V, I, M and L); and
X9 is selected from any amino acid or is absent, designated by Δ (e.g., selected from T, V, I, L, Δ, F, S, A, Y, M and R, or, e.g., selected from T, V, I, L and Δ).

In an embodiment, the N-terminal RuvC-like domain differs from a sequence of SEQ ID NO:8, by as many as 1 but no more than 2, 3, 4, or 5 residues.

In embodiment, the N-terminal RuvC-like domain is cleavage competent.

In embodiment, the N-terminal RuvC-like domain is cleavage incompetent.

In an embodiment, a eaCas9 molecule or eaCas9 polypeptide comprises an N-terminal RuvC-like domain comprising an amino acid sequence of formula II:

(SEQ ID NO: 9)

D-X1-G-X2-X3-S-X5-G-X6-X7-X8-X9,

wherein
X1 is selected from I, V, M, L and T (e.g., selected from I, V, and L);
X2 is selected from T, I, V, S, N, Y, E and L (e.g., selected from T, V, and I);
X3 is selected from N, S, G, A, D, T, R, M and F (e.g., A or N);
X5 is selected from V, I, L, C, T and F (e.g., selected from V, I and L);
X6 is selected from W, F, V, Y, S and L (e.g., W);
X7 is selected from A, S, C, V and G (e.g., selected from A and S);
X8 is selected from V, I, L, A, M and H (e.g., selected from V, I, M and L); and
X9 is selected from any amino acid or is absent (e.g., selected from T, V, I, L, Δ, F, S, A, Y, M and R or selected from e.g., T, V, I, L and Δ).

In an embodiment, the N-terminal RuvC-like domain differs from a sequence of SEQ ID NO:9 by as many as 1 but no more than 2, 3, 4, or 5 residues.

In an embodiment, the N-terminal RuvC-like domain comprises an amino acid sequence of formula III:

(SEQ ID NO: 10)

D-I-G-X2-X3-S-V-G-W-A-X8-X9,

wherein
X2 is selected from T, I, V, S, N, Y, E and L (e.g., selected from T, V, and I);
X3 is selected from N, S, G, A, D, T, R, M and F (e.g., A or N);
X8 is selected from V, I, L, A, M and H (e.g., selected from V, I, M and L); and
X9 is selected from any amino acid or is absent (e.g., selected from T, V, I, L, A, F, S, A, Y, M and R or selected from e.g., T, V, I, L and A).

In an embodiment, the N-terminal RuvC-like domain differs from a sequence of SEQ ID NO:10 by as many as 1 but no more than, 2, 3, 4, or 5 residues.

In an embodiment, the N-terminal RuvC-like domain comprises an amino acid sequence of formula III:

(SEQ ID NO: 11)

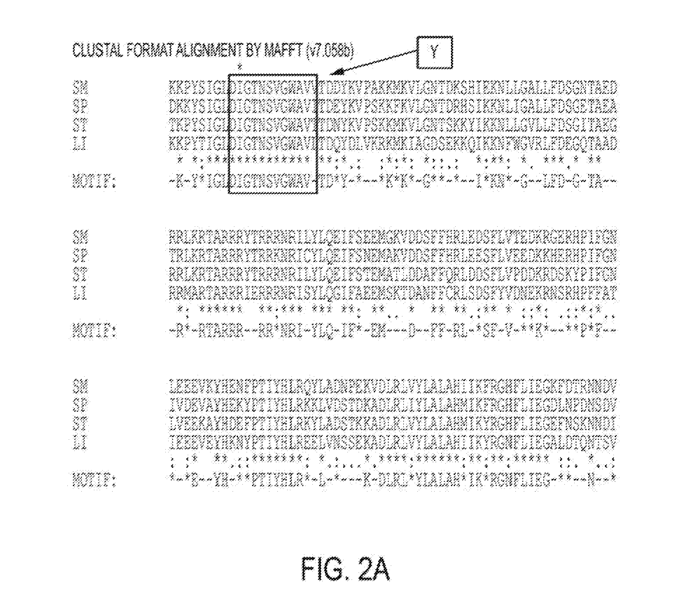

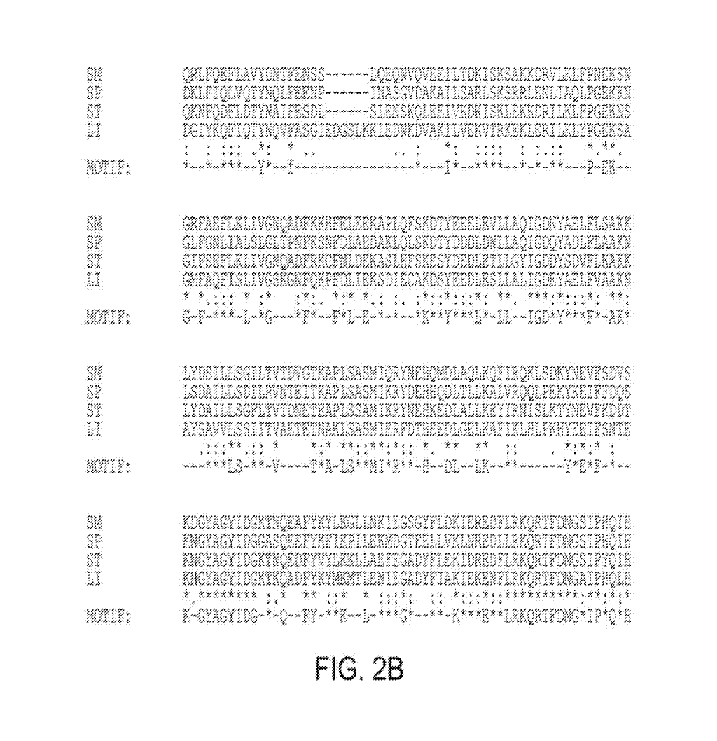

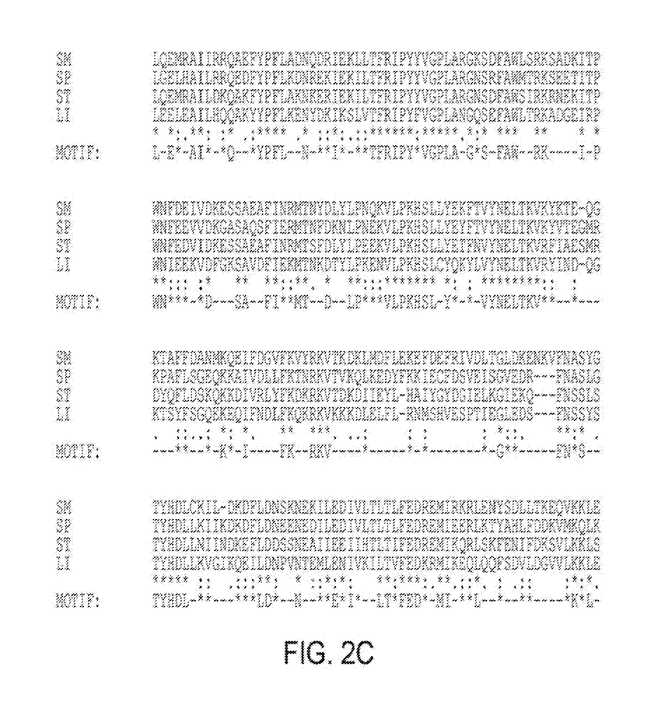

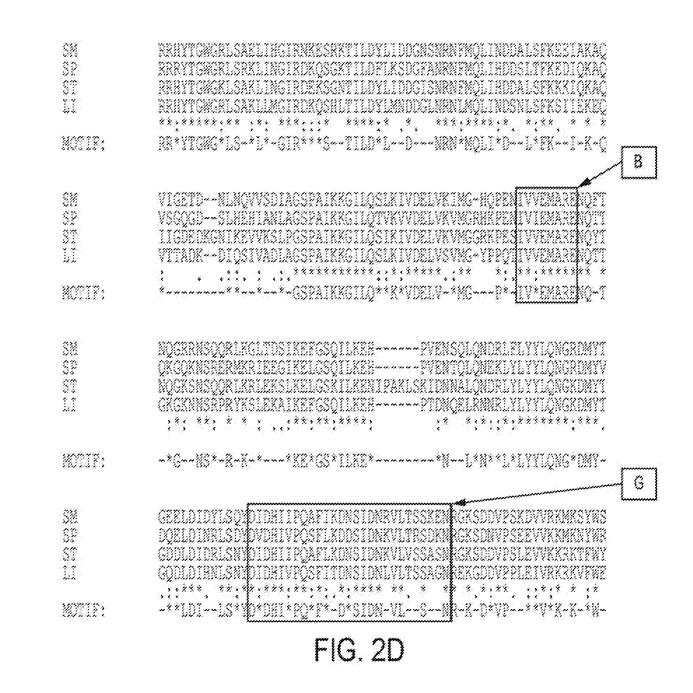

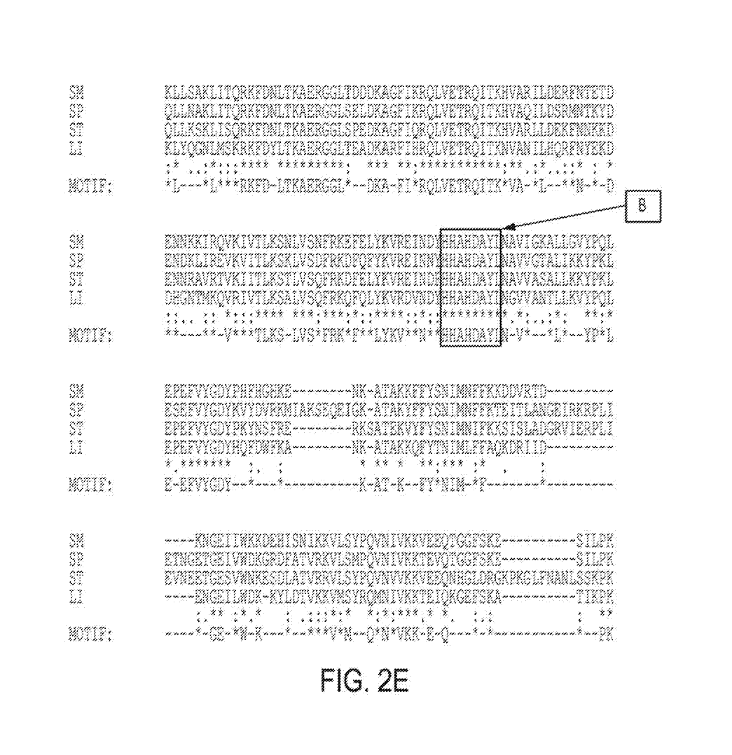

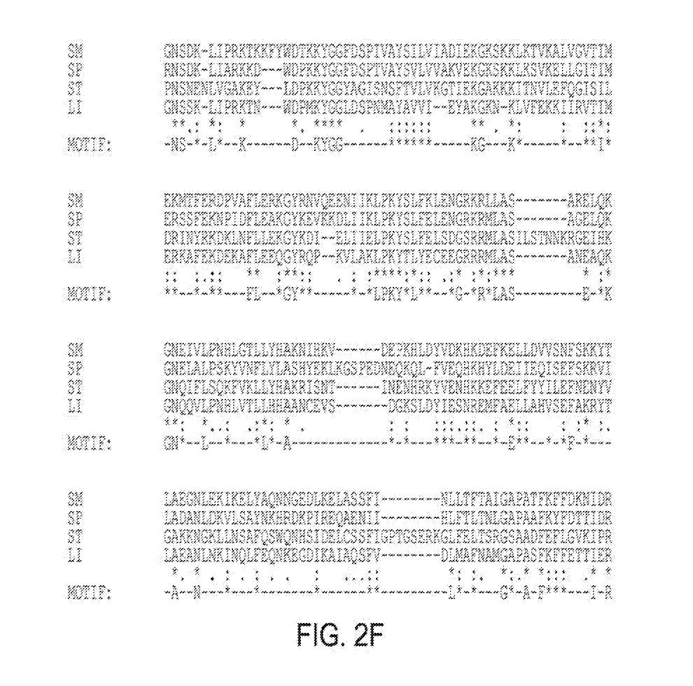

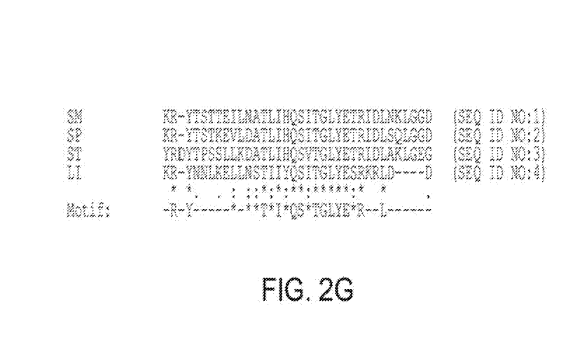

D-I-G-T-N-S-V-G-W-A-V-X,

wherein X is a non-polar alkyl amino acid or a hydroxyl amino acid, e.g., X is selected from V, I, L and T (e.g., the eaCas9 molecule can comprise an N-terminal RuvC-like domain shown in FIGS. 2A-2G (is depicted as Y)).

In an embodiment, the N-terminal RuvC-like domain differs from a sequence of SEQ ID NO:11 by as many as 1 but no more than, 2, 3, 4, or 5 residues.

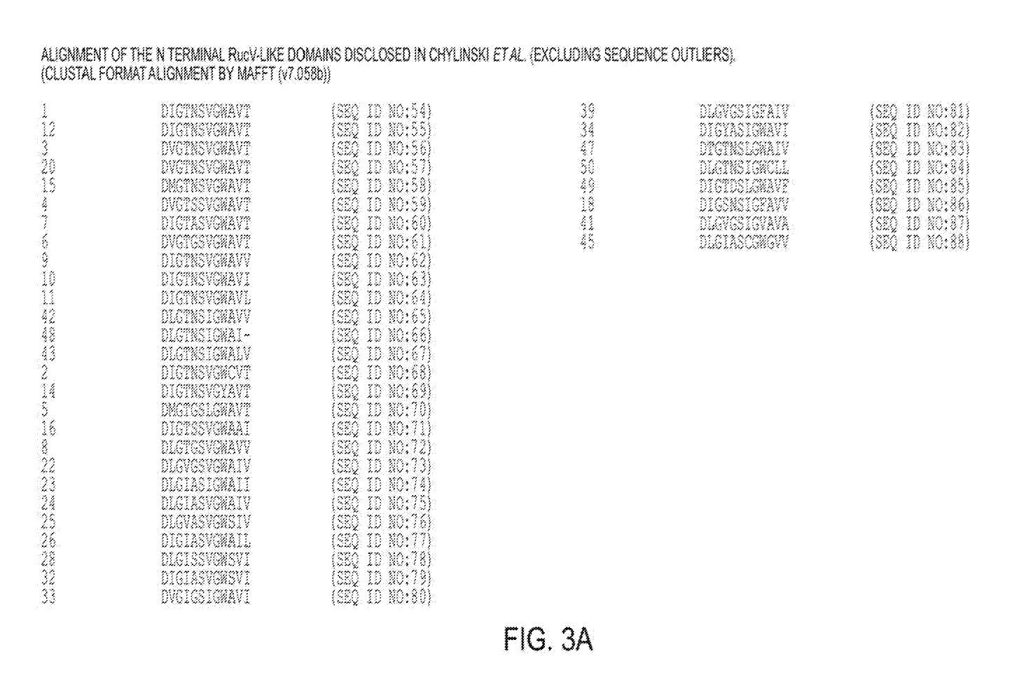

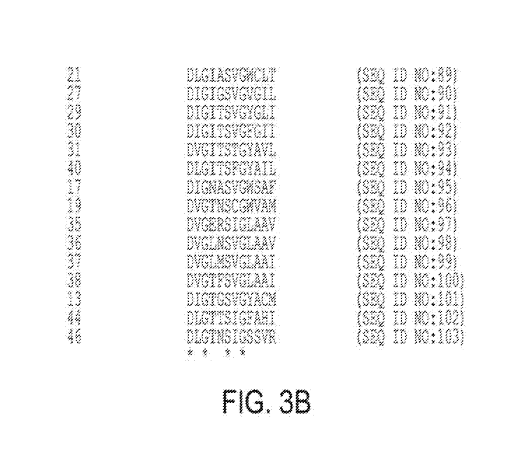

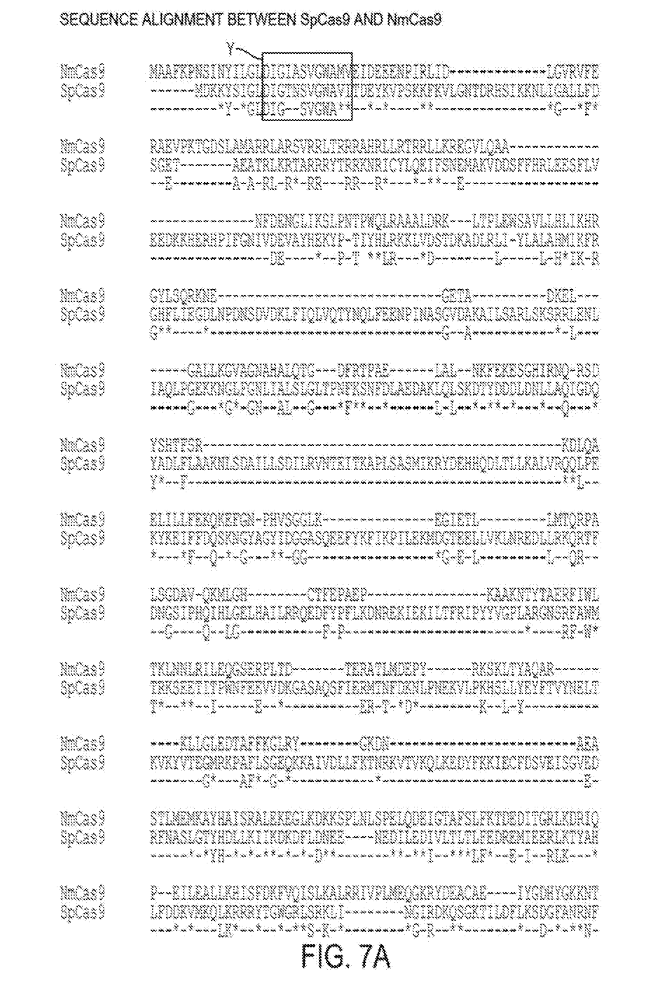

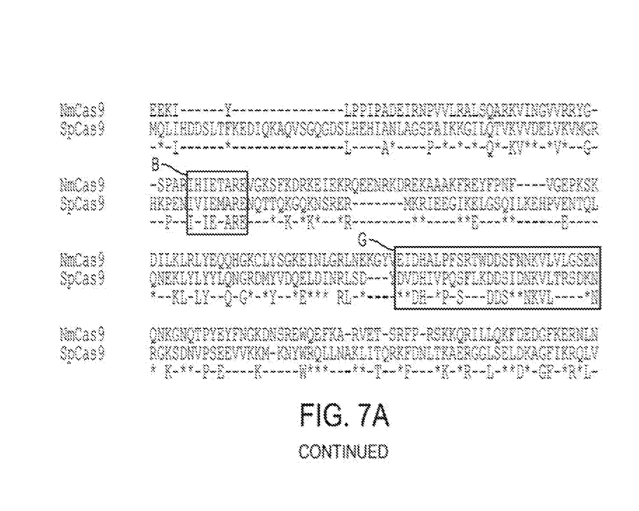

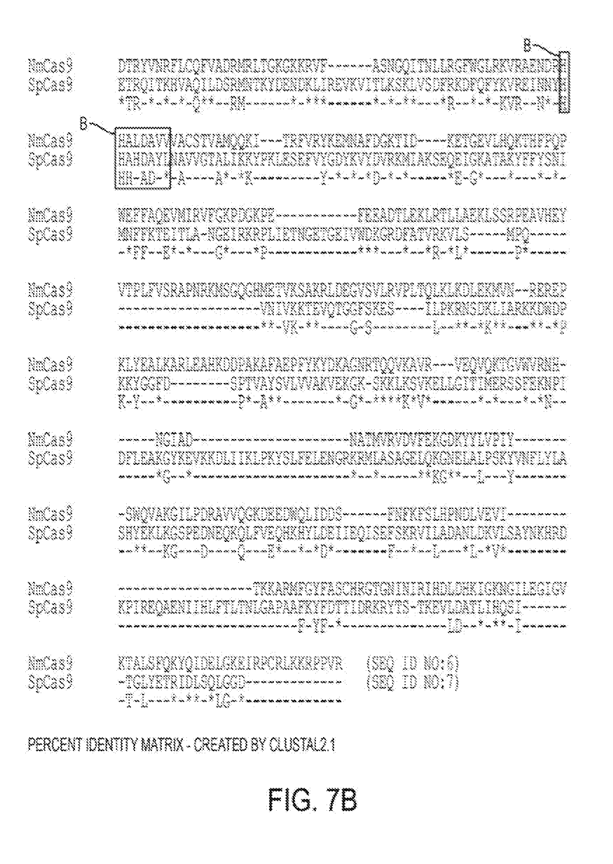

In an embodiment, the N-terminal RuvC-like domain differs from a sequence of an N-terminal RuvC like domain disclosed herein, e.g., in FIGS. 3A-3B or FIGS. 7A-7B, as many as 1 but no more than 2, 3, 4, or 5 residues. In an embodiment, 1, 2, or all 3 of the highly conserved residues identified in FIGS. 3A-3B or FIGS. 7A-7B are present.

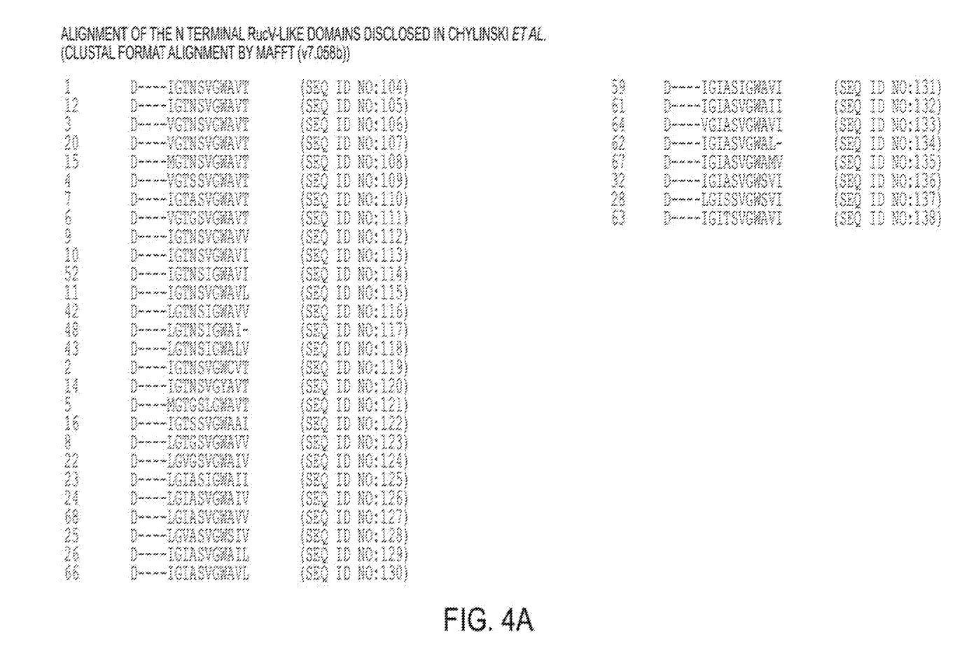

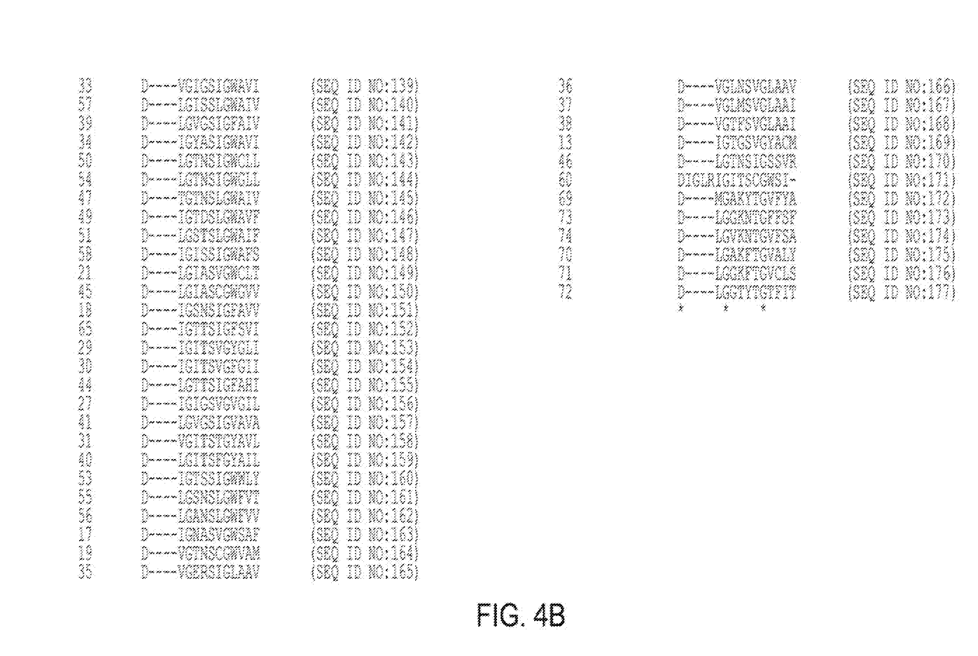

In an embodiment, the N-terminal RuvC-like domain differs from a sequence of an N-terminal RuvC-like domain disclosed herein, e.g., in FIGS. 4A-4B or FIGS. 7A-7B, as many as 1 but no more than 2, 3, 4, or 5 residues. In an embodiment, 1, 2, 3 or all 4 of the highly conserved residues identified in FIGS. 4A-4B or FIGS. 7A-7B are present.

(4) Additional RuvC-Like Domains

In addition to the N-terminal RuvC-like domain, the Cas9 molecule or Cas9 polypeptide, e.g., an eaCas9 molecule or eaCas9 polypeptide, can comprise one or more additional RuvC-like domains. In an embodiment, the Cas9 molecule or Cas9 polypeptide can comprise two additional RuvC-like domains. Preferably, the additional RuvC-like domain is at least 5 amino acids in length and, e.g., less than 15 amino acids in length, e.g., 5 to 10 amino acids in length, e.g., 8 amino acids in length.

An additional RuvC-like domain can comprise an amino acid sequence:

(SEQ ID NO: 12)

I-X1-X2-E-X3-A-R-E,

wherein
X1 is V or H,
X2 is I, L or V (e.g., I or V); and
X3 is M or T.

In an embodiment, the additional RuvC-like domain comprises the amino acid sequence:

(SEQ ID NO: 13)

I-V-X2-E-M-A-R-E,

wherein

X2 is I, L or V (e.g., I or V) (e.g., the eaCas9 molecule or eaCas9 polypeptide can comprise an additional RuvC-like domain shown in FIGS. 2A-2G or FIGS. 7A-7B (depicted as B)).

An additional RuvC-like domain can comprise an amino acid sequence:

(SEQ ID NO: 14)

H-H-A-X1-D-A-X2-X3,

wherein
X1 is H or L;
X2 is R or V; and
X3 is E or V.

In an embodiment, the additional RuvC-like domain comprises the amino acid sequence:

(SEQ ID NO: 15)

H-H-A-H-D-A-Y-L.

In an embodiment, the additional RuvC-like domain differs from a sequence of SEQ ID NO:12, 13, 14 or 15 by as many as 1 but no more than 2, 3, 4, or 5 residues.

In some embodiments, the sequence flanking the N-terminal RuvC-like domain is a sequence of formula V:

(SEQ ID NO: 16)

K-X1′-Y-X2′-X3′-X4′-Z-T-D-X9′-Y,

wherein
X1′ is selected from K and P,
X2′ is selected from V, L, I, and F (e.g., V, I and L);
X3′ is selected from G, A and S (e.g., G),
X4′ is selected from L, I, V and F (e.g., L);
X9′ is selected from D, E, N and Q; and
Z is an N-terminal RuvC-like domain, e.g., as described above.

(5) HNH-Like Domains

In an embodiment, an HNH-like domain cleaves a single stranded complementary domain, e.g., a complementary strand of a double stranded nucleic acid molecule. In an embodiment, an HNH-like domain is at least 15, 20, 25 amino acids in length but not more than 40, 35 or 30 amino acids in length, e.g., 20 to 35 amino acids in length, e.g., 25 to 30 amino acids in length. Exemplary HNH-like domains are described below.

In an embodiment, an eaCas9 molecule or eaCas9 polypeptide comprises an HNH-like domain having an amino acid sequence of formula VI:

(SEQ ID NO: 17)

X1-X2-X3-H-X4-X5-P-X6-X7-X8-X9-X10-X11-X12-X13-
X14-X15-N-X16-X17-X18-X19-X20-X21-X22-X23-N,

wherein
X1 is selected from D, E, Q and N (e.g., D and E);
X2 is selected from L, I, R, Q, V, M and K;
X3 is selected from D and E;
X4 is selected from I, V, T, A and L (e.g., A, I and V);
X5 is selected from V, Y, I, L, F and W (e.g., V, I and L);
X6 is selected from Q, H, R, K, Y, I, L, F and W;
X7 is selected from S, A, D, T and K (e.g., S and A);
X8 is selected from F, L, V, K, Y, M, I, R, A, E, D and Q (e.g., F);
X9 is selected from L, R, T, I, V, S, C, Y, K, F and G;
X10 is selected from K, Q, Y, T, F, L, W, M, A, E, G, and S;
X11 is selected from D, S, N, R, L and T (e.g., D);
X12 is selected from D, N and S;
X13 is selected from S, A, T, G and R (e.g., S);
X14 is selected from I, L, F, S, R, Y, Q, W, D, K and H (e.g., I, L and F);
X15 is selected from D, S, I, N, E, A, H, F, L, Q, M, G, Y and V;
X16 is selected from K, L, R, M, T and F (e.g., L, R and K);
X17 is selected from V, L, I, A and T;
X18 is selected from L, I, V and A (e.g., L and I);
X19 is selected from T, V, C, E, S and A (e.g., T and V);
X20 is selected from R, F, T, W, E, L, N, C, K, V, S, Q, I, Y, H and A;
X21 is selected from S, P, R, K, N, A, H, Q, G and L;
X22 is selected from D, G, T, N, S, K, A, I, E, L, Q, R and Y; and
X23 is selected from K, V, A, E, Y, I, C, L, S, T, G, K, M, D and F.

In an embodiment, a HNH-like domain differs from a sequence of SEQ ID NO:17 by at least one but no more than, 2, 3, 4, or 5 residues.

In an embodiment, the HNH-like domain is cleavage competent.

In an embodiment, the HNH-like domain is cleavage incompetent.

In an embodiment, an eaCas9 molecule or eaCas9 polypeptide comprises an HNH-like domain comprising an amino acid sequence of formula VII:

(SEQ ID NO: 18)

X1-X2-X3-H-X4-X5-P-X6-S-X8-X9-X10-D-D-S-X14-X15-
N-K-V-L-X19-X20-X21-X22-X23-N,

wherein
X1 is selected from D and E;
X2 is selected from L, I, R, Q, V, M and K;
X3 is selected from D and E;
X4 is selected from I, V, T, A and L (e.g., A, I and V);
X5 is selected from V, Y, I, L, F and W (e.g., V, I and L);
X6 is selected from Q, H, R, K, Y, I, L, F and W;
X8 is selected from F, L, V, K, Y, M, I, R, A, E, D and Q (e.g., F);
X9 is selected from L, R, T, I, V, S, C, Y, K, F and G;
X10 is selected from K, Q, Y, T, F, L, W, M, A, E, G, and S;

X14 is selected from I, L, F, S, R, Y, Q, W, D, K and H (e.g., I, L and F);
X15 is selected from D, S, I, N, E, A, H, F, L, Q, M, G, Y and V;
X19 is selected from T, V, C, E, S and A (e.g., T and V);
X20 is selected from R, F, T, W, E, L, N, C, K, V, S, Q, I, Y, H and A;
X21 is selected from S, P, R, K, N, A, H, Q, G and L;
X22 is selected from D, G, T, N, S, K, A, I, E, L, Q, R and Y; and
X23 is selected from K, V, A, E, Y, I, C, L, S, T, G, K, M, D and F.

In an embodiment, the HNH-like domain differs from a sequence of SEQ ID NO:18 by 1, 2, 3, 4, or 5 residues.

In an embodiment, an eaCas9 molecule or eaCas9 polypeptide comprises an HNH-like domain comprising an amino acid sequence of formula VII:

(SEQ ID NO: 19)

X1-V-X3-H-I-V-P-X6-S-X8-X9-X10-D-D-S-X14-X15-N-K-
V-L-T-X20-X21-X22-X23-N,

wherein
X1 is selected from D and E;
X3 is selected from D and E;
X6 is selected from Q, H, R, K, Y, I, L and W;
X8 is selected from F, L, V, K, Y, M, I, R, A, E, D and Q (e.g., F);
X9 is selected from L, R, T, I, V, S, C, Y, K, F and G;
X10 is selected from K, Q, Y, T, F, L, W, M, A, E, G, and S;
X14 is selected from I, L, F, S, R, Y, Q, W, D, K and H (e.g., I, L and F);
X15 is selected from D, S, I, N, E, A, H, F, L, Q, M, G, Y and V;
X20 is selected from R, F, T, W, E, L, N, C, K, V, S, Q, I, Y, H and A;
X21 is selected from S, P, R, K, N, A, H, Q, G and L;
X22 is selected from D, G, T, N, S, K, A, I, E, L, Q, R and Y; and
X23 is selected from K, V, A, E, Y, I, C, L, S, T, G, K, M, D and F.

In an embodiment, the HNH-like domain differs from a sequence of SEQ ID NO:19 by 1, 2, 3, 4, or 5 residues.

In an embodiment, an eaCas9 molecule or eaCas9 polypeptide comprises an HNH-like domain having an amino acid sequence of formula VIII:

(SEQ ID NO: 20)

D-X2-D-H-I-X5-P-Q-X7-F-X9-X10-D-X12-S-I-D-N-X16-V-
L-X19-X20-S-X22-X23-N,

wherein
X2 is selected from I and V;
X5 is selected from I and V;
X7 is selected from A and S;
X9 is selected from I and L;
X10 is selected from K and T;
X12 is selected from D and N;
X16 is selected from R, K and L; X19 is selected from T and V;
X20 is selected from S and R;
X22 is selected from K, D and A; and
X23 is selected from E, K, G and N (e.g., the eaCas9 molecule or eaCas9 polypeptide can comprise an HNH-like domain as described herein).

In an embodiment, the HNH-like domain differs from a sequence of SEQ ID NO:20 by as many as 1 but no more than 2, 3, 4, or 5 residues.

In an embodiment, an eaCas9 molecule or eaCas9 polypeptide comprises the amino acid sequence of formula IX:

(SEQ ID NO: 21)

L-Y-Y-L-Q-N-G-X1′-D-M-Y-X2′-X3′-X4′-X5′-L-D-I-X6′-
X7′-L-S-X8′-Y-Z-N-R-X9′-K-X10′-D-X11′-V-P,

wherein
X1′ is selected from K and R;
X2′ is selected from V and T;
X3′ is selected from G and D;
X4′ is selected from E, Q and D;
X5′ is selected from E and D;
X6′ is selected from D, N and H;
X7′ is selected from Y, R and N;
X8′ is selected from Q, D and N; X9′ is selected from G and E;
X10′ is selected from S and G;
X11′ is selected from D and N; and
Z is an HNH-like domain, e.g., as described above.

In an embodiment, the eaCas9 molecule or eaCas9 polypeptide comprises an amino acid sequence that differs from a sequence of SEQ ID NO:21 by as many as 1 but no more than 2, 3, 4, or 5 residues.

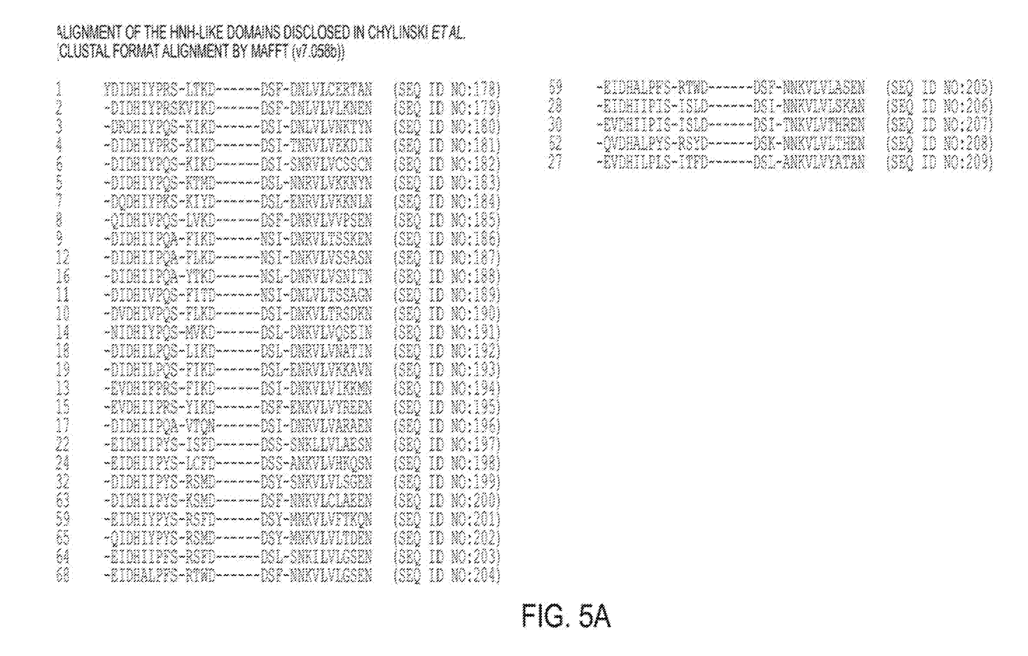

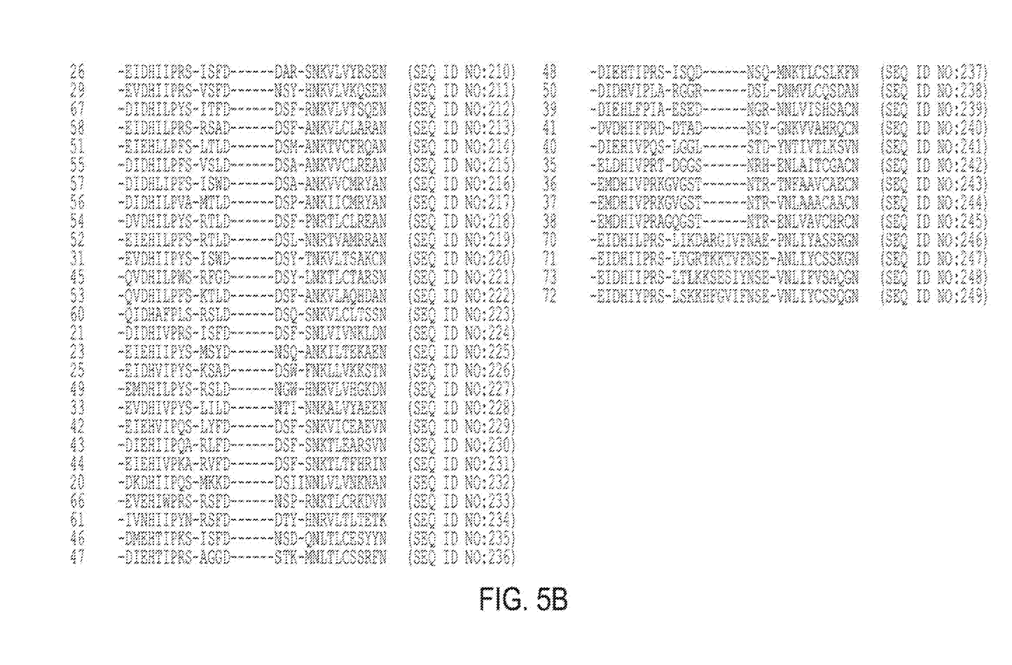

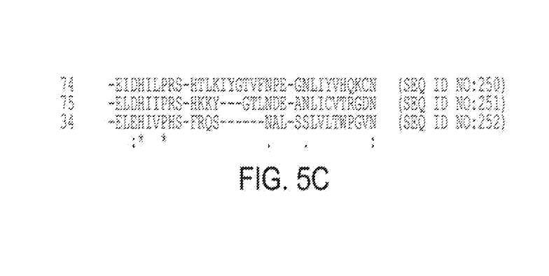

In an embodiment, the HNH-like domain differs from a sequence of an HNH-like domain disclosed herein, e.g., in FIGS. 5A-5C or FIGS. 7A-7B, as many as 1 but no more than 2, 3, 4, or 5 residues. In an embodiment, 1 or both of the highly conserved residues identified in FIGS. 5A-5C or FIGS. 7A-7B are present.

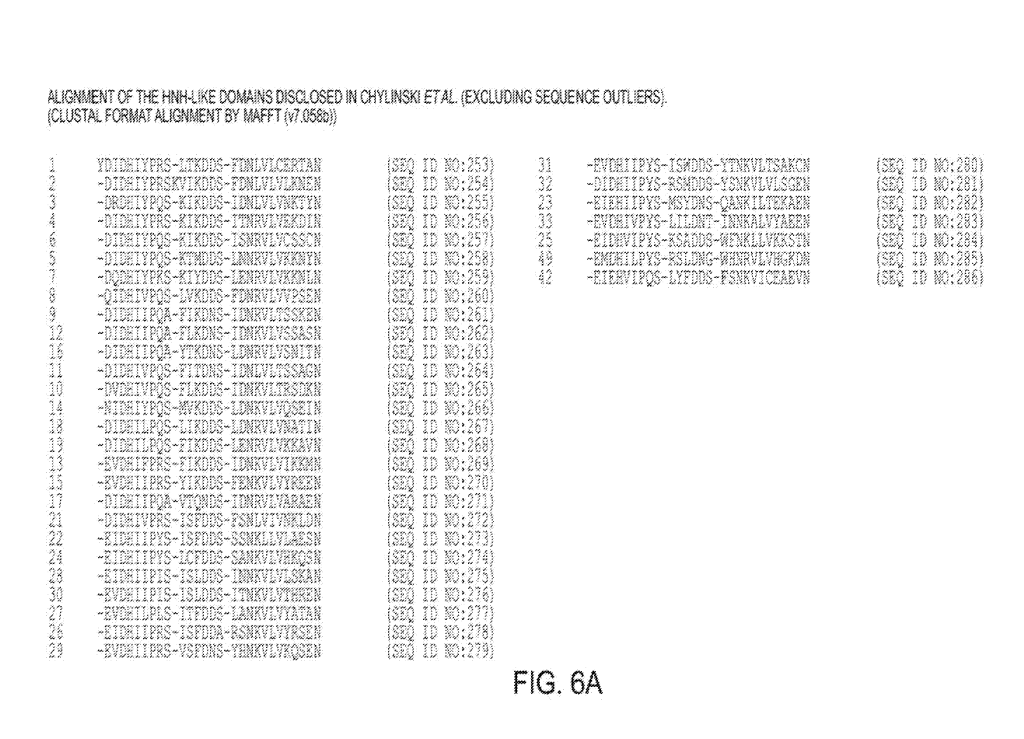

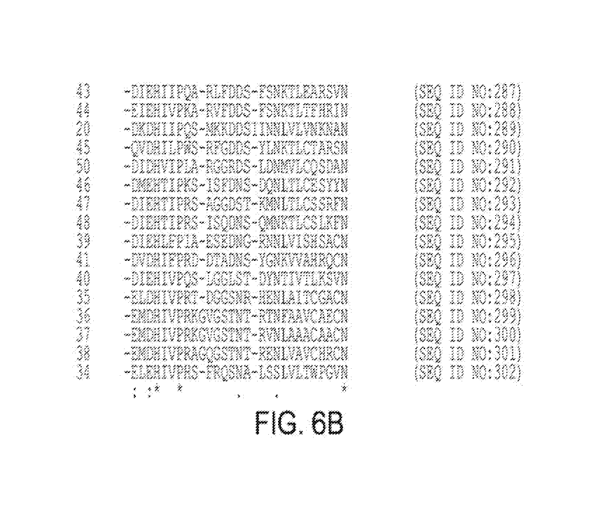

In an embodiment, the HNH-like domain differs from a sequence of an HNH-like domain disclosed herein, e.g., in FIGS. 6A-6B or FIGS. 7A-7B, as many as 1 but no more than 2, 3, 4, or 5 residues. In an embodiment, 1, 2, all 3 of the highly conserved residues identified in FIGS. 6A-6B or FIGS. 7A-7B are present.

b) Cas9 Activities
(1) Nuclease and Helicase Activities

In an embodiment, the Cas9 molecule or Cas9 polypeptide is capable of cleaving a target nucleic acid molecule. Typically wild type Cas9 molecules cleave both strands of a target nucleic acid molecule. Cas9 molecules and Cas9 polypeptides can be engineered to alter nuclease cleavage (or other properties), e.g., to provide a Cas9 molecule or Cas9 peolypeptide which is a nickase, or which lacks the ability to cleave target nucleic acid. A Cas9 molecule or Cas9 polypeptide that is capable of cleaving a target nucleic acid molecule is referred to herein as an eaCas9 molecule or eaCas9 polypeptide

In an embodiment, an eaCas9 molecule or eaCas9 polypeptide comprises one or more of the following activities:
a nickase activity, i.e., the ability to cleave a single strand, e.g., the non-complementary strand or the complementary strand, of a nucleic acid molecule;
a double stranded nuclease activity, i.e., the ability to cleave both strands of a double stranded nucleic acid and create a double stranded break, which in an embodiment is the presence of two nickase activities;
an endonuclease activity;
an exonuclease activity; and
a helicase activity, i.e., the ability to unwind the helical structure of a double stranded nucleic acid.

In an embodiment, an enzymatically active or eaCas9 molecule or eaCas9 polypeptide cleaves both strands and results in a double stranded break. In an embodiment, an eaCas9 molecule cleaves only one strand, e.g., the strand to which the gRNA hybridizes to, or the strand complementary to the strand the gRNA hybridizes with. In an embodiment, an eaCas9 molecule or eaCas9 polypeptide comprises cleavage activity associated with an HNH-like domain. In an embodiment, an eaCas9 molecule or eaCas9 polypeptide comprises cleavage activity associated with an N-terminal RuvC-like domain. In an embodiment, an eaCas9 molecule or eaCas9 polypeptide comprises cleavage activity associated with an HNH-like domain and cleavage activity associated with an N-terminal RuvC-like domain. In an embodiment, an eaCas9 molecule or eaCas9 polypeptide comprises an active, or cleavage competent, HNH-like domain and an inactive, or cleavage incompetent, N-terminal RuvC-like domain. In an embodiment, an eaCas9 molecule or eaCas9 polypeptide comprises an inactive, or cleavage incompetent, HNH-like domain and an active, or cleavage competent, N-terminal RuvC-like domain.

Some Cas9 molecules or Cas9 polypeptides have the ability to interact with a gRNA molecule, and in conjunction with the gRNA molecule localize to a core target domain, but are incapable of cleaving the target nucleic acid, or incapable of cleaving at efficient rates. Cas9 molecules having no, or no substantial, cleavage activity are referred to herein as an eiCas9 molecule or eiCas9 polypeptide. For example, an eiCas9 molecule or eiCas9 polypeptide can lack cleavage activity or have substantially less, e.g., less than 20, 10, 5, 1 or 0.1% of the cleavage activity of a reference Cas9 molecule or eiCas9 polypeptide, as measured by an assay described herein.

(2) Targeting and PAMs

A Cas9 molecule or Cas9 polypeptide, is a polypeptide that can interact with a guide RNA (gRNA) molecule and, in concert with the gRNA molecule, localizes to a site which comprises a target domain and a PAM sequence.

In an embodiment, the ability of an eaCas9 molecule or eaCas9 polypeptide to interact with and cleave a target nucleic acid is PAM sequence dependent. A PAM sequence is a sequence in the target nucleic acid. In an embodiment, cleavage of the target nucleic acid occurs upstream from the PAM sequence. EaCas9 molecules from different bacterial species can recognize different sequence motifs (e.g., PAM sequences). In an embodiment, an eaCas9 molecule of S. pyogenes recognizes the sequence motif NGG, NAG, NGA and directs cleavage of a target nucleic acid sequence 1 to 10, e.g., 3 to 5, base pairs upstream from that sequence. See, e.g., Mali et al., SCIENCE 2013; 339(6121): 823-826. In an embodiment, an eaCas9 molecule of S. thermophilus recognizes the sequence motif NGGNG and/or NNAGAAW (W=A or T) and directs cleavage of a target nucleic acid sequence 1 to 10, e.g., 3 to 5, base pairs upstream from these sequences. See, e.g., Horvath et al., SCIENCE 2010; 327(5962):167-170, and Deveau et al., J BACTERIOL 2008; 190(4): 1390-1400. In an embodiment, an eaCas9 molecule of S. mutans recognizes the sequence motif NGG and/or NAAR (R=A or G)) and directs cleavage of a core target nucleic acid sequence 1 to 10, e.g., 3 to 5 base pairs, upstream from this sequence. See, e.g., Deveau et al., J BACTERIOL 2008; 190(4): 1390-1400. In an embodiment, an eaCas9 molecule of S. aureus recognizes the sequence motif NNGRR (R=A or G) and directs cleavage of a target nucleic acid sequence 1 to 10, e.g., 3 to 5, base pairs upstream from that sequence. In an embodiment, an eaCas9 molecule of S. aureus recognizes the sequence motif NNGRRT (R=A or G) and directs cleavage of a target nucleic acid sequence 1 to 10, e.g., 3 to 5, base pairs upstream from that sequence. In an embodiment, an eaCas9 molecule of S. aureus recognizes the sequence motif NNGRRV (R=A or G) and directs cleavage of a target nucleic acid sequence 1 to 10, e.g., 3 to 5, base pairs upstream from that sequence. In an embodiment, an eaCas9 molecule of N. meningitidis recognizes the sequence motif NNNNGATT or NNNGCTT (R=A or G, V=A, G or C and directs cleavage of a target nucleic acid sequence 1 to 10, e.g., 3 to 5, base pairs upstream from that sequence. See, e.g., Hou et al., PNAS EARLY EDITION 2013, 1-6. The ability of a Cas9 molecule to recognize a PAM sequence can be determined, e.g., using a transformation assay described in Jinek et al., SCIENCE 2012 337:816. In the aforementioned embodiments, N can be any nucleotide residue, e.g., any of A, G, C or T.

As is discussed herein, Cas9 molecules can be engineered to alter the PAM specificity of the Cas9 molecule.

Exemplary naturally occurring Cas9 molecules are described in Chylinski et al., RNA Biology 2013 10:5, 727-737. Such Cas9 molecules include Cas9 molecules of a cluster 1-78 bacterial family.

Exemplary naturally occurring Cas9 molecules include a Cas9 molecule of a cluster 1 bacterial family. Examples include a Cas9 molecule of: S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F0211), S. agalactiae (e.g., strain NEM316, A909), Listeria monocytogenes (e.g., strain F6854), Listeria innocua (L. innocua, e.g., strain Clip11262), Enterococcus italicus (e.g., strain DSM 15952), or Enterococcus faecium (e.g., strain 1,231,408). Another exemplary Cas9 molecule is a Cas9 molecule of Neisseria meningitidis (Hou et al., PNAS Early Edition 2013, 1-6).

In an embodiment, a Cas9 molecule or Cas9 polypeptide, e.g., an eaCas9 molecule or eaCas9 polypeptide, comprises an amino acid sequence:
having 60%, 65%, 70%, 75%, 80%, 85%, 90%, 95%, 96%, 97%, 98% or 99% homology with;
differs at no more than, 2, 5, 10, 15, 20, 30, or 40% of the amino acid residues when compared with;
differs by at least 1, 2, 5, 10 or 20 amino acids but by no more than 100, 80, 70, 60, 50, 40 or 30 amino acids from; or
is identical to any Cas9 molecule sequence described herein, or a naturally occurring Cas9 molecule sequence, e.g., a Cas9 molecule from a species listed herein or described in Chylinski et al., RNA Biology 2013 10:5, 727-737; Hou et al., PNAS Early Edition 2013, 1-6; SEQ ID NOS:1-4. In an embodiment, the Cas9 molecule or Cas9 polypeptide comprises one or more of the following activities: a nickase activity; a double stranded cleavage activity (e.g., an endonuclease and/or exonuclease activity); a helicase activity; or the ability, together with a gRNA molecule, to home to a target nucleic acid.

In an embodiment, a Cas9 molecule or Cas9 polypeptide comprises the amino acid sequence of the consensus sequence of FIGS. 2A-2G, wherein “*” indicates any amino acid found in the corresponding position in the amino acid sequence of a Cas9 molecule of S. pyogenes, S. thermophilus, S. mutans and L. innocua, and “-” indicates any amino acid. In an embodiment, a Cas9 molecule or Cas9 polypeptide differs from the sequence of the consensus sequence disclosed in FIGS. 2A-2G by at least 1, but no more than 2, 3, 4, 5, 6, 7, 8, 9, or 10 amino acid residues. In an embodiment, a Cas9 molecule or Cas9 polypeptide comprises the amino acid sequence of SEQ ID NO:7 of FIGS. 7A-7B, wherein “*” indicates any amino acid found in the corresponding position in the amino acid sequence of a Cas9 molecule of S. pyogenes, or N. meningitidis, “-” indicates any amino acid, and “-” indicates any amino acid or absent. In an embodiment, a Cas9 molecule or Cas9 polypeptide differs from the sequence of SEQ ID NO:6 or 7 disclosed in FIGS. 7A-7B by at least 1, but no more than 2, 3, 4, 5, 6, 7, 8, 9, or 10 amino acid residues.

A comparison of the sequence of a number of Cas9 molecules indicate that certain regions are conserved. These are identified below as:
region 1 (residues 1 to 180, or in the case of region 1′residues 120 to 180)
region 2 (residues 360 to 480);
region 3 (residues 660 to 720);
region 4 (residues 817 to 900); and
region 5 (residues 900 to 960);

In an embodiment, a Cas9 molecule or Cas9 polypeptide comprises regions 1-5, together with sufficient additional Cas9 molecule sequence to provide a biologically active molecule, e.g., a Cas9 molecule having at least one activity described herein. In an embodiment, each of regions 1-6, independently, have, 50%, 60%, 70%, or 80% homology with the corresponding residues of a Cas9 molecule or Cas9 polypeptide described herein, e.g., a sequence from FIGS. 2A-2G or from FIGS. 7A-7B.

In an embodiment, a Cas9 molecule or Cas9 polypeptide, e.g., an eaCas9 molecule or eaCas9 polypeptide, comprises an amino acid sequence referred to as region 1:
having 50%, 60%, 70%, 80%, 85%, 90%, 95%, 96%, 97%, 98% or 99% homology with amino acids 1-180 (the numbering is according to the motif sequence in FIGS. 2A-2G; 52% of residues in the four Cas9 sequences in FIGS. 2A-2G are conserved) of the amino acid sequence of Cas9 of S. pyogenes;
differs by at least 1, 2, 5, 10 or 20 amino acids but by no more than 90, 80, 70, 60, 50, 40 or 30 amino acids from amino acids 1-180 of the amino acid sequence of Cas9 of S. pyogenes, S. thermophilus, S. mutans or L. innocua; or
is identical to 1-180 of the amino acid sequence of Cas9 of S. pyogenes, S. thermophilus, S. mutans or L. innocua.

In an embodiment, a Cas9 molecule or Cas9 polypeptide, e.g., an eaCas9 molecule or eaCas9 polypeptide, comprises an amino acid sequence referred to as region 1′:
having 55%, 60%, 65%, 70%, 75%, 80%, 85%, 90%, 95%, 96%, 97%, 98% or 99% homology with amino acids 120-180 (55% of residues in the four Cas9 sequences in FIGS. 2A-2G are conserved) of the amino acid sequence of Cas9 of S. pyogenes, S. thermophilus, S. mutans or L. innocua;
differs by at least 1, 2, or 5 amino acids but by no more than 35, 30, 25, 20 or 10 amino acids from amino acids 120-180 of the amino acid sequence of Cas9 of S. pyogenes, S. thermophilus, S. mutans or L. innocua; or
is identical to 120-180 of the amino acid sequence of Cas9 of S. pyogenes, S. thermophilus, S. mutans or L. innocua.

In an embodiment, a Cas9 molecule or Cas9 polypeptide, e.g., an eaCas9 molecule or eaCas9 polypeptide, comprises an amino acid sequence referred to as region 2:
having 50%, 55%, 60%, 65%, 70%, 75%, 80%, 85%, 90%, 95%, 96%, 97%, 98% or 99% homology with amino acids 360-480 (52% of residues in the four Cas9 sequences in FIGS. 2A-2G are conserved) of the amino acid sequence of Cas9 of S. pyogenes, S. thermophilus, S. mutans or L. innocua;
differs by at least 1, 2, or 5 amino acids but by no more than 35, 30, 25, 20 or 10 amino acids from amino acids 360-480 of the amino acid sequence of Cas9 of S. pyogenes, S. thermophilus, S. mutans or L. innocua; or
is identical to 360-480 of the amino acid sequence of Cas9 of S. pyogenes, S. thermophilus, S. mutans or L. innocua.

In an embodiment, a Cas9 molecule or Cas9 polypeptide, e.g., an eaCas9 molecule or eaCas9 polypeptide, comprises an amino acid sequence referred to as region 3:
having 55%, 60%, 65%, 70%, 75%, 80%, 85%, 90%, 95%, 96%, 97%, 98%, or 99% homology with amino acids 660-720 (56% of residues in the four Cas9 sequences in FIGS. 2A-2G are conserved) of the amino acid sequence of Cas9 of S. pyogenes, S. thermophilus, S. mutans or L. innocua;
differs by at least 1, 2, or 5 amino acids but by no more than 35, 30, 25, 20 or 10 amino acids from amino acids 660-720 of the amino acid sequence of Cas9 of S. pyogenes, S. thermophilus, S. mutans or L. innocua; or
is identical to 660-720 of the amino acid sequence of Cas9 of S. pyogenes, S. thermophilus, S. mutans or L. innocua.

In an embodiment, a Cas9 molecule or Cas9 polypeptide, e.g., an eaCas9 molecule or eaCas9 polypeptide, comprises an amino acid sequence referred to as region 4:
having 50%, 55%, 60%, 65%, 70%, 75%, 80%, 85%, 90%, 95%, 96%, 97%, 98%, or 99% homology with amino acids 817-900 (55% of residues in the four Cas9 sequences in FIGS. 2A-2G are conserved) of the amino acid sequence of Cas9 of S. pyogenes, S. thermophilus, S. mutans or L. innocua;
differs by at least 1, 2, or 5 amino acids but by no more than 35, 30, 25, 20 or 10 amino acids from amino acids 817-900 of the amino acid sequence of Cas9 of S. pyogenes, S. thermophilus, S. mutans or L. innocua; or
is identical to 817-900 of the amino acid sequence of Cas9 of S. pyogenes, S. thermophilus, S. mutans or L. innocua.

In an embodiment, a Cas9 molecule or Cas9 polypeptide, e.g., an eaCas9 molecule or eaCas9 polypeptide, comprises an amino acid sequence referred to as region 5:
having 50%, 55%, 60%, 65%, 70%, 75%, 80%, 85%, 90%, 95%, 96%, 97%, 98%, or 99% homology with amino acids 900-960 (60% of residues in the four Cas9 sequences in FIGS. 2A-2G are conserved) of the amino acid sequence of Cas9 of S. pyogenes, S. thermophilus, S. mutans or L. innocua;
differs by at least 1, 2, or 5 amino acids but by no more than 35, 30, 25, 20 or 10 amino acids from amino acids 900-960 of the amino acid sequence of Cas9 of S. pyogenes, S. thermophilus, S. mutans or L. innocua; or
is identical to 900-960 of the amino acid sequence of Cas9 of S. pyogenes, S. thermophilus, S. mutans or L. innocua.

c) Engineered or Altered Cas9 Molecules and Cas9 Polypeptides

Cas9 molecules and Cas9 polypeptides described herein, e.g., naturally occurring Cas9 molecules, can possess any of a number of properties, including: nickase activity, nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In an embodiment, a Cas9 molecule or Cas9 polypeptide can include all or a subset of these properties. In typical embodiments, a Cas9 molecule or Cas9 polypeptide has the ability to interact with a gRNA molecule and, in concert with the gRNA molecule, localize to a site in a nucleic acid. Other activities, e.g., PAM specificity, cleavage activity, or helicase activity can vary more widely in Cas9 molecules and Cas9 polypeptides.

Cas9 molecules include engineered Cas9 molecules and engineered Cas9 polypeptides (“engineered,” as used in this context, means merely that the Cas9 molecule or Cas9 polypeptide differs from a reference sequences, and implies no process or origin limitation). An engineered Cas9 molecule or Cas9 polypeptide can comprise altered enzymatic properties, e.g., altered nuclease activity, (as compared with a naturally occurring or other reference Cas9 molecule) or altered helicase activity. As discussed herein, an engineered Cas9 molecule or Cas9 polypeptide can have nickase activity (as opposed to double strand nuclease activity). In an embodiment an engineered Cas9 molecule or Cas9 polypeptide can have an alteration that alters its size, e.g., a deletion of amino acid sequence that reduces its size, e.g., without significant effect on one or more, or any Cas9 activity. In an embodiment, an engineered Cas9 molecule or Cas9 polypeptide can comprise an alteration that affects PAM recognition. E.g., an engineered Cas9 molecule can be altered to recognize a PAM sequence other than that recognized by the endogenous wild-type PI domain. In an embodiment a Cas9 molecule or Cas9 polypeptide can differ in sequence from a naturally occurring Cas9 molecule but not have significant alteration in one or more Cas9 activities.

Cas9 molecules or Cas9 polypeptides with desired properties can be made in a number of ways, e.g., by alteration of a parental, e.g., naturally occurring, Cas9 molecules or Cas9 polypeptides, to provide an altered Cas9 molecule or Cas9 polypeptide having a desired property. For example, one or more mutations or differences relative to a parental Cas9 molecule, e.g., a naturally occurring or engineered Cas9 molecule, can be introduced. Such mutations and differences comprise: substitutions (e.g., conservative substitutions or substitutions of non-essential amino acids); insertions; or deletions. In an embodiment, a Cas9 molecule or Cas9 polypeptide can comprises one or more mutations or differences, e.g., at least 1, 2, 3, 4, 5, 10, 15, 20, 30, 40 or 50 mutations but less than 200, 100, or 80 mutations relative to a reference, e.g., a parental, Cas9 molecule.

In an embodiment, a mutation or mutations do not have a substantial effect on a Cas9 activity, e.g. a Cas9 activity described herein. In an embodiment, a mutation or mutations have a substantial effect on a Cas9 activity, e.g. a Cas9 activity described herein.

(1) Non-Cleaving and Modified-Cleavage Cas9 Molecules and Cas9 Polypeptides

In an embodiment, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S. pyogenes, as follows: its ability to modulate, e.g., decreased or increased, cleavage of a double stranded nucleic acid (endonuclease and/or exonuclease activity), e.g., as compared to a naturally occurring Cas9 molecule (e.g., a Cas9 molecule of S. pyogenes); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S. pyogenes); or the ability to cleave a nucleic acid molecule, e.g., a double stranded or single stranded nucleic acid molecule, can be eliminated.

(2) Modified Cleavage eaCas9 Molecules and eaCas9 Polypeptides

In an embodiment, an eaCas9 molecule or eaCas9 polypeptide comprises one or more of the following activities: cleavage activity associated with an N-terminal RuvC-like domain; cleavage activity associated with an HNH-like domain; cleavage activity associated with an HNH-like domain and cleavage activity associated with an N-terminal RuvC-like domain.

In an embodiment, an eaCas9 molecule or eaCas9 polypeptide comprises an active, or cleavage competent, HNH-like domain (e.g., an HNH-like domain described herein, e.g., SEQ ID NO:17, SEQ ID NO:18, SEQ ID NO:19, SEQ ID NO:20, or SEQ ID NO:21) and an inactive, or cleavage incompetent, N-terminal RuvC-like domain. An exemplary inactive, or cleavage incompetent N-terminal RuvC-like domain can have a mutation of an aspartic acid in an N-terminal RuvC-like domain, e.g., an aspartic acid at position 9 of the consensus sequence disclosed in FIGS. 2A-2G or an aspartic acid at position 10 of SEQ ID NO:7, e.g., can be substituted with an alanine. In an embodiment, the eaCas9 molecule or eaCas9 polypeptide differs from wild type in the N-terminal RuvC-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In an embodiment, the reference Cas9 molecule is the naturally occurring Cas9 molecule having the closest sequence identity or homology.

In an embodiment, an eaCas9 molecule or eaCas9 polypeptide comprises an inactive, or cleavage incompetent, HNH domain and an active, or cleavage competent, N-terminal RuvC-like domain (e.g., an N-terminal RuvC-like domain described herein, e.g., SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16). Exemplary inactive, or cleavage incompetent HNH-like domains can have a mutation at one or more of: a histidine in an HNH-like domain, e.g., a histidine shown at position 856 of FIGS. 2A-2G, e.g., can be substituted with an alanine; and one or more asparagines in an HNH-like domain, e.g., an asparagine shown at position 870 of FIGS. 2A-2G and/or at position 879 of FIGS. 2A-2G, e.g., can be substituted with an alanine. In an embodiment, the eaCas9 differs from wild type in the HNH-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In an embodiment, the reference Cas9 molecule is the naturally occurring Cas9 molecule having the closest sequence identity or homology.

In an embodiment, an eaCas9 molecule or eaCas9 polypeptide comprises an inactive, or cleavage incompetent, HNH domain and an active, or cleavage competent, N-terminal RuvC-like domain (e.g., an N-terminal RuvC-like domain described herein, e.g., SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16). Exemplary inactive, or cleavage incompetent HNH-like domains can have a mutation at one or more of: a histidine in an HNH-like domain, e.g., a histidine shown at position 856 of FIGS. 2A-2G, e.g., can be substituted with an alanine; and one or more asparagines in an HNH-like domain, e.g., an asparagine shown at position 870 of FIGS. 2A-2G and/or at position 879 of FIGS. 2A-2G, e.g., can be substituted with an alanine. In an embodiment, the eaCas9 differs from wild type in the HNH-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In an embodiment, the reference Cas9 molecule is the naturally occurring Cas9 molecule having the closest sequence identity or homology.

d) Alterations in the Ability to Cleave One or Both Strands of a Target Nucleic Acid

In an embodiment, exemplary Cas9 activities comprise one or more of PAM specificity, cleavage activity, and helicase activity. A mutation(s) can be present, e.g., in: one or more RuvC-like domain, e.g., an N-terminal RuvC-like domain; an HNH-like domain; a region outside the RuvC-like domains and the HNH-like domain. In some embodiments, a mutation(s) is present in a RuvC-like domain, e.g., an N-terminal RuvC-like. In some embodiments, a mutation(s) is present in an HNH-like domain. In some embodiments, mutations are present in both a RuvC-like domain, e.g., an N-terminal RuvC-like domain, and an HNH-like domain.

Exemplary mutations that may be made in the RuvC domain or HNH domain with reference to the S. pyogenes sequence include: D10A, E762A, H840A, N854A, N863A and/or D986A.

In an embodiment, a Cas9 molecule or Cas9 polypeptide is an eiCas9 molecule or eiCas9 polypeptide comprising one or more differences in a RuvC domain and/or in an HNH domain as compared to a reference Cas9 molecule, and the eiCas9 molecule or eiCas9 polypeptide does not cleave a nucleic acid, or cleaves with significantly less efficiency than does wildype, e.g., when compared with wild type in a cleavage assay, e.g., as described herein, cuts with less than 50, 25, 10, or 1% of a reference Cas9 molecule, as measured by an assay described herein.

Whether or not a particular sequence, e.g., a substitution, may affect one or more activity, such as targeting activity, cleavage activity, etc, can be evaluated or predicted, e.g., by evaluating whether the mutation is conservative or by the method described in Section IV. In an embodiment, a “non-essential” amino acid residue, as used in the context of a Cas9 molecule, is a residue that can be altered from the wild-type sequence of a Cas9 molecule, e.g., a naturally occurring Cas9 molecule, e.g., an eaCas9 molecule, without abolishing or more preferably, without substantially altering a Cas9 activity (e.g., cleavage activity), whereas changing an “essential” amino acid residue results in a substantial loss of activity (e.g., cleavage activity).

In an embodiment, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S. aureus, S. pyogenes, or C. jejuni as follows: its ability to modulate, e.g., decreased or increased, cleavage of a double stranded break (endonuclease and/or exonuclease activity), e.g., as compared to a naturally occurring Cas9 molecule (e.g., a Cas9 molecule of S. aureus, S. pyogenes, or C. jejuni); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S. aureus, S. pyogenes, or C. jejuni); or the ability to cleave a nucleic acid molecule, e.g., a double stranded or single stranded nucleic acid molecule, can be eliminated.

In an embodiment, the altered Cas9 molecule or Cas9 polypeptide is an eaCas9 molecule or eaCas9 polypeptide comprising one or more of the following activities: cleavage activity associated with a RuvC domain; cleavage activity associated with an HNH domain; cleavage activity associated with an HNH domain and cleavage activity associated with a RuvC domain.

In an embodiment, the altered Cas9 molecule or Cas9 polypeptide is an eiCas9 molecule or eaCas9 polypeptide which does not cleave a nucleic acid molecule (either double stranded or single stranded nucleic acid molecules) or cleaves a nucleic acid molecule with significantly less efficiency, e.g., less than 20, 10, 5, 1 or 0.1% of the cleavage activity of a reference Cas9 molecule, e.g., as measured by an assay described herein. The reference Cas9 molecule can be a naturally occurring unmodified Cas9 molecule, e.g., a naturally occurring Cas9 molecule such as a Cas9 molecule of S. pyogenes, S. thermophilus, S. aureus, C. jejuni or N. meningitidis. In an embodiment, the reference Cas9 molecule is the naturally occurring Cas9 molecule having the closest sequence identity or homology. In an embodiment, the eiCas9 molecule or eiCas9 polypeptide lacks substantial cleavage activity associated with a RuvC domain and cleavage activity associated with an HNH domain.

In an embodiment, the altered Cas9 molecule or Cas9 polypeptide is an eaCas9 molecule or eaCas9 polypeptide comprising the fixed amino acid residues of S. pyogenes shown in the consensus sequence disclosed in FIGS. 2A-2G, and has one or more amino acids that differ from the amino acid sequence of S. pyogenes (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIGS. 2A-2G or SEQ ID NO:7.

In an embodiment, the altered Cas9 molecule or Cas9 polypeptide comprises a sequence in which:
the sequence corresponding to the fixed sequence of the consensus sequence disclosed in FIGS. 2A-2G differs at no more than 1, 2, 3, 4, 5, 10, 15, or 20% of the fixed residues in the consensus sequence disclosed in FIGS. 2A-2G;
the sequence corresponding to the residues identified by “*” in the consensus sequence disclosed in FIGS. 2A-2G differ at no more than 1, 2, 3, 4, 5, 10, 15, 20, 25, 30, 35, or 40% of the “*” residues from the corresponding sequence of naturally occurring Cas9 molecule, e.g., an S. pyogenes Cas9 molecule; and,
the sequence corresponding to the residues identified by “-” in the consensus sequence disclosed in FIGS. 2A-2G differ at no more than 5, 10, 15, 20, 25, 30, 35, 40, 45, 55, or 60% of the “-” residues from the corresponding sequence of naturally occurring Cas9 molecule, e.g., an S. pyogenes Cas9 molecule.

In an embodiment, the altered Cas9 molecule or Cas9 polypeptide is an eaCas9 molecule or eaCas9 polypeptide comprising the fixed amino acid residues of S. thermophilus shown in the consensus sequence disclosed in FIGS. 2A-2G, and has one or more amino acids that differ from the amino acid sequence of S. thermophilus (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIGS. 2A-2G.

In an embodiment the altered Cas9 molecule or Cas9 polypeptide comprises a sequence in which:
the sequence corresponding to the fixed sequence of the consensus sequence disclosed in FIGS. 2A-2G differs at no more than 1, 2, 3, 4, 5, 10, 15, or 20% of the fixed residues in the consensus sequence disclosed in FIGS. 2A-2G;
the sequence corresponding to the residues identified by “*” in the consensus sequence disclosed in FIGS. 2A-2G differ at no more than 1, 2, 3, 4, 5, 10, 15, 20, 25, 30, 35, or 40% of the “*” residues from the corresponding sequence of naturally occurring Cas9 molecule, e.g., an S. thermophilus Cas9 molecule; and,
the sequence corresponding to the residues identified by “-” in the consensus sequence disclosed in FIGS. 2A-2G differ at no more than 5, 10, 15, 20, 25, 30, 35, 40, 45, 55, or 60% of the “-” residues from the corresponding sequence of naturally occurring Cas9 molecule, e.g., an S. thermophilus Cas9 molecule.

In an embodiment, the altered Cas9 molecule or Cas9 polypeptide is an eaCas9 molecule or eaCas9 polypeptide comprising the fixed amino acid residues of S. mutans shown in the consensus sequence disclosed in FIGS. 2A-2G, and has one or more amino acids that differ from the amino acid sequence of S. mutans (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIGS. 2A-2G.

In an embodiment, the altered Cas9 molecule or Cas9 polypeptide comprises a sequence in which:
the sequence corresponding to the fixed sequence of the consensus sequence disclosed in FIGS. 2A-2G differs at no more than 1, 2, 3, 4, 5, 10, 15, or 20% of the fixed residues in the consensus sequence disclosed in FIGS. 2A-2G;
the sequence corresponding to the residues identified by “*” in the consensus sequence disclosed in FIGS. 2A-2G differ at no more than 1, 2, 3, 4, 5, 10, 15, 20, 25, 30, 35, or 40% of the “*” residues from the corresponding sequence of naturally occurring Cas9 molecule, e.g., an S. mutans Cas9 molecule; and,
the sequence corresponding to the residues identified by “-” in the consensus sequence disclosed in FIGS. 2A-2G differ at no more than 5, 10, 15, 20, 25, 30, 35, 40, 45, 55, or 60% of the “-” residues from the corresponding sequence of naturally occurring Cas9 molecule, e.g., an S. mutans Cas9 molecule.

In an embodiment, the altered Cas9 molecule or Cas9 polypeptide is an eaCas9 molecule or eaCas9 polypeptide comprising the fixed amino acid residues of L. innocula shown in the consensus sequence disclosed in FIGS. 2A-2G, and has one or more amino acids that differ from the amino acid sequence of L. innocula (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIGS. 2A-2G.

In an embodiment, the altered Cas9 molecule or Cas9 polypeptide comprises a sequence in which:
the sequence corresponding to the fixed sequence of the consensus sequence disclosed in FIGS. 2A-2G differs at no more than 1, 2, 3, 4, 5, 10, 15, or 20% of the fixed residues in the consensus sequence disclosed in FIGS. 2A-2G;
the sequence corresponding to the residues identified by “*” in the consensus sequence disclosed in FIGS. 2A-2G differ at no more than 1, 2, 3, 4, 5, 10, 15, 20, 25, 30, 35, or 40% of the “*” residues from the corresponding sequence of naturally occurring Cas9 molecule, e.g., an L. innocula Cas9 molecule; and,
the sequence corresponding to the residues identified by “-” in the consensus sequence disclosed in FIGS. 2A-2G differ at no more than 5, 10, 15, 20, 25, 30, 35, 40, 45, 55, or 60% of the “-” residues from the corresponding sequence of naturally occurring Cas9 molecule, e.g., an L. innocula Cas9 molecule.

In an embodiment, the altered Cas9 molecule or Cas9 polypeptide, e.g., an eaCas9 molecule, can be a fusion, e.g., of two of more different Cas9 molecules or Cas9 polypeptides, e.g., of two or more naturally occurring Cas9 molecules of different species. For example, a fragment of a naturally occurring Cas9 molecule of one species can be fused to a fragment of a Cas9 molecule of a second species. As an example, a fragment of Cas9 molecule of S. pyogenes comprising an N-terminal RuvC-like domain can be fused to a fragment of Cas9 molecule of a species other than S. pyogenes (e.g., S. thermophilus) comprising an HNH-like domain.

(1) Cas9 Molecules With Altered PAM Recognition Or No PAM Recognition

Naturally occurring Cas9 molecules can recognize specific PAM sequences, for example the PAM recognition sequences described above for, e.g., S. pyogenes, S. thermophilus, S. mutans, S. aureus and N. meningitidis.

In an embodiment, a Cas9 molecule or Cas9 polypeptide has the same PAM specificities as a naturally occurring Cas9 molecule. In other embodiments, a Cas9 molecule or Cas9 polypeptide has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule or Cas9 polypeptide recognizes to decrease off target sites and/or improve specificity; or eliminate a PAM recognition requirement. In an embodiment, a Cas9 molecule can be altered, e.g., to increase length of PAM recognition sequence and/or improve Cas9 specificity to high level of identity, e.g., to decrease off target sites and increase specificity. In an embodiment, the length of the PAM recognition sequence is at least 4, 5, 6, 7, 8, 9, 10 or 15 amino acids in length.

Cas9 molecules or Cas9 polypeptides that recognize different PAM sequences and/or have reduced off-target activity can be generated using directed evolution. Exemplary methods and systems that can be used for directed evolution of Cas9 molecules are described, e.g., in Esvelt et al. Nature 2011, 472(7344): 499-503. Candidate Cas9 molecules can be evaluated, e.g., by methods described in Section IV.

Alterations of the PI domain, which mediates PAM recognition, are discussed below.

e) Synthetic Cas9 Molecules and Cas9 Polypeptides with Altered PI Domains

Current genome-editing methods are limited in the diversity of target sequences that can be targeted by the PAM sequence that is recognized by the Cas9 molecule utilized. A synthetic Cas9 molecule (or Syn-Cas9 molecule), or synthetic Cas9 polypeptide (or Syn-Cas9 polypeptide), as that term is used herein, refers to a Cas9 molecule or Cas9 polypeptide that comprises a Cas9 core domain from one bacterial species and a functional altered PI domain, i.e., a PI domain other than that naturally associated with the Cas9 core domain, e.g., from a different bacterial species.

In an embodiment, the altered PI domain recognizes a PAM sequence that is different from the PAM sequence recognized by the naturally-occurring Cas9 from which the Cas9 core domain is derived. In an embodiment, the altered PI domain recognizes the same PAM sequence recognized by the naturally-occurring Cas9 from which the Cas9 core domain is derived, but with different affinity or specificity. A Syn-Cas9 molecule or Syn-Cas9 polypeptide can be, respectively, a Syn-eaCas9 molecule or Syn-eaCas9 polypeptide or a Syn-eiCas9 molecule Syn-eiCas9 polypeptide.

An exemplary Syn-Cas9 molecule or Syn-Cas9 polypeptide comprises:
a) a Cas9 core domain, e.g., a Cas9 core domain from Table 2A or 2B, e.g., a S. aureus, S. pyogenes, or C. jejuni Cas9 core domain; and
b) an altered PI domain from a species X Cas9 sequence selected from Tables 4 and 5.

In an embodiment, the RKR motif (the PAM binding motif) of said altered PI domain comprises: differences at 1, 2, or 3 amino acid residues; a difference in amino acid sequence at the first, second, or third position; differences in amino acid sequence at the first and second positions, the first and third positions, or the second and third positions; as compared with the sequence of the RKR motif of the native or endogenous PI domain associated with the Cas9 core domain.

In an embodiment, the Cas9 core domain comprises the Cas9 core domain from a species X Cas9 from Table 2A and said altered PI domain comprises a PI domain from a species Y Cas9 from Table 2A.

In an embodiment, the RKR motif of the species X Cas9 is other than the RKR motif of the species Y Cas9.

In an embodiment, the RKR motif of the altered PI domain is selected from XXY, XNG, and XNQ.

In an embodiment, the altered PI domain has at least 60, 70, 80, 90, 95, or 100% homology with the amino acid sequence of a naturally occurring PI domain of said species Y from Table 2A.

In an embodiment, the altered PI domain differs by no more than 50, 40, 30, 25, 20, 15, 10, 5, 4, 3, 2, or 1 amino acid residue from the amino acid sequence of a naturally occurring PI domain of said second species from Table 2A.

In an embodiment, the Cas9 core domain comprises a S. aureus core domain and altered PI domain comprises: an A. denitrificans PI domain; a C. jejuni PI domain; a H. mustelae PI domain; or an altered PI domain of species X PI domain, wherein species X is selected from Table 5.

In an embodiment, the Cas9 core domain comprises a S. pyogenes core domain and the altered PI domain comprises: an A. denitrificans PI domain; a C. jejuni PI domain; a H. mustelae PI domain; or an altered PI domain of species X PI domain, wherein species X is selected from Table 5.

In an embodiment, the Cas9 core domain comprises a C. jejuni core domain and the altered PI domain comprises: an A. denitrificans PI domain; a H. mustelae PI domain; or an altered PI domain of species X PI domain, wherein species X is selected from Table 5.

In an embodiment, the Cas9 molecule or Cas9 polypeptide further comprises a linker disposed between said Cas9 core domain and said altered PI domain.

In an embodiment, the linker comprises: a linker described elsewhere herein disposed between the Cas9 core domain and the heterologous PI domain. Suitable linkers are further described in Section V.

Exemplary altered PI domains for use in Syn-Cas9 molecules are described in Tables 4 and 5. The sequences for the 83 Cas9 orthologs referenced in Tables 4 and 5 are provided in Table 2A. Table 3 provides the Cas9 orthologs with known PAM sequences and the corresponding RKR motif.

In an embodiment, a Syn-Cas9 molecule or Syn-Cas9 polypeptide may also be size-optimized, e.g., the Syn-Cas9 molecule or Syn-Cas9 polypeptide comprises one or more deletions, and optionally one or more linkers disposed between the amino acid residues flanking the deletions. In an embodiment, a Syn-Cas9 molecule or Syn-Cas9 polypeptide comprises a REC deletion.

f) Size-Optimized Cas9 Molecules and Cas9 Polypeptides

Engineered Cas9 molecules and engineered Cas9 polypeptides described herein include a Cas9 molecule or Cas9 polypeptide comprising a deletion that reduces the size of the molecule while still retaining desired Cas9 properties, e.g., essentially native conformation, Cas9 nuclease activity, and/or target nucleic acid molecule recognition. Provided herein are Cas9 molecules or Cas9 polypeptides comprising one or more deletions and optionally one or more linkers, wherein a linker is disposed between the amino acid residues that flank the deletion. Methods for identifying suitable deletions in a reference Cas9 molecule, methods for generating Cas9 molecules with a deletion and a linker, and methods for using such Cas9 molecules will be apparent to one of ordinary skill in the art upon review of this document.

A Cas9 molecule, e.g., a S. aureus, S. pyogenes, or C. jejuni, Cas9 molecule, having a deletion is smaller, e.g., has reduced number of amino acids, than the corresponding naturally-occurring Cas9 molecule. The smaller size of the Cas9 molecules allows increased flexibility for delivery methods, and thereby increases utility for genome-editing. A Cas9 molecule or Cas9 polypeptide can comprise one or more deletions that do not substantially affect or decrease the activity of the resultant Cas9 molecules or Cas9 polypeptides described herein. Activities that are retained in the Cas9 molecules or Cas9 polypeptides comprising a deletion as described herein include one or more of the following:
a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which in an embodiment is the presence of two nickase activities;
an endonuclease activity;
an exonuclease activity;
a helicase activity, i.e., the ability to unwind the helical structure of a double stranded nucleic acid;
and recognition activity of a nucleic acid molecule, e.g., a target nucleic acid or a gRNA.

Activity of the Cas9 molecules or Cas9 polypeptides described herein can be assessed using the activity assays described herein or in the art.

(1) Identifying Regions Suitable for Deletion

Suitable regions of Cas9 molecules for deletion can be identified by a variety of methods. Naturally-occurring orthologous Cas9 molecules from various bacterial species, e.g., any one of those listed in Table 2A, can be modeled onto the crystal structure of S. pyogenes Cas9 (Nishimasu et al., Cell, 156:935-949, 2014) to examine the level of conservation across the selected Cas9 orthologs with respect to the three-dimensional conformation of the protein. Less conserved or unconserved regions that are spatially located distant from regions involved in Cas9 activity, e.g., interface with the target nucleic acid molecule and/or gRNA, represent regions or domains are candidates for deletion without substantially affecting or decreasing Cas9 activity.

(2) REC-Optimized Cas9 Molecules and Cas9 Polypeptides

A REC-optimized Cas9 molecule, or a REC-optimized Cas9 polypeptide, as that term is used herein, refers to a Cas9 molecule or Cas9 polypeptide that comprises a deletion in one or both of the REC2 domain and the RE1_{CT} domain (collectively a REC deletion), wherein the deletion comprises at least 10% of the amino acid residues in the cognate domain. A REC-optimized Cas9 molecule or Cas9 polypeptide can be an eaCas9 molecule or eaCas9 polypeptide, or an eiCas9 molecule or eiCas9 polypeptide. An exemplary REC-optimized Cas9 molecule or REC-optimized Cas9 polypeptide comprises:
a) a deletion selected from:
   i) a REC2 deletion;
   ii) a REC1_{CT} deletion; or
   iii) a REC1_{SUB} deletion.

Optionally, a linker is disposed between the amino acid residues that flank the deletion. In an embodiment a Cas9 molecule or Cas9 polypeptide includes only one deletion, or only two deletions. A Cas9 molecule or Cas9 polypeptide can comprise a REC2 deletion and a REC1_{CT} deletion. A Cas9 molecule or Cas9 polypeptide can comprise a REC2 deletion and a REC1_{SUB} deletion.

Generally, the deletion will contain at least 10% of the amino acids in the cognate domain, e.g., a REC2 deletion will include at least 10% of the amino acids in the REC2 domain. A deletion can comprise: at least 10, 20, 30, 40, 50, 60, 70, 80, or 90% of the amino acid residues of its cognate domain; all of the amino acid residues of its cognate domain; an amino acid residue outside its cognate domain; a plurality of amino acid residues outside its cognate domain; the amino acid residue immediately N terminal to its cognate domain; the amino acid residue immediately C terminal to its cognate domain; the amino acid residue immediately N terminal to its cognate and the amino acid residue immediately C terminal to its cognate domain; a plurality of, e.g., up to 5, 10, 15, or 20, amino acid residues N terminal to its cognate domain; a plurality of, e.g., up to 5, 10, 15, or 20, amino acid residues C terminal to its cognate domain; a plurality of, e.g., up to 5, 10, 15, or 20, amino acid residues N terminal to to its cognate domain and a plurality of e.g., up to 5, 10, 15, or 20, amino acid residues C terminal to its cognate domain.

In an embodiment, a deletion does not extend beyond: its cognate domain; the N terminal amino acid residue of its cognate domain; the C terminal amino acid residue of its cognate domain.

A REC-optimized Cas9 molecule or REC-optimized Cas9 polypeptide can include a linker disposed between the amino acid residues that flank the deletion. Suitable linkers for use between the amino acid resides that flank a REC deletion in a REC-optimized Cas9 molecule is disclosed in Section V.

In an embodiment, a REC-optimized Cas9 molecule or REC-optimized Cas9 polypeptide comprises an amino acid sequence that, other than any REC deletion and associated linker, has at least 50, 55, 60, 65, 70, 75, 80, 85, 90, 95, 99, or 100% homology with the amino acid sequence of a naturally occurring Cas9, e.g., a Cas9 molecule described in Table 2A, e.g., a S. aureus Cas9 molecule, a S. pyogenes Cas9 molecule, or a C. jejuni Cas9 molecule.

In an embodiment, a a REC-optimized Cas9 molecule or REC-optimized Cas9 polypeptide comprises an amino acid sequence that, other than any REC deletion and associated linker, differs by no more than 1, 2, 3, 4, 5, 6, 7, 8, 9, 10, 15, 20, or 25, amino acid residues from the amino acid sequence of a naturally occurring Cas9, e.g., a Cas9 molecule described in Table 2A, e.g., a S. aureus Cas9 molecule, a S. pyogenes Cas9 molecule, or a C. jejuni Cas9 molecule.

In an embodiment, a REC-optimized Cas9 molecule or REC-optimized Cas9 polypeptide comprises an amino acid sequence that, other than any REC deletion and associate linker, differs by no more than 1, 2, 3, 4, 5, 6, 7, 8, 9, 10, 15, 20, or 25% of the, amino acid residues from the amino acid sequence of a naturally occurring Cas9, e.g., a Cas9 molecule described in Table 2A, e.g., a S. aureus Cas9 molecule, a S. pyogenes Cas9 molecule, or a C. jejuni Cas9 molecule.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Sequence information for exemplary REC deletions are provided for 83 naturally-occurring Cas9 orthologs in Table 2A. The amino acid sequences of exemplary Cas9 molecules from different bacterial species are shown below.

TABLE 2A
Amino Acid Sequence of Cas9 Orthologs

| | | REC2 | | | REC1_{CT} | | | REC1_{SUB} | | |
|---|---|---|---|---|---|---|---|---|---|---|
| | Amino | start | stop | # AA | start | stop | # AA | start | stop | # AA |
| Species/ | acid | (AA | (AA | deleted | (AA | (AA | deleted | (AA | (AA | deleted |
| Composite ID | sequence | pos) | pos) | (n) | pos) | pos) | (n) | pos) | pos) | (n) |
| Staphylococcus | SEQ ID | 126 | 166 | 41 | 296 | 352 | 57 | 296 | 352 | 57 |
| Aureus | NO: 304 | | | | | | | | | |
| tr\|J7RUA5\|J7RUA5_ | | | | | | | | | | |
| STAAU | | | | | | | | | | |
| Streptococcus | SEQ ID | 176 | 314 | 139 | 511 | 592 | 82 | 511 | 592 | 82 |
| Pyogenes | NO: 305 | | | | | | | | | |
| sp\|Q99ZW2\|CAS9_ | | | | | | | | | | |
| STRP1 | | | | | | | | | | |
| Campylobacter | SEQ ID | 137 | 181 | 45 | 316 | 360 | 45 | 316 | 360 | 45 |
| jejuni NCTC | NO: 306 | | | | | | | | | |
| 11168 | | | | | | | | | | |
| gi\|218563121\|ref\| | | | | | | | | | | |
| YP_002344900.1 | | | | | | | | | | |
| Bacteroides | SEQ ID | 148 | 339 | 192 | 524 | 617 | 84 | 524 | 617 | 84 |
| fragilis NCTC | NO: 307 | | | | | | | | | |
| 9343 | | | | | | | | | | |
| gi\|60683389\|ref\| | | | | | | | | | | |
| YP_213533.1\| | | | | | | | | | | |
| Bifidobacterium | SEQ ID | 173 | 335 | 163 | 516 | 607 | 87 | 516 | 607 | 87 |
| bifidum S17 | NO: 308 | | | | | | | | | |
| gi\|310286728\|ref\| | | | | | | | | | | |
| YP_003937986. | | | | | | | | | | |
| Veillonella atypica | SEQ ID | 185 | 339 | 155 | 574 | 663 | 79 | 574 | 663 | 79 |
| ACS-134-V-Col7a | NO: 309 | | | | | | | | | |
| gi\|303229466\|ref\| | | | | | | | | | | |
| ZP_07316256.1 | | | | | | | | | | |
| Lactobacillus | SEQ ID | 169 | 320 | 152 | 559 | 645 | 78 | 559 | 645 | 78 |
| rhamnosus GG | NO: 310 | | | | | | | | | |
| gi\|258509199\|ref\| | | | | | | | | | | |
| YP_003171950.1 | | | | | | | | | | |
| Filifactor alocis | SEQ ID | 166 | 314 | 149 | 508 | 592 | 76 | 508 | 592 | 76 |
| ATCC 35896 | NO: 311 | | | | | | | | | |
| gi\|374307738\|ref\| | | | | | | | | | | |
| YP_005054169.1 | | | | | | | | | | |
| Oenococcus | SEQ ID | 169 | 317 | 149 | 555 | 639 | 80 | 555 | 639 | 80 |
| kitaharae DSM | NO: 312 | | | | | | | | | |
| 17330 | | | | | | | | | | |
| gi\|366983953\|gb\| | | | | | | | | | | |
| EHN59352.1\| | | | | | | | | | | |
| Fructobacillus | SEQ ID | 168 | 314 | 147 | 488 | 571 | 76 | 488 | 571 | 76 |
| fructosus KCTC | NO: 313 | | | | | | | | | |
| 3544 | | | | | | | | | | |
| gi\|339625081\|ref\| | | | | | | | | | | |
| ZP_08660870.1 | | | | | | | | | | |
| Catenibacterium | SEQ ID | 173 | 318 | 146 | 511 | 594 | 78 | 511 | 594 | 78 |
| mitsuokai DSM | NO: 314 | | | | | | | | | |
| 15897 | | | | | | | | | | |
| gi\|224543312\|ref\| | | | | | | | | | | |
| ZP_03683851.1 | | | | | | | | | | |
| Finegoldia magna | SEQ ID | 168 | 313 | 146 | 452 | 534 | 77 | 452 | 534 | 77 |
| ATCC 29328 | NO: 315 | | | | | | | | | |
| gi\|169823755\|ref\| | | | | | | | | | | |
| YP_001691366.1 | | | | | | | | | | |
| CoriobacteriumglomeransPW2 | SEQ ID | 175 | 318 | 144 | 511 | 592 | 82 | 511 | 592 | 82 |
| gi\|328956315\|ref\| | NO: 316 | | | | | | | | | |
| YP_004373648.1 | | | | | | | | | | |
| Eubacterium yurii | SEQ ID | 169 | 310 | 142 | 552 | 633 | 76 | 552 | 633 | 76 |
| ATCC 43715 | NO: 317 | | | | | | | | | |
| gi\|306821691\|ref\| | | | | | | | | | | |
| ZP_07455288.1 | | | | | | | | | | |
| Peptoniphilus | SEQ ID | 171 | 311 | 141 | 535 | 615 | 76 | 535 | 615 | 76 |
| duerdenii ATCC | NO: 318 | | | | | | | | | |
| BAA-1640 | | | | | | | | | | |
| gi\|304438954\|ref\| | | | | | | | | | | |
| ZP_07398877.1 | | | | | | | | | | |
| Acidaminococcus | SEQ ID | 167 | 306 | 140 | 511 | 591 | 75 | 511 | 591 | 75 |
| sp. D21 | NO: 319 | | | | | | | | | |
| gi\|227824983\|ref\| | | | | | | | | | | |
| ZP_03989815.1 | | | | | | | | | | |
| Lactobacillus | SEQ ID | 171 | 310 | 140 | 542 | 621 | 85 | 542 | 621 | 85 |
| farciminis KCTC | NO: 320 | | | | | | | | | |
| 3681 | | | | | | | | | | |
| gi\|336394882\|ref\| | | | | | | | | | | |
| ZP_08576281.1 | | | | | | | | | | |
| Streptococcus | SEQ ID | 185 | 324 | 140 | 411 | 490 | 85 | 411 | 490 | 85 |
| sanguinis SK49 | NO: 321 | | | | | | | | | |
| gi\|422884106\|ref\| | | | | | | | | | | |
| ZP_16930555.1 | | | | | | | | | | |
| Coprococcus catus | SEQ ID | 172 | 310 | 139 | 556 | 634 | 76 | 556 | 634 | 76 |
| GD-7 | NO: 322 | | | | | | | | | |
| gi\|291520705\|emb\| | | | | | | | | | | |
| CBK78998.1\| | | | | | | | | | | |
| Streptococcus | SEQ ID | 176 | 314 | 139 | 392 | 470 | 84 | 392 | 470 | 84 |
| mutans UA159 | NO: 323 | | | | | | | | | |
| gi\|24379809\|ref\| | | | | | | | | | | |
| NP_721764.1\| | | | | | | | | | | |
| Streptococcus | SEQ ID | 176 | 314 | 139 | 523 | 600 | 82 | 523 | 600 | 82 |
| pyogenes M1 GAS | NO: 324 | | | | | | | | | |
| gi\|13622193\|gb\| | | | | | | | | | | |
| AAK33936.1\| | | | | | | | | | | |
| Streptococcus | SEQ ID | 176 | 314 | 139 | 481 | 558 | 81 | 481 | 558 | 81 |
| thermophilus | NO: 325 | | | | | | | | | |
| LMD-9 | | | | | | | | | | |
| gi\|116628213\|ref\| | | | | | | | | | | |
| YP_820832.1\| | | | | | | | | | | |
| Fusobacteriumnucleatum | SEQ ID | 171 | 308 | 138 | 537 | 614 | 76 | 537 | 614 | 76 |
| ATCC49256 | NO: 326 | | | | | | | | | |
| gi\|34762592\|ref\|ZP_ | | | | | | | | | | |
| 00143587.1\| | | | | | | | | | | |
| Planococcus | SEQ ID | 162 | 299 | 138 | 538 | 614 | 94 | 538 | 614 | 94 |
| antarcticus DSM | NO: 327 | | | | | | | | | |
| 14505 | | | | | | | | | | |
| gi\|389815359\|ref\| | | | | | | | | | | |
| ZP_10206685.1 | | | | | | | | | | |
| Treponema | SEQ ID | 169 | 305 | 137 | 524 | 600 | 81 | 524 | 600 | 81 |
| denticola ATCC | NO: 328 | | | | | | | | | |
| 35405 | | | | | | | | | | |
| gi\|42525843\|ref\| | | | | | | | | | | |
| NP_970941.1\| | | | | | | | | | | |
| Solobacterium | SEQ ID | 179 | 314 | 136 | 544 | 619 | 77 | 544 | 619 | 77 |
| moorei F0204 | NO: 329 | | | | | | | | | |
| gi\|320528778\|ref\| | | | | | | | | | | |
| ZP_08029929.1 | | | | | | | | | | |
| Staphylococcus | SEQ ID | 164 | 299 | 136 | 531 | 606 | 92 | 531 | 606 | 92 |
| pseudintermedius | NO: 330 | | | | | | | | | |
| ED99 | | | | | | | | | | |
| gi\|323463801\|gb\| | | | | | | | | | | |
| ADX75954.1\| | | | | | | | | | | |
| Flavobacterium | SEQ ID | 162 | 286 | 125 | 538 | 613 | 63 | 538 | 613 | 63 |
| branchiophilum | NO: 331 | | | | | | | | | |
| FL-15 | | | | | | | | | | |
| gi\|347536497\|ref\| | | | | | | | | | | |
| YP_004843922.1 | | | | | | | | | | |
| Ignavibacterium | SEQ ID | 223 | 329 | 107 | 357 | 432 | 90 | 357 | 432 | 90 |
| album JCM 16511 | NO: 332 | | | | | | | | | |
| gi\|385811609\|ref\| | | | | | | | | | | |
| YP_005848005.1 | | | | | | | | | | |
| Bergeyella | SEQ ID | 165 | 261 | 97 | 529 | 604 | 56 | 529 | 604 | 56 |
| zoohelcum ATCC | NO: 333 | | | | | | | | | |
| 43767 | | | | | | | | | | |
| gi\|423317190\|ref\| | | | | | | | | | | |
| ZP_17295095.1 | | | | | | | | | | |
| Nitrobacter | SEQ ID | 169 | 253 | 85 | 536 | 611 | 48 | 536 | 611 | 48 |
| hamburgensis X14 | NO: 334 | | | | | | | | | |
| gi\|92109262\|ref\| | | | | | | | | | | |
| YP_571550.1\| | | | | | | | | | | |
| Odoribacter laneus | SEQ ID | 164 | 242 | 79 | 535 | 610 | 63 | 535 | 610 | 63 |
| YIT 12061 | NO: 335 | | | | | | | | | |
| gi\|374384763\|ref\| | | | | | | | | | | |
| ZP_09642280.1 | | | | | | | | | | |
| Legionella | SEQ ID | 164 | 239 | 76 | 402 | 476 | 67 | 402 | 476 | 67 |
| pneumophila str. | NO: 336 | | | | | | | | | |
| Paris | | | | | | | | | | |
| gi\|54296138\|ref\| | | | | | | | | | | |
| YP_122507.1\| | | | | | | | | | | |
| Bacteroides sp. 203 | SEQ ID | 198 | 269 | 72 | 530 | 604 | 83 | 530 | 604 | 83 |
| gi\|301311869\|ref\| | NO: 337 | | | | | | | | | |
| ZP_07217791.1 | | | | | | | | | | |
| Akkermansia | SEQ ID | 136 | 202 | 67 | 348 | 418 | 62 | 348 | 418 | 62 |
| muciniphila | NO: 338 | | | | | | | | | |
| ATCC BAA-835 | | | | | | | | | | |
| gi\|187736489\|ref\| | | | | | | | | | | |
| YP_001878601. | | | | | | | | | | |
| Prevotella sp. | SEQ ID | 184 | 250 | 67 | 357 | 425 | 78 | 357 | 425 | 78 |
| C561 | NO: 339 | | | | | | | | | |
| gi\|345885718\|ref\| | | | | | | | | | | |
| ZP_08837074.1 | | | | | | | | | | |
| Wolinella | SEQ ID | 157 | 218 | 36 | 401 | 468 | 60 | 401 | 468 | 60 |
| succinogenes | NO: 340 | | | | | | | | | |
| DSM 1740 | | | | | | | | | | |
| gi\|34557932\|ref\| | | | | | | | | | | |
| NP_907747.1\| | | | | | | | | | | |
| Alicyclobacillus | SEQ ID | 142 | 196 | 55 | 416 | 482 | 61 | 416 | 482 | 61 |
| hesperidum | NO: 341 | | | | | | | | | |
| URH17-3-68 | | | | | | | | | | |
| gi\|403744858\|ref\| | | | | | | | | | | |
| ZP_10953934.1 | | | | | | | | | | |
| Caenispirillum | SEQ ID | 161 | 214 | 54 | 330 | 393 | 68 | 330 | 393 | 68 |
| salinarum AK4 | NO: 342 | | | | | | | | | |
| gi\|427429481\|ref\| | | | | | | | | | | |
| ZP_18919511.1 | | | | | | | | | | |
| Eubacterium | SEQ ID | 133 | 185 | 53 | 322 | 384 | 60 | 322 | 384 | 60 |
| rectale ATCC | NO: 343 | | | | | | | | | |
| 33656 | | | | | | | | | | |
| gi\|238924075\|ref\| | | | | | | | | | | |
| YP_002937591.1 | | | | | | | | | | |
| Mycoplasma | SEQ ID | 187 | 239 | 53 | 319 | 381 | 80 | 319 | 381 | 80 |
| synoviae 53 | NO: 344 | | | | | | | | | |
| gi\|71894592\|ref\| | | | | | | | | | | |
| YP_278700.1\| | | | | | | | | | | |
| Porphyromonas | SEQ ID | 150 | 202 | 53 | 309 | 371 | 60 | 309 | 371 | 60 |
| sp. oral taxon 279 | NO: 345 | | | | | | | | | |
| str. F0450 | | | | | | | | | | |
| gi\|402847315\|ref\| | | | | | | | | | | |
| ZP_10895610.1 | | | | | | | | | | |
| Streptococcus | SEQ ID | 127 | 178 | 139 | 424 | 486 | 81 | 424 | 486 | 81 |
| thermophilus | NO: 346 | | | | | | | | | |
| LMD-9 | | | | | | | | | | |
| gi\|116627542\|ref\| | | | | | | | | | | |
| YP_820161.1\| | | | | | | | | | | |
| Roseburia | SEQ ID | 154 | 204 | 51 | 318 | 380 | 69 | 318 | 380 | 69 |
| inulinivorans | NO: 347 | | | | | | | | | |
| DSM 16841 | | | | | | | | | | |
| gi\|225377804\|ref\| | | | | | | | | | | |
| ZP_03755025.1 | | | | | | | | | | |
| Methylosinus | SEQ ID | 144 | 193 | 50 | 426 | 488 | 64 | 426 | 488 | 64 |
| trichosporium | NO: 348 | | | | | | | | | |
| OB3b | | | | | | | | | | |
| gi\|296446027\|ref\| | | | | | | | | | | |
| ZP_06887976.1 | | | | | | | | | | |
| Ruminococcus | SEQ ID | 139 | 187 | 49 | 351 | 412 | 55 | 351 | 412 | 55 |
| albus 8 | NO: 349 | | | | | | | | | |
| gi\|325677756\|ref\| | | | | | | | | | | |
| ZP_08157403.1 | | | | | | | | | | |
| Bifidobacterium | SEQ ID | 183 | 230 | 48 | 370 | 431 | 44 | 370 | 431 | 44 |
| longum DJO10A | NO: 350 | | | | | | | | | |
| gi\|189440764\|ref\| | | | | | | | | | | |
| YP_001955845. | | | | | | | | | | |
| Enterococcus | SEQ ID | 123 | 170 | 48 | 327 | 387 | 60 | 327 | 387 | 60 |
| faecalis TX0012 | NO: 351 | | | | | | | | | |
| gi\|315149830\|gb\| | | | | | | | | | | |
| EFT93846.1\| | | | | | | | | | | |
| Mycoplasma | SEQ ID | 179 | 226 | 48 | 314 | 374 | 79 | 314 | 374 | 79 |
| mobile 163K | NO: 352 | | | | | | | | | |
| gi\|47458868\|ref\| | | | | | | | | | | |
| YP_015730.1\| | | | | | | | | | | |
| Actinomyces | SEQ ID | 147 | 193 | 47 | 358 | 418 | 40 | 358 | 418 | 40 |
| coleocanis DSM | NO: 353 | | | | | | | | | |
| 15436 | | | | | | | | | | |
| gi\|227494853\|ref\| | | | | | | | | | | |
| ZP_03925169.1 | | | | | | | | | | |
| Dinoroseobacter | SEQ ID | 138 | 184 | 47 | 338 | 398 | 48 | 338 | 398 | 48 |
| shibae DFL 12 | NO: 354 | | | | | | | | | |
| gi\|159042956\|ref\| | | | | | | | | | | |
| YP_001531750.1 | | | | | | | | | | |
| Actinomyces sp. | SEQ ID | 183 | 228 | 46 | 349 | 409 | 40 | 349 | 409 | 40 |
| oral taxon 180 str. | NO: 355 | | | | | | | | | |
| F0310 | | | | | | | | | | |
| gi\|315605738\|ref\| | | | | | | | | | | |
| ZP_07880770.1 | | | | | | | | | | |
| Alcanivorax sp. | SEQ ID | 139 | 183 | 45 | 344 | 404 | 61 | 344 | 404 | 61 |
| W11-5 | NO: 356 | | | | | | | | | |
| gi\|407803669\|ref\| | | | | | | | | | | |
| ZP_11150502.1 | | | | | | | | | | |
| Aminomonas | SEQ ID | 134 | 178 | 45 | 341 | 401 | 63 | 341 | 401 | 63 |
| paucivorans DSM | NO: 357 | | | | | | | | | |
| 12260 | | | | | | | | | | |
| gi\|312879015\|ref\| | | | | | | | | | | |
| ZP_07738815.1 | | | | | | | | | | |
| Mycoplasma canis | SEQ ID | 139 | 183 | 45 | 319 | 379 | 76 | 319 | 379 | 76 |
| PG 14 | NO: 358 | | | | | | | | | |
| gi\|384393286\|gb\| | | | | | | | | | | |
| EIE39736.1\| | | | | | | | | | | |
| Lactobacillus | SEQ ID | 141 | 184 | 44 | 328 | 387 | 61 | 328 | 387 | 61 |
| coryniformis | NO: 359 | | | | | | | | | |
| KCTC 3535 | | | | | | | | | | |
| gi\|336393381\|ref\| | | | | | | | | | | |
| ZP_08574780.1 | | | | | | | | | | |
| Elusimicrobium | SEQ ID | 177 | 219 | 43 | 322 | 381 | 47 | 322 | 381 | 47 |
| minutum Pei191 | NO: 360 | | | | | | | | | |
| gi\|187250660\|ref\| | | | | | | | | | | |
| YP_001875142.1 | | | | | | | | | | |
| Neisseria | SEQ ID | 147 | 189 | 43 | 360 | 419 | 61 | 360 | 419 | 61 |
| meningitidis | NO: 361 | | | | | | | | | |
| Z2491 | | | | | | | | | | |
| gi\|218767588\|ref\| | | | | | | | | | | |
| YP_002342100.1 | | | | | | | | | | |
| Pasteurella | SEQ ID | 139 | 181 | 43 | 319 | 378 | 61 | 319 | 378 | 61 |
| multocida str. | NO: 362 | | | | | | | | | |
| Pm70 | | | | | | | | | | |
| gi\|15602992\|ref\| | | | | | | | | | | |
| NP_246064.1\| | | | | | | | | | | |
| Rhodovulum sp. | SEQ ID | 141 | 183 | 43 | 319 | 378 | 48 | 319 | 378 | 48 |
| PH10 | NO: 363 | | | | | | | | | |
| gi\|402849997\|ref\| | | | | | | | | | | |
| ZP_10898214.1 | | | | | | | | | | |
| Eubacterium | SEQ ID | 131 | 172 | 42 | 303 | 361 | 59 | 303 | 361 | 59 |
| dolichum DSM | NO: 364 | | | | | | | | | |
| 3991 | | | | | | | | | | |
| gi\|160915782\|ref\| | | | | | | | | | | |
| ZP_02077990.1 | | | | | | | | | | |
| Nitratifractor | SEQ ID | 143 | 184 | 42 | 347 | 404 | 61 | 347 | 404 | 61 |
| salsuginis DSM | NO: 365 | | | | | | | | | |
| 16511 | | | | | | | | | | |
| gi\|319957206\|ref\| | | | | | | | | | | |
| YP_004168469.1 | | | | | | | | | | |
| Rhodospirillum | SEQ ID | 139 | 180 | 42 | 314 | 371 | 55 | 314 | 371 | 55 |
| rubrum ATCC | NO: 366 | | | | | | | | | |
| 11170 | | | | | | | | | | |
| gi\|83591793\|ref\| | | | | | | | | | | |
| YP_425545.1\| | | | | | | | | | | |
| Clostridium | SEQ ID | 137 | 176 | 40 | 320 | 376 | 61 | 320 | 376 | 61 |
| cellulolyticum | NO: 367 | | | | | | | | | |
| H10 | | | | | | | | | | |
| gi\|220930482\|ref\| | | | | | | | | | | |
| YP_002507391.1 | | | | | | | | | | |
| Helicobacter | SEQ ID | 148 | 187 | 40 | 298 | 354 | 48 | 298 | 354 | 48 |
| mustelae 12198 | NO: 368 | | | | | | | | | |
| gi\|291276265\|ref\| | | | | | | | | | | |
| YP_003516037.1 | | | | | | | | | | |
| Ilyobacter | SEQ ID | 134 | 173 | 40 | 462 | 517 | 63 | 462 | 517 | 63 |
| polytropus DSM | NO: 369 | | | | | | | | | |
| 2926 | | | | | | | | | | |
| gi\|310780384\|ref\| | | | | | | | | | | |
| YP_003968716.1 | | | | | | | | | | |
| Sphaerochaeta | SEQ ID | 163 | 202 | 40 | 335 | 389 | 45 | 335 | 389 | 45 |
| globus str. Buddy | NO: 370 | | | | | | | | | |
| gi\|325972003\|ref\| | | | | | | | | | | |
| YP_004248194.1 | | | | | | | | | | |
| Staphylococcus | SEQ ID | 128 | 167 | 40 | 337 | 391 | 57 | 337 | 391 | 57 |
| lugdunensis | NO: 371 | | | | | | | | | |
| M23590 | | | | | | | | | | |
| gi\|315659848\|ref\| | | | | | | | | | | |
| ZP_07912707.1 | | | | | | | | | | |
| Treponema sp. | SEQ ID | 144 | 183 | 40 | 328 | 382 | 63 | 328 | 382 | 63 |
| JC4 | NO: 372 | | | | | | | | | |
| gi\|384109266\|ref\| | | | | | | | | | | |
| ZP_10010146.1 | | | | | | | | | | |
| uncultured delta | SEQ ID | 154 | 193 | 40 | 313 | 365 | 55 | 313 | 365 | 55 |
| proteobacterium | NO: 373 | | | | | | | | | |
| HF0070 07E19 | | | | | | | | | | |
| gi\|297182908\|gb\| | | | | | | | | | | |
| ADI19058.1\| | | | | | | | | | | |
| Alicycliphilus | SEQ ID | 140 | 178 | 39 | 317 | 366 | 48 | 317 | 366 | 48 |
| denitrificans K601 | NO: 374 | | | | | | | | | |
| gi\|330822845\|ref\| | | | | | | | | | | |
| YP_004386148.1 | | | | | | | | | | |
| Azospirillum sp. | SEQ ID | 205 | 243 | 39 | 342 | 389 | 46 | 342 | 389 | 46 |
| B510 | NO: 375 | | | | | | | | | |
| gi\|288957741\|ref\| | | | | | | | | | | |
| YP_003448082.1 | | | | | | | | | | |
| Bradyrhizobium | SEQ ID | 143 | 181 | 39 | 323 | 370 | 48 | 323 | 370 | 48 |
| sp. BTAi1 | NO: 376 | | | | | | | | | |
| gi\|148255343\|ref\| | | | | | | | | | | |
| YP_001239928.1 | | | | | | | | | | |
| Parvibaculum | SEQ ID | 138 | 176 | 39 | 327 | 374 | 58 | 327 | 374 | 58 |
| lavamentivorans | NO: 377 | | | | | | | | | |
| DS-1 | | | | | | | | | | |
| gi\|154250555\|ref\| | | | | | | | | | | |
| YP_001411379.1 | | | | | | | | | | |
| Prevotella | SEQ ID | 170 | 208 | 39 | 328 | 375 | 61 | 328 | 375 | 61 |
| timonensis CRIS | NO: 378 | | | | | | | | | |
| 5C-B1 | | | | | | | | | | |
| gi\|282880052\|ref\| | | | | | | | | | | |
| ZP_06288774.1 | | | | | | | | | | |
| Bacillus smithii 7 | SEQ ID | 134 | 171 | 38 | 401 | 448 | 63 | 401 | 448 | 63 |
| 3 47FAA | NO: 379 | | | | | | | | | |
| gi\|365156657\|ref\| | | | | | | | | | | |
| ZP_09352959.1 | | | | | | | | | | |
| Cand. | SEQ ID | 135 | 172 | 38 | 344 | 391 | 53 | 344 | 391 | 53 |
| Puniceispirillum | NO: 380 | | | | | | | | | |
| marinum | | | | | | | | | | |
| IMCC1322 | | | | | | | | | | |
| gi\|294086111\|ref\| | | | | | | | | | | |
| YP_003552871.1 | | | | | | | | | | |
| Barnesiella | SEQ ID | 140 | 176 | 37 | 371 | 417 | 60 | 371 | 417 | 60 |
| intestinihominis | NO: 381 | | | | | | | | | |
| YIT 11860 | | | | | | | | | | |
| gi\|404487228\|ref\| | | | | | | | | | | |
| ZP_11022414.1 | | | | | | | | | | |
| Ralstonia syzygii | SEQ ID | 140 | 176 | 37 | 395 | 440 | 50 | 395 | 440 | 50 |
| R24 | NO: 382 | | | | | | | | | |
| gi\|344171927\|emb\| | | | | | | | | | | |
| CCA84553.1\| | | | | | | | | | | |
| Wolinella | SEQ ID | 145 | 180 | 36 | 348 | 392 | 60 | 348 | 392 | 60 |
| succinogenes | NO: 383 | | | | | | | | | |
| DSM 1740 | | | | | | | | | | |
| gi\|34557790\|ref\| | | | | | | | | | | |
| NP_907605.1\| | | | | | | | | | | |
| Mycoplasma | SEQ ID | 144 | 177 | 34 | 373 | 416 | 71 | 373 | 416 | 71 |
| gallisepticum str. | NO: 384 | | | | | | | | | |
| F | | | | | | | | | | |
| gi\|284931710\|gb\| | | | | | | | | | | |
| ADC31648.1\| | | | | | | | | | | |
| Acidothermus | SEQ ID | 150 | 182 | 33 | 341 | 380 | 58 | 341 | 380 | 58 |
| cellulolyticus 11B | NO: 385 | | | | | | | | | |
| gi\|117929158\|ref\| | | | | | | | | | | |
| YP_873709.1\| | | | | | | | | | | |
| Mycoplasma | SEQ ID | 156 | 184 | 29 | 381 | 420 | 62 | 381 | 420 | 62 |
| ovipneumoniae | NO: 386 | | | | | | | | | |
| SC01 | | | | | | | | | | |
| gi\|363542550\|ref\| | | | | | | | | | | |
| ZP_09312133.1 | | | | | | | | | | |

TABLE 2B
Amino Acid Sequence of Cas9 Core Domains

| | |
|---|---|
| Cas9 Start | Cas9 Stop |
| (AA pos) | (AA pos) |

| | |
|---|---|
| | Start and Stop numbers refer to |
| Strain Name | the sequence in Table 2A |

| | | |
|---|---|---|
| Staphylococcus Aureus | 1 | 772 |
| Streptococcus Pyogenes | 1 | 1099 |
| Campulobacter Jejuni | 1 | 741 |

TABLE 3
Identified PAM sequences and corresponding RKR motifs.

| Strain Name | PAM sequence (NA) | RKR motif (AA) |
|---|---|---|
| Streptococcus pyogenes | NGG | RKR |
| Streptococcus mutans | NGG | RKR |
| Streptococcus thermophilus A | NGGNG | RYR |
| Treponema denticola | NAAAAN | VAK |
| Streptococcus thermophilus B | NNAAAAW | IYK |
| Campylobacter jejuni | NNNNACA | NLK |
| Pasteurella multocida | GNNNCNNA | KDG |
| Neisseria meningitidis | NNNNGATT or | IGK |
| | NNGRRT (R = A or G) | |
| Staphylococcus aureus | NNGRRV (R = A or G; | NDK |
| | V = A. G or C) or | |
| | NNGRRT (R = A or G) | |

PI domains are provided in Tables 4 and 5.

TABLE 4
Altered PI Domains

| | PI Start | PI Stop | | |
|---|---|---|---|---|
| | (AA pos) | (AA pos) | | |
| | Start and Stop | | Length | RKR |
| | numbers refer to the | | of PI | motif |
| Strain Name | sequences in Table 2A | | (AA) | (AA) |
| Alicycliphilus denitrificans K601 | 837 | 1029 | 193 | --Y |
| Campylobacter jejuni NCTC | 741 | 984 | 244 | -NG |
| 11168 | | | | |
| Helicobacter mustelae 12198 | 771 | 1024 | 254 | -NQ |

TABLE 5
Other Altered PI Domains

| | PI Start | PI Stop | | |
|---|---|---|---|---|
| | (AA pos) | (AA pos) | | |
| | Start and Stop | | Length | RKR |
| | numbers refer to the | | of PI | motif |
| Strain Name | sequences in Table 2A | | (AA) | (AA) |
| Akkermansia muciniphila ATCC | 871 | 1101 | 231 | ALK |
| BAA-835 | | | | |
| Ralstonia syzygii R24 | 821 | 1062 | 242 | APY |
| Cand. Puniceispirillum marinum | 815 | 1035 | 221 | AYK |
| IMCC1322 | | | | |
| Fructobacillus fructosus KCTC 3544 | 1074 | 1323 | 250 | DGN |
| Eubacterium yurii ATCC 43715 | 1107 | 1391 | 285 | DGY |
| Eubacterium dolichum DSM 3991 | 779 | 1096 | 318 | DKK |
| Dinoroseobacter shibae DFL 12 | 851 | 1079 | 229 | DPI |
| Clostridium cellulolyticum H10 | 767 | 1021 | 255 | EGK |
| Pasteurella multocida str. Pm70 | 815 | 1056 | 242 | ENN |
| Mycoplasma canis PG 14 | 907 | 1233 | 327 | EPK |
| Porphyromonas sp. oral taxon 279 str. | 935 | 1197 | 263 | EPT |
| F0450 | | | | |
| Filifactor alocis ATCC 35896 | 1094 | 1365 | 272 | EVD |
| Aminomonas paucivorans DSM 12260 | 801 | 1052 | 252 | EVY |
| Wolinella succinogenes DSM 1740 | 1034 | 1409 | 376 | EYK |
| Oenococcus kitaharae DSM 17330 | 1119 | 1389 | 271 | GAL |
| CoriobacteriumglomeransPW2 | 1126 | 1384 | 259 | GDR |
| Peptoniphilus duerdenii ATCC | 1091 | 1364 | 274 | GDS |
| BAA-1640 | | | | |
| Bifidobacterium bifidum S17 | 1138 | 1420 | 283 | GGL |
| Alicyclobacillus hesperidum | 876 | 1146 | 271 | GGR |
| URH17-3-68 | | | | |
| Roseburia inulinivorans DSM 16841 | 895 | 1152 | 258 | GGT |
| Actinomyces coleocanis DSM 15436 | 843 | 1105 | 263 | GKK |
| Odoribacter laneus YIT 12061 | 1103 | 1498 | 396 | GKV |
| Coprococcus catus GD-7 | 1063 | 1338 | 276 | GNQ |
| Enterococcus faecalis TX0012 | 829 | 1150 | 322 | GRK |
| Bacillus smithii 7 3 47FAA | 809 | 1088 | 280 | GSK |
| Legionella pneumophila str. Paris | 1021 | 1372 | 352 | GTM |
| Bacteroides fragilis NCTC 9343 | 1140 | 1436 | 297 | IPV |
| Mycoplasma ovipneumoniae SC01 | 923 | 1265 | 343 | IRI |
| Actinomyces sp. oral taxon 180 str. | 895 | 1181 | 287 | KEK |
| F0310 | | | | |
| Treponema sp. JC4 | 832 | 1062 | 231 | KIS |
| Fusobacteriumnucleatum | 1073 | 1374 | 302 | KKV |
| ATCC49256 | | | | |
| Lactobacillus farciminis KCTC 3681 | 1101 | 1356 | 256 | KKV |
| Nitratifractor salsuginis DSM 16511 | 840 | 1132 | 293 | KMR |
| Lactobacillus coryniformis KCTC 3535 | 850 | 1119 | 270 | KNK |
| Mycoplasma mobile 163K | 916 | 1236 | 321 | KNY |
| Flavobacterium branchiophilum FL-15 | 1182 | 1473 | 292 | KQK |
| Prevotella timonensis CRIS 5C-B1 | 957 | 1218 | 262 | KQQ |
| Methylosinus trichosporium OB3b | 830 | 1082 | 253 | KRP |
| Prevotella sp. C561 | 1099 | 1424 | 326 | KRY |
| Mycoplasma gallisepticum str. F | 911 | 1269 | 359 | KTA |
| Lactobacillus rhamnosus GG | 1077 | 1363 | 287 | KYG |
| Wolinella succinogenes DSM 1740 | 811 | 1059 | 249 | LPN |
| Streptococcus thermophilus LMD-9 | 1099 | 1388 | 290 | MLA |
| Treponema denticola ATCC 35405 | 1092 | 1395 | 304 | NDS |
| Bergeyella zoohelcum ATCC 43767 | 1098 | 1415 | 318 | NEK |
| Veillonella atypica ACS-134-V-Col7a | 1107 | 1398 | 292 | NGF |
| Neisseria meningitidis Z2491 | 835 | 1082 | 248 | NHN |
| Ignavibacterium album JCM 16511 | 1296 | 1688 | 393 | NKK |
| Ruminococcus albus 8 | 853 | 1156 | 304 | NNF |
| Streptococcus thermophilus LMD-9 | 811 | 1121 | 311 | NNK |
| Barnesiella intestinihominis YIT 11860 | 871 | 1153 | 283 | NPV |
| Azospirillum sp. B510 | 911 | 1168 | 258 | PFH |
| Rhodospirillum rubrum ATCC 11170 | 863 | 1173 | 311 | PRG |
| Planococcus antarcticus DSM 14505 | 1087 | 1333 | 247 | PYY |
| Staphylococcus pseudintermedius ED99 | 1073 | 1334 | 262 | QIV |
| Alcanivorax sp. W11-5 | 843 | 1113 | 271 | RIE |
| Bradyrhizobium sp. BTAi1 | 811 | 1064 | 254 | RIY |
| Streptococcus pyogenes M1 GAS | 1099 | 1368 | 270 | RKR |
| Streptococcus mutans UA159 | 1078 | 1345 | 268 | RKR |
| Streptococcus Pyogenes | 1099 | 1368 | 270 | RKR |
| Bacteroides sp. 20 3 | 1147 | 1517 | 371 | RNI |
| S. aureus | 772 | 1053 | 282 | RNK |
| Solobacterium moorei F0204 | 1062 | 1327 | 266 | RSG |
| Finegoldia magna ATCC 29328 | 1081 | 1348 | 268 | RTE |
| uncultured delta proteobacterium | 770 | 1011 | 242 | SGG |
| HF0070 07E19 | | | | |
| Acidaminococcus sp. D21 | 1064 | 1358 | 295 | SIG |
| Eubacterium rectale ATCC 33656 | 824 | 1114 | 291 | SKK |
| Caenispirillum salinarum AK4 | 1048 | 1442 | 395 | SLV |
| Acidothermus cellulolyticus 11B | 830 | 1138 | 309 | SPS |
| Catenibacterium mitsuokai DSM 15897 | 1068 | 1329 | 262 | SPT |
| Parvibaculum lavamentivorans DS-1 | 827 | 1037 | 211 | TGN |
| Staphylococcus lugdunensis M23590 | 772 | 1054 | 283 | TKK |
| Streptococcus sanguinis SK49 | 1123 | 1421 | 299 | TRM |
| Elusimicrobium minutum Pei191 | 910 | 1195 | 286 | TTG |
| Nitrobacter hamburgensis X14 | 914 | 1166 | 253 | VAY |
| Mycoplasma synoviae 53 | 991 | 1314 | 324 | VGF |
| Sphaerochaeta globus str. Buddy | 877 | 1179 | 303 | VKG |
| Ilyobacter polytropus DSM 2926 | 837 | 1092 | 256 | VNG |
| Rhodovulum sp. PH10 | 821 | 1059 | 239 | VPY |
| Bifidobacterium longum DJO10A | 904 | 1187 | 284 | VRK |

g) Nucleic Acids Encoding Cas9 Molecules

Nucleic acids encoding the Cas9 molecules or Cas9 polypeptides, e.g., an eaCas9 molecule or eaCas9 polypeptide, are provided herein.

Exemplary nucleic acids encoding Cas9 molecules or Cas9 polypeptides are described in Cong et al., Science 2013, 399(6121):819-823; Wang et al., Cell 2013, 153(4):910-918; Mali et al., Science 2013, 399(6121):823-826; Jinek et al., Science 2012, 337(6096):816-821. Another exemplary nucleic acid encoding a Cas9 molecule or Cas9 polypeptide is shown in black in FIG. 8.

In an embodiment, a nucleic acid encoding a Cas9 molecule or Cas9 polypeptide can be a synthetic nucleic acid sequence. For example, the synthetic nucleic acid molecule can be chemically modified. In an embodiment, the Cas9 mRNA has one or more (e.g., all of the following properties: it is capped, polyadenylated, substituted with 5-methylcytidine and/or pseudouridine.

In addition, or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein.

In addition, or alternatively, a nucleic acid encoding a Cas9 molecule or Cas9 polypeptide may comprise a nuclear localization sequence (NLS). Nuclear localization sequences are known in the art.

SEQ ID NO:22 is an exemplary codon optimized nucleic acid sequence encoding a Cas9 molecule of S. pyogenes. SEQ ID NO:23 is the corresponding amino acid sequence of a S. pyogenes Cas9 molecule.

SEQ ID NO:24 is an exemplary codon optimized nucleic acid sequence encoding a Cas9 molecule of N. meningitidis. SEQ ID NO:25 is the corresponding amino acid sequence of a N. meningitidis Cas9 molecule.

SEQ ID NO:26 is an amino acid sequence of a S. aureus Cas9 molecule. SEQ ID NO:39 is an exemplary codon optimized nucleic acid sequence encoding a Cas9 molecule of S. aureus Cas9.

If any of the above Cas9 sequences are fused with a peptide or polypeptide at the C-terminus, it is understood that the stop codon will be removed.

h) Other Cas Molecules and Cas Polypeptides

Various types of Cas molecules or Cas polypeptides can be used to practice the inventions disclosed herein. In some embodiments, Cas molecules of Type II Cas systems are used. In other embodiments, Cas molecules of other Cas systems are used. For example, Type I or Type III Cas molecules may be used. Exemplary Cas molecules (and Cas systems) are described, e.g., in Haft et al., PLoS Computational Biology 2005, 1(6): e60 and Makarova et al., Nature Review Microbiology 2011, 9:467-477, the contents of both references are incorporated herein by reference in their entirety. Exemplary Cas molecules (and Cas systems) are also shown in Table 600.

TABLE 600
Cas Systems

| | | | Structure of | Families (and | |
|---|---|---|---|---|---|
| | | | encoded | superfamily) of | |
| Gene | System type or | Name from | protein (PDB | encoded | |
| name^{‡} | subtype | Haft et al.^{§} | accessions)^{¶} | protein^{#}** | Representatives |
| cas1 | Type I | cas1 | 3GOD, 3LFX | COG1518 | SERP2463, |
| | Type II | | and 2YZS | | SPy1047 and ygbT |
| | Type III | | | | |
| cas2 | Type I | cas2 | 2IVY, 2I8E | COG1343 and | SERP2462, |
| | Type II | | and 3EXC | COG3512 | SPy1048, SPy1723 |
| | Type III | | | | (N-terminal |
| | | | | | domain) and ygbF |
| cas3′ | Type I^{‡‡} | cas3 | NA | COG1203 | APE1232 and ygcB |
| cas3″ | Subtype I-A | NA | NA | COG2254 | APE1231 and |
| | Subtype I-B | | | | BH0336 |
| cas4 | Subtype I-A | cas4 and | NA | COG1468 | APE1239 and |
| | Subtype I-B | csa1 | | | BH0340 |
| | Subtype I-C | | | | |
| | Subtype I-D | | | | |
| | Subtype II-B | | | | |
| cas5 | Subtype I-A | cas5a, | 3KG4 | COG1688 | APE1234, BH0337, |
| | Subtype I-B | cas5d, | | (RAMP) | devS and ygcI |
| | Subtype I-C | cas5e, | | | |
| | Subtype I-E | cas5h, | | | |
| | | cas5p, | | | |
| | | cas5t and | | | |
| | | cmx5 | | | |
| cas6 | Subtype I-A | cas6 and | 3I4H | COG1583 and | PF1131 and slr7014 |
| | Subtype I-B | cmx6 | | COG5551 | |
| | Subtype I-D | | | (RAMP) | |
| | Subtype III-A | | | | |
| | Subtype III-B | | | | |
| cas6e | Subtype I-E | cse3 | 1WJ9 | (RAMP) | ygcH |
| cas6f | Subtype I-F | csy4 | 2XLJ | (RAMP) | y1727 |
| cas7 | Subtype I-A | csa2, csd2, | NA | COG1857 and | devR and ygcJ |
| | Subtype I-B | cse4, csh2, | | COG3649 | |
| | Subtype I-C | csp1 and | | (RAMP) | |
| | Subtype I-E | cst2 | | | |
| cas8a1 | Subtype I-A^{‡‡} | cmx1, cst1, | NA | BH0338-like | LA3191^{§§} and |
| | | csx8, csx13 | | | PG2018^{§§} |
| | | and CXXC- | | | |
| | | CXXC | | | |
| cas8a2 | Subtype I-A^{‡‡} | csa4 and | NA | PH0918 | AF0070, AF1873, |
| | | csx9 | | | MJ0385, PF0637, |
| | | | | | PH0918 and |
| | | | | | SSO1401 |
| cas8b | Subtype I-B^{‡‡} | csh1 and | NA | BH0338-like | MTH1090 and |
| | | TM1802 | | | TM1802 |
| cas8c | Subtype I-C^{‡‡} | csd1 and | NA | BH0338-like | BH0338 |
| | | csp2 | | | |
| cas9 | Type II^{‡‡} | csn1 and | NA | COG3513 | FTN_0757 and |
| | | csx12 | | | SPy1046 |
| cas10 | Type III^{‡‡} | cmr2, csm1 | NA | COG1353 | MTH326, |
| | | and csx11 | | | Rv2823c^{§§} and |
| | | | | | TM1794^{§§} |
| cas10d | Subtype I-D^{‡‡} | csc3 | NA | COG1353 | slr7011 |
| csy1 | Subtype I-F^{‡‡} | csy1 | NA | y1724-like | y1724 |
| csy2 | Subtype I-F | csy2 | NA | (RAMP) | y1725 |
| csy3 | Subtype I-F | csy3 | NA | (RAMP) | y1726 |
| cse1 | Subtype I-E^{‡‡} | cse1 | NA | YgcL-like | ygcL |
| cse2 | Subtype I-E | cse2 | 2ZCA | YgcK-like | ygcK |
| csc1 | Subtype I-D | csc1 | NA | alr1563-like | alr1563 |
| | | | | (RAMP) | |
| csc2 | Subtype I-D | csc1 and | NA | COG1337 | slr7012 |
| | | csc2 | | (RAMP) | |
| csa5 | Subtype I-A | csa5 | NA | AF1870 | AF1870, MJ0380, |
| | | | | | PF0643 and |
| | | | | | SSO1398 |
| csn2 | Subtype II-A | csn2 | NA | SPy1049-like | SPy1049 |
| csm2 | Subtype III-A^{‡‡} | csm2 | NA | COG1421 | MTH1081 and |
| | | | | | SERP2460 |
| csm3 | Subtype III-A | csc2 and | NA | COG1337 | MTH1080 and |
| | | csm3 | | (RAMP) | SERP2459 |
| csm4 | Subtype III-A | csm4 | NA | COG1567 | MTH1079 and |
| | | | | (RAMP) | SERP2458 |
| csm5 | Subtype III-A | csm5 | NA | COG1332 | MTH1078 and |
| | | | | (RAMP) | SERP2457 |
| csm6 | Subtype III-A | APE2256 | 2WTE | COG1517 | APE2256 and |
| | | and csm6 | | | SSO1445 |
| cmr1 | Subtype III-B | cmr1 | NA | COG1367 | PF1130 |
| | | | | (RAMP) | |
| cmr3 | Subtype III-B | cmr3 | NA | COG1769 | PF1128 |
| | | | | (RAMP) | |
| cmr4 | Subtype III-B | cmr4 | NA | COG1336 | PF1126 |
| | | | | (RAMP) | |
| cmr5 | Subtype III-B^{‡‡} | cmr5 | 2ZOP and | COG3337 | MTH324 and |
| | | | 2OEB | | PF1125 |
| cmr6 | Subtype III-B | cmr6 | NA | COG1604 | PF1124 |
| | | | | (RAMP) | |
| csb1 | Subtype I-U | GSU0053 | NA | (RAMP) | Balac_1306 and |
| | | | | | GSU0053 |
| csb2 | Subtype I-U^{§§} | NA | NA | (RAMP) | Balac_1305 and |
| | | | | | GSU0054 |
| csb3 | Subtype I-U | NA | NA | (RAMP) | Balac_1303^{§§} |
| csx17 | Subtype I-U | NA | NA | NA | Btus_2683 |
| csx14 | Subtype I-U | NA | NA | NA | GSU0052 |
| csx10 | Subtype I-U | csx10 | NA | (RAMP) | Caur_2274 |
| csx16 | Subtype III-U | VVA1548 | NA | NA | VVA1548 |
| csaX | Subtype III-U | csaX | NA | NA | SSO1438 |
| csx3 | Subtype III-U | csx3 | NA | NA | AF1864 |
| csx1 | Subtype III-U | csa3, csx1, | 1XMX and | COG1517 and | MJ1666, NE0113, |
| | | csx2, | 2I71 | COG4006 | PF1127 and |
| | | DXTHG, | | | TM1812 |
| | | NE0113 | | | |
| | | and | | | |
| | | TIGR02710 | | | |
| csx15 | Unknown | NA | NA | TTE2665 | TTE2665 |
| csf1 | Type U | csf7 | NA | NA | AFE_1038 |
| csf2 | Type U | csf2 | NA | (RAMP) | AFE_1039 |
| csf3 | Type U | csf3 | NA | (RAMP) | AFE_1040 |
| csf4 | Type U | csf4 | NA | NA | AFE_1037 |

4. Genome Editing Methods and Methods of Delivery

a) Genome Editing Approaches

In general, it is to be understood that the alteration of any gene according to the methods described herein can be mediated by any mechanism and that any methods are not limited to a particular mechanism. Exemplary mechanisms that can be associated with the alteration of a gene include, but are not limited to, non-homologous end joining (e.g., classical or alternative), microhomology-mediated end joining (MMEJ), homology-directed repair (e.g., endogenous donor template mediated), synthesis dependent strand annealing (SDSA), single strand annealing, single strand invasion, single strand break repair (SSBR), mismatch repair (MMR), base excision repair (BER), Interstrand Crosslink (ICL) Translesion synthesis (TLS), or Error-free postreplication repair (PRR). Described herein are exemplary methods for targeted knockout of one or both alleles of PDCD1 encoding the protein PD-1.

(1) NHEJ Approaches for Gene Targeting

As described herein, nuclease-induced non-homologous end-joining (NHEJ) can be used to target gene-specific knockouts. Nuclease-induced NHEJ can also be used to remove (e.g., delete) sequence insertions in a gene of interest.

While not wishing to be bound by theory, it is believed that, in an embodiment, the genomic alterations associated with the methods described herein rely on nuclease-induced NHEJ and the error-prone nature of the NHEJ repair pathway. NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over represented in the population, likely due to small regions of microhomology. The lengths of deletions can vary widely; most commonly in the 1-50 bp range, but they can easily reach greater than 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it can also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. In some embodiments, a pair of gRNAs can be used to introduce two double-strand breaks, resulting in a deletion of intervening sequences between the two breaks.

Both of these approaches can be used to delete specific DNA sequences; however, the error-prone nature of NHEJ may still produce indel mutations at the site of repair.

Both double strand cleaving eaCas9 molecules and single strand, or nickase, eaCas9 molecules can be used in the methods and compositions described herein to generate NHEJ-mediated indels. NHEJ-mediated indels targeted to the gene, e.g., a coding region, e.g., an early coding region of a gene, of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

In an embodiment, NHEJ-mediated indels are introduced into one or more T-cell expressed genes, such as PDCD1. Individual gRNAs or gRNA pairs targeting the gene are provided together with the Cas9 double-stranded nuclease or single-stranded nickase.

(2) Placement of Double Strand or Single Strand Breaks Relative to the Target Position

In an embodiment, in which a gRNA and Cas9 nuclease generate a double strand break for the purpose of inducing NHEJ-mediated indels, a gRNA, e.g., a unimolecular (or chimeric) or modular gRNA molecule, is configured to position one double-strand break in close proximity to a nucleotide of the target position. In an embodiment, the cleavage site is between 0-30 bp away from the target position (e.g., less than 30, 25, 20, 15, 10, 9, 8, 7, 6, 5, 4, 3, 2 or 1 bp from the target position).

In an embodiment, in which two gRNAs complexing with Cas9 nickases induce two single strand breaks for the purpose of inducing NHEJ-mediated indels, two gRNAs, e.g., independently, unimolecular (or chimeric) or modular gRNA, are configured to position two single-strand breaks to provide for NHEJ repair a nucleotide of the target position. In an embodiment, the gRNAs are configured to position cuts at the same position, or within a few nucleotides of one another, on different strands, essentially mimicking a double strand break. In an embodiment, the closer nick is between 0-30 bp away from the target position (e.g., less than 30, 25, 20, 15, 10, 9, 8, 7, 6, 5, 4, 3, 2 or 1 bp from the target position), and the two nicks are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp). In an embodiment, the gRNAs are configured to place a single strand break on either side of a nucleotide of the target position.

Both double strand cleaving eaCas9 molecules and single strand, or nickase, eaCas9 molecules can be used in the methods and compositions described herein to generate breaks both sides of a target position. Double strand or paired single strand breaks may be generated on both sides of a target position to remove the nucleic acid sequence between the two cuts (e.g., the region between the two breaks in deleted). In an embodiment, two gRNAs, e.g., independently, unimolecular (or chimeric) or modular gRNA, are configured to position a double-strand break on both sides of a target position. In an alternate embodiment, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of the target position. In another embodiment, four gRNAs, e.g., independently, unimolecular (or chimeric) or modular gRNA, are configured to generate two pairs of single stranded breaks (i.e., two pairs of two gRNAs complex with Cas9 nickases) on either side of the target position. The double strand break(s) or the closer of the two single strand nicks in a pair will ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp).

b) Targeted Knockdown

Unlike CRISPR/Cas-mediated gene knockout, which permanently eliminates or reduces expression by mutating the gene at the DNA level, CRISPR/Cas knockdown allows for temporary reduction of gene expression through the use of artificial transcription factors. Mutating key residues in both DNA cleavage domains of the Cas9 protein (e.g., the D10A and H840A mutations) results in the generation of a catalytically inactive Cas9 (eiCas9 which is also known as dead Cas9 or dCas9). A catalytically inactive Cas9 complexes with a gRNA and localizes to the DNA sequence specified by that gRNA's targeting domain, however, it does not cleave the target DNA. Fusion of the dCas9 to an effector domain, e.g., a transcription repression domain, enables recruitment of the effector to any DNA site specified by the gRNA. While it has been shown that the eiCas9 itself can block transcription when recruited to early regions in the coding sequence, more robust repression can be achieved by fusing a transcriptional repression domain (for example KRAB, SID or ERD) to the Cas9 and recruiting it to the promoter region of a gene. It is likely that targeting DNAseI hypersensitive regions of the promoter may yield more efficient gene repression or activation because these regions are more likely to be accessible to the Cas9 protein and are also more likely to harbor sites for endogenous transcription factors. Especially for gene repression, it is contemplated herein that blocking the binding site of an endogenous transcription factor would aid in downregulating gene expression. In another embodiment, an eiCas9 can be fused to a chromatin modifying protein. Altering chromatin status can result in decreased expression of the target gene.

In an embodiment, a gRNA molecule can be targeted to a known transcription response elements (e.g., promoters, enhancers, etc.), a known upstream activating sequences (UAS), and/or sequences of unknown or known function that are suspected of being able to control expression of the target DNA.

In an embodiment, CRISPR/Cas-mediated gene knockdown can be used to reduce expression one or more T-cell expressed genes. In an embodiment, in which a eiCas9 or an eiCas9 fusion protein described herein is used to knockdown two T-cell expressed genes, e.g., any two of FAS, BID, CTLA4, PDCD1, CBLB, or PTPN6 genes, individual gRNAs or gRNA pairs targeting both genes are provided together with the eiCas9 or eiCas9 fusion protein. In an embodiment, in which a eiCas9 or eiCas9 fusion protein is used to knockdown three T-cell expressed genes, e.g., any three of FAS, BID, CTLA4, PDCD1, CBLB, or PTPN6 genes, individual gRNAs or gRNA pairs targeting all three genes are provided together with the eiCas9 or eiCas9 fusion protein. In an embodiment, in which a eiCas9 or eiCas9 fusion protein is used to knockdown four T-cell expressed genes, e.g., any four of FAS, BID, CTLA4, PDCD1, CBLB, or PTPN6 genes, individual gRNAs or gRNA pairs targeting all four genes are provided together with the eiCas9 or eiCas9 fusion protein. In an embodiment, in which a eiCas9 or eiCas9 fusion protein is used to knockdown five T-cell expressed genes, e.g., any five of FAS, BID, CTLA4, PDCD1, CBLB, or PTPN6 genes, individual gRNAs or gRNA pairs targeting all five genes are provided together with the eiCas9 or eiCas9 fusion protein. In an embodiment, in which a eiCas9 or eiCas9 fusion protein is used to knockdown six T-cell expressed genes, e.g., each of FAS, BID, CTLA4, PDCD1, CBLB, or PTPN6 genes, individual gRNAs or gRNA pairs targeting all six genes are provided together with the eiCas9 or eiCas9 fusion protein.

c) Single-Strand Annealing

Single strand annealing (SSA) is another DNA repair process that repairs a double-strand break between two repeat sequences present in a target nucleic acid. Repeat sequences utilized by the SSA pathway are generally greater than 30 nucleotides in length. Resection at the break ends occurs to reveal repeat sequences on both strands of the target nucleic acid. After resection, single strand overhangs containing the repeat sequences are coated with RPA protein to prevent the repeats sequences from inappropriate annealing, e.g., to themselves. RAD52 binds to and each of the repeat sequences on the overhangs and aligns the sequences to enable the annealing of the complementary repeat sequences. After annealing, the single-strand flaps of the overhangs are cleaved. New DNA synthesis fills in any gaps, and ligation restores the DNA duplex. As a result of the processing, the DNA sequence between the two repeats is deleted. The length of the deletion can depend on many factors including the location of the two repeats utilized, and the pathway or processivity of the resection.

In contrast to HDR pathways, SSA does not require a template nucleic acid to alter or correct a target nucleic acid sequence. Instead, the complementary repeat sequence is utilized.

d) Other DNA Repair Pathways

(1) SSBR (Single Strand Break Repair)

Single-stranded breaks (SSB) in the genome are repaired by the SSBR pathway, which is a distinct mechanism from the DSB repair mechanisms discussed above. The SSBR pathway has four major stages: SSB detection, DNA end processing, DNA gap filling, and DNA ligation. A more detailed explanation is given in Caldecott, Nature Reviews Genetics 9, 619-631 (August 2008), and a summary is given here.

In the first stage, when a SSB forms, PARP1 and/or PARP2 recognize the break and recruit repair machinery. The binding and activity of PARP1 at DNA breaks is transient and it seems to accelerate SSBr by promoting the focal accumulation or stability of SSBr protein complexes at the lesion. Arguably the most important of these SSBr proteins is XRCC1, which functions as a molecular scaffold that interacts with, stabilizes, and stimulates multiple enzymatic components of the SSBr process including the protein responsible for cleaning the DNA 3′ and 5′ ends. For instance, XRCC1 interacts with several proteins (DNA polymerase beta, PNK, and three nucleases, APE1, APTX, and APLF) that promote end processing. APE1 has endonuclease activity. APLF exhibits endonuclease and 3′ to 5′ exonuclease activities. APTX has endonuclease and 3′ to 5′ exonuclease activity.

This end processing is an important stage of SSBR since the 3′- and/or 5′-termini of most, if not all, SSBs are ‘damaged’. End processing generally involves restoring a damaged 3′-end to a hydroxylated state and and/or a damaged 5′ end to a phosphate moiety, so that the ends become ligation-competent. Enzymes that can process damaged 3′ termini include PNKP, APE1, and TDP1. Enzymes that can process damaged 5′ termini include PNKP, DNA polymerase beta, and APTX. LIG3 (DNA ligase III) can also participate in end processing. Once the ends are cleaned, gap filling can occur.

At the DNA gap filling stage, the proteins typically present are PARP1, DNA polymerase beta, XRCC1, FEN1 (flap endonculease 1), DNA polymerase delta/epsilon, PCNA, and LIG1. There are two ways of gap filling, the short patch repair and the long patch repair. Short patch repair involves the insertion of a single nucleotide that is missing. At some SSBs, “gap filling” might continue displacing two or more nucleotides (displacement of up to 12 bases have been reported). FEN1 is an endonuclease that removes the displaced 5′-residues. Multiple DNA polymerases, including Pol (3, are involved in the repair of SSBs, with the choice of DNA polymerase influenced by the source and type of SSB.

In the fourth stage, a DNA ligase such as LIG1 (Ligase I) or LIG3 (Ligase III) catalyzes joining of the ends. Short patch repair uses Ligase III and long patch repair uses Ligase I.

Sometimes, SSBR is replication-coupled. This pathway can involve one or more of CtIP, MRN, ERCC1, and FEN1. Additional factors that may promote SSBR include: aPARP, PARP1, PARP2, PARG, XRCC1, DNA polymerase b, DNA polymerase d, DNA polymerase e, PCNA, LIG1, PNK, PNKP, APE1, APTX, APLF, TDP1, LIG3, FEN1, CtIP, MRN, and ERCC1.

(2) MMR (Mismatch Repair)

Cells contain three excision repair pathways: MMR, BER, and NER. The excision repair pathways hace a common feature in that they typically recognize a lesion on one strand of the DNA, then exo/endonucleaseases remove the lesion and leave a 1-30 nucleotide gap that is sub-sequentially filled in by DNA polymerase and finally sealed with ligase. A more complete picture is given in Li, Cell Research (2008) 18:85-98, and a summary is provided here.

Mismatch repair (MMR) operates on mispaired DNA bases.

The MSH2/6 or MSH2/3 complexes both have ATPases activity that plays an important role in mismatch recognition and the initiation of repair. MSH2/6 preferentially recognizes base-base mismatches and identifies mispairs of 1 or 2 nucleotides, while MSH2/3 preferentially recognizes larger ID mispairs.

hMLH1 heterodimerizes with hPMS2 to form hMutLα which possesses an ATPase activity and is important for multiple steps of MMR. It possesses a PCNA/replication factor C (RFC)-dependent endonuclease activity which plays an important role in 3′ nick-directed MMR involving EXO1. (EXO1 is a participant in both HR and MMR.) It regulates termination of mismatch-provoked excision. Ligase I is the relevant ligase for this pathway. Additional factors that may promote MMR include: EXO1, MSH2, MSH3, MSH6, MLH1, PMS2, MLH3, DNA Pol d, RPA, HMGB1, RFC, and DNA ligase I.

(3) Base Excision Repair (BER)

The base excision repair (BER) pathway is active throughout the cell cycle; it is responsible primarily for removing small, non-helix-distorting base lesions from the genome. In contrast, the related Nucleotide Excision Repair pathway (discussed in the next section) repairs bulky helix-distorting lesions. A more detailed explanation is given in Caldecott, Nature Reviews Genetics 9, 619-631 (August 2008), and a summary is given here.

Upon DNA base damage, base excision repair (BER) is initiated and the process can be simplified into five major steps: (a) removal of the damaged DNA base; (b) incision of the subsequent a basic site; (c) clean-up of the DNA ends; (d) insertion of the correct nucleotide into the repair gap; and (e) ligation of the remaining nick in the DNA backbone. These last steps are similar to the SSBR.

In the first step, a damage-specific DNA glycosylase excises the damaged base through cleavage of the N-glycosidic bond linking the base to the sugar phosphate backbone. Then AP endonuclease-1 (APE1) or bifunctional DNA glycosylases with an associated lyase activity incised the phosphodiester backbone to create a DNA single strand break (SSB). The third step of BER involves cleaning-up of the DNA ends. The fourth step in BER is conducted by Pol β that adds a new complementary nucleotide into the repair gap and in the final step XRCC1/Ligase III seals the remaining nick in the DNA backbone. This completes the short-patch BER pathway in which the majority (˜80%) of damaged DNA bases are repaired. However, if the 5′-ends in step 3 are resistant to end processing activity, following one nucleotide insertion by Pol β there is then a polymerase switch to the replicative DNA polymerases, Pol δ/ε, which then add ˜2-8 more nucleotides into the DNA repair gap. This creates a 5′-flap structure, which is recognized and excised by flap endonuclease-1 (FEN-1) in association with the processivity factor proliferating cell nuclear antigen (PCNA). DNA ligase I then seals the remaining nick in the DNA backbone and completes long-patch BER. Additional factors that may promote the BER pathway include: DNA glycosylase, APE1, Polb, Pold, Pole, XRCC1, Ligase III, FEN-1, PCNA, RECQL4, WRN, MYH, PNKP, and APTX.

(4) Nucleotide Excision Repair (NER)

Nucleotide excision repair (NER) is an important excision mechanism that removes bulky helix-distorting lesions from DNA. Additional details about NER are given in Marteijn et al., Nature Reviews Molecular Cell Biology 15, 465-481 (2014), and a summary is given here. NER a broad pathway encompassing two smaller pathways: global genomic NER (GG-NER) and transcription coupled repair NER (TC-NER). GG-NER and TC-NER use different factors for recognizing DNA damage. However, they utilize the same machinery for lesion incision, repair, and ligation.

Once damage is recognized, the cell removes a short single-stranded DNA segment that contains the lesion. Endonucleases XPF/ERCC1 and XPG (encoded by ERCC5) remove the lesion by cutting the damaged strand on either side of the lesion, resulting in a single-strand gap of 22-30 nucleotides. Next, the cell performs DNA gap filling synthesis and ligation. Involved in this process are: PCNA, RFC, DNA Pol δ, DNA Pol ε or DNA Pol κ, and DNA ligase I or XRCC1/Ligase III. Replicating cells tend to use DNA pol ε and DNA ligase I, while non-replicating cells tend to use DNA Pol δ, DNA Pol κ, and the XRCC1/Ligase III complex to perform the ligation step.

NER can involve the following factors: XPA-G, POLH, XPF, ERCC1, XPA-G, and LIG1. Transcription-coupled NER (TC-NER) can involve the following factors: CSA, CSB, XPB, XPD, XPG, ERCC1, and TTDA. Additional factors that may promote the NER repair pathway include XPA-G, POLH, XPF, ERCC1, XPA-G, LIG1, CSA, CSB, XPA, XPB, XPC, XPD, XPF, XPG, TTDA, UVSSA, USP7, CETN2, RAD23B, UV-DDB, CAK subcomplex, RPA, and PCNA.

(5) Intrastrand Crosslink (ICL)

A dedicated pathway called the ICL repair pathway repairs interstrand crosslinks. Interstrand crosslinks, or covalent crosslinks between bases in different DNA strand, can occur during replication or transcription. ICL repair involves the coordination of multiple repair processes, in particular, nucleolytic activity, translesion synthesis (TLS), and HDR. Nucleases are recruited to excise the ICL on either side of the crosslinked bases, while TLS and HDR are coordinated to repair the cut strands. ICL repair can involve the following factors: endonucleases, e.g., XPF and RAD51C, endonucleases such as RAD51, translesion polymerases, e.g., DNA polymerase zeta and Rev1), and the Fanconi anemia (FA) proteins, e.g., FancJ.

(6) Other Pathways

Several other DNA repair pathways exist in mammals.

Translesion synthesis (TLS) is a pathway for repairing a single stranded break left after a defective replication event and involves translesion polymerases, e.g., DNA pol□ and Rev1.

Error-free postreplication repair (PRR) is another pathway for repairing a single stranded break left after a defective replication event.
e) Examples of gRNAs in Genome Editing Methods

Any of the gRNA molecules as described herein can be used with any Cas9 molecules that generate a double strand break or a single strand break to alter the sequence of a target nucleic acid, e.g., a target position or target genetic signature. In some examples, the target nucleic acid is at or near the PDCD1 locus, such as any as described. In some embodiments, a ribonucleic acid molecule, such as a gRNA molecule, and a protein, such as a Cas9 protein or variants thereof, are introduced to any of the engineered cells provided herein. gRNA molecules useful in these methods are described below.

In an embodiment, the gRNA, e.g., a chimeric gRNA, is configured such that it comprises one or more of the following properties;
a) it can position, e.g., when targeting a Cas9 molecule that makes double strand breaks, a double strand break (i) within 50, 100, 150, 200, 250, 300, 350, 400, 450, or 500 nucleotides of a target position, or (ii) sufficiently close that the target position is within the region of end resection;
b) it has a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and
c)
(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5; 6, 7, 8, 9 or 10 nucleotides therefrom;
(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain, or a sequence that differs by no more than 1, 2, 3, 4, 5; 6, 7, 8, 9 or 10 nucleotides therefrom; or
(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain.

In an embodiment, the gRNA is configured such that it comprises properties: a and b(i).

In an embodiment, the gRNA is configured such that it comprises properties: a and b(ii).

In an embodiment, the gRNA is configured such that it comprises properties: a and b(iii).

In an embodiment, the gRNA is configured such that it comprises properties: a and b(iv).

In an embodiment, the gRNA is configured such that it comprises properties: a and b(v).

In an embodiment, the gRNA is configured such that it comprises properties: a and b(vi).

In an embodiment, the gRNA is configured such that it comprises properties: a and b(vii).

In an embodiment, the gRNA is configured such that it comprises properties: a and b(viii).

In an embodiment, the gRNA is configured such that it comprises properties: a and b(ix).

In an embodiment, the gRNA is configured such that it comprises properties: a and b(x).

In an embodiment, the gRNA is configured such that it comprises properties: a and b(xi).

In an embodiment, the gRNA is configured such that it comprises properties: a and c.

In an embodiment, the gRNA is configured such that in comprises properties: a, b, and c.

In an embodiment, the gRNA is configured such that in comprises properties: a(i), b(i), and c(i).

In an embodiment, the gRNA is configured such that in comprises properties: a(i), b(i), and c(ii).

In an embodiment, the gRNA is configured such that in comprises properties: a(i), b(ii), and c(i).

In an embodiment, the gRNA is configured such that in comprises properties: a(i), b(ii), and c(ii).

In an embodiment, the gRNA is configured such that in comprises properties: a(i), b(iii), and c(i).

In an embodiment, the gRNA is configured such that in comprises properties: a(i), b(iii), and c(ii).

In an embodiment, the gRNA is configured such that in comprises properties: a(i), b(iv), and c(i).

In an embodiment, the gRNA is configured such that in comprises properties: a(i), b(iv), and c(ii).

In an embodiment, the gRNA is configured such that in comprises properties: a(i), b(v), and c(i).

In an embodiment, the gRNA is configured such that in comprises properties: a(i), b(v), and c(ii).

In an embodiment, the gRNA is configured such that in comprises properties: a(i), b(vi), and c(i).

In an embodiment, the gRNA is configured such that in comprises properties: a(i), b(vi), and c(ii).

In an embodiment, the gRNA is configured such that in comprises properties: a(i), b(vii), and c(i).

In an embodiment, the gRNA is configured such that in comprises properties: a(i), b(vii), and c(ii).

In an embodiment, the gRNA is configured such that in comprises properties: a(i), b(viii), and c(i).

In an embodiment, the gRNA is configured such that in comprises properties: a(i), b(viii), and c(ii).

In an embodiment, the gRNA is configured such that in comprises properties: a(i), b(ix), and c(i).

In an embodiment, the gRNA is configured such that in comprises properties: a(i), b(ix), and c(ii).

In an embodiment, the gRNA is configured such that in comprises properties: a(i), b(x), and c(i).

In an embodiment, the gRNA is configured such that in comprises properties: a(i), b(x), and c(ii).

In an embodiment, the gRNA is configured such that in comprises properties: a(i), b(xi), and c(i).

In an embodiment, the gRNA is configured such that in comprises properties: a(i), b(xi), and c(ii).

In an embodiment, the gRNA, e.g., a chimeric gRNA, is configured such that it comprises one or more of the following properties;
a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;
b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and
c)
(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5; 6, 7, 8, 9 or 10 nucleotides therefrom;
(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain, or a sequence that differs by no more than 1, 2, 3, 4, 5; 6, 7, 8, 9 or 10 nucleotides therefrom; or
(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain.

In an embodiment, the gRNA is configured such that it comprises properties: a and b(i).

In an embodiment, the gRNA is configured such that it comprises properties: a and b(ii).

In an embodiment, the gRNA is configured such that it comprises properties: a and b(iii).

In an embodiment, the gRNA is configured such that it comprises properties: a and b(iv).

In an embodiment, the gRNA is configured such that it comprises properties: a and b(v).

In an embodiment, the gRNA is configured such that it comprises properties: a and b(vi).

In an embodiment, the gRNA is configured such that it comprises properties: a and b(vii).

In an embodiment, the gRNA is configured such that it comprises properties: a and b(viii).

In an embodiment, the gRNA is configured such that it comprises properties: a and b(ix).

In an embodiment, the gRNA is configured such that it comprises properties: a and b(x).

In an embodiment, the gRNA is configured such that it comprises properties: a and b(xi).

In an embodiment, the gRNA is configured such that it comprises properties: a and c.

In an embodiment, the gRNA is configured such that in comprises properties: a, b, and c.

In an embodiment, the gRNA is configured such that in comprises properties: a(i), b(i), and c(i).

In an embodiment, the gRNA is configured such that in comprises properties: a(i), b(i), and c(ii).

In an embodiment, the gRNA is configured such that in comprises properties: a(i), b(ii), and c(i).

In an embodiment, the gRNA is configured such that in comprises properties: a(i), b(ii), and c(ii).

In an embodiment, the gRNA is configured such that in comprises properties: a(i), b(iii), and c(i).

In an embodiment, the gRNA is configured such that in comprises properties: a(i), b(iii), and c(ii).

In an embodiment, the gRNA is configured such that in comprises properties: a(i), b(iv), and c(i).

In an embodiment, the gRNA is configured such that in comprises properties: a(i), b(iv), and c(ii).

In an embodiment, the gRNA is configured such that in comprises properties: a(i), b(v), and c(i).

In an embodiment, the gRNA is configured such that in comprises properties: a(i), b(v), and c(ii).

In an embodiment, the gRNA is configured such that in comprises properties: a(i), b(vi), and c(i).

In an embodiment, the gRNA is configured such that in comprises properties: a(i), b(vi), and c(ii).

In an embodiment, the gRNA is configured such that in comprises properties: a(i), b(vii), and c(i).

In an embodiment, the gRNA is configured such that in comprises properties: a(i), b(vii), and c(ii).

In an embodiment, the gRNA is configured such that in comprises properties: a(i), b(viii), and c(i).

In an embodiment, the gRNA is configured such that in comprises properties: a(i), b(viii), and c(ii).

In an embodiment, the gRNA is configured such that in comprises properties: a(i), b(ix), and c(i).

In an embodiment, the gRNA is configured such that in comprises properties: a(i), b(ix), and c(ii).

In an embodiment, the gRNA is configured such that in comprises properties: a(i), b(x), and c(i).

In an embodiment, the gRNA is configured such that in comprises properties: a(i), b(x), and c(ii).

In an embodiment, the gRNA is configured such that in comprises properties: a(i), b(xi), and c(i).

In an embodiment, the gRNA is configured such that in comprises properties: a(i), b(xi), and c(ii).

In an embodiment, the gRNA is used with a Cas9 nickase molecule having HNH activity, e.g., a Cas9 molecule having the RuvC activity inactivated, e.g., a Cas9 molecule having a mutation at D10, e.g., the D10A mutation.

In an embodiment, the gRNA is used with a Cas9 nickase molecule having RuvC activity, e.g., a Cas9 molecule having the HNH activity inactivated, e.g., a Cas9 molecule having a mutation at H840, e.g., a H840A.

In an embodiment, a pair of gRNAs, e.g., a pair of chimeric gRNAs, comprising a first and a second gRNA, is configured such that they comprises one or more of the following properties;
a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;
b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides;
c) for one or both:
(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5; 6, 7, 8, 9 or 10 nucleotides therefrom;
(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;
(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain; or, or a sequence that differs by no more than 1, 2, 3, 4, 5; 6, 7, 8, 9 or 10 nucleotides therefrom; or
(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain;
d) the gRNAs are configured such that, when hybridized to target nucleic acid, they are separated by 0-50, 0-100, 0-200, at least 10, at least 20, at least 30 or at least 50 nucleotides;
e) the breaks made by the first gRNA and second gRNA are on different strands; and
f) the PAMs are facing outwards.

In an embodiment, one or both of the gRNAs is configured such that it comprises properties: a and b(i).

In an embodiment, one or both of the gRNAs is configured such that it comprises properties: a and b(ii).

In an embodiment, one or both of the gRNAs is configured such that it comprises properties: a and b(iii).

In an embodiment, one or both of the gRNAs is configured such that it comprises properties: a and b(iv).

In an embodiment, one or both of the gRNAs is configured such that it comprises properties: a and b(v).

In an embodiment, one or both of the gRNAs is configured such that it comprises properties: a and b(vi).

In an embodiment, one or both of the gRNAs is configured such that it comprises properties: a and b(vii).

In an embodiment, one or both of the gRNAs is configured such that it comprises properties: a and b(viii).

In an embodiment, one or both of the gRNAs is configured such that it comprises properties: a and b(ix).

In an embodiment, one or both of the gRNAs is configured such that it comprises properties: a and b(x).

In an embodiment, one or both of the gRNAs is configured such that it comprises properties: a and b(xi).

In an embodiment, one or both of the gRNAs configured such that it comprises properties: a and c.

In an embodiment, one or both of the gRNAs is configured such that it comprises properties: a, b, and c.

In an embodiment, one or both of the gRNAs is configured such that it comprises properties: a(i), b(i), and c(i).

In an embodiment, one or both of the gRNAs is configured such that it comprises properties: a(i), b(i), and c(ii).

In an embodiment, one or both of the gRNAs is configured such that it comprises properties: a(i), b(i), c, and d.

In an embodiment, one or both of the gRNAs is configured such that it comprises properties: a(i), b(i), c, and e.

In an embodiment, one or both of the gRNAs is configured such that it comprises properties: a(i), b(i), c, d, and e.

In an embodiment, one or both of the gRNAs is configured such that it comprises properties: a(i), b(ii), and c(i).

In an embodiment, one or both of the gRNAs is configured such that it comprises properties: a(i), b(ii), and c(ii).

In an embodiment, one or both of the gRNAs is configured such that it comprises properties: a(i), b(ii), c, and d.

In an embodiment, one or both of the gRNAs is configured such that it comprises properties: a(i), b(ii), c, and e.

In an embodiment, one or both of the gRNAs is configured such that it comprises properties: a(i), b(ii), c, d, and e.

In an embodiment, one or both of the gRNAs is configured such that it comprises properties: a(i), b(iii), and c(i).

In an embodiment, one or both of the gRNAs is configured such that it comprises properties: a(i), b(iii), and c(ii).

In an embodiment, one or both of the gRNAs is configured such that it comprises properties: a(i), b(iii), c, and d.

In an embodiment, one or both of the gRNAs is configured such that it comprises properties: a(i), b(iii), c, and e.

In an embodiment, one or both of the gRNAs is configured such that it comprises properties: a(i), b(iii), c, d, and e.

In an embodiment, one or both of the gRNAs is configured such that it comprises properties: a(i), b(iv), and c(i).

In an embodiment, one or both of the gRNAs is configured such that it comprises properties: a(i), b(iv), and c(ii).

In an embodiment, one or both of the gRNAs is configured such that it comprises properties: a(i), b(iv), c, and d.

In an embodiment, one or both of the gRNAs is configured such that it comprises properties: a(i), b(iv), c, and e.

In an embodiment, one or both of the gRNAs is configured such that it comprises properties: a(i), b(iv), c, d, and e.

In an embodiment, one or both of the gRNAs is configured such that it comprises properties: a(i), b(v), and c(i).

In an embodiment, one or both of the gRNAs is configured such that it comprises properties: a(i), b(v), and c(ii).

In an embodiment, one or both of the gRNAs is configured such that it comprises properties: a(i), b(v), c, and d.

In an embodiment, one or both of the gRNAs is configured such that it comprises properties: a(i), b(v), c, and e.

In an embodiment, one or both of the gRNAs is configured such that it comprises properties: a(i), b(v), c, d, and e.

In an embodiment, one or both of the gRNAs is configured such that it comprises properties: a(i), b(vi), and c(i).

In an embodiment, one or both of the gRNAs is configured such that it comprises properties: a(i), b(vi), and c(ii).

In an embodiment, one or both of the gRNAs is configured such that it comprises properties: a(i), b(vi), c, and d.

In an embodiment, one or both of the gRNAs is configured such that it comprises properties: a(i), b(vi), c, and e.

In an embodiment, one or both of the gRNAs is configured such that it comprises properties: a(i), b(vi), c, d, and e.

In an embodiment, one or both of the gRNAs is configured such that it comprises properties: a(i), b(vii), and c(i).

In an embodiment, one or both of the gRNAs is configured such that it comprises properties: a(i), b(vii), and c(ii).

In an embodiment, one or both of the gRNAs is configured such that it comprises properties: a(i), b(vii), c, and d.

In an embodiment, one or both of the gRNAs is configured such that it comprises properties: a(i), b(vii), c, and e.

In an embodiment, one or both of the gRNAs is configured such that it comprises properties: a(i), b(vii), c, d, and e.

In an embodiment, one or both of the gRNAs is configured such that it comprises properties: a(i), b(viii), and c(i).

In an embodiment, one or both of the gRNAs is configured such that it comprises properties: a(i), b(viii), and c(ii).

In an embodiment, one or both of the gRNAs is configured such that it comprises properties: a(i), b(viii), c, and d.

In an embodiment, one or both of the gRNAs is configured such that it comprises properties: a(i), b(viii), c, and e.

In an embodiment, one or both of the gRNAs is configured such that it comprises properties: a(i), b(viii), c, d, and e.

In an embodiment, one or both of the gRNAs is configured such that it comprises properties: a(i), b(ix), and c(i).

In an embodiment, one or both of the gRNAs is configured such that it comprises properties: a(i), b(ix), and c(ii).

In an embodiment, one or both of the gRNAs is configured such that it comprises properties: a(i), b(ix), c, and d.

In an embodiment, one or both of the gRNAs is configured such that it comprises properties: a(i), b(ix), c, and e.

In an embodiment, one or both of the gRNAs is configured such that it comprises properties: a(i), b(ix), c, d, and e.

In an embodiment, one or both of the gRNAs is configured such that it comprises properties: a(i), b(x), and c(i).

In an embodiment, one or both of the gRNAs is configured such that it comprises properties: a(i), b(x), and c(ii).

In an embodiment, one or both of the gRNAs is configured such that it comprises properties: a(i), b(x), c, and d.

In an embodiment, one or both of the gRNAs is configured such that it comprises properties: a(i), b(x), c, and e.

In an embodiment, one or both of the gRNAs is configured such that it comprises properties: a(i), b(x), c, d, and e.

In an embodiment, one or both of the gRNAs is configured such that it comprises properties: a(i), b(xi), and c(i).

In an embodiment, one or both of the gRNAs is configured such that it comprises properties: a(i), b(xi), and c(ii).

In an embodiment, one or both of the gRNAs is configured such that it comprises properties: a(i), b(xi), c, and d.

In an embodiment, one or both of the gRNAs is configured such that it comprises properties: a(i), b(xi), c, and e.

In an embodiment, one or both of the gRNAs is configured such that it comprises properties: a(i), b(xi), c, d, and e.

In an embodiment, the gRNAs are used with a Cas9 nickase molecule having HNH activity, e.g., a Cas9 molecule having the RuvC activity inactivated, e.g., a Cas9 molecule having a mutation at D10, e.g., the D10A mutation.

In an embodiment, the gRNAs are used with a Cas9 nickase molecule having RuvC activity, e.g., a Cas9 molecule having the HNH activity inactivated, e.g., a Cas9 molecule having a mutation at H840, e.g., a H840A. In an embodiment, the gRNAs are used with a Cas9 nickase molecule having RuvC activity, e.g., a Cas9 molecule having the HNH activity inactivated, e.g., a Cas9 molecule having a mutation at N863, e.g., N863A.

(1) Functional Analysis of Agents for Gene Editing

Any of the Cas9 molecules, gRNA molecules, Cas9 molecule/gRNA molecule complexes, can be evaluated by art-known methods or as described herein. For example, exemplary methods for evaluating the endonuclease activity of Cas9 molecule are described, e.g., in Jinek et al., SCIENCE 2012, 337(6096):816-821.

(a) Binding and Cleavage Assay: Testing the Endonuclease Activity of Cas9 Molecule

The ability of a Cas9 molecule/gRNA molecule complex to bind to and cleave a target nucleic acid can be evaluated in a plasmid cleavage assay. In this assay, synthetic or in vitro-transcribed gRNA molecule is pre-annealed prior to the reaction by heating to 95° C. and slowly cooling down to room temperature. Native or restriction digest-linearized plasmid DNA (300 ng (˜8 nM)) is incubated for 60 min at 37° C. with purified Cas9 protein molecule (50-500 nM) and gRNA (50-500 nM, 1:1) in a Cas9 plasmid cleavage buffer (20 mM HEPES pH 7.5, 150 mM KCl, 0.5 mM DTT, 0.1 mM EDTA) with or without 10 mM MgCl₂. The reactions are stopped with 5×DNA loading buffer (30% glycerol, 1.2% SDS, 250 mM EDTA), resolved by a 0.8 or 1% agarose gel electrophoresis and visualized by ethidium bromide staining. The resulting cleavage products indicate whether the Cas9 molecule cleaves both DNA strands, or only one of the two strands. For example, linear DNA products indicate the cleavage of both DNA strands. Nicked open circular products indicate that only one of the two strands is cleaved.

Alternatively, the ability of a Cas9 molecule/gRNA molecule complex to bind to and cleave a target nucleic acid can be evaluated in an oligonucleotide DNA cleavage assay. In this assay, DNA oligonucleotides (10 pmol) are radiolabeled by incubating with 5 units T4 polynucleotide kinase and ˜3-6 pmol (˜20-40 mCi) [γ-32P]-ATP in 1× T4 polynucleotide kinase reaction buffer at 37° C. for 30 min, in a 50 μL reaction. After heat inactivation (65° C. for 20 min), reactions are purified through a column to remove unincorporated label. Duplex substrates (100 nM) are generated by annealing labeled oligonucleotides with equimolar amounts of unlabeled complementary oligonucleotide at 95° C. for 3 min, followed by slow cooling to room temperature. For cleavage assays, gRNA molecules are annealed by heating to 95° C. for 30 s, followed by slow cooling to room temperature. Cas9 (500 nM final concentration) is pre-incubated with the annealed gRNA molecules (500 nM) in cleavage assay buffer (20 mM HEPES pH 7.5, 100 mM KCl, 5 mM MgCl2, 1 mM DTT, 5% glycerol) in a total volume of 9 μl. Reactions are initiated by the addition of 1 μl target DNA (10 nM) and incubated for 1 h at 37° C. Reactions are quenched by the addition of 20 μl of loading dye (5 mM EDTA, 0.025% SDS, 5% glycerol in formamide) and heated to 95° C. for 5 min. Cleavage products are resolved on 12% denaturing polyacrylamide gels containing 7 M urea and visualized by phosphorimaging. The resulting cleavage products indicate that whether the complementary strand, the non-complementary strand, or both, are cleaved.

One or both of these assays can be used to evaluate the suitability of any of the gRNA molecule or Cas9 molecule provided.

(b) Binding Assay: Testing the Binding of Cas9 Molecule to Target DNA

Exemplary methods for evaluating the binding of Cas9 molecule to target DNA are described, e.g., in Jinek et al., SCIENCE 2012; 337(6096):816-821.

For example, in an electrophoretic mobility shift assay, target DNA duplexes are formed by mixing of each strand (10 nmol) in deionized water, heating to 95° C. for 3 min and slow cooling to room temperature. All DNAs are purified on 8% native gels containing 1× TBE. DNA bands are visualized by UV shadowing, excised, and eluted by soaking gel pieces in DEPC-treated H₂O. Eluted DNA is ethanol precipitated and dissolved in DEPC-treated H₂O. DNA samples are 5′ end labeled with [γ-32P]-ATP using T4 polynucleotide kinase for 30 min at 37° C. Polynucleotide kinase is heat denatured at 65° C. for 20 min, and unincorporated radiolabel is removed using a column. Binding assays are performed in buffer containing 20 mM HEPES pH 7.5, 100 mM KCl, 5 mM MgCl₂, 1 mM DTT and 10% glycerol in a total volume of 10 μl. Cas9 protein molecule is programmed with equimolar amounts of pre-annealed gRNA molecule and titrated from 100 pM to 1 μM. Radiolabeled DNA is added to a final concentration of 20 μM. Samples are incubated for 1 h at 37° C. and resolved at 4° C. on an 8% native polyacrylamide gel containing 1×TBE and 5 mM MgCl₂. Gels are dried and DNA visualized by phosphorimaging.

(c) Techniques for Measuring Thermostability of Cas9/gRNA Complexes

The thermostability of Cas9-gRNA ribonucleoprotein (RNP) complexes can be detected by differential scanning fluorimetry (DSF) and other techniques. The thermostability of a protein can increase under favorable conditions such as the addition of a binding RNA molecule, e.g., a gRNA. Thus, information regarding the thermostability of a Cas9/gRNA complex is useful for determining whether the complex is stable.

(d) Differential Scanning Flourimetry (DSF)

The thermostability of Cas9-gRNA ribonucleoprotein (RNP) complexes can be measured via DSF. RNP complexes, as described below, include a sequence of ribonucleotides, such as an RNA or a gRNA, and a protein, such as a Cas9 protein or variant thereof. This technique measures the thermostability of a protein, which can increase under favorable conditions such as the addition of a binding RNA molecule, e.g., a gRNA.

The assay can be applied in a number of ways. Exemplary protocols include, but are not limited to, a protocol to determine the desired solution conditions for RNP formation (assay 1, see below), a protocol to test the desired stoichiometric ratio of gRNA:Cas9 protein (assay 2, see below), a protocol to screen for effective gRNA molecules for Cas9 molecules, e.g., wild-type or mutant Cas9 molecules (assay 3, see below), and a protocol to examine RNP formation in the presence of target DNA (assay 4). In some embodiments, the assay is performed using two different protocols, one to test the best stoichiometric ratio of gRNA:Cas9 protein and another to determine the best solution conditions for RNP formation.

To determine the best solution to form RNP complexes, a 2 uM solution of Cas9 in water+10× SYPRO Orange® (Life Technologies cat#S-6650) and dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° increase in temperature every 10 seconds.

The second assay consists of mixing various concentrations of gRNA with 2 uM Cas9 in optimal buffer from assay 1 above and incubating at RT for 10′ in a 384 well plate. An equal volume of optimal buffer+10× SYPRO Orange® (Life Technologies cat#S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° increase in temperature every 10 seconds.

In the third assay, a Cas9 molecule (e.g., a Cas9 protein, e.g., a Cas9 variant protein) of interest is purified. A library of variant gRNA molecules is synthesized and resuspended to a concentration of 20 μM. The Cas9 molecule is incubated with the gRNA molecule at a final concentration of 1 μM each in a predetermined buffer in the presence of 5× SYPRO Orange® (Life Technologies cat#S-6650). After incubating at room temperature for 10 minutes and centrifugation at 2000 rpm for 2 minutes to remove any bubbles, a Bio-Rad CFX384™ Real-Time System C1000 Touch™ Thermal Cycler with the Bio-Rad CFX Manager software is used to run a gradient from 20° C. to 90° C. with an increase of 1° C. in temperature every 10 seconds.

In the fourth assay, a DSF experiment is performed with the following samples: Cas9 protein alone, Cas9 protein with gRNA, Cas9 protein with gRNA and target DNA, and Cas9 protein with target DNA. The order of mixing components is: reaction solution, Cas9 protein, gRNA, DNA, and SYPRO Orange. The reaction solution contains 10 mM HEPES pH 7.5, 100 mM NaCl, in the absence or presence of MgCl2. Following centrifugation at 2000 rpm for 2 minutes to remove any bubbles, a Bio-Rad CFX384™ Real-Time System C1000 Touch™ Thermal Cycler with the Bio-Rad CFX Manager software is used to run a gradient from 20° C. to 90° C. with a 1° increase in temperature every 10 seconds.

5. Target Cells

Cas9 molecules and gRNA molecules, e.g., a Cas9 molecule/gRNA molecule complex, can be used to manipulate a cell, e.g., to edit a target nucleic acid, in a wide variety of cells.

In an embodiment, a cell is manipulated by editing (e.g., inducing a mutation in) one or more target genes, e.g., as described herein. In some embodiments, the expression of one or more target genes (e.g., FAS, BID, CTLA4, PDCD1, CBLB, PTPN6, TRAC or TRBC gene) is modulated. In another embodiment, a cell is manipulated ex vivo by editing (e.g., inducing a mutation in) one or more target genes and/or modulating the expression of one or more target genes, e.g., FAS, BID, CTLA4, PDCD1, CBLB, PTPN6, TRAC or TRBC gene, and administered to a subject. Sources of target cells for ex vivo manipulation may include, e.g., the subject's blood, the subject's cord blood, or the subject's bone marrow. Sources of target cells for ex vivo manipulation may also include, e.g., heterologous donor blood, cord blood, or bone marrow.

The Cas9 and gRNA molecules described herein can be delivered to a target cell. In an embodiment, the target cell is a T cell, e.g., a CD8+ T cell (e.g., a CD8+ naïve T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, e.g., FAS, BID, CTLA4, PDCD1, CBLB, PTPN6, TRAC or TRBC gene, and differentiated into, e.g., a T cell, e.g., a CD8+ T cell (e.g., a CD8+naïve T cell, central memory T cell, or effector memory T cell), a CD4+ T cell, a stem cell memory T cell, a lymphoid progenitor cell or a hematopoietic stem cell.

In an embodiment, the target cell has been altered to contain specific T cell receptor (TCR) genes (e.g., a TRAC and TRBC gene). In another embodiment, the TCR has binding specificity for a tumor associated antigen, e.g., carcinoembryonic antigen (CEA), GP100, melanoma antigen recognized by T cells 1 (MART1), melanoma antigen A3 (MAGEA3), NYESO1 or p53.

In an embodiment, the target cell has been altered to contain a specific chimeric antigen receptor (CAR). In an embodiment, the CAR has binding specificity for a tumor associated antigen, e.g., CD19, CD20, carbonic anhydrase IX (CAIX), CD171, CEA, ERBB2, GD2, alpha-folate receptor, Lewis Y antigen, prostate specific membrane antigen (PSMA) or tumor associated glycoprotein 72 (TAG72).

In another embodiment, the target cell has been altered to bind one or more of the following tumor antigens, e.g., by a TCR or a CAR. Tumor antigens may include, but are not limited to, AD034, AKT1, BRAP, CAGE, CDX2, CLP, CT-7, CT8/HOM-TES-85, cTAGE-1, Fibulin-1, HAGE, HCA587/MAGE-C2, hCAP-G, HCE661, HER2/neu, HLA-Cw, HOM-HD-21/Galectin9, HOM-MEEL-40/SSX2, HOM-RCC-3.1.3/CAXII, HOXA7, HOXB6, Hu, HUB1, KM-HN-3, KM-KN-1, KOC1, KOC2, KOC3, KOC3, LAGE-1, MAGE-1, MAGE-4a, MPP11, MSLN, NNP-1, NY-BR-1, NY-BR-62, NY-BR-85, NY-CO-37, NY-CO-38, NY-ESO-1, NY-ESO-5, NY-LU-12, NY-REN-10, NY-REN-19/LKB/STK11, NY-REN-21, NY-REN-26/BCR, NY-REN-3/NY-CO-38, NY-REN-33/SNC6, NY-REN-43, NY-REN-65, NY-REN-9, NY-SAR-35, OGFr, PLU-1, Rab38, RBPJkappa, RHAMM, SCP1, SCP-1, SSX3, SSX4, SSX5, TOP2A, TOP2B, or Tyrosinase.

a) Methods of Ex Vivo Delivery of Components to Target Cells

The components, e.g., a Cas9 molecule and gRNA molecule can be introduced into target cells in a variety of forms using a variety of delivery methods and formulations, see, e.g., Tables 6 and 7. When a Cas9 or gRNA component is encoded as DNA for delivery, the DNA may typically but not necessarily include a control region, e.g., comprising a promoter, to effect expression. Useful promoters for Cas9 molecule sequences include, e.g., CMV, EF-1a, EFS, MSCV, PGK, or CAG promoters. Useful promoters for gRNAs include, e.g., H1, EF-1a, tRNA or U6 promoters. Promoters with similar or dissimilar strengths can be selected to tune the expression of components. Sequences encoding a Cas9 molecule may comprise a nuclear localization signal (NLS), e.g., an SV40 NLS. In an embodiment a promoter for a Cas9 molecule or a gRNA molecule may be, independently, inducible, tissue specific, or cell specific. In some embodiments, an agent capable of inducing a genetic disruption is introduced RNP complexes. RNP complexes include a sequence of ribonucleotides, such as an RNA or a gRNA molecule, and a protein, such as a Cas9 protein or variant thereof. In some embodiments, the Cas9 protein is delivered as a ribonucleoprotein (RNP) complex that comprises a Cas9 protein provided herein and a gRNA molecule provided herein, e.g., a gRNA targeted for PDCD1. In some embodiments, the RNP that includes one or more gRNA molecules targeted for PDCD1, such as any as described, and a Cas9 enzyme or variant thereof, is directly introduced into the cell via physical delivery (e.g., electroporation, particle gun, Calcium Phosphate transfection, cell compression or squeezing), liposomes or nanoparticles. In particular embodiments, the RNP includes one or more gRNA molecules targeted for PDCD1 and a Cas9 enzyme or variant thereof is introduced via electroporation.

Table 6 provides examples of the form in which the components can be delivered to a target cell.

TABLE 6

| Elements | | |
|---|---|---|
| Cas9 | gRNA | |
| Molecule(s) | molecule(s) | Comments |
| DNA | DNA | In this embodiment, a Cas9 molecule, typically an eaCas9 |
| | | molecule, and a gRNA are transcribed from DNA. In this |
| | | embodiment, they are encoded on separate molecules. |
| DNA | | In this embodiment, a Cas9 molecule, typically an eaCas9 |
| | | molecule, and a gRNA are transcribed from DNA, here from a |
| | | single molecule. |
| DNA | RNA | In this embodiment, a Cas9 molecule, typically an eaCas9 |
| | | molecule, is transcribed from DNA, and a gRNA is provided as in |
| | | vitro transcribed or synthesized RNA |
| mRNA | RNA | In this embodiment, a Cas9 molecule, typically an eaCas9 |
| | | molecule, is translated from in vitro transcribed mRNA, and a |
| | | gRNA is provided as in vitro transcribed or synthesized RNA. |
| mRNA | DNA | In this embodiment, a Cas9 molecule, typically an eaCas9 |
| | | molecule, is translated from in vitro transcribed mRNA, and a |
| | | gRNA is transcribed from DNA. |
| Protein | DNA | In this embodiment, a Cas9 molecule, typically an eaCas9 |
| | | molecule, is provided as a protein, and a gRNA is transcribed |
| | | from DNA. |
| Protein | RNA | In this embodiment, an eaCas9 molecule is provided as a protein, |
| | | and a gRNA is provided as transcribed or synthesized RNA. |

Table 7 summarizes various delivery methods for the components of a Cas system, e.g., the Cas9 molecule component and the gRNA molecule component, as described herein.

TABLE 7

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (eg, electroporation, | | YES | Transient | NO | Nucleic Acids |
| particle gun, Calcium Phosphate | | | | | and Proteins |
| transfection, cell compression or | | | | | |
| squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

(1) DNA-Based Delivery of a Cas9 Molecule and/or a gRNA Molecule

DNA encoding Cas9 molecules (e.g., eaCas9 molecules) and/or gRNA molecules, can be delivered into cells by art-known methods or as described herein. For example, Cas9-encoding and/or gRNA-encoding DNA can be delivered, e.g., by vectors (e.g., viral or non-viral vectors), non-vector based methods (e.g., using naked DNA or DNA complexes), or a combination thereof.

In some embodiments, the Cas9- and/or gRNA-encoding DNA is delivered by a vector (e.g., viral vector/virus or plasmid).

A vector may comprise a sequence that encodes a Cas9 molecule and/or a gRNA molecule. A vector may also comprise a sequence encoding a signal peptide (e.g., for nuclear localization, nucleolar localization, mitochondrial localization), fused, e.g., to a Cas9 molecule sequence. For example, a vector may comprise a nuclear localization sequence (e.g., from SV40) fused to the sequence encoding the Cas9 molecule.

One or more regulatory/control elements, e.g., a promoter, an enhancer, an intron, a polyadenylation signal, a Kozak consensus sequence, internal ribosome entry sites (IRES), a 2A sequence, and splice acceptor or donor can be included in the vectors. In an embodiment, the promoter is recognized by RNA polymerase II (e.g., a CMV promoter). In another embodiment, the promoter is recognized by RNA polymerase III (e.g., a U6 promoter). In another embodiment, the promoter is a regulated promoter (e.g., inducible promoter). In another embodiment, the promoter is a constitutive promoter. In another embodiment, the promoter is a tissue specific promoter. In another embodiment, the promoter is a viral promoter. In another embodiment, the promoter is a non-viral promoter.

In an embodiment, the vector or delivery vehicle is a viral vector (e.g., for generation of recombinant viruses). In an embodiment, the virus is a DNA virus (e.g., dsDNA or ssDNA virus). In an embodiment, the virus is an RNA virus (e.g., an ssRNA virus). Exemplary viral vectors/viruses include, e.g., retroviruses, lentiviruses, adenovirus, adeno-associated virus (AAV), vaccinia viruses, poxviruses, and herpes simplex viruses.

In an embodiment, the virus infects dividing cells. In another embodiment, the virus infects non-dividing cells. In another embodiment, the virus infects both dividing and non-dividing cells. In another embodiment, the virus can integrate into the host genome. In another embodiment, the virus is engineered to have reduced immunity, e.g., in human. In another embodiment, the virus is replication-competent. In another embodiment, the virus is replication-defective, e.g., having one or more coding regions for the genes necessary for additional rounds of virion replication and/or packaging replaced with other genes or deleted. In another embodiment, the virus causes transient expression of the Cas9 molecule and/or the gRNA molecule. In another embodiment, the virus causes long-lasting, e.g., at least 1 week, 2 weeks, 1 month, 2 months, 3 months, 6 months, 9 months, 1 year, 2 years, or permanent expression, of the Cas9 molecule and/or the gRNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In an embodiment, the Cas9- and/or gRNA-encoding DNA is delivered by a recombinant retrovirus. In another embodiment, the retrovirus (e.g., Moloney murine leukemia virus) comprises a reverse transcriptase, e.g., that allows integration into the host genome. In an embodiment, the retrovirus is replication-competent. In another embodiment, the retrovirus is replication-defective, e.g., having one of more coding regions for the genes necessary for additional rounds of virion replication and packaging replaced with other genes, or deleted.

In an embodiment, the Cas9- and/or gRNA-encoding DNA is delivered by a recombinant lentivirus. For example, the lentivirus is replication-defective, e.g., does not comprise one or more genes required for viral replication.

In an embodiment, the Cas9- and/or gRNA-encoding DNA is delivered by a recombinant adenovirus. In another embodiment, the adenovirus is engineered to have reduced immunity in humans.

In an embodiment, the Cas9- and/or gRNA-encoding DNA is delivered by a recombinant AAV. In an embodiment, the AAV can incorporate its genome into that of a host cell, e.g., a target cell as described herein. In another embodiment, the AAV is a self-complementary adeno-associated virus (scAAV), e.g., a scAAV that packages both strands which anneal together to form double stranded DNA. AAV serotypes that may be used in the disclosed methods, include AAV1, AAV2, modified AAV2 (e.g., modifications at Y444F, Y500F, Y730F and/or S662V), AAV3, modified AAV3 (e.g., modifications at Y705F, Y731F and/or T492V), AAV4, AAV5, AAV6, modified AAV6 (e.g., modifications at S663V and/or T492V), AAV8, AAV 8.2, AAV9, AAV rh 10, and pseudotyped AAV, such as AAV2/8, AAV2/5 and AAV2/6 can also be used in the disclosed methods.

In an embodiment, the Cas9- and/or gRNA-encoding DNA is delivered by a hybrid virus, e.g., a hybrid of one or more of the viruses described herein.

A packaging cell is used to form a virus particle that is capable of infecting a target cell. Such a cell includes a 293 cell, which can package adenovirus, and a w2 cell or a PA317 cell, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed, eg. Cas9. For example, an AAV vector used in gene therapy typically only possesses inverted terminal repeat (ITR) sequences from the AAV genome which are required for packaging and gene expression in the host or target cell. The missing viral functions are supplied in trans by the packaging cell line. Henceforth, the viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In an embodiment, the viral vector has the ability of cell type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as a peptide ligand, a single chain antibody, a growth factor); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In an embodiment, the viral vector achieves cell type specific expression. For example, a tissue-specific promoter can be constructed to restrict expression of the transgene (Cas 9 and gRNA) in only a specific target cell. The specificity of the vector can also be mediated by microRNA-dependent control of transgene expression. In an embodiment, the viral vector has increased efficiency of fusion of the viral vector and a target cell membrane. For example, a fusion protein such as fusion-competent hemagglutinin (HA) can be incorporated to increase viral uptake into cells. In an embodiment, the viral vector has the ability of nuclear localization. For example, a virus that requires the breakdown of the nuclear membrane (during cell division) and therefore will not infect a non-diving cell can be altered to incorporate a nuclear localization peptide in the matrix protein of the virus thereby enabling the transduction of non-proliferating cells.

In an embodiment, the Cas9- and/or gRNA-encoding DNA is delivered by a non-vector based method (e.g., using naked DNA or DNA complexes). For example, the DNA can be delivered, e.g., by organically modified silica or silicate (Ormosil), electroporation, transient cell compression or squeezing (eg, as described in Lee, et al [2012] Nano Lett 12: 6322-27), gene gun, sonoporation, magnetofection, lipid-mediated transfection, dendrimers, inorganic nanoparticles, calcium phosphates, or a combination thereof.

In an embodiment, delivery via electroporation comprises mixing the cells with the Cas9- and/or gRNA-encoding DNA in a cartridge, chamber or cuvette and applying one or more electrical impulses of defined duration and amplitude. In an embodiment, delivery via electroporation is performed using a system in which cells are mixed with the Cas9- and/or gRNA-encoding DNA in a vessel connected to a device (eg, a pump) which feeds the mixture into a cartridge, chamber or cuvette wherein one or more electrical impulses of defined duration and amplitude are applied, after which the cells are delivered to a second vessel.

In an embodiment, the Cas9- and/or gRNA-encoding DNA is delivered by a combination of a vector and a non-vector based method. For example, a virosome comprises a liposome combined with an inactivated virus (e.g., HIV or influenza virus), which can result in more efficient gene transfer than either a viral or a liposomal method alone.

In an embodiment, the delivery vehicle is a non-viral vector. In an embodiment, the non-viral vector is an inorganic nanoparticle. Exemplary inorganic nanoparticles include, e.g., magnetic nanoparticles (e.g., Fe₃MnO₂) and silica. The outer surface of the nanoparticle can be conjugated with a positively charged polymer (e.g., polyethylenimine, polylysine, polyserine) which allows for attachment (e.g., conjugation or entrapment) of payload. In an embodiment, the non-viral vector is an organic nanoparticle. Exemplary organic nanoparticles include, e.g., SNALP liposomes that contain cationic lipids together with neutral helper lipids which are coated with polyethylene glycol (PEG), and protamine-nucleic acid complexes coated with lipid.

Exemplary lipids for gene transfer are shown below in Table 8.

TABLE 8
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl-1- | DOSPA | Cationic |
| propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]-dimethylammonium | CLIP-1 | Cationic |
| chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] imidazolinium | DOTIM | Cationic |
| chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

Exemplary polymers for gene transfer are shown below in Table 9.

TABLE 9
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

In an embodiment, the vehicle has targeting modifications to increase target cell update of nanoparticles and liposomes, e.g., cell specific antigens, monoclonal antibodies, single chain antibodies, aptamers, polymers, sugars, and cell penetrating peptides. In an embodiment, the vehicle uses fusogenic and endosome-destabilizing peptides/polymers. In an embodiment, the vehicle undergoes acid-triggered conformational changes (e.g., to accelerate endosomal escape of the cargo). In an embodiment, a stimulus-cleavable polymer is used, e.g., for release in a cellular compartment. For example, disulfide-based cationic polymers that are cleaved in the reducing cellular environment can be used.

In an embodiment, the delivery vehicle is a biological non-viral delivery vehicle. In an embodiment, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific cells, bacteria having modified surface proteins to alter target cell specificity). In an embodiment, the vehicle is a genetically modified bacteriophage (e.g., engineered phages having large packaging capacity, less immunogenicity, containing mammalian plasmid maintenance sequences and having incorporated targeting ligands). In an embodiment, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In an embodiment, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject-derived membrane-bound nanovescicles (30-100 nm) of endocytic origin (e.g., can be produced from various cell types and can therefore be taken up by cells without the need for targeting ligands).

In an embodiment, one or more nucleic acid molecules (e.g., DNA molecules) other than the components of a Cas system, e.g., the Cas9 molecule component and/or the gRNA molecule component described herein, are delivered. In an embodiment, the nucleic acid molecule is delivered at the same time as one or more of the components of the Cas system. In an embodiment, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In an embodiment, the nucleic acid molecule is delivered by a different means from one or more of the components of the Cas system, e.g., the Cas9 molecule component and/or the gRNA molecule component. The nucleic acid molecule can be delivered by any of the delivery methods described herein. For example, the nucleic acid molecule can be delivered by a viral vector, e.g., a retrovirus or a lentivirus, and the Cas9 molecule component and/or the gRNA molecule component can be delivered by electroporation. In an embodiment, the nucleic acid molecule encodes a TRAC gene, a TRBC gene or a CAR gene.

(2) Delivery of RNA Encoding a Cas9 Molecule

RNA encoding Cas9 molecules (e.g., eaCas9 molecules, eiCas9 molecules or eiCas9 fusion proteins) and/or gRNA molecules, can be delivered into cells, e.g., target cells described herein, by art-known methods or as described herein. For example, Cas9-encoding and/or gRNA-encoding RNA can be delivered, e.g., by microinjection, electroporation, transient cell compression or squeezing (eg, as described in Lee, et al [2012] Nano Lett 12: 6322-27), lipid-mediated transfection, peptide-mediated delivery, or a combination thereof.

In an embodiment, delivery via electroporation comprises mixing the cells with the RNA encoding Cas9 molecules (e.g., eaCas9 molecules, eiCas9 molecules or eiCas9 fusion protiens) and/or gRNA molecules in a cartridge, chamber or cuvette and applying one or more electrical impulses of defined duration and amplitude. In an embodiment, delivery via electroporation is performed using a system in which cells are mixed with the RNA encoding Cas9 molecules (e.g., eaCas9 molecules, eiCas9 molecules or eiCas9 fusion protiens) and/or gRNA molecules in a vessel connected to a device (eg, a pump) which feeds the mixture into a cartridge, chamber or cuvette wherein one or more electrical impulses of defined duration and amplitude are applied, after which the cells are delivered to a second vessel.

(3) Delivery of Cas9 Protein and Ribonucleoprotein (RNP)

Cas9 molecules (e.g., eaCas9 molecules, eiCas9 molecules or eiCas9 fusion proteins) can be delivered into cells by art-known methods or as described herein. For example, Cas9 protein molecules can be delivered, e.g., by microinjection, electroporation, transient cell compression or squeezing (eg, as described in Lee, et al [2012] Nano Lett 12: 6322-27), lipid-mediated transfection, peptide-mediated delivery, or a combination thereof. Delivery can be accompanied by DNA encoding a gRNA or by a gRNA. In some embodiments, the Cas9 protein is delivered as a ribonucleoprotein (RNP) complex that comprises a Cas9 protein provided herein and a gRNA molecule provided herein, e.g., a gRNA targeted for PDCD1. In some embodiments, a RNP complex includes a sequence of ribonucleotides, such as an RNA or a gRNA molecule, and a protein, such as a Cas9 protein or variant thereof. In some embodiments, the RNP that includes one or more gRNA molecules targeted for PDCD1 such as any as described and a Cas9 enzyme or variant thereof, is directly introduced into the cell via physical delivery (e.g., electroporation, particle gun, Calcium Phosphate transfection, cell compression or squeezing), liposomes or nanoparticles. In particular embodiments, the RNP includes one or more gRNA molecules targeted for PDCD1, such as any as described, and a Cas9 enzyme or variant thereof is introduced via electroporation.

In an embodiment, delivery via electroporation comprises mixing the cells with the Cas9 molecules (e.g., eaCas9 molecules, eiCas9 molecules or eiCas9 fusion proteins) with or without gRNA molecules in a cartridge, chamber or cuvette and applying one or more electrical impulses of defined duration and amplitude. In an embodiment, delivery via electroporation is performed using a system in which cells are mixed with the Cas9 molecules (e.g., eaCas9 molecules, eiCas9 molecules or eiCas9 fusion protiens) with or without gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

6. Modified Nucleosides, Nucleotides, and Nucleic Acids

Modified nucleosides and modified nucleotides can be present in nucleic acids, e.g., particularly gRNA, but also other forms of RNA, e.g., mRNA, RNAi, or siRNA. As described herein, “nucleoside” is defined as a compound containing a five-carbon sugar molecule (a pentose or ribose) or derivative thereof, and an organic base, purine or pyrimidine, or a derivative thereof. As described herein, “nucleotide” is defined as a nucleoside further comprising a phosphate group.

Modified nucleosides and nucleotides can include one or more of:
(i) alteration, e.g., replacement, of one or both of the non-linking phosphate oxygens and/or of one or more of the linking phosphate oxygens in the phosphodiester backbone linkage;
(ii) alteration, e.g., replacement, of a constituent of the ribose sugar, e.g., of the 2′ hydroxyl on the ribose sugar;
(iii) wholesale replacement of the phosphate moiety with “dephospho” linkers;
(iv) modification or replacement of a naturally occurring nucleobase;
(v) replacement or modification of the ribose-phosphate backbone;
(vi) modification of the 3′ end or 5′ end of the oligonucleotide, e.g., removal, modification or replacement of a terminal phosphate group or conjugation of a moiety; and
(vii) modification of the sugar.

The modifications listed above can be combined to provide modified nucleosides and nucleotides that can have two, three, four, or more modifications. For example, a modified nucleoside or nucleotide can have a modified sugar and a modified nucleobase. In an embodiment, every base of a gRNA is modified, e.g., all bases have a modified phosphate group, e.g., all are phosphorothioate groups. In an embodiment, all, or substantially all, of the phosphate groups of a unimolecular or modular gRNA molecule are replaced with phosphorothioate groups.

In an embodiment, modified nucleotides, e.g., nucleotides having modifications as described herein, can be incorporated into a nucleic acid, e.g., a “modified nucleic acid.” In some embodiments, the modified nucleic acids comprise one, two, three or more modified nucleotides. In some embodiments, at least 5% (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the positions in a modified nucleic acid are a modified nucleotides.

Unmodified nucleic acids can be prone to degradation by, e.g., cellular nucleases. For example, nucleases can hydrolyze nucleic acid phosphodiester bonds. Accordingly, in one aspect the modified nucleic acids described herein can contain one or more modified nucleosides or nucleotides, e.g., to introduce stability toward nucleases.

a) Phosphate Backbone Modifications

(1) The Phosphate Group

In some embodiments, the phosphate group of a modified nucleotide can be modified by replacing one or more of the oxygens with a different substituent. Further, the modified nucleotide, e.g., modified nucleotide present in a modified nucleic acid, can include the wholesale replacement of an unmodified phosphate moiety with a modified phosphate as described herein. In some embodiments, the modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or OR (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

(2) Replacement of the Phosphate Group

The phosphate group can be replaced by non-phosphorus containing connectors. In some embodiments, the charge phosphate group can be replaced by a neutral moiety.

Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

(3) Replacement of the Ribophosphate Backbone

Scaffolds that can mimic nucleic acids can also be constructed wherein the phosphate linker and ribose sugar are replaced by nuclease resistant nucleoside or nucleotide surrogates. In some embodiments, the nucleobases can be tethered by a surrogate backbone. Examples can include, without limitation, the morpholino, cyclobutyl, pyrrolidine and peptide nucleic acid (PNA) nucleoside surrogates.

b) Sugar Modifications

The modified nucleosides and modified nucleotides can include one or more modifications to the sugar group. For example, the 2′ hydroxyl group (OH) can be modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom.

Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH2CH2O)nCH2CH2OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include “locked” nucleic acids (LNA) in which the 2′ hydroxyl can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH2; alkylamino, dialkylamino, heterocyclyl, arylamino, diarylamino, heteroarylamino, or diheteroarylamino, ethylenediamine, or polyamino) and aminoalkoxy, O(CH2)n-amino, (wherein amino can be, e.g., NH2; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH2CH2OCH3, e.g., a PEG derivative).

“Deoxy” modifications can include hydrogen (i.e. deoxyribose sugars, e.g., at the overhang portions of partially ds RNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH2CH2NH)nCH2CH2-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the 1′ position on the sugar, e.g., alpha-nucleosides. The modified nucleic acids can also include “abasic” sugars, which lack a nucleobase at C-1′. These abasic sugars can also be further modified at one or more of the constituent sugar atoms. The modified nucleic acids can also include one or more sugars that are in the L form, e.g. L-nucleosides.

Generally, RNA includes the sugar group ribose, which is a 5-membered ring having an oxygen. Exemplary modified nucleosides and modified nucleotides can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replaced with α-L-threofuranosyl-(3′→2′)).

c) Modifications on the Nucleobase

The modified nucleosides and modified nucleotides described herein, which can be incorporated into a modified nucleic acid, can include a modified nucleobase. Examples of nucleobases include, but are not limited to, adenine (A), guanine (G), cytosine (C), and uracil (U). These nucleobases can be modified or wholly replaced to provide modified nucleosides and modified nucleotides that can be incorporated into modified nucleic acids. The nucleobase of the nucleotide can be independently selected from a purine, a pyrimidine, a purine or pyrimidine analog. In some embodiments, the nucleobase can include, for example, naturally-occurring and synthetic derivatives of a base.

(1) Uracil

In some embodiments, the modified nucleobase is a modified uracil. Exemplary nucleobases and nucleosides having a modified uracil include without limitation pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm 5s2U), 5-propynyl-uridine, 1-propynyl-pseudouridine, 5-taurinomethyl-uridine (τcm5U), 1-taurinomethyl-pseudouridine, 5-taurinomethyl-2-thio-uridine (τm5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm 5Um), 5-carbamoylmethyl-2′-O-methyl-uridine (ncm 5Um), 5-carboxymethylaminomethyl-2′-O-methyl-uridine (cmnm 5Um), 3,2′-O-dimethyl-uridine (m3Um), 5-(isopentenylaminomethyl)-2′-O-methyl-uridine (inm 5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

(2) Cytosine

In some embodiments, the modified nucleobase is a modified cytosine. Exemplary nucleobases and nucleosides having a modified cytosine include without limitation 5-aza-cytidine, 6-aza-cytidine, pseudoisocytidine, 3-methyl-cytidine (m3C), N4-acetyl-cytidine (act), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f 5Cm), N4,N4,2′-O-trimethyl-cytidine (m42Cm), 1-thio-cytidine, 2′-F-ara-cytidine, 2′-F-cytidine, and 2′-OH-ara-cytidine.

(3) Adenine

In some embodiments, the modified nucleobase is a modified adenine. Exemplary nucleobases and nucleosides having a modified adenine include without limitation 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenosine, 7-deaza-8-aza-adenosine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenosine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenosine, 2-methylthio-adenosine, 2-methoxy-adenosine, α-thio-adenosine, 2′-O-methyl-adenosine (Am), N6,2′-O-dimethyl-adenosine (m6Am), N6-Methyl-2′-deoxyadenosine, N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adeno sine, 2′-F-adenosine, 2′-OH-ara-adenosine, and N6-(19-amino-pentaoxanonadecyl)-adenosine.

(4) Guanine

In some embodiments, the modified nucleobase is a modified guanine. Exemplary nucleobases and nucleosides having a modified guanine include without limitation inosine (I), 1-methyl-inosine (m1I), wyo sine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m′G), N2-methyl-guanosine (m2G), N2,N2-dimethyl-guanosine (m2 2G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m2 2Gm), 1-methyl-2′-O-methyl-guanosine (m′Gm), N2,7-dimethyl-2′-O-methyl-guanosine (m2,7Gm), 2′-O-methyl-inosine (Im), 1,2′-O-dimethyl-inosine (m′Im), O6-phenyl-2′-deoxyinosine, 2′-O-ribosylguanosine (phosphate) (Gr(p)), 1-thio-guanosine, O6-methyl-guanosine, O6-Methyl-2′-deoxyguanosine, 2′-F-ara-guanosine, and 2′-F-guanosine.

d) Exemplary Modified gRNAs

In some embodiments, the modified nucleic acids can be modified gRNAs. It is to be understood that any of the gRNAs described herein can be modified in accordance with this section. As discussed herein, transiently expressed or delivered nucleic acids can be prone to degradation by, e.g., cellular nucleases. Accordingly, in one aspect the modified gRNAs described herein can contain one or more modified nucleosides or nucleotides which introduce stability toward nucleases. While not wishing to be bound by theory it is believed that these and other modified gRNAs described herein exhibit enhanced stability with certain cell types (e.g., circulating cells such as T cells) and that this might be responsible for the observed improvements.

For example, as discussed herein, we have seen improvements in ex vivo editing of genes in certain cell types (e.g., T cells) when the 5′ end of a gRNA is modified by the inclusion of a eukaryotic mRNA cap structure or cap analog. The present invention encompasses the realization that the improvements observed with a 5′ capped gRNA can be extended to gRNAs that have been modified in other ways to achieve the same type of structural or functional result (e.g., by the inclusion of modified nucleosides or nucleotides, or when an in vitro transcribed gRNA is modified by treatment with a phosphatase such as calf intestinal alkaline phosphatase to remove the 5′ triphosphate group). While not wishing to be bound by theory, in some embodiments, the modified gRNAs described herein may contain one or more modifications (e.g., modified nucleosides or nucleotides) which introduce stability toward nucleases (e.g., by the inclusion of modified nucleosides or nucleotides and/or a 3′ polyA tail).

Thus, in one aspect, methods and compositions discussed herein provide methods and compositions for gene editing of certain cells (e.g., ex vivo gene editing) by using gRNAs which have been modified at or near their 5′ end (e.g., within 1-10, 1-5, or 1-2 nucleotides of their 5′ end).

In some embodiments, the 5′ end of the gRNA molecule lacks a 5′ triphosphate group. In some embodiments, the 5′ end of the targeting domain lacks a 5′ triphosphate group. In some embodiments, the 5′ end of the gRNA molecule includes a 5′ cap. In some embodiments, the 5′ end of the targeting domain includes a 5′ cap. In some embodiments, the gRNA molecule lacks a 5′ triphosphate group. In some embodiments, the gRNA molecule comprises a targeting domain and the 5′ end of the targeting domain lacks a 5′ triphosphate group. In some embodiments, gRNA molecule includes a 5′ cap. In some embodiments, the gRNA molecule comprises a targeting domain and the 5′ end of the targeting domain includes a 5′ cap.

In an embodiment, the 5′ end of a gRNA is modified by the inclusion of a eukaryotic mRNA cap structure or cap analog (e.g., without limitation a G(5′)ppp(5′)G cap analog, a m7G(5′)ppp(5′)G cap analog, or a 3′-O-Me-m7G(5′)ppp(5′)G anti reverse cap analog (ARCA)). In certain embodiments the 5′ cap comprises a modified guanine nucleotide that is linked to the remainder of the gRNA molecule via a 5′-5′ triphosphate linkage. In some embodiments, the 5′ cap comprises two optionally modified guanine nucleotides that are linked via a 5′-5′ triphosphate linkage. In some embodiments, the 5′ end of the gRNA molecule has the chemical formula:

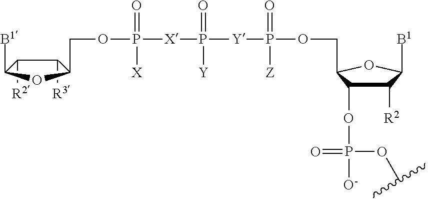

wherein:
each of B¹ and B^{1′} is independently

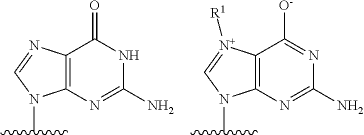

each R¹ is independently C₁₋₄ alkyl, optionally substituted by a phenyl or a 6-membered heteroaryl;
each of R², R^{2′}, and R^{3′} is independently H, F, OH, or O—C₁₋₄ alkyl;
each of X, Y, and Z is independently O or S; and
each of X′ and Y′ is independently O or CH₂.

In an embodiment, each R¹ is independently —CH₃, —CH₂CH₃, or —CH₂C₆H₅.

In an embodiment, R¹ is —CH₃.

In an embodiment, B^{1′} is

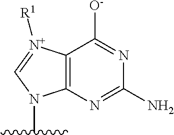

In an embodiment, each of R², R^{2′}, and R^{3′} is independently H, OH, or O—CH₃.

In an embodiment, each of X, Y, and Z is O.

In an embodiment, X′ and Y′ are O.

In an embodiment, the 5′ end of the gRNA molecule has the chemical formula:

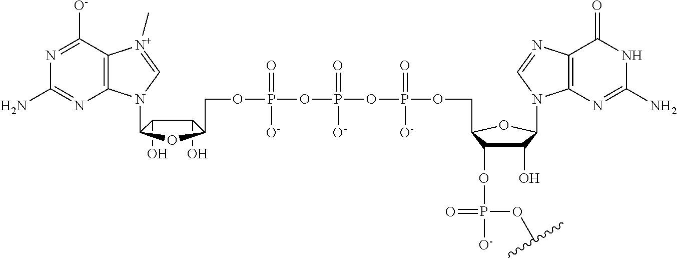

In an embodiment, the 5′ end of the gRNA molecule has the chemical formula:

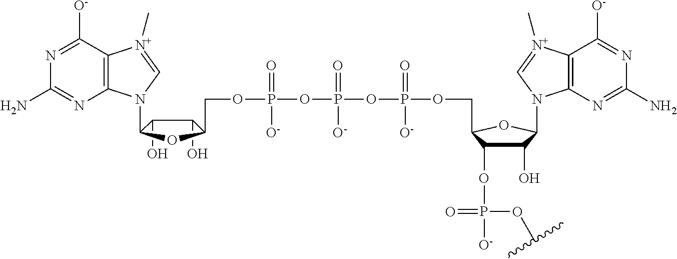

In an embodiment, the 5′ end of the gRNA molecule has the chemical formula:

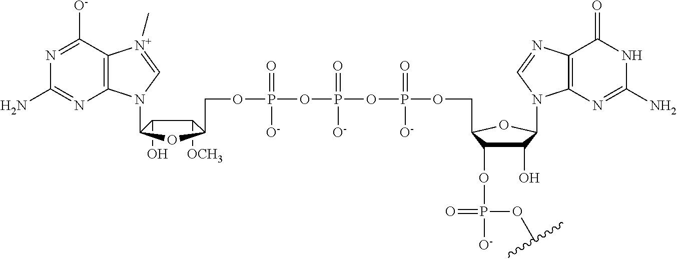

In an embodiment, the 5′ end of the gRNA molecule has the chemical formula:

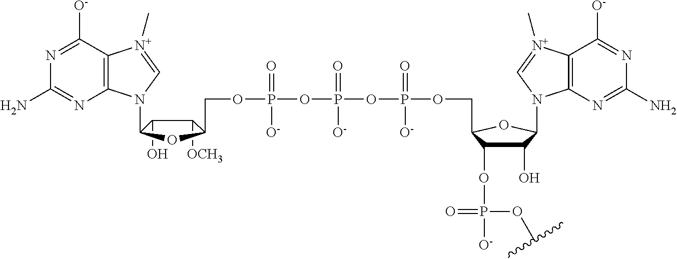

In an embodiment, X is S, and Y and Z are O.

In an embodiment, Y is S, and X and Z are O.

In an embodiment, Z is S, and X and Y are O.

In an embodiment, the phosphorothioate is the Sp diastereomer.

In an embodiment, X′ is CH₂, and Y′ is O.

In an embodiment, X′ is O, and Y′ is CH₂.

In an embodiment, the 5′ cap comprises two optionally modified guanine nucleotides that are linked via an optionally modified 5′-5′ tetraphosphate linkage.

In an embodiment, the 5′ end of the gRNA molecule has the chemical formula:

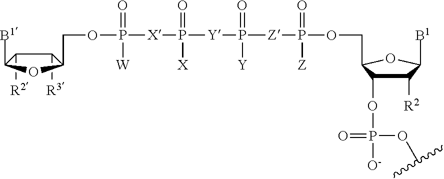

wherein:
each of B¹ and B^{1′} is independently

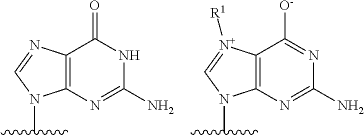

each R¹ is independently C₁₋₄ alkyl, optionally substituted by a phenyl or a 6-membered heteroaryl;
each of R², R^{2′}, and R^{3′} is independently H, F, OH, or O—C₁₋₄ alkyl;
each of W, X, Y, and Z is independently O or S; and
each of X′, Y′, and Z′ is independently O or CH₂.

In an embodiment, each R¹ is independently —CH₃, —CH₂CH₃, or —CH₂C₆H₅.

In an embodiment, R¹ is —CH₃.

In an embodiment, B^{1′} is

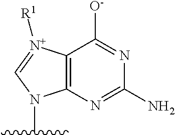

In an embodiment, each of R², R^{2′}, and R^{3′} is independently H, OH, or O—CH₃.

In an embodiment, each of W, X, Y, and Z is O.

In an embodiment, each of X′, Y′, and Z′ are O.

In an embodiment, X′ is CH₂, and Y′ and Z′ are O.

In an embodiment, Y′ is CH₂, and X′ and Z′ are O.

In an embodiment, Z′ is CH₂, and X′ and Y′ are O.

In an embodiment, the 5′ cap comprises two optionally modified guanine nucleotides that are linked via an optionally modified 5′-5′ pentaphosphate linkage.

In an embodiment, the 5′ end of the gRNA molecule has the chemical formula:

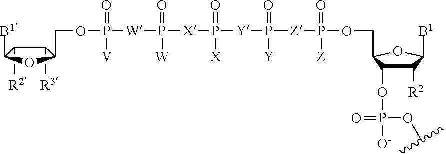

wherein:

each of B1 and B1′ is independently

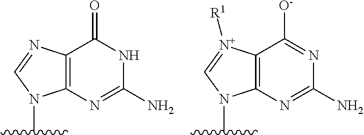

each R¹ is independently C₁₋₄ alkyl, optionally substituted by a phenyl or a 6-membered heteroaryl;
each of R², R^{2′}, and R^{3′} is independently H, F, OH, or O—C₁₋₄ alkyl;
each of V, W, X, Y, and Z is independently O or S; and
each of W′, X′, Y′, and Z′ is independently O or CH₂.

In an embodiment, each R¹ is independently —CH₃, —CH₂CH₃, or —CH₂C₆H₅.

In an embodiment, R¹ is —CH₃.

In an embodiment, B^{1′} is

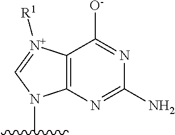

In an embodiment, each of R², R^{2′}, and R^{3′} is independently H, OH, or O—CH₃.

In an embodiment, each of V, W, X, Y, and Z is O.

In an embodiment, each of W′, X′, Y′, and Z′ is O.

It is to be understood that as used herein, the term “5′ cap” encompasses traditional mRNA 5′ cap structures but also analogs of these. For example, in addition to the 5′ cap structures that are encompassed by the chemical structures shown above, one may use, e.g., tetraphosphate analogs having a methylene-bis(phosphonate) moiety (e.g., see Rydzik, A M et al., (2009) Org Biomol Chem 7(22):4763-76), analogs having a sulfur substitution for a non-bridging oxygen (e.g., see Grudzien-Nogalska, E. et al, (2007) RNA 13(10): 1745-1755), N7-benzylated dinucleoside tetraphosphate analogs (e.g., see Grudzien, E. et al., (2004) RNA 10(9): 1479-1487), or anti-reverse cap analogs (e.g., see U.S. Pat. No. 7,074,596 and Jemielity, J. et al., (2003) RNA 9(9): 1108-1122 and Stepinski, J. et al., (2001) RNA 7(10):1486-1495). The present application also encompasses the use of cap analogs with halogen groups instead of OH or OMe (e.g., see U.S. Pat. No. 8,304,529); cap analogs with at least one phosphorothioate (PS) linkage (e.g., see U.S. Pat. No. 8,153,773 and Kowalska, J. et al., (2008) RNA 14(6): 1119-1131); and cap analogs with at least one boranophosphate or phosphoroselenoate linkage (e.g., see U.S. Pat. No. 8,519,110); and alkynyl-derivatized 5′ cap analogs (e.g., see U.S. Pat. No. 8,969,545).

In general, the 5′ cap can be included during either chemical synthesis or in vitro transcription of the gRNA. In an embodiment, a 5′ cap is not used and the gRNA (e.g., an in vitro transcribed gRNA) is instead modified by treatment with a phosphatase (e.g., calf intestinal alkaline phosphatase) to remove the 5′ triphosphate group.

Methods and compositions discussed herein also provide methods and compositions for gene editing by using gRNAs which comprise a 3′ polyA tail. Such gRNAs may, for example, be prepared by adding a polyA tail to a gRNA molecule precursor using a polyadenosine polymerase following in vitro transcription of the gRNA molecule precursor. For example, in one embodiment, a polyA tail may be added enzymatically using a polymerase such as E. coli polyA polymerase (E-PAP). gRNAs including a polyA tail may also be prepared by in vitro transcription from a DNA template. In one embodiment, a polyA tail of defined length is encoded on a DNA template and transcribed with the gRNA via an RNA polymerase (such as T7 RNA polymerase). gRNAs with a polyA tail may also be prepared by ligating a polyA oligonucleotide to a gRNA molecule precursor following in vitro transcription using an RNA ligase or a DNA ligase with or without a splinted DNA oligonucleotide complementary to the gRNA molecule precursor and the polyA oligonucleotide. For example, in one embodiment, a polyA tail of defined length is synthesized as a synthetic oligonucleotide and ligated on the 3′ end of the gRNA with either an RNA ligase or a DNA ligase with or without a splinted DNA oligonucleotide complementary to the guide RNA and the polyA oligonucleotide. gRNAs including the polyA tail may also be prepared synthetically, in one or several pieces that are ligated together by either an RNA ligase or a DNA ligase with or without one or more splinted DNA oligonucleotides.

In some embodiments, the polyA tail is comprised of fewer than 50 adenine nucleotides, for example, fewer than 45 adenine nucleotides, fewer than 40 adenine nucleotides, fewer than 35 adenine nucleotides, fewer than 30 adenine nucleotides, fewer than 25 adenine nucleotides or fewer than 20 adenine nucleotides. In some embodiments the polyA tail is comprised of between 5 and 50 adenine nucleotides, for example between 5 and 40 adenine nucleotides, between 5 and 30 adenine nucleotides, between 10 and 50 adenine nucleotides, or between 15 and 25 adenine nucleotides. In some embodiments, the polyA tail is comprised of about 20 adenine nucleotides.

Methods and compositions discussed herein also provide methods and compositions for gene editing (e.g., ex vivo gene editing) by using gRNAs which include one or more modified nucleosides or nucleotides that are described herein.

While some of the exemplary modifications discussed in this section may be included at any position within the gRNA sequence, in some embodiments, a gRNA comprises a modification at or near its 5′ end (e.g., within 1-10, 1-5, or 1-2 nucleotides of its 5′ end). In some embodiments, a gRNA comprises a modification at or near its 3′ end (e.g., within 1-10, 1-5, or 1-2 nucleotides of its 3′ end). In some embodiments, a gRNA comprises both a modification at or near its 5′ end and a modification at or near its 3′ end. For example, in some embodiments, a gRNA molecule (e.g., an in vitro transcribed gRNA) comprises a targeting domain which is complementary with a target domain from a gene expressed in a eukaryotic cell, wherein the gRNA molecule is modified at its 5′ end and comprises a 3′ polyA tail. The gRNA molecule may, for example, lack a 5′ triphosphate group (e.g., the 5′ end of the targeting domain lacks a 5′ triphosphate group). In an embodiment, a gRNA (e.g., an in vitro transcribed gRNA) is modified by treatment with a phosphatase (e.g., calf intestinal alkaline phosphatase) to remove the 5′ triphosphate group and comprises a 3′ polyA tail as described herein. The gRNA molecule may alternatively include a 5′ cap (e.g., the 5′ end of the targeting domain includes a 5′ cap). In an embodiment, a gRNA (e.g., an in vitro transcribed gRNA) contains both a 5′ cap structure or cap analog and a 3′ polyA tail as described herein. In some embodiments, the 5′ cap comprises a modified guanine nucleotide that is linked to the remainder of the gRNA molecule via a 5′-5′ triphosphate linkage. In some embodiments, the 5′ cap comprises two optionally modified guanine nucleotides that are linked via an optionally modified 5′-5′ triphosphate linkage (e.g., as described above). In some embodiments the polyA tail is comprised of between 5 and 50 adenine nucleotides, for example between 5 and 40 adenine nucleotides, between 5 and 30 adenine nucleotides, between 10 and 50 adenine nucleotides, between 15 and 25 adenine nucleotides, fewer than 30 adenine nucleotides, fewer than 25 adenine nucleotides or about 20 adenine nucleotides.

In yet other embodiments, the present invention provides a gRNA molecule comprising a targeting domain which is complementary with a target domain from a gene expressed in a eukaryotic cell, wherein the gRNA molecule comprises a 3′ polyA tail which is comprised of fewer than 30 adenine nucleotides (e.g., fewer than 25 adenine nucleotides, between 15 and 25 adenine nucleotides, or about 20 adenine nucleotides). In some embodiments, these gRNA molecules are further modified at their 5′ end (e.g., the gRNA molecule is modified by treatment with a phosphatase to remove the 5′ triphosphate group or modified to include a 5′ cap as described herein).

In some embodiments, gRNAs can be modified at a 3′ terminal U ribose. In some embodiments, the 5′ end and a 3′ terminal U ribose of the gRNA are modified (e.g., the gRNA is modified by treatment with a phosphatase to remove the 5′ triphosphate group or modified to include a 5′ cap as described herein). For example, the two terminal hydroxyl groups of the U ribose can be oxidized to aldehyde groups and a concomitant opening of the ribose ring to afford a modified nucleoside as shown below:

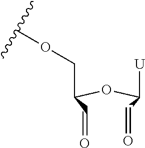

wherein “U” can be an unmodified or modified uridine.

In another embodiment, the 3′ terminal U can be modified with a 2′3′ cyclic phosphate as shown below:

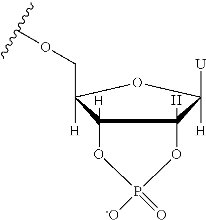

wherein “U” can be an unmodified or modified uridine.

In some embodiments, the gRNA molecules may contain 3′ nucleotides which can be stabilized against degradation, e.g., by incorporating one or more of the modified nucleotides described herein. In this embodiment, e.g., uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In some embodiments, a gRNA comprises both a modification at or near its 5′ end and a modification at or near its 3′ end. In an embodiment, in vitro transcribed gRNA contains both a 5′ cap structure or cap analog and a 3′ polyA tail. In an embodiment, an in vitro transcribed gRNA is modified by treatment with a phosphatase (e.g., calf intestinal alkaline phosphatase) to remove the 5′ triphosphate group and comprises a 3′ polyA tail.

While the foregoing has focused on terminal modifications, it is to be understood that methods and compositions discussed herein may use gRNAs that include one or more modified nucleosides or nucleotides at one or more non-terminal positions and/or one or more terminal positions within the gRNA sequence.

In some embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guano sine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

In some embodiments, a gRNA can include “locked” nucleic acids (LNA) in which the 2′ OH-group can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In some embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNA molecules include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In an embodiment, a gRNA comprises a 4′-S, 4′-Se or a 4′-C-aminomethyl-2′-O-Me modification.

In some embodiments, deaza nucleotides, e.g., 7-deaza-adenosine, can be incorporated into the gRNA. In some embodiments, 0- and N-alkylated nucleotides, e.g., N6-methyl adenosine, can be incorporated into the gRNA. In some embodiments, one or more or all of the nucleotides in a gRNA molecule are deoxynucleotides.

e) miRNA Binding Sites

microRNAs (or miRNAs) are naturally occurring cellular 19-25 nucleotide long noncoding RNAs. They bind to nucleic acid molecules having an appropriate miRNA binding site, e.g., in the 3′ UTR of an mRNA, and down-regulate gene expression. While not wishing to be bound by theory it is believed that the down regulation is either by reducing nucleic acid molecule stability or by inhibiting translation. An RNA species disclosed herein, e.g., an mRNA encoding Cas9 can comprise an miRNA binding site, e.g., in its 3′UTR. The miRNA binding site can be selected to promote down regulation of expression is a selected cell type. By way of example, the incorporation of a binding site for miR-122, a microRNA abundant in liver, can inhibit the expression of the gene of interest in the liver.

D. Governing gRNA Molecules and the Use Thereof to Limit the Activity of a Cas9 System

Methods and compositions that use, or include, a nucleic acid, e.g., DNA, that encodes a Cas9 molecule or a gRNA molecule, can, in addition, use or include a “governing gRNA molecule.” The governing gRNA can limit the activity of the other CRISPR/Cas components introduced into a cell or subject. In an embodiment, a gRNA molecule comprises a targeting domain that is complementary to a target domain on a nucleic acid that comprises a sequence that encodes a component of the CRISPR/Cas system that is introduced into a cell or subject. In an embodiment, a governing gRNA molecule comprises a targeting domain that is complementary with a target sequence on: (a) a nucleic acid that encodes a Cas9 molecule; (b) a nucleic acid that encodes a gRNA which comprises a targeting domain that targets the FAS, BID, CTLA4, PDCD1, CBLB, PTPN6, TRAC or TRBC gene (a target gene gRNA); or on more than one nucleic acid that encodes a CRISPR/Cas component, e.g., both (a) and (b). The governing gRNA molecule can complex with the Cas9 molecule to inactivate a component of the system. In an embodiment, a Cas9 molecule/governing gRNA molecule complex inactivates a nucleic acid that comprises the sequence encoding the Cas9 molecule. In an embodiment, a Cas9 molecule/governing gRNA molecule complex inactivates the nucleic acid that comprises the sequence encoding a target gene gRNA molecule. In an embodiment, a Cas9 molecule/governing gRNA molecule complex places temporal, level of expression, or other limits, on activity of the Cas9 molecule/target gene gRNA molecule complex. In an embodiment, a Cas9 molecule/governing gRNA molecule complex reduces off-target or other unwanted activity. In an embodiment, a governing gRNA molecule targets the coding sequence, or a control region, e.g., a promoter, for the CRISPR/Cas system component to be negatively regulated. For example, a governing gRNA can target the coding sequence for a Cas9 molecule, or a control region, e.g., a promoter, that regulates the expression of the Cas9 molecule coding sequence, or a sequence disposed between the two. In an embodiment, a governing gRNA molecule targets the coding sequence, or a control region, e.g., a promoter, for a target gene gRNA. In an embodiment, a governing gRNA, e.g., a Cas9-targeting or target gene gRNA-targeting, governing gRNA molecule, or a nucleic acid that encodes it, is introduced separately, e.g., later, than is the Cas9 molecule or a nucleic acid that encodes it. For example, a first vector, e.g., a viral vector, e.g., an AAV vector, can introduce nucleic acid encoding a Cas9 molecule and one or more target gene gRNA molecules, and a second vector, e.g., a viral vector, e.g., an AAV vector, can introduce nucleic acid encoding a governing gRNA molecule, e.g., a Cas9-targeting or target gene gRNA targeting, gRNA molecule. In an embodiment, the second vector can be introduced after the first. In other embodiments, a governing gRNA molecule, e.g., a Cas9-targeting or target gene gRNA targeting, governing gRNA molecule, or a nucleic acid that encodes it, can be introduced together, e.g., at the same time or in the same vector, with the Cas9 molecule or a nucleic acid that encodes it, but, e.g., under transcriptional control elements, e.g., a promoter or an enhancer, that are activated at a later time, e.g., such that after a period of time the transcription of Cas9 is reduced. In an embodiment, the transcriptional control element is activated intrinsically. In an embodiment, the transcriptional element is activated via the introduction of an external trigger.

Typically a nucleic acid sequence encoding a governing gRNA molecule, e.g., a Cas9-targeting gRNA molecule, is under the control of a different control region, e.g., promoter, than is the component it negatively modulates, e.g., a nucleic acid encoding a Cas9 molecule. In an embodiment, “different control region” refers to simply not being under the control of one control region, e.g., promoter, that is functionally coupled to both controlled sequences. In an embodiment, different refers to “different control region” in kind or type of control region. For example, the sequence encoding a governing gRNA molecule, e.g., a Cas9-targeting gRNA molecule, is under the control of a control region, e.g., a promoter, that has a lower level of expression, or is expressed later than the sequence which encodes is the component it negatively modulates, e.g., a nucleic acid encoding a Cas9 molecule.

By way of example, a sequence that encodes a governing gRNA molecule, e.g., a Cas9-targeting governing gRNA molecule, can be under the control of a control region (e.g., a promoter) described herein, e.g., human U6 small nuclear promoter, or human H1 promoter. In an embodiment, a sequence that encodes the component it negatively regulates, e.g., a nucleic acid encoding a Cas9 molecule, can be under the control of a control region (e.g., a promoter) described herein, e.g., CMV, EF-1a, MSCV, PGK, CAG control promoters.

### III. COMPOSITIONS AND FORMULATIONS

Also provided are populations of such cells, compositions containing such cells and/or enriched for such cells, such as in which cells expressing the recombinant receptor make up at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions may be administered using standard administration techniques, formulations, and/or devices. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

### IV. METHODS OF ADMINISTRATION AND USES IN ADOPTIVE CELL THERAPY

Provided are methods of administering the cells, populations, and compositions, and uses of such cells, populations, and compositions to treat or prevent diseases, conditions, and disorders, including cancers. In some embodiments, the cells, populations, and compositions are administered to a subject or patient having the particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

As used herein, a “subject” is a mammal, such as a human or other animal, and typically is human. In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject.

In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse.

In some aspects, the subject has not received prior treatment with another therapeutic agent.

Among the diseases, conditions, and disorders for treatment with the provided compositions, cells, methods and uses are tumors, including solid tumors, hematologic malignancies, and melanomas, and infectious diseases, such as infection with a virus or other pathogen, e.g., HIV, HCV, HBV, CMV, and parasitic disease. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or a disease or condition associated with transplant.

In some embodiments, antigen associated with the disease, disorder or condition is selected from ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen.

In some embodiments, the antigen associated with the disease or disorder is selected from the group consisting of orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, OEPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3 and/or biotinylated molecules, and/or molecules expressed by HIV, HCV, HBV or other pathogens.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight.

In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 111 (1995), and U.S. Pat. No. 5,087,616.

### V. DEFINITIONS

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

“Domain”, as used herein, is used to describe segments of a protein or nucleic acid. Unless otherwise indicated, a domain is not required to have any specific functional property.

As used herein, “percent (%) amino acid sequence identity” and “percent identity” when used with respect to an amino acid sequence (reference polypeptide sequence) is defined as the percentage of amino acid residues in a candidate sequence (e.g., a streptavidin mutein) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

An amino acid substitution may include replacement of one amino acid in a polypeptide with another amino acid. Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

“Modulator”, as used herein, refers to an entity, e.g., a drug, which can alter the activity (e.g., enzymatic activity, transcriptional activity, or translational activity), amount, distribution, or structure of a subject molecule or genetic sequence. In an embodiment, modulation comprises cleavage, e.g., breaking of a covalent or non-covalent bond, or the forming of a covalent or non-covalent bond, e.g., the attachment of a moiety, to the subject molecule. In an embodiment, a modulator alters the, three dimensional, secondary, tertiary, or quaternary structure, of a subject molecule. A modulator can increase, decrease, initiate, or eliminate a subject activity.

“Large molecule”, as used herein, refers to a molecule having a molecular weight of at least 2, 3, 5, 10, 20, 30, 40, 50, 60, 70, 80, 90, or 100 kD. Large molecules include proteins, polypeptides, nucleic acids, biologics, and carbohydrates.

“Polypeptide”, as used herein, refers to a polymer of amino acids having less than 100 amino acid residues. In an embodiment, it has less than 50, 20, or 10 amino acid residues.

“Non-homologous end joining” or “NHEJ”, as used herein, refers to ligation mediated repair and/or non-template mediated repair including, e.g., canonical NHEJ (cNHEJ), alternative NHEJ (altNHEJ), microhomology-mediated end joining (MMEJ), single-strand annealing (SSA), and synthesis-dependent microhomology-mediated end joining (SD-MMEJ).

“Reference molecule”, e.g., a reference Cas9 molecule or reference gRNA, as used herein, refers to a molecule to which a subject molecule, e.g., a subject Cas9 molecule of subject gRNA molecule, e.g., a modified or candidate Cas9 molecule is compared. For example, a Cas9 molecule can be characterized as having no more than 10% of the nuclease activity of a reference Cas9 molecule. Examples of reference Cas9 molecules include naturally occurring unmodified Cas9 molecules, e.g., a naturally occurring Cas9 molecule such as a Cas9 molecule of S. pyogenes, S. aureus, or S. thermophilus. In an embodiment, the reference Cas9 molecule is the naturally occurring Cas9 molecule having the closest sequence identity or homology with the Cas9 molecule to which it is being compared. In an embodiment, the reference Cas9 molecule is a sequence, e.g., a naturally occurring or known sequence, which is the parental form on which a change, e.g., a mutation has been made.

“Replacement”, or “replaced”, as used herein with reference to a modification of a molecule does not require a process limitation but merely indicates that the replacement entity is present.

“Small molecule”, as used herein, refers to a compound having a molecular weight less than about 2 kD, e.g., less than about 2 kD, less than about 1.5 kD, less than about 1 kD, or less than about 0.75 kD.

As used herein, a subject includes any living organism, such as humans and other mammals. Mammals include, but are not limited to, humans, and non-human animals, including farm animals, sport animals, rodents and pets. The term includes, but is not limited to, mammals (e.g., humans, other primates, pigs, rodents (e.g., mice and rats or hamsters), rabbits, guinea pigs, cows, horses, cats, dogs, sheep, and goats). In an embodiment, the subject is a human. In other embodiments, the subject is poultry.

As used herein, a composition refers to any mixture of two or more products, substances, or compounds, including cells. It may be a solution, a suspension, liquid, powder, a paste, aqueous, non-aqueous or any combination thereof.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control or fluorescence minus one (FMO) gating control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control or fluorescence minus one (FMO) gating control under otherwise identical conditions, and/or at a level substantially lower than that for cell known to be positive for the marker, and/or at a level substantially similar as compared to that for a cell known to be negative for the marker.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

“X” as used herein in the context of an amino acid sequence, refers to any amino acid (e.g., any of the twenty natural amino acids) unless otherwise specified.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### VI. EXEMPLARY EMBODIMENTS

Among the provided embodiments are:

1. A composition, comprising (a) an engineered immune cell comprising a recombinant receptor that specifically binds to an antigen; and (b) an agent capable of inducing a genetic disruption of a PDCD1 gene encoding a PD-1 polypeptide, wherein said agent is capable of inducing said genetic disruption in, and/or preventing or reducing PD-1 expression in, at least 70%, at least 75%, at least 80%, or at least or greater than 90% of the cells in the composition and/or at least 70%, at least 75%, at least 80%, or at least or greater than 90% of the cells in the composition that express the recombinant receptor.

2. A composition, comprising (a) an engineered immune cell comprising a nucleic acid encoding a recombinant receptor that specifically binds to an antigen; and (b) an agent capable of inducing a genetic disruption of a PDCD1 gene encoding a PD-1 polypeptide, wherein said agent is capable of inducing said genetic disruption in, and/or preventing or reducing PD-1 expression in, at least 70%, at least 75%, at least 80%, or at least or greater than 90% of the cells in the composition and/or at least 70%, at least 75%, at least 80%, or at least or greater than 90%, of the cells in the composition that express the recombinant receptor.

3. The composition of embodiment 1 or embodiment 2, wherein the engineered immune cell expresses the recombinant receptor on its surface.

4. A composition, comprising a cell population containing an engineered immune cell that comprises (a) a recombinant receptor that specifically binds to an antigen; and (b) a genetic disruption of a PDCD1 gene encoding a PD-1 polypeptide, said genetic disruption preventing or reducing the expression of said PD-1 polypeptide, wherein:
at least about 70%, at least about 75%, or at least about 80% or at least or greater than about 90% of the cells in the composition contain the genetic disruption; do not express the endogenous PD-1 polypeptide; do not contain a contiguous PDCD1 gene, do not contain a PDCD1 gene, and/or do not contain a functional PDCD1 gene; and/or do not express a PD-1 polypeptide; and/or
at least about 70%, at least about 75%, or at least about 80% or at least or greater than about 90% of the cells in the composition that express the recombinant receptor contain the genetic disruption, do not express the endogenous PD-1 polypeptide, and/or do not express a PD-1 polypeptide.

5. A composition, comprising a cell population containing an engineered immune cell that comprises (a) a recombinant receptor that specifically binds to an antigen, wherein the engineered immune cell is capable of inducing cytotoxicity, proliferating and/or secreting a cytokine upon binding of the recombinant receptor to said antigen; and (b) a genetic disruption of a PDCD1 gene encoding a PD-1 polypeptide, said genetic disruption capable of preventing or reducing the expression of said PD-1 polypeptide, optionally wherein said prevention or reduction is in at least at or about or greater than at or about 70%, 75%, 80%, 85%, or 90% of the cells in the composition and/or of the cells in the composition that express the recombinant receptor.

6. A composition comprising a cell population containing a population of engineered immune cells, each comprising (a) a recombinant receptor that specifically binds to an antigen; and (b) a genetic disruption of a PDCD1 gene encoding a PD-1 polypeptide, wherein said genetic disruption is capable of preventing or reducing the expression of said PD-1 polypeptide, wherein:
the engineered immune cells, on average, exhibit expression and/or surface expression of the receptor at a level that is the same, about the same or substantially the same, as compared to the average expression and/or surface expression level, respectively, of said recombinant receptor in other cells in the composition that comprise the recombinant receptor and do not comprise the genetic disruption, or
the engineered immune cells do not express the PD-1 polypeptide and on average, exhibit expression and/or surface expression of the receptor at a level is the same, about the same, or substantially the same as compared to the average expression and/or surface level, respectively, in cells of the composition that comprise the recombinant receptor and that express the PD-1 polypeptide.

7. The composition of any of embodiments 1-4 and 6, wherein the recombinant receptor is capable, upon incubation with the antigen, a cell expressing the antigen, and/or an antigen-receptor activating substance, of specifically binding to the antigen, of activating or stimulating the engineered T cell, of inducing cytotoxicity, or of inducing proliferation, survival, and/or cytokine secretion by the immune cell, optionally as measured in an in vitro assay, optionally in an in vitro assay, which optionally comprises incubation for 12, 24, 36, 48, or 60 hours, optionally in the presence of one or more cytokines.

8. The composition of any of embodiments 1-4, 6 and 7, wherein the engineered immune cell is capable, upon incubation with the antigen, a cell expressing the antigen, and/or an antigen-receptor activating substance, of specifically binding to the antigen, of inducing cytotoxicity, proliferating, surviving, and/or secreting a cytokine, optionally as measured in an in vitro assay, which optionally comprises incubation for 12, 24, 36, 48, or 60 hours, optionally in the presence of one or more cytokines and optionally does or does not comprise exposing the immune cell to a PD-L1-expressing cell.

9. The composition of embodiment 7 or embodiment 8, wherein:
the level or degree or extent or duration of the binding, cytotoxicity, proliferation, survival, or cytokine secretion is the same, about the same or substantially the same as compared to that detected or observed for an immune cell comprising the recombinant receptor but not comprising the genetic disruption of a PDCD1 gene, when assessed under the same conditions.

10. The composition of any of embodiments 6 and 8-9, wherein the binding, cytotoxicity, proliferation, survival, and/or cytokine secretion is as measured, optionally in an in vitro assay, following withdrawal and re-exposure to the antigen, antigen-expressing cell, and/or substance.

11. The composition of any of embodiments 1-10, wherein the immune cell is a primary cell from a subject.

12. The composition of any of embodiments 1-11, wherein the immune cell is a human cell.

13. The composition of any of embodiments 1-12, wherein the immune cell is a white blood cell.

14. The composition of any of embodiments 1-13, wherein the immune cell is an NK cell or a T cell.

15. The composition of embodiment 14, wherein the immune cell comprises a plurality of T cells comprising unfractionated T cells, comprises isolated CD8+ cells or is enriched for CD8+ T cells, or comprises isolated CD4+ T cells or is enriched for CD4+ cells, and/or is enriched for a subset thereof selected from the group consisting of naïve cells, effector memory cells, central memory cells, stem central memory cells, effector memory cells, and long-lived effector memory cells.

16. The composition of embodiment 14 or embodiment 15, wherein the percentage, of T cells, or T cells expressing the receptor, and comprising the genetic disruption in the composition, that exhibit a non-activated, long-lived memory, or central memory phenotype, is the same or substantially the same as a population of cells the same or substantially the same as the composition but not containing the genetic disruption or but expressing the PD-1 polypeptide.

17. The composition of any of embodiments 1-16, wherein the percentage of T cells in the composition exhibiting a non-activated, long-lived memory, or central memory phenotype is the same, about the same or substantially the same as compared to the percentage of T cells exhibiting the phenotype in a composition comprising T cells, comprising the recombinant receptor but not comprising the genetic disruption of a PDCD1 gene encoding a PD-1 polypeptide when assessed under the same conditions, which optionally is compared in the absence or presence of contacting or exposing the immune cell to PD-L1.

18. The composition of embodiment 16 or embodiment 17, wherein the phenotype is as assessed following incubation of the composition at or about 37° C.±2° C. for at least 12 hours, 24 hours, 48 hours, 96 hours, 6 days, 12 days, 24 days, 36 days, 48 days or 60 days.

19. The composition of embodiment 18, wherein the incubation is in vitro.

20. The composition of embodiment 18 or embodiment 19, wherein at least a portion of the incubation is performed in the presence of a stimulating agent, which at least a portion is optionally for up to 1 hour, 6 hours, 24 hours, or 48 hours of the incubation.

21. The composition of embodiment 20, wherein the stimulating agent is an agent capable of inducing proliferation of T cells, CD4+ T cells and/or CD8+ T cells.

22. The composition of embodiment 20 or embodiment 21, wherein the stimulating agent is or comprises an antibody specific for CD3 an antibody specific for CD28 and/or a cytokine.

23. The composition of any of embodiments 16-22, wherein the T cell comprising the recombinant receptor comprises one or more phenotypic markers selected from CCR7+, 4-1BB+(CD137+), TIM3+, CD27+, CD62L+, CD127+, CD45RA+, CD45RO−, t-bet^{low}, IL-7Ra+, CD95+, IL-2Rβ+, CXCR3+ or LFA-1+.

24. The composition of any of embodiments 1-23, wherein the recombinant receptor is a functional non-TCR antigen receptor or a transgenic TCR.

25. The composition of any of embodiments 1-23, wherein the recombinant receptor is a chimeric antigen receptor (CAR).

26. The composition of embodiment 25, wherein the CAR comprises an antigen-binding domain that is an antibody or an antibody fragment.

27. The composition of embodiment 26, wherein the antibody fragment is a single chain fragment.

28. The composition of embodiment 26 or embodiment 27, wherein the antibody fragment comprises antibody variable regions joined by a flexible immunoglobulin linker.

29. The composition of any of embodiments 26-28, wherein the fragment comprises an scFv.

30. The composition of any of embodiments 1-29, wherein the antigen is associated with a disease or disorder.

31. The composition of embodiment 30, wherein the disease or disorder is an infectious disease or condition, an autoimmune disease, an inflammatory disease or a tumor or a cancer.

32. The composition of any of embodiments 1-31, wherein the recombinant receptor specifically binds to a tumor antigen.

33. The composition of any of embodiments 1-32, wherein the antigen is selected from ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acethycholine receptor, GD2, GD3, HMW-MAA, IL-22R-alpha, IL-13R-alpha2, kdr, kappa light chain, Lewis Y, L1-cell adhesion molecule (CD171), MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1), cyclin A1 (CCNA1), BCMA and interleukin 12.

34. The composition of any of embodiments 1-33, wherein the recombinant receptor comprises an intracellular signaling domain comprising an ITAM.

35. The composition of embodiment 34, wherein the intracellular signaling domain comprises an intracellular domain of a CD3-zeta (CD3) chain.

36. The composition of embodiment 34 or embodiment 35, wherein the recombinant receptor further comprises a costimulatory signaling region.

37. The composition of embodiment 36, wherein the costimulatory signaling region comprises a signaling domain of CD28 or 4-1BB.

38. The composition of any of embodiments 1-3 and 7-37, wherein the agent comprises at least one of (a) a least one gRNA having a targeting domain that is complementary with a target domain of a PDCD1 gene or (b) at least one nucleic acid encoding the at least one gRNA.

39. The composition of any of embodiments 1-3 and 7-38, wherein the agent comprises at least one complex of a Cas9 molecule and a gRNA having a targeting domain that is complementary with a target domain of a PDCD1 gene.

40. The composition of embodiment 38 or embodiment 39, wherein the guide RNA further comprises a first complementarity domain, a second complementarity domain that is complementary to the first complementarity domain, a proximal domain and optionally a tail domain.

41. The composition of embodiment 40, wherein the first complementarity domain and second complementarity domain are joined by a linking domain.

42. The composition of any of embodiments 41, wherein the guide RNA comprises a 3′ poly-A tail and a 5′ Anti-Reverse Cap Analog (ARCA) cap.

43. The composition any of embodiments 39-42, wherein the Cas9 molecule is an enzymatically active Cas9.

44. The composition of any of embodiments 38-43, wherein the at least one gRNA includes a targeting domain comprising a sequence selected from the group consisting of GUCUGGGCGGUGCUACAACU (SEQ ID NO:508), GCCCUGGCCAGUCGUCU (SEQ ID NO: 514), CGUCUGGGCGGUGCUACAAC (SEQ ID NO:1533), UGUAGCACCGCCCAGACGAC (SEQ ID NO:579), CGACUGGCCAGGGCGCCUGU (SEQ ID NO:582) and CACCUACCUAAGAACCAUCC (SEQ ID NO:723).

45. The composition of any of embodiments 38-44, wherein the at least one gRNA includes a targeting domain comprising the sequence CGACUGGCCAGGGCGCCUGU (SEQ ID NO:582).

46. The composition of any of embodiments 38-45, wherein the Cas9 molecule is a nickase and two Cas9 molecule/gRNA molecule complexes are guided by two different gRNA molecules to cleave the target domain with two single stranded breaks on opposing strands of the target domain.

47. The composition of any of embodiments 39-46, wherein the Cas9 molecule is an S. aureus Cas9 molecule.

48. The composition of any of embodiments 39-46, wherein the Cas9 molecule is an S. pyogenes Cas9.

49. The composition of any of embodiments 39-48, wherein the Cas9 molecule lacks an active RuvC domain or an active HNH domain.

50. The composition of any of embodiments 39-46, 48 and 49, wherein the Cas9 molecule is an S. pyogenes Cas9 molecule comprising a D10A mutation.

51. The composition of any of embodiments 46-50, wherein the two gRNA molecules comprise targeting domains that are selected from the following pairs of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;

(SEQ ID NO: 582)

CGACUGGCCAGGGCGCCUGU
and

(SEQ ID NO: 511)

GGCCAGGAUGGUUCUUAGGU;

(SEQ ID NO: 582)

CGACUGGCCAGGGCGCCUGU
and

(SEQ ID NO: 512)

GGAUGGUUCUUAGGUAGGUG;

(SEQ ID NO: 582)

CGACUGGCCAGGGCGCCUGU
and

(SEQ ID NO: 576)

CGUCUGGGCGGUGCUACAAC;

(SEQ ID NO: 582)

CGACUGGCCAGGGCGCCUGU
and

(SEQ ID NO: 766)

CUACAACUGGGCUGGCGGCC;

(SEQ ID NO: 579)

UGUAGCACCGCCCAGACGAC
and

(SEQ ID NO: 511)

GGCCAGGAUGGUUCUUAGGU;

(SEQ ID NO: 579)

UGUAGCACCGCCCAGACGAC
and

(SEQ ID NO: 512)

GGAUGGUUCUUAGGUAGGUG;
or

(SEQ ID NO: 581)

ACCGCCCAGACGACUGGCCA
and

(SEQ ID NO: 511)

GGCCAGGAUGGUUCUUAGGU.

52. The composition of any of embodiments 39-46 and 48-51, wherein the Cas9 molecule is an S. pyogenes Cas9 molecule comprising an N863A mutation.

53. The composition of embodiment 52, wherein the two gRNA molecules comprise targeting domains that are selected from the following pairs of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;
or

(SEQ ID NO: 582)

CGACUGGCCAGGGCGCCUGU
and

(SEQ ID NO: 511)

GGCCAGGAUGGUUCUUAGGU.

54. The composition of any of embodiments 1-53, wherein the genetic disruption comprises creation of a double strand break which is repaired by non-homologous end joining (NHEJ) to effect insertions and deletions (indels) in the PDCD1 gene.

55. The composition of any of embodiments 1-54, wherein:
at least about 70%, at least about 75%, or at least about 80% of the cells in the composition contain the genetic disruption; do not express the endogenous PD-1 polypeptide; do not contain a contiguous PDCD1 gene, a PDCD1 gene, and/or a functional PDCD1 gene; and/or do not express a PD-1 polypeptide; and/or
at least about 70%, at least about 75%, or at least about 80% of the cells in the composition that express the recombinant receptor contain the genetic disruption, do not express the endogenous PD-1 polypeptide, or do not express a PD-1 polypeptide

56. The composition of embodiment 4 or embodiment 55, wherein:
greater than 80%, 81%, 82%, 83%, 84%, 85%, 86%, 87%, 88%, 89%, 90%, 91%, 92%, 93%, 94%, or 95% of the cells in the composition contain the genetic disruption; do not express the endogenous PD-1 polypeptide; do not contain a contiguous PDCD1 gene, a PDCD1 gene, and/or a functional PDCD1 gene; and/or do not express a PD-1 polypeptide; and/or
greater than 80%, 81%, 82%, 83%, 84%, 85%, 86%, 87%, 88%, 89%, 90%, 91%, 92%, 93%, 94%, or 95% of the cells in the composition that express the recombinant receptor contain the genetic disruption, do not express the endogenous PD-1 polypeptide, or do not express a PD-1 polypeptide.

57. The composition of any of embodiments 1-56, wherein:
at least at or about 90% of the cells in the composition, or cells in the composition that express the recombinant receptor, optionally as assessed by flow cytometry, contain the genetic disruption; do not express the endogenous PD-1 polypeptide; do not contain a contiguous PDCD1 gene, a PDCD1 gene, and/or a functional PDCD1 gene; and/or do not express a PD-1 polypeptide.

58. The composition of any of embodiments 1-57, wherein both alleles of the gene in the genome are disrupted.

59. The composition of any of embodiments 1-58, wherein cells in the composition and/or the cells in the composition that express the recombinant receptor are not enriched or selected for cells that contain the genetic disruption; do not express the endogenous PD-1 polypeptide; do not contain a contiguous PDCD1 gene, a PDCD1 gene, and/or a functional PDCD1 gene; and/or do not express a PD-1 polypeptide.

60. The composition of any of embodiments 1-59, wherein no more than 2, no more than 5 or no more than 10 other genes in each cell in the composition, or each cell in the composition that expresses the recombinant receptor, on average, are disrupted or are disrupted by the agent.

61. The composition of any of embodiments 1-60, wherein no other genes in each cell in the composition or each cell in the composition that expresses the recombinant receptor are disrupted in the cell or are disrupted by the agent.

62. The composition of any of embodiments 1-61, further comprising a pharmaceutically acceptable buffer.

63. The composition of any of embodiments 1-62, wherein, at a point in time following administration of the composition to a subject, optionally having the disease or condition;
cells expressing the recombinant receptor and not expressing PD-1 are detectable in the blood of the subject or blood-derived sample from the subject or in a tissue or biological sample of the subject;
cells containing the genetic disruption are detectable in the blood of the subject or a blood-derived sample from the subject or in a tissue or biological sample of the subject;
cells containing the genetic disruption and expressing the recombinant receptor are detectable in the blood of the subject or a blood-derived sample from the subject or in a tissue or biological sample of the subject.

64. The composition of embodiment 63, wherein the time-point is at or about 7, 8, 9, 10, 11, 12, 13, or 14 days or 2, 3, 4, 5, 6, 7, 8, 9, 10, 11, or 12 weeks following administration.

65. The composition of embodiment 63 or 64, wherein said cells detectable in the blood or sample are present at a concentration of at or about or at least at or about 1, 2, 3, 4, 5, 6, 7, 8, 9, 10, or 100 cells/microliter of blood and/or represent at least 10, 20, 25, 30, 35, 40, 45, or 50% or more of T cells in the blood.

66. The composition of any of embodiments 1-65, wherein, following administration of the composition to a subject:
cells in the composition comprising the genetic disruption expand and/or persist in the subject at a rate, and/or for a time that, is at least as great as, optionally greater than, the expansion and/or persistence of T cells in the composition without the genetic disruption and/or as the expansion and/or persistence of T cells of a reference composition in which T cells express the recombinant receptor but do not comprising the deletion; and/or
cells in the composition comprising the genetic disruption and comprising the recombinant receptor expand and/or persist in the subject at a rate, and/or for a time that, is at least as great as, optionally greater than, the expansion and/or persistence of T cells in the composition without the genetic disruption and/or as the expansion and/or persistence of T cells of a reference composition in which T cells express the recombinant receptor but do not comprising the deletion.

67. The composition of embodiment 66, wherein the rate or time is at least at or about 1.5-fold or 2-fold or 3-fold greater.

68. A method of producing a genetically engineered immune cell, comprising:
(a) introducing into an immune cell a nucleic acid molecule encoding a recombinant receptor that specifically binds to an antigen; and
(b) introducing into the immune cell an agent capable of inducing a genetic disruption of a PDCD1 gene encoding a PD-1 polypeptide comprising one of (i) at least one gRNA having a targeting domain that is complementary with a target domain of the PDCD1 gene or (ii) at least one nucleic acid encoding the at least one gRNA.

69. A method of producing a genetically engineered immune cell, comprising introducing into an immune cell expressing a recombinant receptor that specifically binds to an antigen an agent capable of inducing a genetic disruption of a PDCD1 gene encoding a PD-1 polypeptide comprising one of (i) at least one gRNA having a targeting domain that is complementary with a target domain of the PDCD1 gene or (ii) at least one nucleic acid encoding the at least one gRNA.

70. The method of embodiment 68 or embodiment 69, wherein the agent comprises at least one complex of a Cas9 molecule and a gRNA having a targeting domain that is complementary with a target domain of a PDCD1 gene.

71. The method of any of embodiments 68-70, wherein the guide RNA further comprises a first complementarity domain, a second complementarity domain that is complementary to the first complementarity domain, a proximal domain and optionally a tail domain.

72. The method of embodiment 71, wherein the first complementarity domain and second complementarity domain are joined by a linking domain.

73. The method of any of embodiments 68-72, wherein the guide RNA comprises a 3′ poly-A tail and a 5′ Anti-Reverse Cap Analog (ARCA) cap.

74. The method of any of embodiments 68-73, wherein introduction comprises contacting the cells with the agent or a portion thereof, in vitro.

75. The method of any of embodiments 68-74, wherein introduction of the agent comprises electroporation.

76. The method of embodiment 74 or embodiment 75, wherein the introduction further comprises incubating the cells, in vitro prior to, during or subsequent to the contacting of the cells with the agent or prior to, during or subsequent to the electroporation.

77. The method of any of embodiments 68-76, wherein the introduction in (a) comprises transduction and the introduction further comprises incubating the cells, in vitro, prior to, during or subsequent to the transduction.

78. The method of embodiment 76 or embodiment 77, wherein at least a portion of the incubation is in the presence of (i) a cytokine selected from the group consisting of IL-2, IL-7, and IL-15, and/or (ii) a stimulating or activating agent or agents, optionally comprising anti-CD3 and/or anti-CD28 antibodies.

79. The method of embodiment 77 or embodiment 78, wherein the introduction in (a) comprises: prior to transduction, incubating the cells with IL-2 at a concentration of 20 U/mL to 200 U/mL, optionally about 100 U/mL; IL-7 at a concentration of 1 ng/mL to 50 ng/mL, optionally about 10 ng/mL and/or IL-15 at a concentration of 0.5 ng/mL to 20 ng/mL, optionally about 5 ng/mL; and subsequent to transduction, incubating the cells with IL-2 at a concentration of 10 U/mL to 200 U/mL, optionally about 50 U/mL; IL-7 at a concentration of 0.5 ng/mL to 20 ng/mL, optionally about 5 ng/mL and/or IL-15 at a concentration of 0.1 ng/mL to 10 ng/mL, optionally about 0.5 ng/mL.

80. The method of any of embodiments 76-79, wherein the incubation independently is carried out for up to or approximately 24, 36, 48 hours, 3, 4, 5, 6, 7, 8, 9, 10, 11, 12, 13, 14, 15, 16, 17, 18, 19, 20 or 21 days.

81. The method of any of embodiments 76-80, wherein the incubation is independently carried out for 24-48 hours or 36-48 hours.

82. The method of any of embodiments 74-81, wherein the cells are contacted with the agent at a ratio of approximately 1 microgram per 100,000, 200,000, 300,000, 400,000, or 500,000 cells.

83. The method of any of embodiments 76-82, wherein:
the incubation is at a temperature of 30° C.±2° C. to 39° C.±2° C.; or
the incubation is at a temperature that is at least or about at least 30° C.±2° C., 32° C.±2° C., 34° C.±2° C. or 37° C.±2° C.

84. The method of any of embodiments 76-83, wherein at least a portion of the incubation is at 30° C.±2° C. and at least a portion of the incubation is at 37° C.±2° C.

85. The method of any of embodiments 68-84, wherein the method further comprises resting the cells between the introducing in (a) and the introducing in (b).

86. The method of any of embodiments 70-85, wherein the Cas9 molecule is an enzymatically active Cas9.

87. The method of any of embodiments 68-86, wherein the at least one gRNA includes a targeting domain comprising a sequence selected from the group consisting of GUCUGGGCGGUGCUACAACU (SEQ ID NO:508), GCCCUGGCCAGUCGUCU (SEQ ID NO: 514), CGUCUGGGCGGUGCUACAAC (SEQ ID NO:1533), UGUAGCACCGCCCAGACGAC (SEQ ID NO:579), CGACUGGCCAGGGCGCCUGU (SEQ ID NO:582) and CACCUACCUAAGAACCAUCC (SEQ ID NO:723).

88. The method of any of embodiments 68-87, wherein the at least one gRNA includes a targeting domain comprising the sequence CGACUGGCCAGGGCGCCUGU (SEQ ID NO:582).

89. The method of any of embodiments 68-79, wherein the Cas9 molecule is a nickase and two Cas9 molecule/gRNA molecule complexes are guided by two different gRNA molecules to cleave the target domain with two single stranded breaks on opposing strands of the target domain.

90. The method of any of embodiments 70-89, wherein the Cas9 molecule is an S. aureus Cas9 molecule.

91. The method of any of embodiments 70-90, wherein the Cas9 molecule is an S. pyogenes Cas9.

92. The method of any of embodiments 70-91, wherein the Cas9 molecule lacks an active RuvC domain or an active HNH domain.

93. The method of any of embodiments 70-89, 91 and 92, wherein the Cas9 molecule is an S. pyogenes Cas9 molecule comprising a D10A mutation.

94. The method of any of embodiments 89-93, wherein the two gRNA molecules comprise targeting domains that are selected from the following pairs of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;

(SEQ ID NO: 582)

CGACUGGCCAGGGCGCCUGU
and

(SEQ ID NO: 511)

GGCCAGGAUGGUUCUUAGGU;

(SEQ ID NO: 582)

CGACUGGCCAGGGCGCCUGU
and

(SEQ ID NO: 512)

GGAUGGUUCUUAGGUAGGUG;

(SEQ ID NO: 582)

CGACUGGCCAGGGCGCCUGU
and

(SEQ ID NO: 576)

CGUCUGGGCGGUGCUACAAC;

(SEQ ID NO: 582)

CGACUGGCCAGGGCGCCUGU
and

(SEQ ID NO: 766)

CUACAACUGGGCUGGCGGCC;

(SEQ ID NO: 579)

UGUAGCACCGCCCAGACGAC
and

(SEQ ID NO: 511)

GGCCAGGAUGGUUCUUAGGU;

(SEQ ID NO: 579)

UGUAGCACCGCCCAGACGAC
and

(SEQ ID NO: 512)

GGAUGGUUCUUAGGUAGGUG;
or

(SEQ ID NO: 581)

ACCGCCCAGACGACUGGCCA
and

(SEQ ID NO: 511)

GGCCAGGAUGGUUCUUAGGU.

95. The method of any of embodiments 70-89 and 91-94, wherein the Cas9 molecule is an S. pyogenes Cas9 molecule comprising an N863A mutation.

96. The method of embodiment 94, wherein the two gRNA molecules comprise targeting domains that are selected from the following pairs of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;
or

(SEQ ID NO: 582)

CGACUGGCCAGGGCGCCUGU
and

(SEQ ID NO: 511)

GGCCAGGAUGGUUCUUAGGU.

97. The method of any of embodiments 68-96, wherein the genetic disruption comprises creation of a double strand break which is repaired by non-homologous end joining (NHEJ) to effect insertions and deletions (indels) in the PDCD1 gene.

98. The method of any of embodiments 68-97, wherein the recombinant receptor is a functional non-TCR antigen receptor or a transgenic TCR.

99. The method of any of embodiments 68-98, wherein the recombinant receptor is a chimeric antigen receptor (CAR).

100. The method of embodiment 99, wherein the CAR comprises an antigen-binding domain that is an antibody or an antibody fragment.

101. The method of embodiment 100, wherein the antibody fragment is a single chain fragment.

102. The method of embodiment 100 or embodiment 101, wherein the antibody fragment comprises antibody variable regions joined by a flexible immunoglobulin linker.

103. The method of any of embodiments 100-102, wherein the fragment comprises an scFv.

104. The method of any of embodiments 100-103, wherein the antigen is associated with a disease or disorder.

105. The method of embodiment 104, wherein the disease or disorder is an infectious disease or condition, an autoimmune disease, an inflammatory disease or a tumor or a cancer.

106. The method of any of embodiments 68-105, wherein the recombinant receptor specifically binds to a tumor antigen.

107. The method of any of embodiments 68-106, wherein the antigen is selected from ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acethycholine e receptor, GD2, GD3, HMW-MAA, IL-22R-alpha, IL-13R-alpha2, kdr, kappa light chain, Lewis Y, L1-cell adhesion molecule (CD171), MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1), cyclin A1 (CCNA1), BCMA and interleukin 12.

108. The method of any of embodiments 68-107, wherein the recombinant receptor comprises an intracellular signaling domain comprising an ITAM.

109. The method of embodiment 108, wherein the intracellular signaling domain comprises an intracellular domain of a CD3-zeta (CD3) chain.

110. The method of embodiment 108 or embodiment 109, wherein the recombinant receptor further comprises a costimulatory signaling region.

111. The method of embodiment 110, wherein the costimulatory signaling region comprises a signaling domain of CD28 or 4-1BB.

112. The method of any of embodiments 68-111, wherein the nucleic acid encoding the recombinant receptor is a viral vector.

113. The method of embodiment 112, wherein the viral vector is a retroviral vector. 114. The method of embodiment 112 or embodiment 113, wherein the viral vector is a lentiviral vector or a gammaretroviral vector.

115. The method of any of embodiments 68-114, wherein introduction of the nucleic acid encoding the recombinant vector is by transduction, which optionally is retroviral transduction.

116. The method of any of embodiments 68-115, wherein the immune cell is a primary cell from a subject.

117. The method of any of embodiments 68-116, wherein the immune cell is a human cell.

118. The method of any of embodiments 68-117, wherein the immune cell is a white blood cell.

119. The method of any of embodiments 68-118, wherein the immune cell is an NK cell or T cell.

120. The method of embodiment 119, wherein the immune cell is a T cell that is an unfractionated T cell, isolated CD8+ T cell, or isolated CD4+ T cell.

121. The method of any of embodiments 68-120, that is performed on a plurality of immune cells.

122. The method of any of embodiments 68-121, wherein subsequent to introducing the agent and introducing the recombinant receptor, cells are not enriched or selected for (a) cells containing the genetic disruption or not expressing the endogenous PD-1 polypeptide, (b) cells expressing the recombinant receptor or both (a) and (b).

123. The method of any of embodiments 68-122, further comprising enriching or selecting for (a) cells containing the genetic disruption or not expressing the endogenous PD-1 polypeptide, (b) cells expressing the recombinant receptor or for both (a) and (b).

124. The method of any of embodiments 68-123, further comprising incubating the cells at or at about 37° C.±2° C.

125. The method of embodiment 124, wherein the incubation is carried out for a time between at or about 1 hour and at or about 96 hours, between at or about 4 hours and at or about 72 hours, between at or about 8 hours and at or about 48 hours, between at or about 12 hours and at or about 36 hours, between at or about 6 hours and at or about 24 hours, between at or about 36 hours and at or about 96 hours, inclusive.

126. The method of embodiment 125, wherein the incubation or a portion of the incubation is performed in the presence of a stimulating agent.

127. The method of embodiment 126, wherein the stimulating agent is an agent capable of inducing proliferation of T cells, CD4+ T cells and/or CD8+ T cells.

128. The method of embodiment 126 or embodiment 127, wherein the stimulating agent is or comprises an antibody specific for CD3 an antibody specific for CD28 and/or a cytokine.

129. The method of any of embodiments 68-128, further comprising formulating cells produced by the method in a pharmaceutically acceptable buffer.

130. The method of any of embodiments 68-129, wherein the method produces a population of cells in which:
at least about 70%, at least about 75%, or at least about 80% of the cells both 1) contain the genetic disruption; do not express the endogenous PD-1 polypeptide; do not contain a contiguous PDCD1 gene, a PDCD1 gene, and/or a functional PDCD1 gene; and/or do not express a PD-1 polypeptide; and 2) express the recombinant receptor; or
at least about 70%, at least about 75%, or at least about 80% of the cells that express the recombinant receptor contain the genetic disruption, do not express the endogenous PD-1 polypeptide, or do not express a PD-1 polypeptide.

131. The method of any of embodiments 68-130, wherein the method produces a population of cells in which:
greater than 80%, 81%, 82%, 83%, 84%, 85%, 86%, 87%, 88%, 89%, 90%, 91%, 92%, 93%, 94%, or 95% of the cells both 1) contain the genetic disruption; do not express the endogenous PD-1 polypeptide; do not contain a contiguous PDCD1 gene, a PDCD1 gene, and/or a functional PDCD1 gene; and/or do not express a PD-1 polypeptide and 2) express the recombinant receptor; and/or
greater than 80%, 81%, 82%, 83%, 84%, 85%, 86%, 87%, 88%, 89%, 90%, 91%, 92%, 93%, 94%, or 95% of the cells that express the recombinant receptor contain the genetic disruption, do not express the endogenous PD-1 polypeptide, or do not express a PD-1 polypeptide.

132. The method of any of embodiments 68-131, wherein both alleles of the gene in the genome are disrupted.

133. A genetically engineered immune cell produced by the method of any of embodiments 68-132.

134. A plurality of genetically engineered immune cells produced by the method of any of embodiments 68-132.

135. The plurality of genetically engineered immune cells of embodiment 134, wherein:
at least about 70%, at least about 75%, or at least about 80% of the cells both 1) contain the genetic disruption; do not express the endogenous PD-1 polypeptide; do not contain a contiguous PDCD1 gene, a PDCD1 gene, and/or a functional PDCD1 gene; and/or do not express a PD-1 polypeptide; and 2) express the recombinant receptor; or
at least about 70%, at least about 75%, or at least about 80% of the cells that express the recombinant receptor contain the genetic disruption, do not express the endogenous PD-1 polypeptide, or do not express a PD-1 polypeptide.

136. The plurality of genetically engineered immune cells of embodiment 134 or embodiment 135, wherein:
greater than 80%, 81%, 82%, 83%, 84%, 85%, 86%, 87%, 88%, 89%, 90%, 91%, 92%, 93%, 94%, or 95% of the cells both 1) contain the genetic disruption; do not express the endogenous PD-1 polypeptide; do not contain a contiguous PDCD1 gene, a PDCD1 gene, and/or a functional PDCD1 gene; and/or do not express a PD-1 polypeptide and 2) express the recombinant receptor; and/or
greater than 80%, 81%, 82%, 83%, 84%, 85%, 86%, 87%, 88%, 89%, 90%, 91%, 92%, 93%, 94%, or 95% of the cells that express the recombinant receptor contain the genetic disruption, do not express the endogenous PD-1 polypeptide, or do not express a PD-1 polypeptide.

137. A composition comprising the genetically engineered immune cell of embodiment 133 or the plurality of genetically engineered immune cells of any of embodiments 134-136, and optionally a pharmaceutically acceptable buffer.

138. A method of treatment, comprising administering the composition of any of embodiments 1-67 and 137 to a subject having a disease or condition.

139. The method of embodiment 138, wherein the recombinant receptor specifically binds to an antigen associated with the disease or condition.

140. The method of embodiment 138 or embodiment 139, wherein the disease or condition is a cancer, a tumor, an autoimmune disease or disorder, or an infectious disease.

141. The method of embodiment 140, wherein the cancer or tumor is leukemia, lymphoma, chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, colon cancer, lung cancer, liver cancer, breast cancer, prostate cancer, ovarian cancer, skin cancer, melanoma cancer, bone cancer, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

142. The method of any of embodiments 139-141, wherein the antigen is selected from the group consisting of orphan tyrosine kinase receptor ROR1, tEGFR, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, MAGE A3, CE7, Wilms Tumor 1 (WT-1), cyclin A1 (CCNA1), BCMA and interleukin 12.

143. The method of any of embodiments 139-142, wherein the antigen is CD19 or BCMA.

144. The method of any of embodiments 138-143, wherein the engineered cell administered to the subject has reduced and/or eliminated expression of PD-1 1 day, 2 days, 3 days, 4 days, 5 days, 6 days, 7 days, 10 days, 14 days, one month, two months, or more after administration.

145. The method of any of embodiments 138-144, wherein the engineered cell administered to the subject persists in the subject for at least 1 day, 2 days, 3 days, 4 days, 5 days, 6 days, 7 days, 10 days, 14 days, one month, two months, or more after administration.

146 The method of embodiment any of embodiments 138-145, wherein, at a point in time following administration of the composition:
cells expressing the recombinant receptor and not expressing PD-1 are detectable in the blood of the subject or blood-derived sample from the subject or in a tissue or biological sample of the subject;
cells containing the genetic disruption are detectable in the blood of the subject or a blood-derived sample from the subject or in a tissue or biological sample of the subject;
cells containing the genetic disruption and expressing the recombinant receptor are detectable in the blood of the subject or a blood-derived sample from the subject or in a tissue or biological sample of the subject.

147. The method of any of embodiments 138-146, wherein the time-point is at or about 7, 8, 9, 10, 11, 12, 13, or 14 days or 2, 3, 4, 5, 6, 7, 8, 9, 10, 11, or 12 weeks following administration.

148. The method of embodiment 138-147, wherein said cells detectable in the blood or sample are present at a concentration of at or about or at least at or about 1, 2, 3, 4, 5, 6, 7, 8, 9, 10, or 100 cells/microliter of blood and/or represent at least 10, 20, 25, 30, 35, 40, 45, or 50% or more of T cells in the blood.

149. The method of any of embodiments 138-148, wherein, following administration:
cells in the composition comprising the genetic disruption expand and/or persist in the subject at a rate, and/or for a time that, is at least as great as, optionally greater than, the expansion and/or persistence of T cells in the composition without the genetic disruption and/or as the expansion and/or persistence of T cells of a reference composition in which T cells express the recombinant receptor but do not comprising the deletion; and/or
cells in the composition comprising the genetic disruption and comprising the recombinant receptor expand and/or persist in the subject at a rate, and/or for a time that, is at least as great as, optionally greater than, the expansion and/or persistence of T cells in the composition without the genetic disruption and/or as the expansion and/or persistence of T cells of a reference composition in which T cells express the recombinant receptor but do not comprising the deletion.

150. The method of embodiment 149, wherein the rate or time is at least at or about 1.5-fold or 2-fold or 3-fold greater.

151. The method of any of embodiments 140-150, wherein the tumor is a solid tumor.

152. The method of any of embodiments 140-151, wherein the tumor is not a B cell-derived tumor, is not a leukemia and/or is not a lymphoma.

153. The method of any of embodiments 140-152, wherein the tumor or cells thereof express or have been observed to express a ligand for PD-1.

154. A pharmaceutical composition comprising an engineered immune cell that comprises (a) a recombinant receptor that specifically binds to an antigen; and (b) a genetic disruption of a PDCD1 gene encoding a PD-1 polypeptide, said genetic disruption preventing or reducing the expression of said PD-1 polypeptide, wherein the engineered cell has a phenotype of reduced and/or eliminated expression of PD-1 prior to administration to a subject and wherein the cells maintain the phenotype for 1 day, 2 days, 3 days, 4 days, 5 days, 6 days, 7 days, 10 days, 14 days, one month, two months, or more after administration to the subject.

155. A composition of any of embodiments 1-67, 137 and 154 for use in treating a disease or condition in a subject.

156. The composition for use of embodiment 155, wherein the recombinant receptor specifically binds to an antigen associated with the disease or condition.

157. The composition for use of embodiment 155 or embodiment 156, wherein the disease or condition is a cancer, a tumor, an autoimmune disease or disorder, or an infectious disease.

158. The composition for use of any of embodiments 155-157, wherein the disease or condition is a cancer or tumor, which is a leukemia, lymphoma, chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, colon cancer, lung cancer, liver cancer, breast cancer, prostate cancer, ovarian cancer, skin cancer, melanoma cancer, bone cancer, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

159. The composition for use of any of embodiments 155-158, wherein the antigen is selected from the group consisting of orphan tyrosine kinase receptor ROR1, tEGFR, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, MAGE A3, CE7, Wilms Tumor 1 (WT-1), cyclin A1 (CCNA1), BCMA and interleukin 12.

160. The composition for use of any of embodiments 155-159, wherein the antigen is CD19 or BCMA.

161. The composition for use of any of embodiments 155-160, wherein, following administration of the composition to subject, one or more cells containing the genetic disruption, and optionally containing the recombinant receptor, persists in, and/or is detectable in a tissue or biological sample of, the subject at a time that is at least at or about 1 day, 2 days, 3 days, 4 days, 5 days, 6 days, 7 days, 10 days, 14 days, one month, two months, or more after administration; and/or at least 50, 60, 70, 80, 85, or 90% of the T cells, or T cells expressing the recombinant receptor, that are detectable in a biological sample or tissue from the subject at a time that is at least at or about 1 day, 2 days, 3 days, 4 days, 5 days, 6 days, 7 days, 10 days, 14 days, one month, two months, or more after administration contain the genetic disruption.

162. A method of altering a T cell comprising contacting the T cell with one or more Cas9 molecule/gRNA molecule complexes, wherein the gRNA molecule(s) in the one or more Cas9 molecule/gRNA molecule complexes comprise a targeting domain which is complementary with a target domain from the PDCD1 gene.

163. A method of altering a T cell comprising contacting the T cell with at least two Cas9 molecule/gRNA molecule complexes, each complex comprising a gRNA molecule comprising a targeting domain which is complementary with a target domain from the PDCD1 gene.

164. The method of embodiment 162 or embodiment 163, wherein the T cell is from a subject suffering from cancer.

165. The method of embodiment 164, wherein the cancer is selected from the group consisting of: lymphoma, chronic lymphocytic leukemia (CLL), B cell acute lymphocytic leukemia (B-ALL), acute lymphoblastic leukemia, acute myeloid leukemia, non-Hodgkin's lymphoma (NHL), diffuse large cell lymphoma (DLCL), multiple myeloma, renal cell carcinoma (RCC), neuroblastoma, colorectal cancer, breast cancer, ovarian cancer, melanoma, sarcoma, prostate cancer, lung cancer, esophageal cancer, hepatocellular carcinoma, pancreatic cancer, astrocytoma, mesothelioma, head and neck cancer, and medulloblastoma.

166. The method of any of embodiments 162-165, wherein the T cell is from a subject having cancer or which could otherwise benefit from a mutation at a T cell target position of the PDCD1 gene.

167. The method of any of embodiments 162-166, wherein the contacting is performed ex vivo.

168. The method of any of embodiments 162-167, wherein the T cell comprises a recombinant receptor.

169. The method of any of embodiments 162-168, further comprising contacting the T cell with a nucleic acid encoding a recombinant receptor under conditions to introduce the nucleic acid into the cell.

170. The method of embodiment 168 or embodiment 169, wherein the recombinant receptor is a functional non-TCR antigen receptor or a transgenic TCR.

171. The method of any of embodiments 168-170, wherein the recombinant receptor is a chimeric antigen receptor (CAR).

172. The method of embodiment 171, wherein the CAR comprises an antigen-binding domain that is an antibody or an antibody fragment.

173. The method of embodiment 172, wherein the antibody fragment is a single chain fragment.

174. The method of embodiment 172 or embodiment 173, wherein the antibody fragment comprises antibody variable regions joined by a flexible immunoglobulin linker.

175. The method of any of embodiments 172-174, wherein the fragment comprises an scFv.

176. The method of any of embodiments 172-175, wherein the antigen is associated with a disease or disorder.

177. The method of embodiment 176, wherein the disease or disorder is an infectious disease or condition, an autoimmune disease, an inflammatory disease or a tumor or a cancer.

178. The method of any of embodiments 168-177, wherein the recombinant receptor specifically binds to a tumor antigen.

179. The method of any of embodiments 171-178, wherein the antigen is selected from ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acethycholine e receptor, GD2, GD3, HMW-MAA, IL-22R-alpha, IL-13R-alpha2, kdr, kappa light chain, Lewis Y, L1-cell adhesion molecule (CD171), MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1), cyclin A1 (CCNA1), BCMA and interleukin 12.

180. The method of any of embodiments 168-179, wherein the recombinant receptor comprises an intracellular signaling domain comprising an ITAM.

181. The method of embodiment 180, wherein the intracellular signaling domain comprises an intracellular domain of a CD3-zeta (CD3) chain.

182. The method of embodiment 180 or embodiment 181, wherein the recombinant receptor further comprises a costimulatory signaling region.

183. The method of embodiment 182, wherein the costimulatory signaling region comprises a signaling domain of CD28 or 4-1BB.

184. The method of any of embodiments 164-183, wherein the altered T cell is returned to the subject's body after the step of contacting.

185. The method of any of embodiments 162-184, wherein the T cell is from a subject suffering from cancer, the contacting is performed ex vivo and the altered T cell is returned to the subject's body after the step of contacting.

186. The method of any of embodiments 162-185, wherein the one or more Cas9 molecule/gRNA molecule complexes are formed prior to the contacting.

187. The method of any of embodiments 163-186, wherein the at least two Cas9 molecule/gRNA molecule complexes are formed prior to the contacting.

188. The method of any of embodiments 162-187, wherein the gRNA molecule(s) comprise a targeting domain that is the same as, or differs by no more than 3 nucleotides from, a targeting domain from any of SEQ ID NOS: 481-555, 563-1516, 1517-3748, 14657-16670, and 16671-21037

189. The method of embodiment 188, wherein the gRNA molecule(s) comprise a targeting domain that is selected from SEQ ID NOS: 563-1516.

190. The method of embodiment 188, wherein the gRNA molecule(s) comprise a targeting domain that is selected from SEQ ID NOS: 1517-3748.

191. The method of embodiment 188, wherein the gRNA molecule(s) comprise a targeting domain that is selected from SEQ ID NOS: 14657-16670.

192. The method of embodiment 188, wherein the gRNA molecule(s) comprise a targeting domain that is selected from SEQ ID NOS: 16671-21037.

193. The method of embodiment 188, wherein the gRNA molecule(s) comprise a targeting domain that is selected from SEQ ID NOS: 481-500 and 508-547.

194. The method of embodiment 188, wherein the gRNA molecule(s) comprise a targeting domain that is selected from SEQ ID NOS: 501-507 and 548-555.

195. The method of embodiment 188, wherein the gRNA molecule(s) comprise a targeting domain that is selected from SEQ ID NOS: 508, 514, 576, 579, 582, and 723.

196. The method of embodiment 188, wherein the gRNA molecule(s) comprise a targeting domain that is selected from SEQ ID NOS: 508, 510, 511, 512, 514, 576, 579, 581, 582, 766, and 723.

197. The method of any of embodiments 162-196, wherein the gRNA molecule(s) are modified at their 5′ end or comprise a 3′ polyA tail.

198. The method of any of embodiments 162-196, wherein the gRNA molecule(s) are modified at their 5′ end and comprise a 3′ polyA tail.

199. The method of embodiment 197 or embodiment 198, wherein the gRNA molecule(s) lack a 5′ triphosphate group.

200. The method of embodiment 197 or embodiment 198, wherein the gRNA molecule(s) include a 5′ cap.

201. The method of embodiment 200, wherein the 5′ cap comprises a modified guanine nucleotide that is linked to the remainder of the gRNA molecule via a 5′-5′ triphosphate linkage.

202. The method of embodiment 200, wherein the 5′ cap comprises two optionally modified guanine nucleotides that are linked via an optionally modified 5′-5′ triphosphate linkage.

203. The method of any of embodiments 197-202, wherein the 3′ polyA tail is comprised of about 10 to about 30 adenine nucleotides.

204. The method of any of embodiments 197-202, wherein the 3′ polyA tail is comprised of about 20 adenine nucleotides.

205. The method of embodiment 203 or embodiment 204, wherein the gRNA molecule(s) including the 3′ polyA tail were prepared by in vitro transcription from a DNA template.

206. The method of embodiment 205, wherein the 5′ nucleotide of the targeting domain is a guanine nucleotide, the DNA template comprises a T7 promoter sequence located immediately upstream of the sequence that corresponds to the targeting domain, and the 3′ nucleotide of the T7 promoter sequence is not a guanine nucleotide.

207. The method of embodiment 205, wherein the 5′ nucleotide of the targeting domain is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

208. The method of any of embodiments 162-207, wherein the one or more Cas9 molecule/gRNA molecule complexes are delivered into the T cell via electroporation.

209. The method of any of embodiments 163-208, wherein the at least two Cas9 molecule/gRNA molecule complexes are delivered into the T cell via electroporation.

210. The method of any of embodiments 162-209, wherein the gRNA molecule(s) comprise a targeting domain which is complementary with a target domain from the PDCD1 gene and wherein the gRNA molecule(s) guide the Cas9 molecule to cleave the target domain with an efficiency of cleavage of at least 40%.

211. The method of embodiment 210, wherein the efficiency of cleavage is determined using a labeled anti-PDCD1 antibody and a flow cytometry assay.

212. The method of any of embodiments 162-211, wherein the Cas9 molecule is guided by a single gRNA molecule and cleaves the target domain with a single double stranded break.

213. The method of embodiment 212, wherein the Cas9 molecule is a S. pyogenes Cas9 molecule.

214. The method of any of embodiments 162-213, wherein the targeting domain is selected from:

(SEQ ID NO: 508)

GUCUGGGCGGUGCUACAACU;

(SEQ ID NO: 514)

GCCCUGGCCAGUCGUCU;

(SEQ ID NO: 576)

CGUCUGGGCGGUGCUACAAC;

(SEQ ID NO: 579)

UGUAGCACCGCCCAGACGAC;

(SEQ ID NO: 582)

CGACUGGCCAGGGCGCCUGU;
or

(SEQ ID NO: 723)

CACCUACCUAAGAACCAUCC.

215. The method of any of embodiments 162-211, wherein the Cas9 molecule is a nickase and two Cas9 molecule/gRNA molecule complexes are guided by two different gRNA molecules to cleave the target domain with two single stranded breaks on opposing strands of the target domain.

216. The method of embodiment 215, wherein the Cas9 molecule is a S. pyogenes Cas9 molecule.

217. The method of any of embodiments 162-216, wherein the S. pyogenes Cas9 molecule has a D10A mutation.

218. The method of any of embodiments 162-217, wherein the two gRNA molecules comprise targeting domains that are selected from the following pairs of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;

(SEQ ID NO: 582)

CGACUGGCCAGGGCGCCUGU
and

(SEQ ID NO: 511)

GGCCAGGAUGGUUCUUAGGU;

(SEQ ID NO: 582)

CGACUGGCCAGGGCGCCUGU
and

(SEQ ID NO: 512)

GGAUGGUUCUUAGGUAGGUG;

(SEQ ID NO: 582)

CGACUGGCCAGGGCGCCUGU
and

(SEQ ID NO: 576)

CGUCUGGGCGGUGCUACAAC;

(SEQ ID NO: 582)

CGACUGGCCAGGGCGCCUGU
and

(SEQ ID NO: 766)

CUACAACUGGGCUGGCGGCC;

(SEQ ID NO: 579)

UGUAGCACCGCCCAGACGAC
and

(SEQ ID NO: 511)

GGCCAGGAUGGUUCUUAGGU;

(SEQ ID NO: 579)

UGUAGCACCGCCCAGACGAC
and

(SEQ ID NO: 512)

GGAUGGUUCUUAGGUAGGUG;
or

(SEQ ID NO: 581)

ACCGCCCAGACGACUGGCCA
and

(SEQ ID NO: 511)

GGCCAGGAUGGUUCUUAGGU.

219. The method of any of embodiments 162-218, wherein the S. pyogenes Cas9 molecule has a N863A mutation.

220. The method of embodiment 219, wherein the two gRNA molecules comprise targeting domains that are selected from the following pairs of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;
or

(SEQ ID NO: 582)

CGACUGGCCAGGGCGCCUGU
and

(SEQ ID NO: 511)

GGCCAGGAUGGUUCUUAGGU.

221. The method of any of embodiments 162-220, wherein the gRNA molecule(s) are modular gRNA molecule(s).

222. The method of any of embodiments 162-220, wherein the gRNA molecule(s) are chimeric gRNA molecule(s).

223. The method of embodiment 222, wherein the gRNA molecule(s) comprise from 5′ to 3′: a targeting domain; a first complementarity domain; a linking domain; a second complementarity domain; a proximal domain; and a tail domain.

224. The method of embodiment 222 or embodiment 223, wherein the gRNA molecule(s) comprise a linking domain of no more than 25 nucleotides in length and a proximal and tail domain, that taken together, are at least 20 nucleotides in length.

225. The method of any one of embodiments 210-224, wherein the method is characterized by an efficiency of cleavage of at least 60%.

226. The method of any one of embodiments 210-224, wherein the method is characterized by an efficiency of cleavage of at least 80%.

227. The method of any one of embodiments 210-224, wherein the method is characterized by an efficiency of cleavage of at least 90%.

228. The method of any one of embodiments 210-227, wherein the gRNA molecule is characterized by fewer than 5 off-targets.

229. The method of any one of embodiments 210-228, wherein the gRNA molecule is characterized by fewer than 2 exonic off-targets.

230. The method of embodiment 228 or embodiment 229, wherein off-targets are identified by GUIDE-seq.

231. The method of embodiment 228 or embodiment 229, wherein off-targets are identified by Amp-seq.

232. A Cas9 molecule/gRNA molecule complex, wherein the gRNA molecule comprises a targeting domain which is complementary with a target domain from the PDCD1 gene, and the gRNA molecule is modified at its 5′ end and/or comprises a 3′ polyA tail.

233. The Cas9 molecule/gRNA molecule complex of embodiment 232, wherein the gRNA molecule comprises a targeting domain that is the same as, or differs by no more than 3 nucleotides from, a targeting domain from any of SEQ ID NOS: 481-555, 563-1516, 1517-3748, 14657-16670, and 16671-21037 234. The Cas9 molecule/gRNA molecule complex of embodiment 232, wherein the gRNA molecule comprises a targeting domain that is selected from SEQ ID NOS: 563-1516.

235. The Cas9 molecule/gRNA molecule complex of embodiment 232, wherein the gRNA molecule comprises a targeting domain that is selected from SEQ ID NOS: 1517-3748.

236. The Cas9 molecule/gRNA molecule complex of embodiment 232, wherein the gRNA molecule comprises a targeting domain that is selected from SEQ ID NOS: 14657-16670.

237. The Cas9 molecule/gRNA molecule complex of embodiment 232, wherein the gRNA molecule comprises a targeting domain that is selected from SEQ ID NOS: 16671-21037.

238. The Cas9 molecule/gRNA molecule complex of embodiment 232, wherein the gRNA molecule comprises a targeting domain that is selected from SEQ ID NOS: 481-500 and 508-547.

239. The Cas9 molecule/gRNA molecule complex of embodiment 232, wherein the gRNA molecule comprises a targeting domain that is selected from SEQ ID NOS: 501-507 and 548-555.

240. The Cas9 molecule/gRNA molecule complex of embodiment 232, wherein the gRNA molecule comprises a targeting domain that is selected from SEQ ID NOS: 508, 514, 576, 579, 582, and 723.

241. The Cas9 molecule/gRNA molecule complex of embodiment 232, wherein the gRNA molecule comprises a targeting domain that is selected from SEQ ID NOS: 508, 510, 511, 512, 514, 576, 579, 581, 582, 766, and 723.

242. The Cas9 molecule/gRNA molecule complex of any of embodiments 232-241, wherein the gRNA molecule is modified at its 5′ end.

243. The Cas9 molecule/gRNA molecule complex of embodiment 242, wherein the gRNA molecule lacks a 5′ triphosphate group.

244. The Cas9 molecule/gRNA molecule complex of embodiment 242, wherein the gRNA molecule includes a 5′ cap.

245. The Cas9 molecule/gRNA molecule complex of embodiment 244, wherein the 5′ cap comprises a modified guanine nucleotide that is linked to the remainder of the gRNA molecule via a 5′-5′ triphosphate linkage.

246. The Cas9 molecule/gRNA molecule complex of embodiment 244, wherein the 5′ cap comprises two optionally modified guanine nucleotides that are linked via an optionally modified 5′-5′ triphosphate linkage.

247. The Cas9 molecule/gRNA molecule complex of any of embodiments 232-246, wherein the 3′ polyA tail is comprised of about 10 to about 30 adenine nucleotides.

248. The Cas9 molecule/gRNA molecule complex of any of embodiments 232-246, wherein the 3′ polyA tail is comprised of about 20 adenine nucleotides.

249. The Cas9 molecule/gRNA molecule complex of embodiment 247 or embodiment 248, wherein the gRNA molecule including the 3′ polyA tail was prepared by in vitro transcription from a DNA template.

250. The Cas9 molecule/gRNA molecule complex of embodiment 249, wherein the 5′ nucleotide of the targeting domain is a guanine nucleotide, the DNA template comprises a T7 promoter sequence located immediately upstream of the sequence that corresponds to the targeting domain, and the 3′ nucleotide of the T7 promoter sequence is not a guanine nucleotide.

251. The Cas9 molecule/gRNA molecule complex of embodiment 249, wherein the 5′ nucleotide of the targeting domain is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

252. The Cas9 molecule/gRNA molecule complex of any of embodiments 232-251, wherein the Cas9 molecule cleaves a target domain with a double stranded break.

253. The Cas9 molecule/gRNA molecule complex of embodiment 252, wherein the Cas9 molecule is a S. pyogenes Cas9 molecule.

254. The Cas9 molecule/gRNA molecule complex of any of embodiments 232-253, wherein the targeting domain is selected from the following group of targeting domains:

(SEQ ID NO: 508)

GUCUGGGCGGUGCUACAACU;

(SEQ ID NO: 514)

GCCCUGGCCAGUCGUCU;

(SEQ ID NO: 576)

CGUCUGGGCGGUGCUACAAC;

(SEQ ID NO: 579)

UGUAGCACCGCCCAGACGAC;

(SEQ ID NO: 582)

CGACUGGCCAGGGCGCCUGU;
or

(SEQ ID NO: 723)

CACCUACCUAAGAACCAUCC.

255. The Cas9 molecule/gRNA molecule complex of any of embodiments 232-251, wherein the Cas9 molecule cleaves a target domain with a single stranded break.

256. The Cas9 molecule/gRNA molecule complex of embodiment 255, wherein the Cas9 molecule is a S. pyogenes Cas9 molecule.

257. The Cas9 molecule/gRNA molecule complex of any of embodiments 232-embodiment 256, wherein the S. pyogenes Cas9 molecule has a D10A mutation.

258. The Cas9 molecule/gRNA molecule complex of any of embodiments 232-embodiment 257, wherein the targeting domain is selected from the following group of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;

(SEQ ID NO: 582)

CGACUGGCCAGGGCGCCUGU
and

(SEQ ID NO: 511)

GGCCAGGAUGGUUCUUAGGU;

(SEQ ID NO: 582)

CGACUGGCCAGGGCGCCUGU
and

(SEQ ID NO: 512)

GGAUGGUUCUUAGGUAGGUG;

(SEQ ID NO: 582)

CGACUGGCCAGGGCGCCUGU
and

(SEQ ID NO: 576)

CGUCUGGGCGGUGCUACAAC;

(SEQ ID NO: 582)

CGACUGGCCAGGGCGCCUGU
and

(SEQ ID NO: 766)

CUACAACUGGGCUGGCGGCC;

(SEQ ID NO: 579)

UGUAGCACCGCCCAGACGAC
and

(SEQ ID NO: 511)

GGCCAGGAUGGUUCUUAGGU;

(SEQ ID NO: 579)

UGUAGCACCGCCCAGACGAC
and

(SEQ ID NO: 512)

GGAUGGUUCUUAGGUAGGUG;
or

(SEQ ID NO: 581)

ACCGCCCAGACGACUGGCCA
and

(SEQ ID NO: 511)

GGCCAGGAUGGUUCUUAGGU.

259. The Cas9 molecule/gRNA molecule complex of any of embodiment 232-256, wherein the S. pyogenes Cas9 molecule has a N863A mutation.

260. The Cas9 molecule/gRNA molecule complex of embodiment 259, wherein the targeting domain is selected from the following group of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;
or

(SEQ ID NO: 582)

CGACUGGCCAGGGCGCCUGU
and

(SEQ ID NO: 511)

GGCCAGGAUGGUUCUUAGGU.

261. The Cas9 molecule/gRNA molecule complex of any of embodiments 232-260, wherein the gRNA molecule is a modular gRNA molecule.

262. The Cas9 molecule/gRNA molecule complex of any of embodiments 232-261, wherein the gRNA molecule is a chimeric gRNA molecule.

263. The Cas9 molecule/gRNA molecule complex of embodiment 262, wherein the gRNA molecule comprises from 5′ to 3′: a targeting domain; a first complementarity domain; a linking domain; a second complementarity domain; a proximal domain; and a tail domain.

264. The Cas9 molecule/gRNA molecule complex of embodiment 262 or embodiment 263, wherein the gRNA molecule comprises a linking domain of no more than 25 nucleotides in length and a proximal and tail domain, that taken together, are at least 20 nucleotides in length.

265. A composition comprising at least two Cas9 molecule/gRNA complexes, each complex comprising a gRNA molecule comprising a targeting domain that is complementary to a target domain from a PDCD1 gene.

266. The composition of embodiment 265, wherein the gRNA molecule comprises a targeting domain that is the same as, or differs by no more than 3 nucleotides from, a targeting domain from any of SEQ ID NOS: 481-555, 563-1516, 1517-3748, 14657-16670, and 16671-21037

267. The composition of embodiment 265, wherein the gRNA molecule comprises a targeting domain that is selected from SEQ ID NOS: 563-1516.

268. The composition of embodiment 265, wherein the gRNA molecule comprises a targeting domain that is selected from SEQ ID NOS: 1517-3748.

269. The composition of embodiment 265, wherein the gRNA molecule comprises a targeting domain that is selected from SEQ ID NOS: 14657-16670.

270. The composition of embodiment 265, wherein the gRNA molecule comprises a targeting domain that is selected from SEQ ID NOS: 16671-21037.

271. The composition of embodiment 265, wherein the gRNA molecule comprises a targeting domain that is selected from SEQ ID NOS: 481-500 and 508-547.

272. The composition of embodiment 265, wherein the gRNA molecule comprises a targeting domain that is selected from SEQ ID NOS: 501-507 and 548-555.

273. The composition of embodiment 265, wherein the gRNA molecule comprises a targeting domain that is selected from SEQ ID NOS: 508, 514, 576, 579, 582, and 723.

274. The composition of embodiment 265, wherein the gRNA molecule comprises a targeting domain that is selected from SEQ ID NOS: 508, 510, 511, 512, 514, 576, 579, 581, 582, 766, and 723.

275. The composition of any of embodiments 265-741, wherein the gRNA molecule is modified at its 5′ end.

276. The composition of embodiment 275, wherein the gRNA molecule lacks a 5′ triphosphate group.

277. The composition of embodiment 275, wherein the gRNA molecule includes a 5′ cap.

278. The composition of embodiment 277, wherein the 5′ cap comprises a modified guanine nucleotide that is linked to the remainder of the gRNA molecule via a 5′-5′ triphosphate linkage.

279. The composition of embodiment 277, wherein the 5′ cap comprises two optionally modified guanine nucleotides that are linked via an optionally modified 5′-5′ triphosphate linkage.

280. The composition of any of embodiments 265-279, wherein the 3′ polyA tail is comprised of about 10 to about 30 adenine nucleotides.

281. The composition of any of embodiments 265-279, wherein the 3′ polyA tail is comprised of about 20 adenine nucleotides.

282. The composition of embodiment 280 or embodiment 281, wherein the gRNA molecule including the 3′ polyA tail was prepared by in vitro transcription from a DNA template.

283. The composition of embodiment 282, wherein the 5′ nucleotide of the targeting domain is a guanine nucleotide, the DNA template comprises a T7 promoter sequence located immediately upstream of the sequence that corresponds to the targeting domain, and the 3′ nucleotide of the T7 promoter sequence is not a guanine nucleotide.

284. The composition of embodiment 282, wherein the 5′ nucleotide of the targeting domain is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

285. The composition of any of embodiments 265-284, wherein the Cas9 molecule cleaves a target domain with a double stranded break.

286. The composition of embodiment 285, wherein the Cas9 molecule is a S. pyogenes Cas9 molecule.

287. The composition of any of embodiments 265-286, wherein the targeting domain is selected from the following group of targeting domains:

(SEQ ID NO: 508)

GUCUGGGCGGUGCUACAACU;

(SEQ ID NO: 514)

GCCCUGGCCAGUCGUCU;

(SEQ ID NO: 576)

CGUCUGGGCGGUGCUACAAC;

(SEQ ID NO: 579)

UGUAGCACCGCCCAGACGAC;

(SEQ ID NO: 582)

CGACUGGCCAGGGCGCCUGU;
or

(SEQ ID NO: 723)

CACCUACCUAAGAACCAUCC.

288. The composition of any of embodiments 265-287, wherein the Cas9 molecule cleaves a target domain with a single stranded break.

289. The composition of embodiment 288, wherein the Cas9 molecule is a S. pyogenes Cas9 molecule.

290. The composition of any of embodiments 265-289, wherein the S. pyogenes Cas9 molecule has a D10A mutation.

291. The composition of any of embodiments 265-290, wherein the targeting domain is selected from the following group of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;

(SEQ ID NO: 582)

CGACUGGCCAGGGCGCCUGU
and

(SEQ ID NO: 511)

GGCCAGGAUGGUUCUUAGGU;

(SEQ ID NO: 582)

CGACUGGCCAGGGCGCCUGU
and

(SEQ ID NO: 512)

GGAUGGUUCUUAGGUAGGUG;

(SEQ ID NO: 582)

CGACUGGCCAGGGCGCCUGU
and

(SEQ ID NO: 576)

CGUCUGGGCGGUGCUACAAC;

(SEQ ID NO: 582)

CGACUGGCCAGGGCGCCUGU
and

(SEQ ID NO: 766)

CUACAACUGGGCUGGCGGCC;

(SEQ ID NO: 579)

UGUAGCACCGCCCAGACGAC
and

(SEQ ID NO: 511)

GGCCAGGAUGGUUCUUAGGU;

(SEQ ID NO: 579)

UGUAGCACCGCCCAGACGAC
and

(SEQ ID NO: 512)

GGAUGGUUCUUAGGUAGGUG;
or

(SEQ ID NO: 581)

ACCGCCCAGACGACUGGCCA
and

(SEQ ID NO: 511)

GGCCAGGAUGGUUCUUAGGU.

292. The composition of any of embodiment 265-291, wherein the S. pyogenes Cas9 molecule has a N863A mutation.

293. The composition of embodiment 292, wherein the targeting domain is selected from the following group of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;
or

(SEQ ID NO: 582)

CGACUGGCCAGGGCGCCUGU
and

(SEQ ID NO: 511)

GGCCAGGAUGGUUCUUAGGU.

294. The composition of any of embodiments 265-293, wherein the gRNA molecule is a modular gRNA molecule.

295. The composition of any of embodiments 265-294, wherein the gRNA molecule is a chimeric gRNA molecule.

296. The composition of embodiment 295, wherein the gRNA molecule comprises from 5′ to 3′: a targeting domain; a first complementarity domain; a linking domain; a second complementarity domain; a proximal domain; and a tail domain.

297. The composition of embodiment 295 or embodiment 296, wherein the gRNA molecule comprises a linking domain of no more than 25 nucleotides in length and a proximal and tail domain, that taken together, are at least 20 nucleotides in length.

298. A gRNA molecule that comprises a targeting domain which is complementary with a target domain from the PDCD1 gene, wherein the gRNA molecule is modified at its 5′ end and/or comprises a 3′ polyA tail.

299. The gRNA molecule of embodiment 298, wherein the gRNA molecule comprises a targeting domain that is the same as, or differs by no more than 3 nucleotides from, a targeting domain from any of SEQ ID NOS: 481-555, 563-1516, 1517-3748, 14657-16670, and 16671-21037

300. The gRNA molecule of embodiment 298, wherein the gRNA molecule comprises a targeting domain that is selected from SEQ ID NOS: 563-1516.

301. The gRNA molecule of embodiment 298, wherein the gRNA molecule comprises a targeting domain that is selected from SEQ ID NOS: 1517-3748.

302. The gRNA molecule of embodiment 298, wherein the gRNA molecule comprises a targeting domain that is selected from SEQ ID NOS: 14657-16670.

303. The gRNA molecule of embodiment 298, wherein the gRNA molecule comprises a targeting domain that is selected from SEQ ID NOS: 16671-21037.

304. The gRNA molecule of embodiment 298, wherein the gRNA molecule comprises a targeting domain that is selected from SEQ ID NOS: 481-500 and 508-547.

305. The gRNA molecule of embodiment 298, wherein the gRNA molecule comprises a targeting domain that is selected from SEQ ID NOS: 501-507 and 548-555.

306. The gRNA molecule of embodiment 298, wherein the gRNA molecule comprises a targeting domain that is selected from SEQ ID NOS: 508, 514, 576, 579, 582, and 723.

307. The gRNA molecule of embodiment 298, wherein the gRNA molecule comprises a targeting domain that is selected from SEQ ID NOS: 508, 510, 511, 512, 514, 576, 579, 581, 582, 766, and 723.

308. The gRNA molecule of any of embodiments embodiment 298-94, wherein the gRNA molecule is modified at its 5′ end.

309. The gRNA molecule of embodiment 308, wherein the gRNA molecule lacks a 5′ triphosphate group.

310. The gRNA molecule of embodiment 308, wherein the gRNA molecule includes a 5′ cap.

311. The gRNA molecule of embodiment 310, wherein the 5′ cap comprises a modified guanine nucleotide that is linked to the remainder of the gRNA molecule via a 5′-5′ triphosphate linkage.

312. The gRNA molecule of embodiment 310, wherein the 5′ cap comprises two optionally modified guanine nucleotides that are linked via an optionally modified 5′-5′ triphosphate linkage.

313. The gRNA molecule of any of embodiments 298-312, wherein the gRNA molecule comprises a 3′ polyA tail which is comprised of about 10 to about 30 adenine nucleotides.

314. The gRNA molecule of any of embodiments 298-312, wherein the gRNA molecule comprises a 3′ polyA tail which is comprised of about 20 adenine nucleotides.

315. The gRNA molecule of embodiment 313 or 314, wherein the gRNA molecule including the 3′ polyA tail was prepared by in vitro transcription from a DNA template.

316. The gRNA molecule of embodiment 315, wherein the 5′ nucleotide of the targeting domain is a guanine nucleotide, the DNA template comprises a T7 promoter sequence located immediately upstream of the sequence that corresponds to the targeting domain, and the 3′ nucleotide of the T7 promoter sequence is not a guanine nucleotide.

316. The gRNA molecule of embodiment 315, wherein the 5′ nucleotide of the targeting domain is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

317. The gRNA molecule of any of embodiments 298-316, wherein the gRNA molecule is a S. pyogenes gRNA molecule.

318. The gRNA molecule of any of embodiments 298-317, wherein the targeting domain is selected from the following group of targeting domains:

(SEQ ID NO: 508)

GUCUGGGCGGUGCUACAACU;

(SEQ ID NO: 514)

GCCCUGGCCAGUCGUCU;

(SEQ ID NO: 576)

CGUCUGGGCGGUGCUACAAC;

(SEQ ID NO: 579)

UGUAGCACCGCCCAGACGAC;

(SEQ ID NO: 582)

CGACUGGCCAGGGCGCCUGU;
or

(SEQ ID NO: 723)

CACCUACCUAAGAACCAUCC.

319. The gRNA molecule of embodiment 318, wherein the targeting domain is selected from the following group of targeting domains:

(SEQ ID NO: 514)

GCCCUGGCCAGUCGUCU;

(SEQ ID NO: 723)

CACCUACCUAAGAACCAUCC.

320. The gRNA molecule of embodiment 318, wherein the targeting domain is selected from the following group of targeting domains:

(SEQ ID NO: 510)

GGGCGGUGCUACAACUGGGC;

(SEQ ID NO: 511)

GGCCAGGAUGGUUCUUAGGU;

(SEQ ID NO: 512)

GGAUGGUUCUUAGGUAGGUG;

(SEQ ID NO: 581)

ACCGCCCAGACGACUGGCCA
and

(SEQ ID NO: 766)

CUACAACUGGGCUGGCGGCC;

321. The gRNA molecule of embodiment 318, wherein the targeting domain is selected from the following group of targeting domains:

(SEQ ID NO: 511)

GGCCAGGAUGGUUCUUAGGU;

(SEQ ID NO: 512)

GGAUGGUUCUUAGGUAGGUG;

(SEQ ID NO: 766)

CUACAACUGGGCUGGCGGCC;

322. The gRNA molecule of any of embodiments 298-321, wherein the gRNA molecule is a modular gRNA molecule.

323. The gRNA molecule of any of embodiments 298-322, wherein the gRNA molecule is a chimeric gRNA molecule.

324. The gRNA molecule of embodiment 323, wherein the gRNA molecule comprises from 5′ to 3′: a targeting domain; a first complementarity domain; a linking domain; a second complementarity domain; a proximal domain; and a tail domain.

325. The gRNA molecule of embodiment 323 or embodiment 324, wherein the gRNA molecule comprises a linking domain of no more than 25 nucleotides in length and a proximal and tail domain, that taken together, are at least 20 nucleotides in length.

326. A method of making a cell for implantation, comprising contacting the cell with one or more Cas9 molecule/gRNA molecule complexes, wherein the gRNA molecule(s) in the one or more Cas9 molecule/gRNA molecule complexes comprise a targeting domain which is complementary with a target domain from the PDCD1 gene.

327. The method of embodiment 326, wherein the gRNA molecule(s) comprise a targeting domain which is complementary with a target domain from the PDCD1 gene and wherein the gRNA molecule(s) guide the Cas9 molecule to cleave the target domain with an efficiency of cleavage of at least 40%.

328. The method of embodiment 327, wherein the efficiency of cleavage is determined using a labeled anti-PDCD1 antibody and a flow cytometry assay

329. The method of any of embodiments 326-328, wherein the gRNA molecule(s) are modified at their 5′ end or comprise a 3′ polyA tail.

330. The method of any of embodiments 326-328, wherein the gRNA molecule(s) are modified at their 5′ end and comprise a 3′ polyA tail.

331. The method of embodiment 329 or embodiment 330, wherein the gRNA molecule(s) lack a 5′ triphosphate group.

332. The method of embodiment 329 or embodiment 330, wherein the gRNA molecule(s) include a 5′ cap.

333. The method of embodiment 332, wherein the 5′ cap comprises a modified guanine nucleotide that is linked to the remainder of the gRNA molecule via a 5′-5′ triphosphate linkage.

334. The method of embodiment 332, wherein the 5′ cap comprises two optionally modified guanine nucleotides that are linked via an optionally modified 5′-5′ triphosphate linkage.

335. The method of any of embodiments 329-334, wherein the 3′ polyA tail is comprised of about 10 to about 30 adenine nucleotides.

336. The method of any of embodiments 329-334, wherein the 3′ polyA tail is comprised of about 20 adenine nucleotides.

337. The method of embodiment 335 or embodiment 336, wherein the gRNA molecule(s) including the 3′ polyA tail were prepared by in vitro transcription from a DNA template.

338. The method of embodiment 337, wherein the 5′ nucleotide of the targeting domain is a guanine nucleotide, the DNA template comprises a T7 promoter sequence located immediately upstream of the sequence that corresponds to the targeting domain, and the 3′ nucleotide of the T7 promoter sequence is not a guanine nucleotide.

339. The method of embodiment 337, wherein the 5′ nucleotide of the targeting domain is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

340. The method of any of embodiments 326-339, wherein the one or more Cas9 molecule/gRNA molecule complexes are delivered into the cell via electroporation.

341. The method of any of embodiments 326-340, wherein the Cas9 molecule is guided by a single gRNA molecule and cleaves the target domain with a single double stranded break.

342. The method of embodiment 341, wherein the Cas9 molecule is a S. pyogenes Cas9 molecule.

343. The method of any of embodiments 326-342, wherein the single gRNA molecule comprises a targeting domain selected from the following targeting domains:

(SEQ ID NO: 508)

GUCUGGGCGGUGCUACAACU;

(SEQ ID NO: 514)

GCCCUGGCCAGUCGUCU;

(SEQ ID NO: 576)

CGUCUGGGCGGUGCUACAAC;

(SEQ ID NO: 579)

UGUAGCACCGCCCAGACGAC;

(SEQ ID NO: 582)

CGACUGGCCAGGGCGCCUGU;
or

(SEQ ID NO: 723)

CACCUACCUAAGAACCAUCC.

344. The method of any of embodiments 326-343, wherein the Cas9 molecule is a nickase and two Cas9 molecule/gRNA molecule complexes are guided by two different gRNA molecules to cleave the target domain with two single stranded breaks on opposing strands of the target domain.

345. The method of any of embodiments 326-344, wherein the Cas9 molecule is a S. pyogenes Cas9 molecule having a D10A mutation.

346. The method of any of embodiments 326-345, wherein the two gRNA molecules comprise targeting domains that are selected from the following pairs of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;

(SEQ ID NO: 582)

CGACUGGCCAGGGCGCCUGU
and

(SEQ ID NO: 511)

GGCCAGGAUGGUUCUUAGGU;

(SEQ ID NO: 582)

CGACUGGCCAGGGCGCCUGU
and

(SEQ ID NO: 512)

GGAUGGUUCUUAGGUAGGUG;

(SEQ ID NO: 582)

CGACUGGCCAGGGCGCCUGU
and

(SEQ ID NO: 576)

CGUCUGGGCGGUGCUACAAC;

(SEQ ID NO: 582)

CGACUGGCCAGGGCGCCUGU
and

(SEQ ID NO: 766)

CUACAACUGGGCUGGCGGCC;

(SEQ ID NO: 579)

UGUAGCACCGCCCAGACGAC
and

(SEQ ID NO: 511)

GGCCAGGAUGGUUCUUAGGU;

(SEQ ID NO: 579)

UGUAGCACCGCCCAGACGAC
and

(SEQ ID NO: 512)

GGAUGGUUCUUAGGUAGGUG;
or

(SEQ ID NO: 581)

ACCGCCCAGACGACUGGCCA
and

(SEQ ID NO: 511)

GGCCAGGAUGGUUCUUAGGU.

347. The method of any of embodiments 326-346, wherein the S. pyogenes Cas9 molecule has a N863A mutation.

348. The method of embodiment 347, wherein the two gRNA molecules comprise targeting domains that are selected from the following pairs of targeting domains:

(SEQ ID NO: 582)

CGACUGGCCAGGGCGCCUGU
and

(SEQ ID NO: 508)

GUCUGGGCGGUGCUACAACU;

(SEQ ID NO: 582)

CGACUGGCCAGGGCGCCUGU
and

(SEQ ID NO: 510)

GGGCGGUGCUACAACUGGGC;
or

(SEQ ID NO: 582)

CGACUGGCCAGGGCGCCUGU
and

(SEQ ID NO: 511)

GGCCAGGAUGGUUCUUAGGU.

349. The method of any of embodiments 326-348, wherein the gRNA molecule(s) are modular gRNA molecule(s).

350. The method of any of embodiments 326-349, wherein the gRNA molecule(s) are chimeric gRNA molecule(s).

351. The method of embodiment 350, wherein the gRNA molecule(s) comprise from 5′ to 3′: a targeting domain; a first complementarity domain; a linking domain; a second complementarity domain; a proximal domain; and a tail domain.

352. The method of embodiment 350 or embodiment 351, wherein the gRNA molecule(s) comprise a linking domain of no more than 25 nucleotides in length and a proximal and tail domain, that taken together, are at least 20 nucleotides in length.

353. The method of any of embodiments 326-352, wherein the gRNA molecule(s) guide the Cas9 molecule to cleave the target domain with an efficiency of cleavage of at least 60%.

354. The method of any of embodiments 326-352, wherein the gRNA molecule(s) guide the Cas9 molecule to cleave the target domain with an efficiency of cleavage of at least 80%.

355. The method of any of embodiments 326-352, wherein the gRNA molecule(s) guide the Cas9 molecule to cleave the target domain with an efficiency of cleavage of at least 90%.

356. The method of any of embodiments 326-355, wherein the one or more Cas9 molecule/gRNA molecule complexes produce fewer than 5 off-targets.

357. The method of any of embodiments 326-356, wherein the one or more Cas9 molecule/gRNA molecule complexes produce fewer than 2 exonic off-targets.

358. The method of embodiment 356 or embodiment 357, wherein off-targets are identified by GUIDE-seq.

359. The method of embodiment 356 or embodiment 357, wherein off-targets are identified by Amp-seq.

360. The method of any of embodiments 326-359, wherein the contacting is performed ex vivo.

361. The method of any of embodiments 326-360, wherein the cell is an immune cell.

362. The method of embodiment 361, wherein the cell is a lymphocyte or antigen presenting cell.

363. The method of embodiment 362, wherein the cell is a T cell, B cell or antigen presenting cell.

364. The method of any of embodiments 326-363, wherein the cell is a T cell.

365. The method of any of embodiments 326-364, wherein the cell comprises a recombinant receptor.

366. The method of any of embodiments 326-365, further comprising contacting the cell with a nucleic acid encoding a recombinant receptor under conditions to introduce the nucleic acid into the cell.

367. The method of embodiment 365 or embodiment 366, wherein the recombinant receptor is a functional non-TCR antigen receptor or a transgenic TCR.

368. The method of any of embodiments 365-367, wherein the recombinant receptor is a chimeric antigen receptor (CAR).

369. The method of embodiment 368, wherein the CAR comprises an antigen-binding domain that is an antibody or an antibody fragment.

370. The method of embodiment 369, wherein the antibody fragment is a single chain fragment.

371. The method of embodiment 369 or embodiment 370, wherein the antibody fragment comprises antibody variable regions joined by a flexible immunoglobulin linker.

372. The method of any of embodiments 369-371, wherein the fragment comprises an scFv.

373. The method of any of embodiments 369-372, wherein the antigen is associated with a disease or disorder.

374. The method of embodiment 373, wherein the disease or disorder is an infectious disease or condition, an autoimmune disease, an inflammatory disease or a tumor or a cancer.

375. The method of any of embodiments 365-374, wherein the recombinant receptor specifically binds to a tumor antigen.

376. The method of any of embodiments 369-375, wherein the antigen is selected from ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acethycholine e receptor, GD2, GD3, HMW-MAA, IL-22R-alpha, IL-13R-alpha2, kdr, kappa light chain, Lewis Y, L1-cell adhesion molecule (CD171), MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1), cyclin A1 (CCNA1), BCMA and interleukin 12.

377. The method of any of embodiments 365-376, wherein the recombinant receptor comprises an intracellular signaling domain comprising an ITAM.

378. The method of embodiment 377, wherein the intracellular signaling domain comprises an intracellular domain of a CD3-zeta (CD3) chain.

379. The method of embodiment 377 or 378, wherein the recombinant receptor further comprises a costimulatory signaling region.

380. The method of embodiment 379, wherein the costimulatory signaling region comprises a signaling domain of CD28 or 4-1BB.

381. A T cell made by the method of any of embodiments 162-231 and 326-380.

382. A T cell comprising the Cas9 molecule/gRNA molecule complex of any of embodiments 232-264.

383. A T cell comprising the composition of any of embodiments 265-297.

384. A method of treating a subject, comprising administering to the subject the T cell of any of embodiments 381-383.

385. The Cas9 molecule/gRNA molecule complex of any of embodiments 232-264, the composition of any of embodiments 265-297, or the T cell of any of embodiments 381-383, for use in therapy.

386. Use of the Cas9 molecule/gRNA molecule complex of any of embodiments 232-264 or the composition of any of embodiments 265-297 in the manufacture of a medicament for treating cancer.

### VII. EXAMPLES

The following examples are included for illustrative purposes only and are not intended to limit the scope of the invention.

### Example 1: Screening of gRNAs for PDCD1 in Primary T Cells

In order to evaluate certain gRNAs for targeting PDCD1 (the gene encoding programmed death-1, PD-1), ribonucleoprotein complexes of gRNA and Cas9 (RNP) comprising different labeled gRNAs targeting the PDCD1 locus were generated and delivered to activated primary T cells by electroporation. S. pyogenes Cas9 protein purified essentially as described in published PCT application NO. WO2015161276 was complexed with the respective in vitro transcribed gRNA (prepared essentially as described in published PCT application NO. WO2015161276) for at least 15 min at a 1:1, 1:1.25 or 1:5 Cas9:gRNA ratio depending on the gRNA.

After verification that the protein was fully complexed with gRNA using differential scanning fluorimetry (DSF), the RNP was administered to activated CD4+ T cells from a healthy human donor using electroporation. RNP was added to 500,000 cells using electroporation in a 96-well format at a dose of 1 μg of RNP/100,000 cells. The cells were cultured post electroporation in T cell media containing IL-2, IL-7 and IL-15.

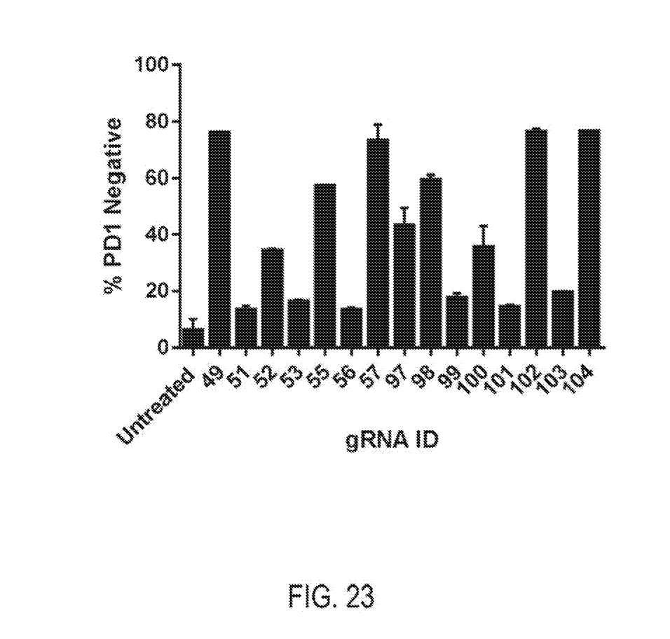

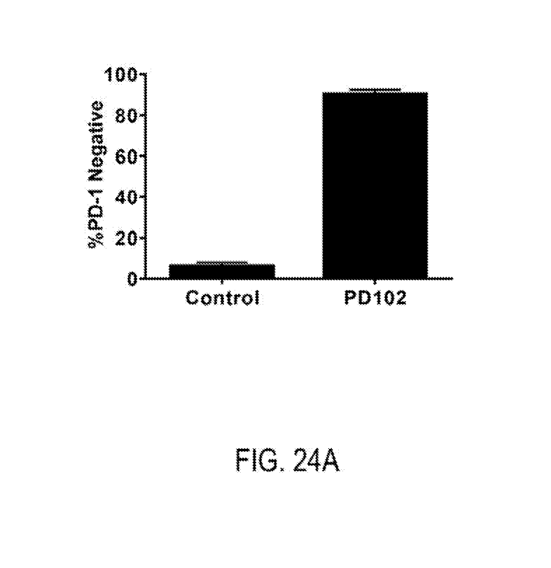

In order to assess the efficiency of PDCD1 knockout, the T cells were reactivated using anit-CD3/anti-CD28 beads for 48 hours while cultured in T cell media. At day 7 post electroporation, cells were analyzed by flow cytometry using a PE-conjugated anti-PD1 antibody as described in Example 2. The percentage of PD-1 negative cells is shown in FIG. 23. Table 1000 provides the sequence of the targeting domain for the gRNAs from the six exemplary RNPs with greater than 45% PDCD1 knockout efficiency that were identified in this manner.

TABLE 1000

| | | | Cas9 | SEQ |
|---|---|---|---|---|
| ID | gRNA Name | Targeting Domain | species | ID NO: |
| 49 | PDCD1-108 | GUCUGGGCGGUGCUACA | S. pyogenes | 508 |
| | | ACU | | |
| 55 | PDCD1-232 | GCCCUGGCCAGUCGUCU | S. pyogenes | 514 |
| 97 | PDCD1-107 | CGUCUGGGCGGUGCUAC | S. pyogenes | 1533 |
| | | AAC | | |
| 98 | PDCD1-122 | UGUAGCACCGCCCAGAC | S. pyogenes | 579 |
| | | GAC | | |
| 102 | PDCD1-126 | CGACUGGCCAGGGCGCC | S. pyogenes | 582 |
| | | UGU | | |
| 104 | PDCD1-121 | CACCUACCUAAGAACCA | S. pyogenes | 723 |
| | | UCC | | |

The specificity of each the 6 gRNAs identified above was assessed in primary T cells by GUIDE-seq (see Nature Biotechnology 33:187-197, 2015, which is incorporated herein by reference in its entirety). The results of four independent gDNA samples derived from 2 separate experiments is summarized in Table 2000. An off target was called if bidirectional reads were present in at least one of the 4 samples or unidirectional reads were present in at least 2 of the 4 samples. To confirm the GUIDE-seq results, Amp-seq was performed on 6 independent gDNAs from T cells treated with S. Pyogenes RNP prepared using a gRNA with the targeting domain GUCUGGGCGGUGCUACAACU (SEQ ID NO:508). Amp-seq results were similar to the GUIDE-seq results, and confirmed both (a) the off targets identified and (b) the rank ordering of the guides generated by GUIDE-seq.

TABLE 2000

| Guide ID | 49 | 55 | 97 | 98 | 102 | 104 |
|---|---|---|---|---|---|---|
| SEQ ID NO | 508 | 514 | 1533 | 579 | 582 | 723 |
| # Off Targets | 4 | 2 | 0 | 1 | 5 | 3 |
| # Exonic | 1 | 0 | 0 | 0 | 1 | 0 |
| # Intronic | 0 | 1 | 0 | 0 | 2 | 2 |
| Experimental | 10.5 | 7.9 | 12 | 9.5 | 9.9 | 8 |
| Reads (M) | | | | | | |

### Example 2: Assessment of PDCD1 Knockout Efficiency Across Multiple Donors

In order to assess cutting efficiency across multiple donors, RNP targeting PDCD1 prepared using gRNA with the targeting domain CGACUGGCCAGGGCGCCUGU (SEQ ID NO:582) was electroporated into activated primary CD4+ T cells from multiple donors. As a control, RNP prepared using control RNP prepared using gRNA with an AAVS1 targeting domain (SEQ ID NO:387) also was electroporated into activated primary CD4+ T cells. PD-1 expression was then assessed by flow cytometry (FACS) using a PE-conjugated anti-PD-1 antibody.

Primary CD4+ T cells that were previously isolated from healthy donors were thawed and activated using anti-CD3/anti-CD28 beads while cultured in T cell media containing IL-2, IL-7 and IL-15. After 48 hours of activation, beads were removed from the cells and cultivated for an additional 24 hours prior to electroporation with RNP at a dose of 1 μg/100,000 cells. After several days of cultivation (between 3 to 4 days), the cells were re-stimulated with either anti-CD3/anti-CD28 beads or PMA/Ionomycin (PMA/IO). In the case of anti-CD3/anti-CD28 activation, the cells were incubated with the beads for 48 hours and assessed for PD-1 expression by FACS 24 hours later. In the case of PMA/IO activation, the cells were cultured in the presence of PMA/IO for 24 hours followed by assessment of PD-1 expression by FACS. PD-1 expression was assessed using a PE-conjugated anti-PD-1 antibody (available from BioLegend, CA) in accordance with the “Cell Surface Immunofluorescence Staining Protocol” which is available on the BioLegend website: www.biolegend.com/media_assets/support_protocol/BioLegend_Surface_Staining_Flow_Protocol_091012.pdf and which is incorporated herein by reference in its entirety. Gating parameters for T-cell sorting were set based on fluorescent signals in one or more channels and forward and side scatter as described in the literature, e.g., see D. Davies, Cell Sorting by Flow Cytometry, pp. 257-276 in Flow Cytometry: Principles and Applications, Edited by: M. G. Macey, 2007 (Humana Press Inc., Totowa, N.J.) which is incorporated herein by reference in its entirety. Regardless of activation conditions, the expression of PD-1 was assessed in the AAVS1 edited or untreated control population.

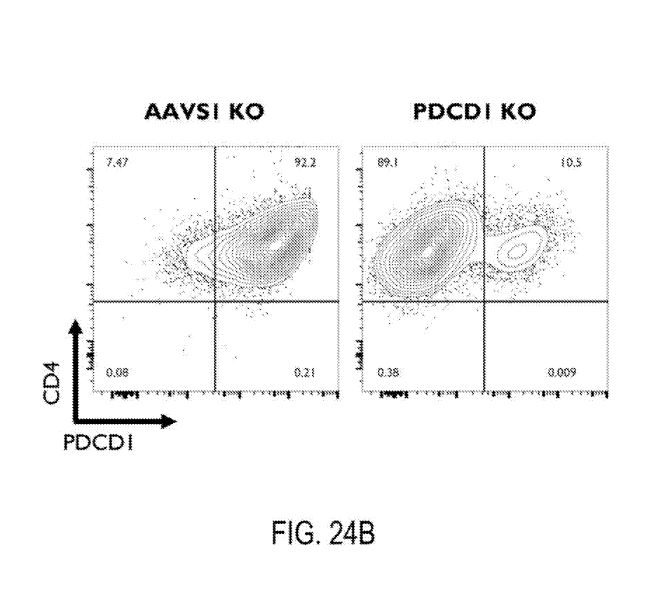

In FIG. 24A, the percentage of cells with PDCD1 knockout is plotted with the error bars depicting the standard deviation over the multiple donors. An example of a PD-1 expression as detected by FACS (flow cytometry) in the aforementioned experiments is shown in FIG. 24B. Upregulation of PD-1 was observed in the AAVS1 edited or untreated control population. In cells treated with RNP targeting PDCD1, compositions were observed to contain approximately 90% PD-1 negative T cells, with >90% PD-1 negative cells observed in cells generated from some donors.

### Example 3: PDCD1 Knockout Does Not Alter the Composition of the T Cell Culture

In order to assess whether deletion of PDCD1 resulted in changes to the composition of CD8+ T cell culture, RNP targeting PDCD1 prepared using gRNA with the targeting domain CGACUGGCCAGGGCGCCUGU (SEQ ID NO:582) was delivered to a co-culture of CD4+/CD8+ T cells. CD8+ T cells treated with RNP prepared using gRNA with an AAVS1 targeting domain (SEQ ID NO:387) were used as a control.

Isolated CD4+ and CD8+ T cells were activated with anti-CD3/anti-CD28 beads and cultured in T cell media containing IL-2, IL-7 and IL-15. 48 hours after activation, the activation beads were removed and cells were cultured overnight. The next day the cells were electroporated with the PDCD1- or AAVS1-targeting RNP and cultured in T cell media containing IL-2, IL-7 and IL-15.

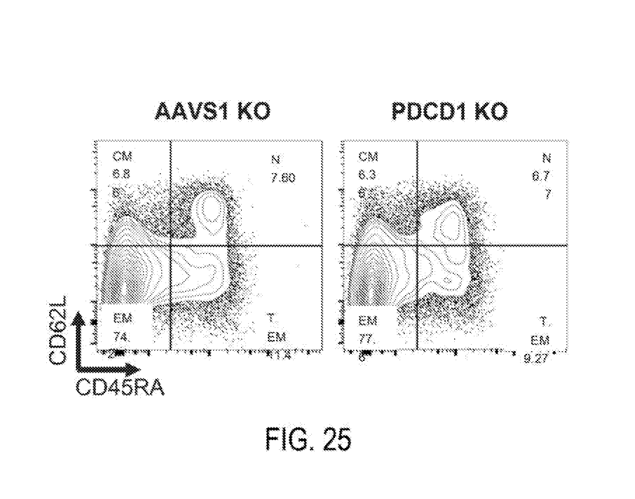

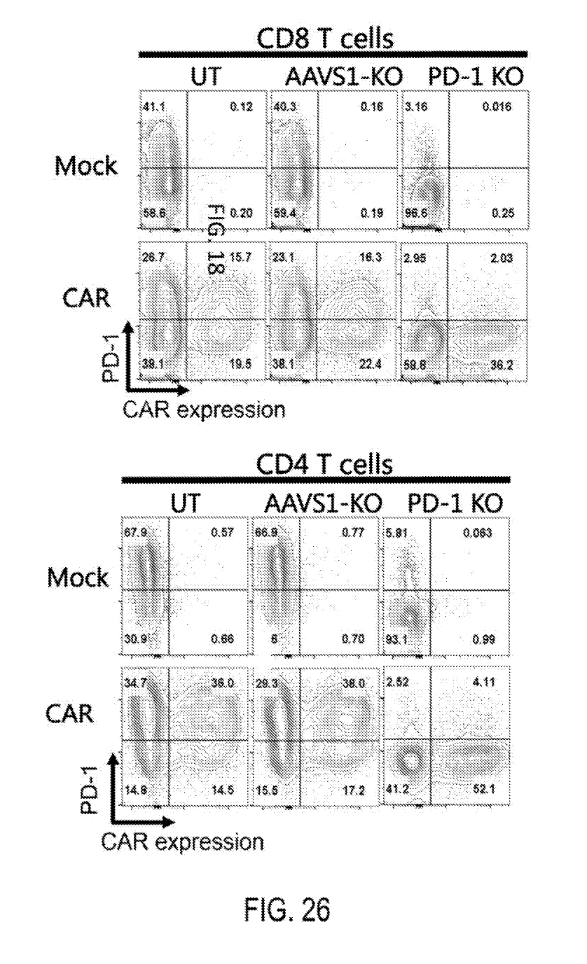

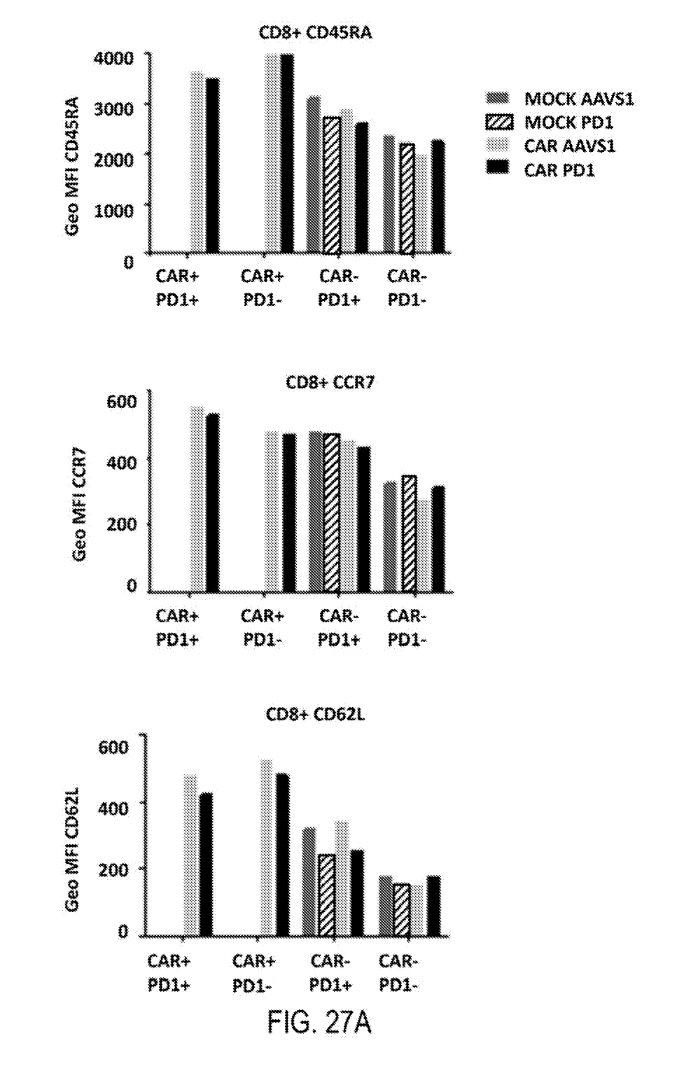

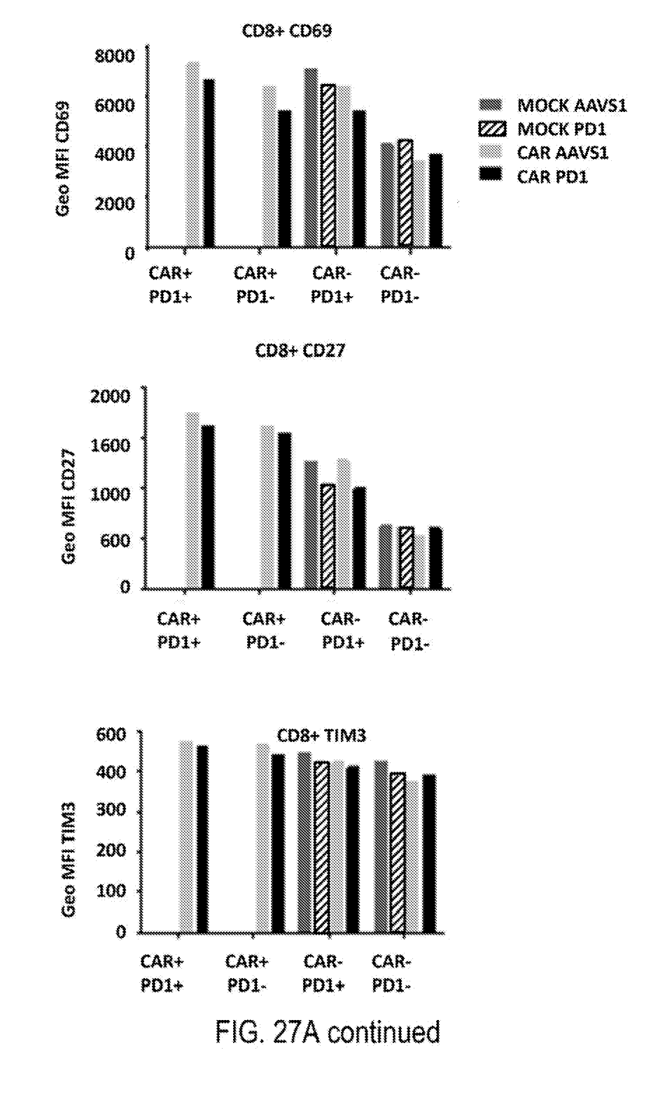

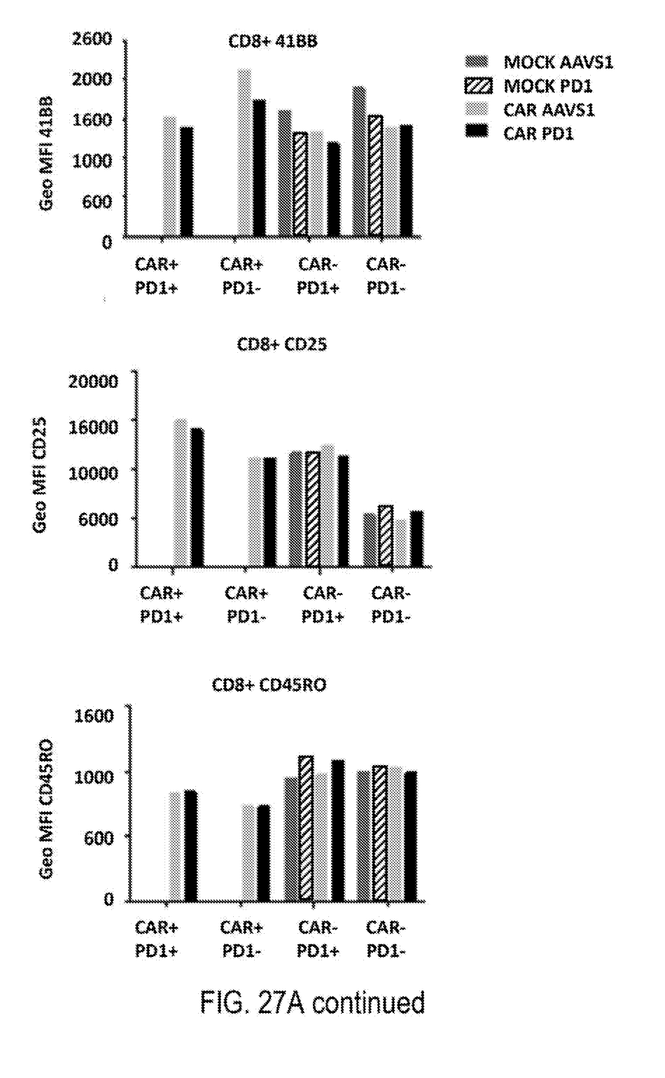

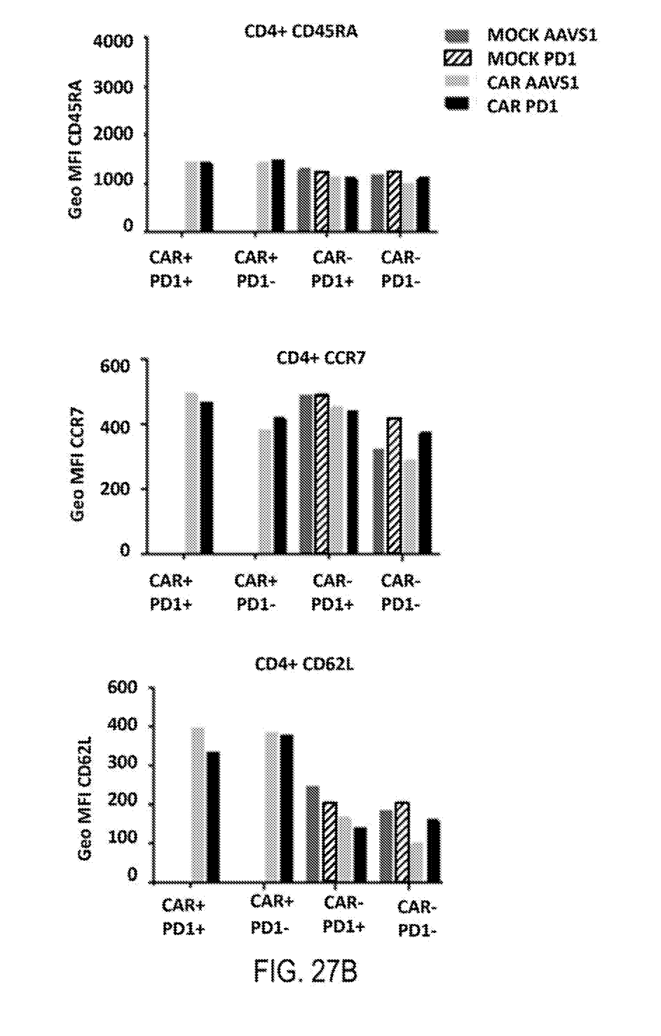

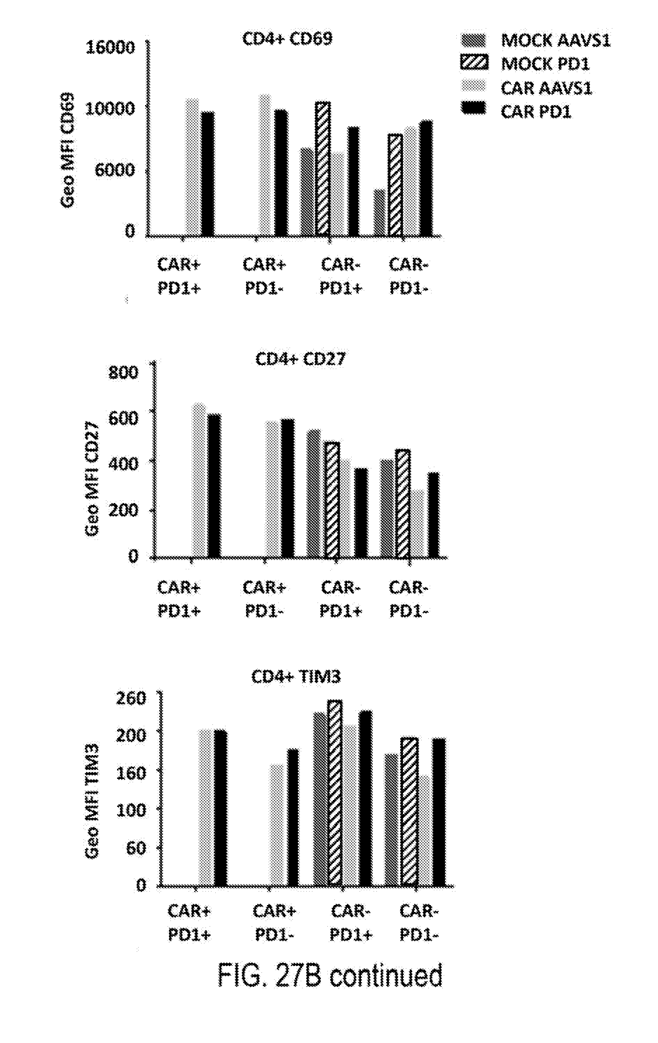

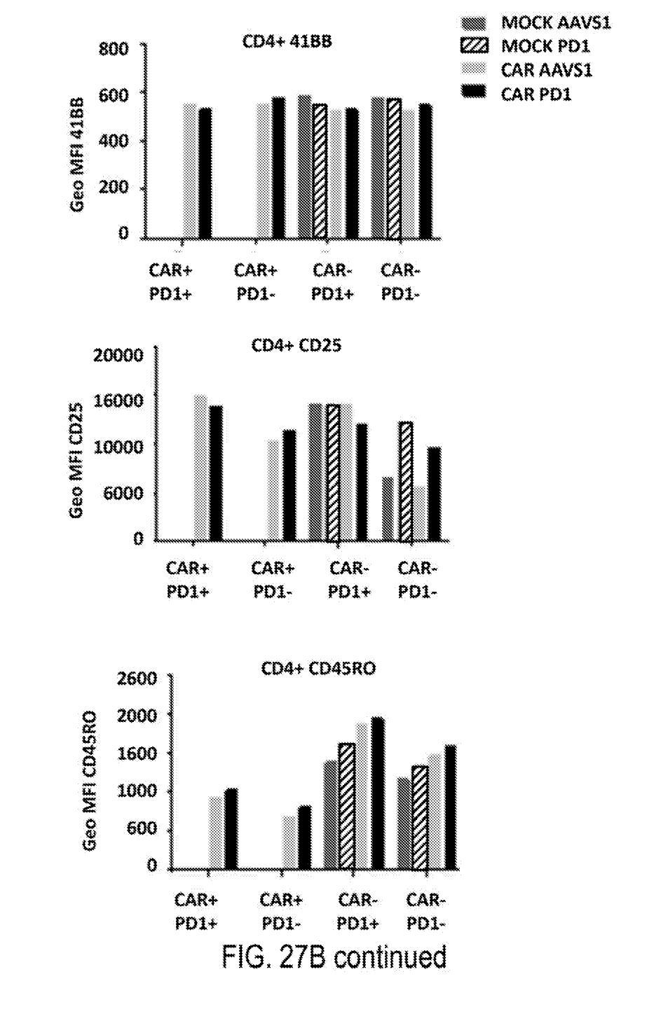

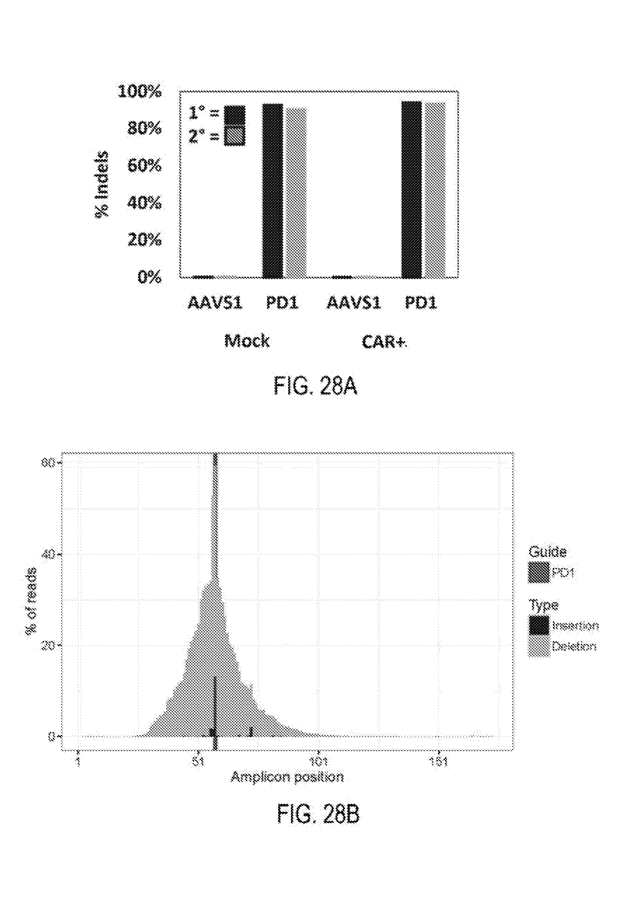

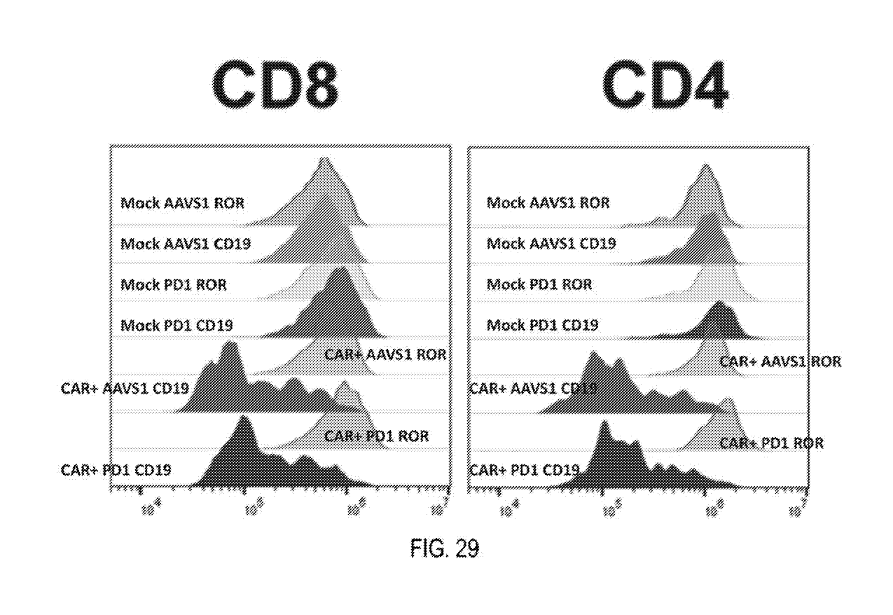

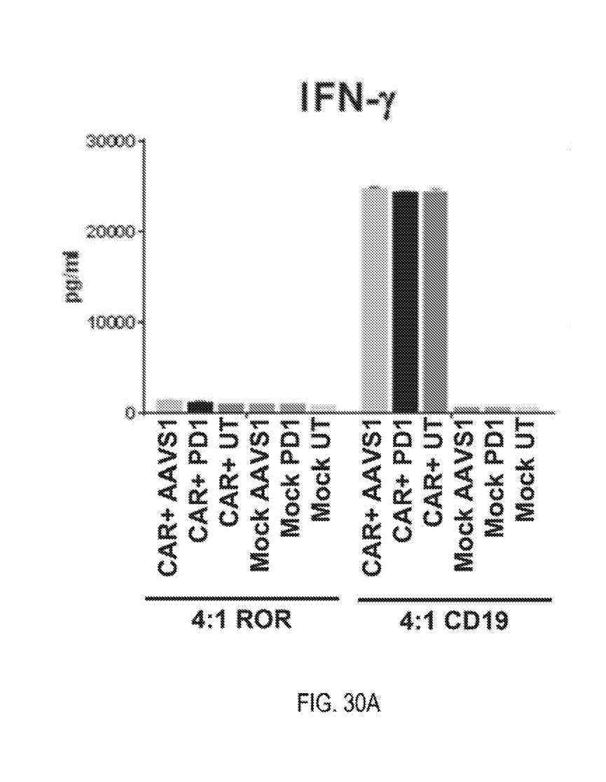

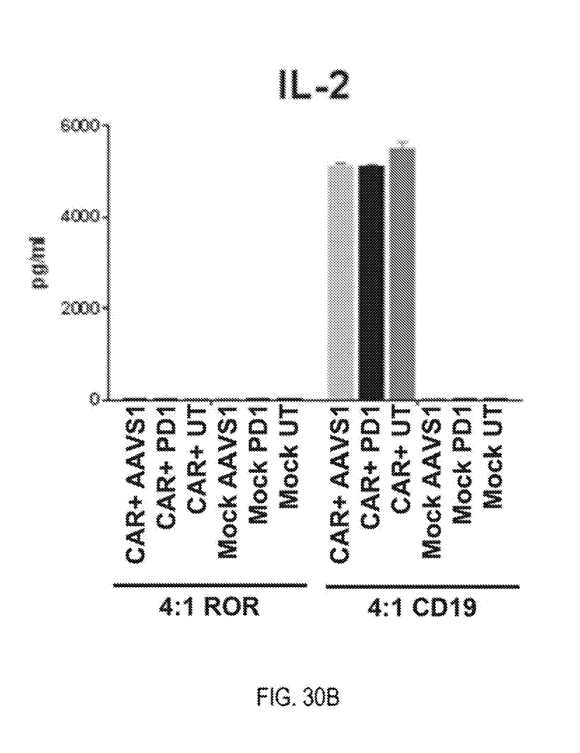

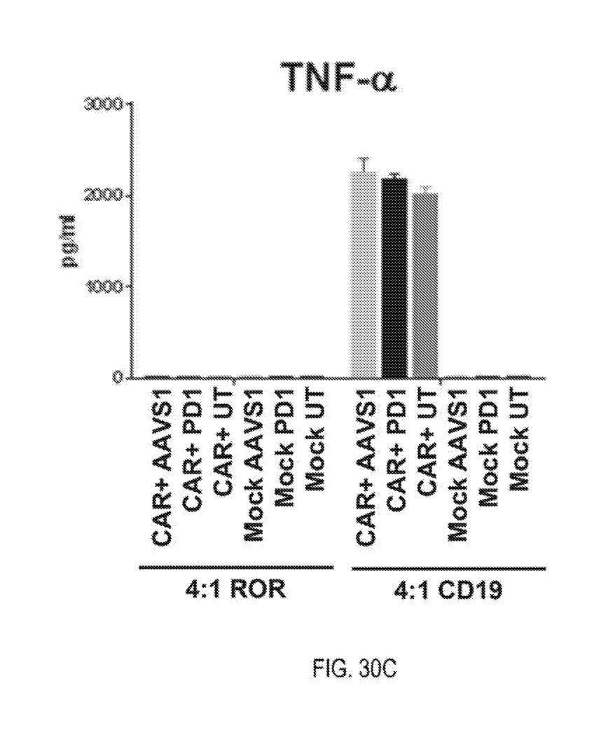

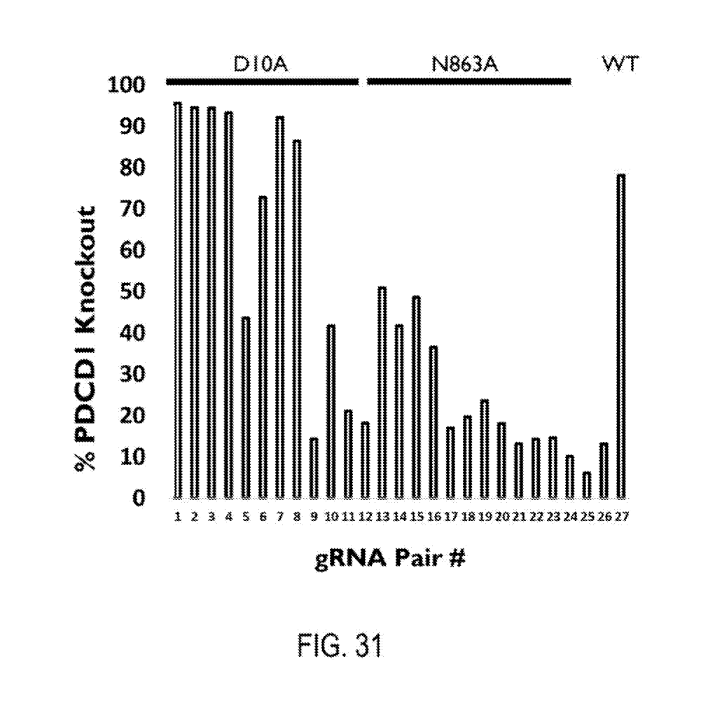

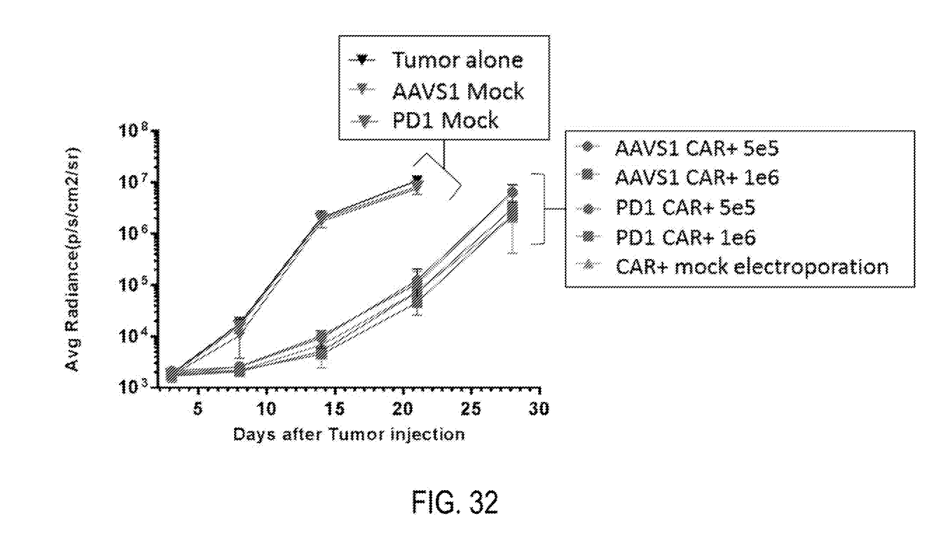

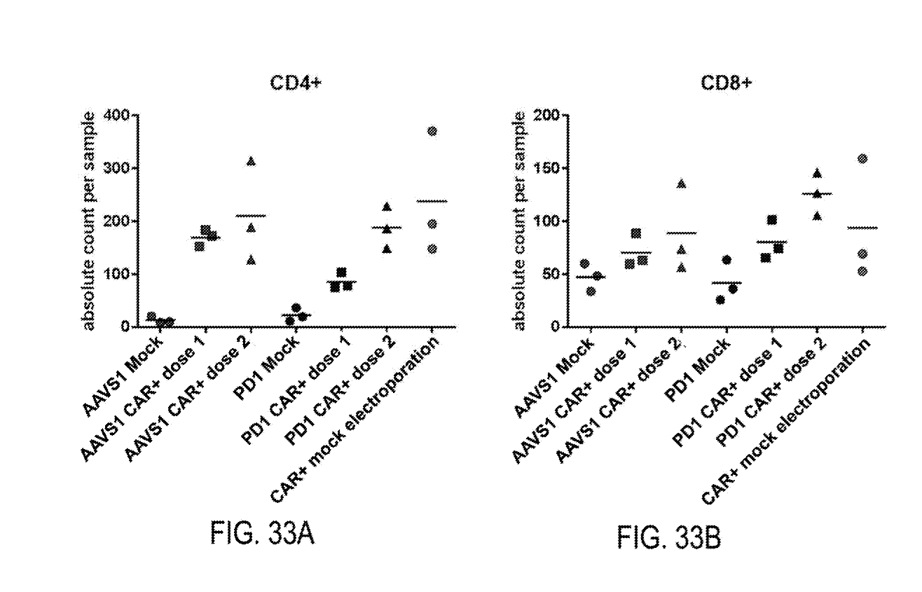

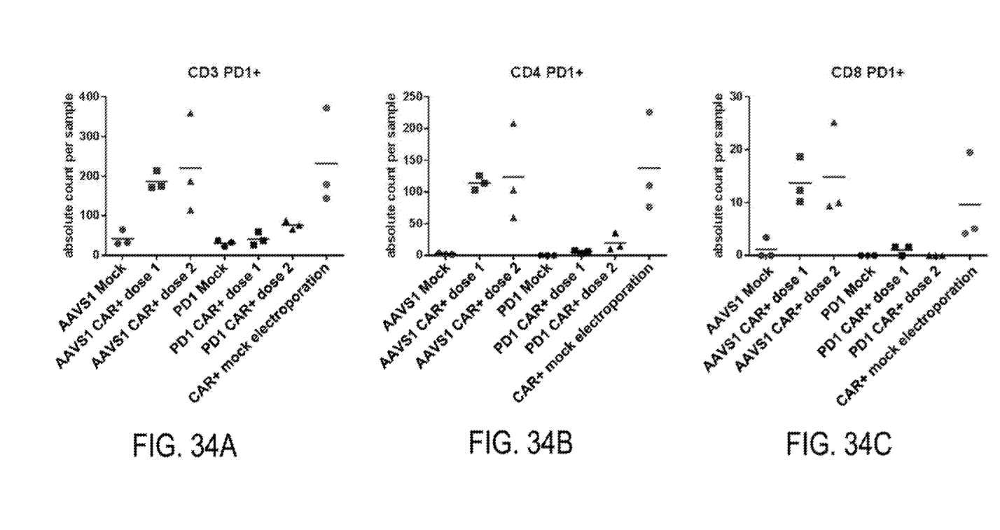

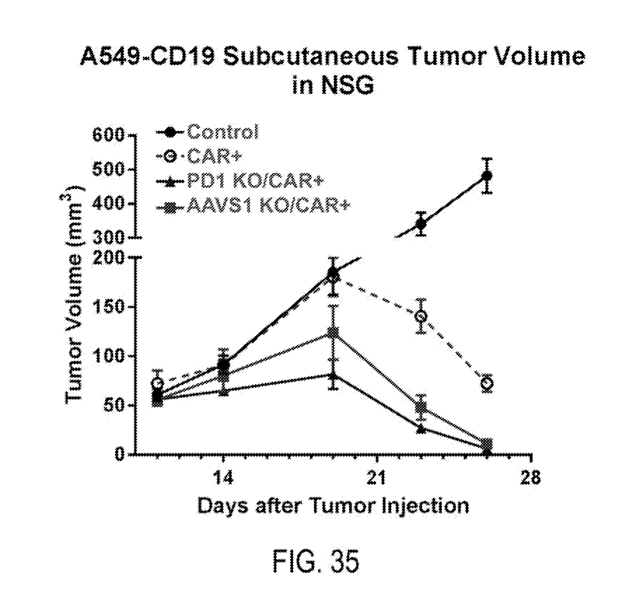

A subset of the cells were split off at day 4 to assess the level of PD-1 expression by flow cytometry after reactivation with anti-CD3/anti-CD28 beads. The remainder of the cells (non-activated cells) were frozen down in T cell freezing media. To determine whether the composition of the cells was altered by the deletion of PDCD1, the AAVS1 guide- and PDCD1 guide-treated cells were thawed into T cell media containing IL-2, IL-7 and IL-15. The cells were then stained with antibodies against CD8, CD62L, and CD45RA in order to assess sub-populations within the CD8+ cell population (including, for example, naïve, central memory, effector memory and terminally differentiated effector memory). The CD62L and CD45RA surface expression levels detected on live (based on forward/side scatter) CD8+ cells is shown in FIG. 25. The subpopulations based on the expression of these two markers are marked in the quadrants of the contour plots with the percentages of cells in each quadrant labeled. The plot shows that there were no major changes observed in the populations in the cells treated with PDCD1-targeting RNP as compared to cells treated with the control AAVS1-targeting RNP.

### Example 4: Genetic Disruption of PDCD1 in Cells Genetically Engineered to Express a Chimeric Antigen Receptor (CAR)

Primary human CD4+ and CD8+ T cells were isolated by immunoaffinity-based selection from human PBMC samples obtained from healthy donors. The resulting cells were stimulated by culturing with an anti-CD3/anti-CD28 reagent in media containing human serum, IL-2 (100 U/mL), IL-7 (10 ng/mL) and IL-15 (5 ng/mL) at 37° C. prior to engineering with a chimeric antigen receptor (CAR) by lentiviral transduction for 24-48 hours. The cells were transduced using a lentiviral vector containing a nucleic acid molecule encoding an exemplary anti-CD19 CAR and a nucleic acid encoding a truncated EGFR (EGFRt), for use as a surrogate marker for transduction, separated by a sequence encoding a T2A ribosome switch. The CAR included an anti-CD19 scFv, an Ig-derived spacer, a human CD28-derived transmembrane domain, a human 4-1BB-derived intracellular signaling domain and a human CD3 zeta-derived signaling domain. A mock transduction was used as a negative control.

Following transduction, the cells were cultured for 36-48 hours in media containing human serum and IL-2 (50 U/mL), IL-7 (5 ng/mL) and IL-15 (0.5 ng/mL). The cells then were electroporated with RNP prepared using the PDCD1-targeted gRNA with the targeting domain CGACUGGCCAGGGCGCCUGU (SEQ ID NO:582) (or the AAVS1 control gRNA with the targeting domain GUCCCCUCCACCCCACAGUG (SEQ ID NO:387) and S. pyogenes Cas9. The cells then were cultured in the same media containing IL-2, IL-7 and IL-15 at the same concentrations, overnight at 30° C., and then at 37° C. through day 12-15 post-electroporation.

A. CAR and PD-1 Expression

Cell surface expression of PD-1 and CAR expression (as indicated via the surrogate marker) was assessed on day 12 after electroportation, following a 24 hour re-stimulation with beads conjugated with anti-CD3/anti-CD28 antibodies. Cells were stained with anti-EGFR antibody or an anti-PD1 antibody to verify CAR expression (as indicated by surface expression of the surrogate marker, EGFRt) and PD-1 expression on the surface by flow cytometry. The results are shown in FIG. 26.

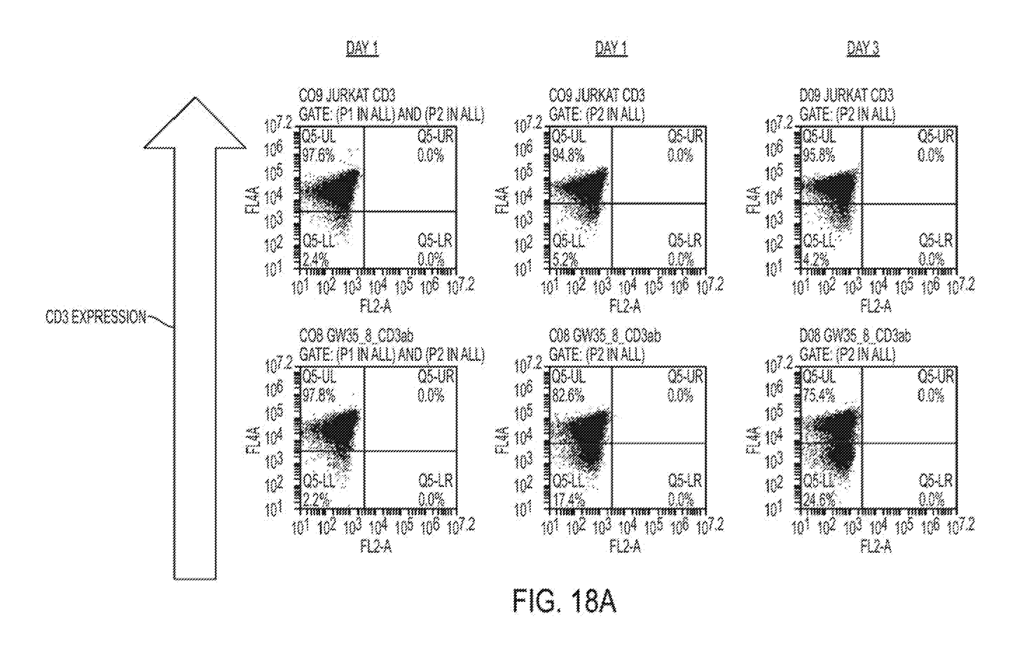

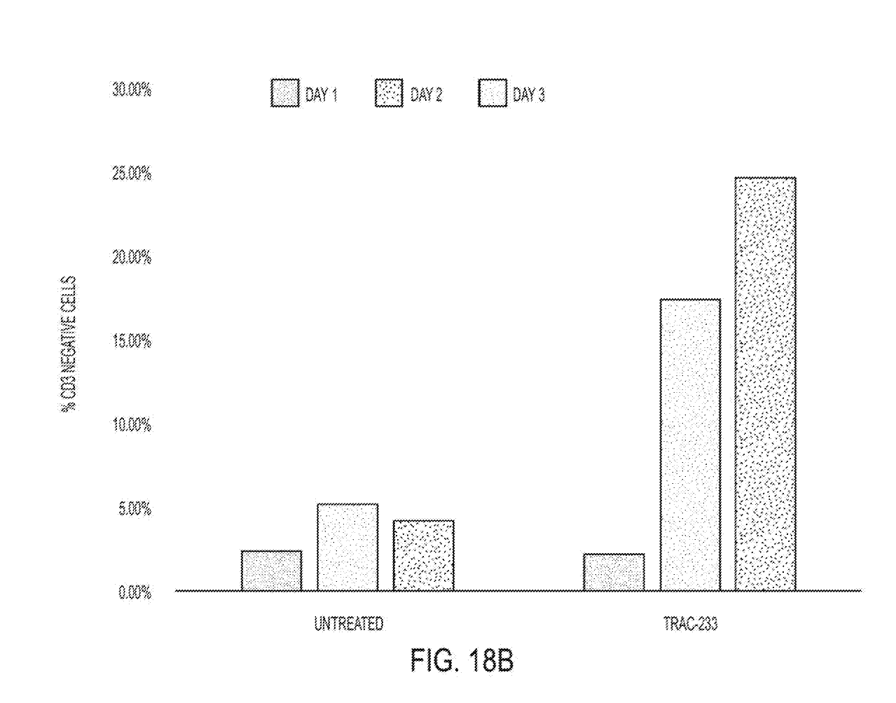

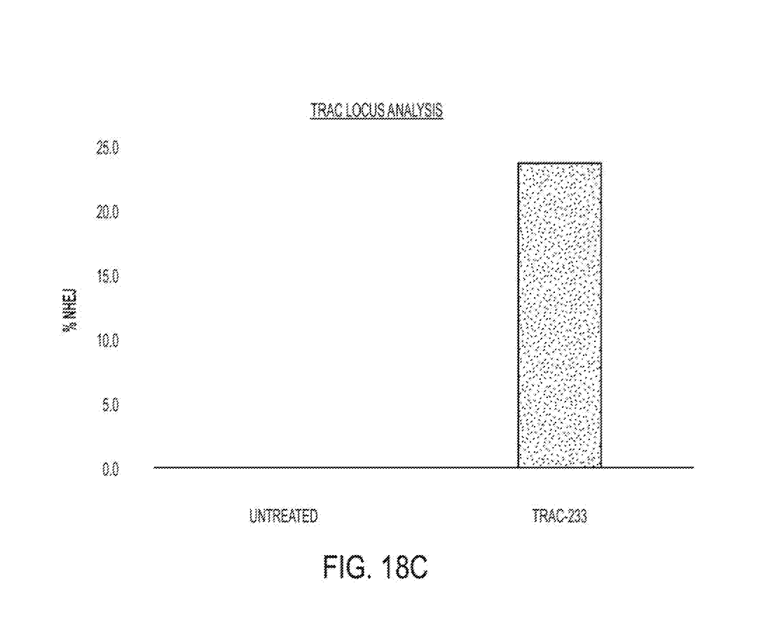

As shown in FIG. 18, greater than 90% of the CD8+ T cells and greater than 90% of the CD4+ T cells subjected to electroporation with the PDCD1-targeting RNP under these conditions were observed to be negative for surface expression of PD-1, including in the CAR-expressing populations (as indicated by the EGFRt marker). This result was consistent with the efficient deletion of PDCD1 at both alleles, in both CAR-transduced and non-CAR-expressing CD8+ and CD4+ cells. Surface expression of the surrogate marker also was observed in both control and PD-1-negative cells, indicating that PD-1 knockout did not prevent surface expression of the recombinant surrogate marker protein.

B. Phenotypic Assessment of CAR+ PD-1 KO Cells

Phenotypic characteristics of the modified engineered CD4+ and CD8+ T cells also were assessed by flow cytometry assessing surface expression of various markers, including those indicative of phenotype, differentiation state and/or activation state. Cells were stained with antibodies specific for CCR7, 41BB, TIM3, CD27, CD45RA, CD45RO, Lag3, CD62L, CD25 and CD69 in addition to those for recognizing PD-1 and the EGFRt marker (surrogate for CAR expression) as described above. The mean fluorescence intensity detected for each marker in each subpopulation (CAR+/PD1+; CAR+/PD1-; CAR-/PD1+; and CAR-/PD1-) of each T cell sbutype CD4+ and CD8+ was determined.

The results, set forth in FIG. 27A (CD4+) and FIG. 27B (CD8+), indicated that under the conditions tested, expression levels of the various markers were similar among PD-1-negative CAR-expressing cells (CAR+/PD1−) and the PD-1-positive CAR-expressing cells (CAR+/PD1+).

C. PDCD1 Deletion in CAR-T Cells

The disruption of the PDCD1 locus by nuclease-induced non-homologous end-joining (NHEJ) can result in the presence of insertion and/or deletion (indel) mutations in the DNA sequence at the site of the NHEJ repair. T cells engineered and subject to deletion as described above were analyzed for the presence of indels at day 20 postprimary expansion and 10 days after a secondary expansion using MiSeq sequencer (Illumina). The number of indels at the PDCD1 locus and their relative positions compared to the PDCD1 cut site introduced by the PDCD1 targeted gRNA were determined.

As shown in FIG. 28A, more than 90% of the CAR+ T cells and the mock-transduced T cells that had been electroporated with the PDCD1-targeted Cas9/gRNA RNP contained indels at the PDCD1 locus after primary and after secondary expansion. By comparison, PDCD1 indels were not detected in cells electroporated with the control gAASV1-targeted Cas9/gRNA RNP. As shown in FIG. 28B, the insertions and deletions occurred on or near (i.e., within 50 base pairs upstream or downstream) the cut site directed by the PDCD1 targeted gRNA. The results demonstrated that the PDCD1 targeted gRNA under these conditions effected genetic disruption of PDCD1 in more than 90% of the CAR-expressing T cells, and the disruption was stable through multiple expansions.

### Example 5: Functional Activity of PDCD1-Deleted, CAR-Expressing T Cells

Genetically engineered human T cells (either CD8+ or CD4+) expressing an exemplary anti-CD19 CAR and knocked out for expression of the PD-1 gene, produced as described in Example 4, were assessed for various functional responses.

A. Cytolytic Activity

Transduction (and mock transduction) and PDCD1 (or control) deletion was carried out as described in Example 4 above. The cells then were assessed for cytolytic activity against K562 target cells expressing the CD19 antigen (K562-CD19) or non-specific CD19-negative K562 control cells expressing a control antigen (ROR1) (K562-ROR). The T cells were incubated with the target cells (K562-CD19 or K562-ROR1) at a 4:1 effector:target ratio in the presence of NucRed dye. Lysis of target cells was measured over 70 hours using the Incucyte quantitative cell analysis system (Essen BioScience) by assessing the staining intensity of cells for the NucRed dye. Cells in which lysis occurred exhibited reduced staining intensity for the dye.

The results showed that CAR-expressing T cells (e.g. CAR+/PD1+, CAR+/PD1− and CAR+/AAVS1−) were able to kill CD19-expressing target cells in a target antigen specific manner to a similar degree. No cell lysis was observed by any of these cells following incubation with target cells expressing the non-specific antigen. The results demonstrated that deletion of PDCD1 did not, under these conditions, affect the CAR-mediated cytotoxic activity of the anti-CD19 CAR-expressing T cells.

B. T Cell Expansion

Proliferation of the T cells following incubation with CD19-expressing target cells was assessed by flow cytometry. CD8+ or CD4+ CAR-expressing T cells (or mock control) subject to deletion using RNPs with a guide targeting PDCD1 (or the AAVS1 control) generated as described above were labeled with CellTrace™ violet (ThermoFisher) cell proliferation assay dye. Cells were washed and incubated for 96 hours in triplicate with the same target cells (K562-CD19 or K562-ROR) at a 1:1 effector:target ratio. Division of live T cells was indicated by CellTrace™ violet dye dilution, as assessed by flow cytometry.

As shown in FIG. 29, CD4+ and CD8+ T cells expressing anti-CD19 CAR (with or without PDCD1 deletion) proliferated after co-culture with K562-CD19 in an antigen-specific manner, to a similar degree. Thus, the results demonstrated that, under these conditions, CAR+ T cells could proliferate in a CAR antigen-specific manner following deletion of PDCD1.

C. Cytokine Release

Cytokine release also was assessed following incubation of the various cells with antigen-expressing and control target cells. CAR-expressing T cells (and mock controls) subjected to PDCD1-targeted or AAVS1-targeted deletion or not-transfected (UT), generated as described above, were co-cultured in triplicate with target cells (K562-CD19 or K562-ROR) a 4:1 effector:target ratio. The co-cultured cells were incubated for about 24 hours, and then supernatants were collected for measurement of IFN-γ, TNF-α, or IL-2 using a multiplex cytokine immunoassay (Meso Scale Discovery).

The results are set forth in FIG. 30A (IFN-γ), FIG. 30B (TNF-α) and FIG. 30C (IL-2). The results showed that, under these conditions, the PDCD1-deleted and control CAR-expressing T cells secreted similar levels cytokines in an antigen-specific manner, following incubation with CD19-expressing target cells.

### Example 6: Cloning and Initial Screening of gRNAs

The suitability of candidate gRNAs can be evaluated as described in this example. Although described for a chimeric gRNA, the approach can also be used to evaluate modular gRNAs.

Cloning gRNAs into Vectors

For each gRNA, a pair of overlapping oligonucleotides is designed and obtained. Oligonucleotides are annealed and ligated into a digested vector backbone containing an upstream U6 promoter and the remaining sequence of a long chimeric gRNA. Plasmid is sequence-verified and prepped to generate sufficient amounts of transfection-quality DNA. Alternate promoters may be used to drive in vivo transcription (e.g., H1 promoter) or for in vitro transcription (e.g., a T7 promoter).

Cloning gRNAs in Linear dsDNA Molecule (STITCHR)

For each gRNA, a single oligonucleotide is designed and obtained. The U6 promoter and the gRNA scaffold (e.g. including everything except the targeting domain, e.g., including sequences derived from the crRNA and tracrRNA, e.g., including a first complementarity domain; a linking domain; a second complementarity domain; a proximal domain; and a tail domain) are separately PCR amplified and purified as dsDNA molecules. The gRNA-specific oligonucleotide is used in a PCR reaction to stitch together the U6 and the gRNA scaffold, linked by the targeting domain specified in the oligonucleotide. Resulting dsDNA molecule (STITCHR product) is purified for transfection. Alternate promoters may be used to drive in vivo transcription (e.g., H1 promoter) or for in vitro transcription (e.g., T7 promoter). Any gRNA scaffold may be used to create gRNAs compatible with Cas9s from any bacterial species

Initial gRNA Screen

Each gRNA to be tested is transfected, along with a plasmid expressing Cas9 and a small amount of a GFP-expressing plasmid into human cells. In preliminary experiments, these cells can be immortalized human cell lines such as 293T, K562 or U2OS. Alternatively, primary human cells may be used. In this case, cells may be relevant to the eventual therapeutic cell target (for example, an erythroid cell). The use of primary cells similar to the potential therapeutic target cell population may provide important information on gene targeting rates in the context of endogenous chromatin and gene expression.

Transfection may be performed using lipid transfection (such as Lipofectamine or Fugene) or by electroporation (such as Lonza Nucleofection). Following transfection, GFP expression can be determined either by fluorescence microscopy or by flow cytometry to confirm consistent and high levels of transfection. These preliminary transfections can comprise different gRNAs and different targeting approaches (17-mers, 20-mers, nuclease, dual-nickase, etc.) to determine which gRNAs/combinations of gRNAs give the greatest activity.

Efficiency of cleavage with each gRNA may be assessed by measuring NHEJ-induced indel formation at the target locus by a T7E1-type assay or by sequencing. Alternatively, other mismatch-sensitive enzymes, such as Cell/Surveyor nuclease, may also be used.

For the T7E1 assay, PCR amplicons are approximately 500-700 bp with the intended cut site placed asymmetrically in the amplicon. Following amplification, purification and size-verification of PCR products, DNA is denatured and re-hybridized by heating to 95° C. and then slowly cooling. Hybridized PCR products are then digested with T7 Endonuclease I (or other mismatch-sensitive enzyme) which recognizes and cleaves non-perfectly matched DNA. If indels are present in the original template DNA, when the amplicons are denatured and re-annealed, this results in the hybridization of DNA strands harboring different indels and therefore lead to double-stranded DNA that is not perfectly matched. Digestion products may be visualized by gel electrophoresis or by capillary electrophoresis. The fraction of DNA that is cleaved (density of cleavage products divided by the density of cleaved and uncleaved) may be used to estimate a percent NHEJ using the following equation: % NHEJ=(1-(1-fraction cleaved)^{1/2}). The T7E1 assay is sensitive down to about 2-5% NHEJ.

Sequencing may be used instead of, or in addition to, the T7E1 assay. For Sanger sequencing, purified PCR amplicons are cloned into a plasmid backbone, transformed, miniprepped and sequenced with a single primer. Sanger sequencing may be used for determining the exact nature of indels after determining the NHEJ rate by T7E1.

Sequencing may also be performed using next generation sequencing techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq). This method allows for detection of very low NHEJ rates.

### Example 7: Assessment of Gene Targeting by NHEJ

The gRNAs that induce the greatest levels of NHEJ in initial tests can be selected for further evaluation of gene targeting efficiency. In this case, cells are derived from disease subjects and, therefore, harbor the relevant mutation.

Following transfection (usually 2-3 days post-transfection) genomic DNA may be isolated from a bulk population of transfected cells and PCR may be used to amplify the target region. Following PCR, gene targeting efficiency to generate the desired mutations (either knockout of a target gene or removal of a target sequence motif) may be determined by sequencing. For Sanger sequencing, PCR amplicons may be 500-700 bp long. For next generation sequencing, PCR amplicons may be 300-500 bp long. If the goal is to knockout gene function, sequencing may be used to assess what percent of alleles have undergone NHEJ-induced indels that result in a frameshift or large deletion or insertion that would be expected to destroy gene function. If the goal is to remove a specific sequence motif, sequencing may be used to assess what percent of alleles have undergone NHEJ-induced deletions that span this sequence.

### Example 8: Screening of gRNAs in 293 Cells

Screening of gRNAs for T Cell Receptor Beta (TRBC)

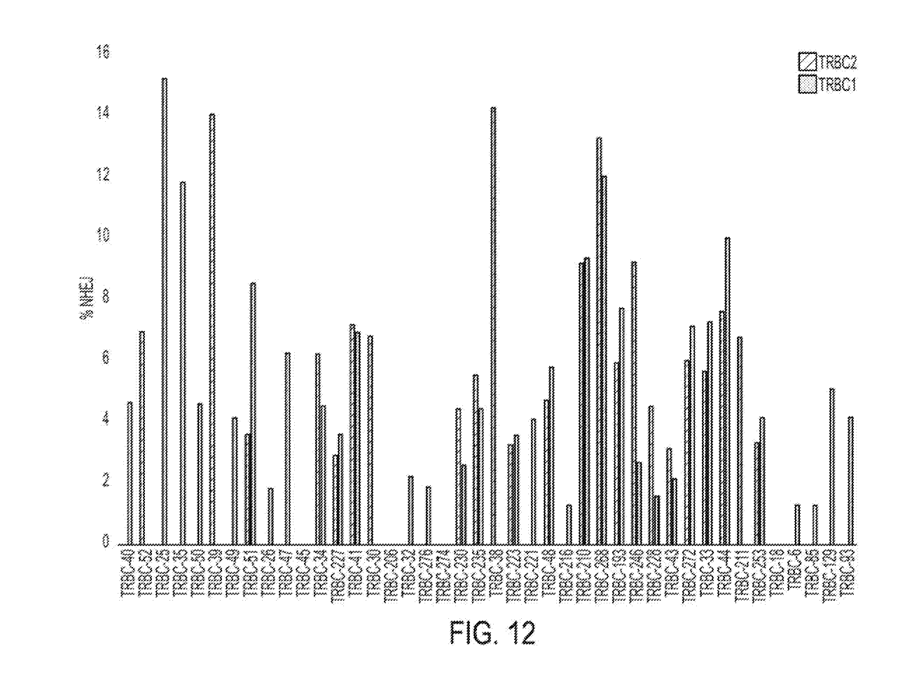

In order to identify gRNAs with the highest on target NHEJ efficiency, 42 S. pyogenes and 27 S. aureus gRNAs were selected (Table 3000). A DNA template comprised of a U6 promoter, the gRNA target region and appropriate TRACR sequence (S. pyogenes or S. aureus) was generated by a PCR STITCHR reaction. This DNA template was subsequently transfected into 293 cells using Lipofectamine 3000 along with a DNA plasmid encoding the appropriate Cas9 (S. pyogenes or S. aureus) downstream of a CMV promoter. Genomic DNA was isolated from the cells 48-72 hours post transfection. To determine the rate of modification at the T cell receptor beta gene (TRBC), the target region was amplified using a locus PCR with the primers listed in Table 4000. After PCR amplification, a T7E1 assay was performed on the PCR product. Briefly, this assay involves melting the PCR product followed by a re-annealing step. If gene modification has occurred, there will exist double stranded products that are not perfect matches due to some frequency of insertions or deletions. These double stranded products are sensitive to cleavage by a T7 endonuclease 1 enzyme at the site of mismatch. It is possible, therefore, to determine the efficiency of cutting by the Cas9/gRNA complex by analyzing the amount of T7E1 cleavage. The formula that is used to provide a measure of % NHEJ from the T7E1 cutting is the following: (100*(1−((1−(fraction cleaved))̂0.5))). The results of this analysis are shown in FIG. 11 and FIG. 12.

TABLE 3000

| | | | SEQ |
|---|---|---|---|
| | | | ID |
| gRNA Name | Targeting Domain | Cas9 species | NO: |
| TRBC-40 | CACCCAGAUCGUCAGCGCCG | S. pyogenes | 387 |
| TRBC-52 | CAAACACAGCGACCUCGGGU | S. pyogenes | 388 |
| TRBC-25 | UGACGAGUGGACCCAGGAUA | S. pyogenes | 389 |
| TRBC-35 | GGCUCUCGGAGAAUGACGAG | S. pyogenes | 390 |
| TRBC-50 | GGCCUCGGCGCUGACGAUCU | S. pyogenes | 391 |
| TRBC-39 | GAAAAACGUGUUCCCACCCG | S. pyogenes | 392 |
| TRBC-49 | AUGACGAGUGGACCCAGGAU | S. pyogenes | 393 |
| TRBC-51 | AGUCCAGUUCUACGGGCUCU | S. pyogenes | 394 |
| TRBC-26 | CGCUGUCAAGUCCAGUUCUA | S. pyogenes | 395 |
| TRBC-47 | AUCGUCAGCGCCGAGGCCUG | S. pyogenes | 396 |
| TRBC-45 | UCAAACACAGCGACCUCGGG | S. pyogenes | 397 |
| TRBC-34 | CGUAGAACUGGACUUGACAG | S. pyogenes | 398 |
| TRBC-227 | AGGCCUCGGCGCUGACGAUC | S. pyogenes | 399 |
| TRBC-41 | UGACAGCGGAAGUGGUUGCG | S. pyogenes | 400 |
| TRBC-30 | UUGACAGCGGAAGUGGUUGC | S. pyogenes | 401 |
| TRBC-206 | UCUCCGAGAGCCCGUAGAAC | S. pyogenes | 402 |
| TRBC-32 | CGGGUGGGAACACGUUUUUC | S. pyogenes | 403 |
| TRBC-276 | GACAGGUUUGGCCCUAUCCU | S. pyogenes | 404 |
| TRBC-274 | GAUCGUCAGCGCCGAGGCCU | S. pyogenes | 405 |
| TRBC-230 | GGCUCAAACACAGCGACCUC | S. pyogenes | 406 |
| TRBC-235 | UGAGGGUCUCGGCCACCUUC | S. pyogenes | 407 |
| TRBC-38 | AGGCUUCUACCCCGACCACG | S. pyogenes | 408 |
| TRBC-223 | CCGACCACGUGGAGCUGAGC | S. pyogenes | 409 |
| TRBC-221 | UGACAGGUUUGGCCCUAUCC | S. pyogenes | 410 |
| TRBC-48 | CUUGACAGCGGAAGUGGUUG | S. pyogenes | 411 |
| TRBC-216 | AGAUCGUCAGCGCCGAGGCC | S. pyogenes | 412 |
| TRBC-210 | GCGCUGACGAUCUGGGUGAC | S. pyogenes | 413 |
| TRBC-268 | UGAGGGCGGGCUGCUCCUUG | S. pyogenes | 414 |
| TRBC-193 | GUUGCGGGGGUUCUGCCAGA | S. pyogenes | 415 |
| TRBC-246 | AGCUCAGCUCCACGUGGUCG | S. pyogenes | 416 |
| TRBC-228 | GCGGCUGCUCAGGCAGUAUC | S. pyogenes | 417 |
| TRBC-43 | GCGGGGGUUCUGCCAGAAGG | S. pyogenes | 418 |
| TRBC-272 | UGGCUCAAACACAGCGACCU | S. pyogenes | 419 |
| TRBC-33 | ACUGGACUUGACAGCGGAAG | S. pyogenes | 420 |
| TRBC-44 | GACAGCGGAAGUGGUUGCGG | S. pyogenes | 421 |
| TRBC-211 | GCUGUCAAGUCCAGUUCUAC | S. pyogenes | 422 |
| TRBC-253 | GUAUCUGGAGUCAUUGAGGG | S. pyogenes | 423 |
| TRBC-18 | CUCGGCGCUGACGAUCU | S. pyogenes | 424 |
| TRBC-6 | CCUCGGCGCUGACGAUC | S. pyogenes | 425 |
| TRBC-85 | CCGAGAGCCCGUAGAAC | S. pyogenes | 426 |
| TRBC-129 | CCAGAUCGUCAGCGCCG | S. pyogenes | 427 |
| TRBC-93 | GAAUGACGAGUGGACCC | S. pyogenes | 428 |
| TRBC-415 | GGGUGACAGGUUUGGCCCUAUC | S. aureus | 429 |
| TRBC-414 | GGUGACAGGUUUGGCCCUAUC | S. aureus | 430 |
| TRBC-310 | GUGACAGGUUUGGCCCUAUC | S. aureus | 431 |
| TRBC-308 | GACAGGUUUGGCCCUAUC | S. aureus | 432 |
| TRBC-401 | GAUACUGCCUGAGCAGCCGCCU | S. aureus | 433 |
| TRBC-468 | GACCACGUGGAGCUGAGCUGGUGG | S. aureus | 434 |
| TRBC-462 | GUGGAGCUGAGCUGGUGG | S. aureus | 435 |
| TRBC-424 | GGGCGGGCUGCUCCUUGAGGGGCU | S. aureus | 436 |
| TRBC-423 | GGCGGGCUGCUCCUUGAGGGGCU | S. aureus | 437 |
| TRBC-422 | GCGGGCUGCUCCUUGAGGGGCU | S. aureus | 438 |
| TRBC-420 | GGGCUGCUCCUUGAGGGGCU | S. aureus | 439 |
| TRBC-419 | GGCUGCUCCUUGAGGGGCU | S. aureus | 440 |
| TRBC-418 | GCUGCUCCUUGAGGGGCU | S. aureus | 441 |
| TRBC-445 | GGUGAAUGGGAAGGAGGUGCACAG | S. aureus | 442 |
| TRBC-444 | GUGAAUGGGAAGGAGGUGCACAG | S. aureus | 443 |
| TRBC-442 | GAAUGGGAAGGAGGUGCACAG | S. aureus | 444 |

TABLE 4000

| | | | SEQ |
|---|---|---|---|
| Primer | | | ID |
| name | Sequence | Exon | NO: |
| GWED133 | ACATCACCTGGAATGTTAGGCA | TRBC2 5′ primer | 445 |
| GWED134 | GTGAACCTTGATCATCCCACCT | TRBC2 3′ Primer | 446 |
| GWED137 | CCCCAAAACAATGAGGGCCT | TRBC1 5′ primer | 447 |
| GWED138 | AGCACCTCCTTTCTTCTGCC | TRBC1 3′ primer | 448 |

Screening of gRNAs for T Cell Receptor Alpha (TRAC)

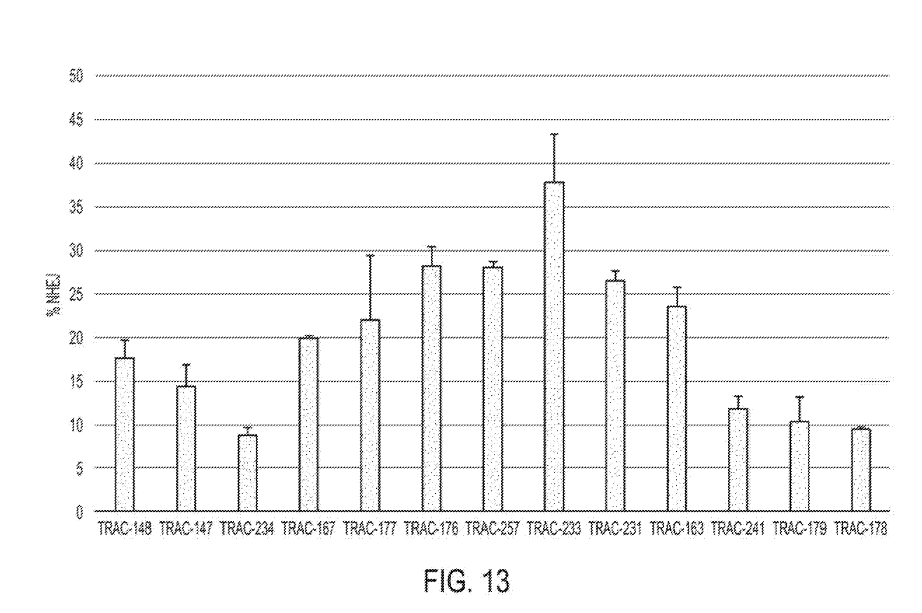

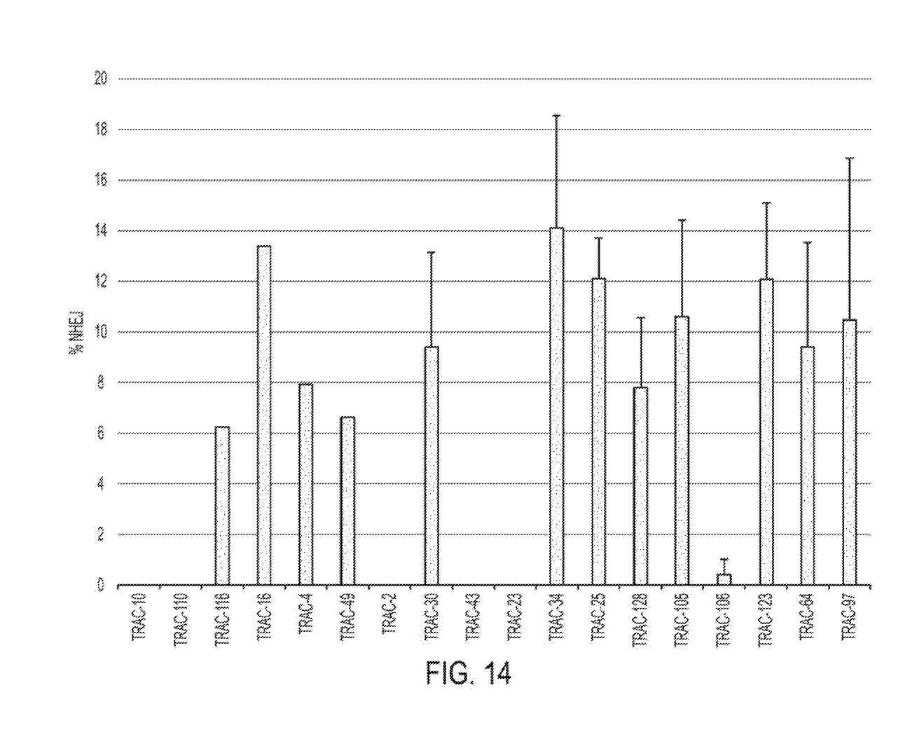

In order to identify gRNAs with the highest on target NHEJ efficiency, 18 S. pyogenes and 13 S. aureus gRNAs were selected (Table 5000). A DNA template comprised of a U6 promoter, the gRNA target region and appropriate TRACR sequence (S. pyogenes or S. aureus) was generated by a PCR STITCHR reaction. This DNA template was subsequently transfected into 293 cells using Lipofectamine 3000 along with a DNA plasmid encoding the appropriate Cas9 (S. pyogenes or S. aureus) downstream of a CMV promoter. Genomic DNA was isolated from the cells 48-72 hours post transfection. To determine the rate of modification at the T cell receptor alpha gene (TRAC), the target region was amplified using a locus PCR with the primers listed in Table 6000. After PCR amplification, a T7E1 assay was performed on the PCR product. Briefly, this assay involves melting the PCR product followed by a re-annealing step. If gene modification has occurred, there will exist double stranded products that are not perfect matches due to some frequency of insertions or deletions. These double stranded products are sensitive to cleavage by a T7 endonuclease 1 enzyme at the site of mismatch. It is possible, therefore, to determine the efficiency of cutting by the Cas9/gRNA complex by analyzing the amount of T7E1 cleavage. The formula that is used to provide a measure of % NHEJ from the T7E1 cutting is the following: (100*(1−((1−(fraction cleaved))̂0.5))). The results of this analysis are shown in FIG. 13 and FIG. 14.

TABLE 5000

| gRNA | | Cas9 | SEQ |
|---|---|---|---|
| Name | Targeting Domain | species | ID NO: |
| TRAC-10 | UCUCUCAGCUGGUACACGGC | S. pyogenes | 449 |
| TRAC-110 | UGGAUUUAGAGUCUCUCAGC | S. pyogenes | 450 |
| TRAC-116 | ACACGGCAGGGUCAGGGUUC | S. pyogenes | 451 |
| TRAC-16 | GAGAAUCAAAAUCGGUGAAU | S. pyogenes | 452 |
| TRAC-4 | GCUGGUACACGGCAGGGUCA | S. pyogenes | 453 |
| TRAC-49 | CUCAGCUGGUACACGGC | S. pyogenes | 454 |
| TRAC-2 | UGGUACACGGCAGGGUC | S. pyogenes | 455 |
| TRAC-30 | GCUAGACAUGAGGUCUA | S. pyogenes | 456 |
| TRAC-43 | GUCAGAUUUGUUGCUCC | S. pyogenes | 457 |
| TRAC-23 | UCAGCUGGUACACGGCA | S. pyogenes | 458 |
| TRAC-34 | GCAGACAGACUUGUCAC | S. pyogenes | 459 |
| TRAC-25 | GGUACACGGCAGGGUCA | S. pyogenes | 460 |
| TRAC-128 | CUUCAAGAGCAACAGUGCUG | S. pyogenes | 461 |
| TRAC-105 | AGAGCAACAGUGCUGUGGCC | S. pyogenes | 462 |
| TRAC-106 | AAAGUCAGAUUUGUUGCUCC | S. pyogenes | 463 |
| TRAC-123 | ACAAAACUGUGCUAGACAUG | S. pyogenes | 464 |
| TRAC-64 | AAACUGUGCUAGACAUG | S. pyogenes | 465 |
| TRAC-97 | UGUGCUAGACAUGAGGUCUA | S. pyogenes | 466 |
| TRAC-148 | GGCUGGGGAAGAAGGUGUCUUC | S. aureus | 467 |
| TRAC-147 | GCUGGGGAAGAAGGUGUCUUC | S. aureus | 468 |
| TRAC-234 | GGGGAAGAAGGUGUCUUC | S. aureus | 469 |
| TRAC-167 | GUUUUGUCUGUGAUAUACACAU | S. aureus | 470 |
| TRAC-177 | GGCAGACAGACUUGUCACUGGAUU | S. aureus | 471 |
| TRAC-176 | GCAGACAGACUUGUCACUGGAUU | S. aureus | 472 |
| TRAC-257 | GACAGACUUGUCACUGGAUU | S. aureus | 473 |
| TRAC-233 | GUGAAUAGGCAGACAGACUUGUCA | S. aureus | 474 |
| TRAC-231 | GAAUAGGCAGACAGACUUGUCA | S. aureus | 475 |
| TRAC-163 | GAGUCUCUCAGCUGGUACACGG | S. aureus | 476 |
| TRAC-241 | GUCUCUCAGCUGGUACACGG | S. aureus | 477 |
| TRAC-179 | GGUACACGGCAGGGUCAGGGUU | S. aureus | 478 |
| TRAC-178 | GUACACGGCAGGGUCAGGGUU | S. aureus | 49451 |

TABLE 6000

| | | | SEQ |
|---|---|---|---|
| | | | ID |
| Primer name | Sequence | Exon | NO: |
| GWED141 | GTTTGCTTTGCTGGGCCTTT | TRAC 5′ primer | 479 |
| GWED142 | CCTCTCCTGCCACCTTCTCT | TRAC 3′ primer | 480 |

Screening of gRNAs for PDCD1 Gene

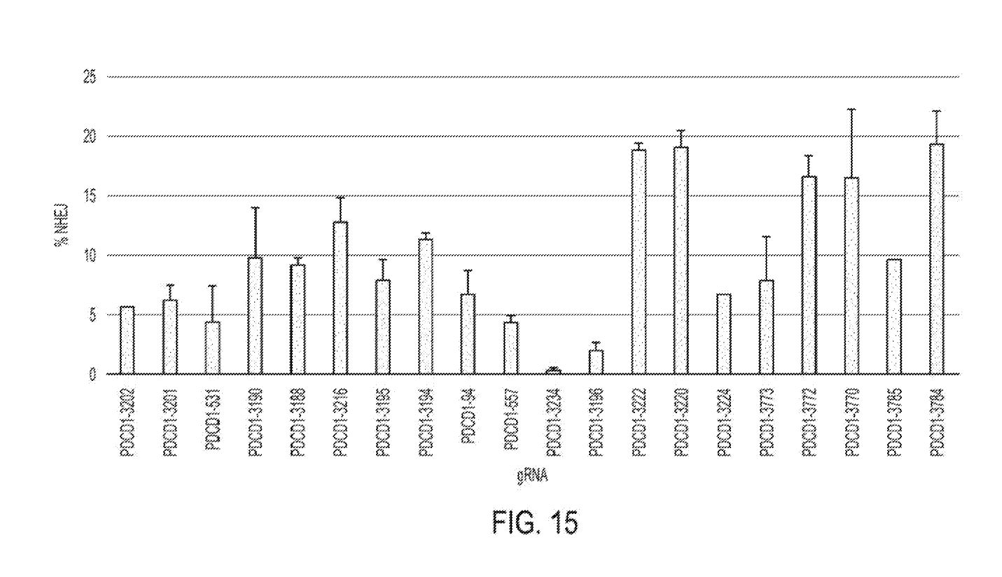

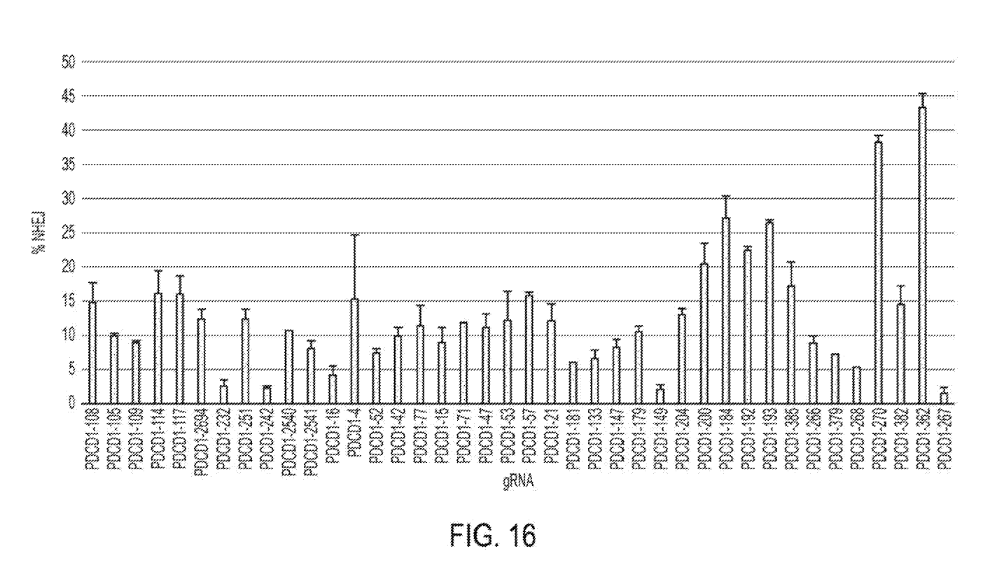

In order to identify gRNAs with the highest on target NHEJ efficiency, 48 S. pyogenes and 27 S. aureus gRNAs were selected (see Tables 7000A and 7000B). A DNA template comprised of a U6 promoter, the gRNA target region and appropriate TRACR sequence (S. pyogenes or S. aureus) was generated by a PCR STITCHR reaction. This DNA template was subsequently transfected into 293 cells using Lipofectamine 3000 along with a DNA plasmid encoding the appropriate Cas9 (S. pyogenes or S. aureus) downstream of a CMV promoter. Genomic DNA was isolated from the cells 48-72 hours post transfection. To determine the rate of modification at the PD-1 gene (PDCD1), the target region was amplified using a locus PCR with the primers listed in Table 7000C. After PCR amplification, a T7E1 assay was performed on the PCR product. Briefly, this assay involves melting the PCR product followed by a re-annealing step. If gene modification has occurred, there will exist double stranded products that are not perfect matches due to some frequency of insertions or deletions. These double stranded products are sensitive to cleavage by a T7 endonuclease 1 enzyme at the site of mismatch. It is possible, therefore, to determine the efficiency of cutting by the Cas9/gRNA complex by analyzing the amount of T7E1 cleavage. The formula that is used to provide a measure of % NHEJ from the T7E1 cutting is the following: (100*(1−((1−(fraction cleaved))̂0.5))). The results of this analysis for the gRNAs shown in Table 7000A are shown in FIG. 15 and FIG. 16. Similar experiments can be performed with other gRNAs described herein, including those shown in Table 7000B.

TABLE 7000A

| | | | SEQ |
|---|---|---|---|
| | | Cas9 | ID |
| gRNA Name | Targeting Domain | species | NO: |
| PDCD1-3202 | GGUGCUACAACUGGGCUGGCGGC | S. aureus | 481 |
| PDCD1-3201 | GUGCUACAACUGGGCUGGCGGC | S. aureus | 482 |
| PDCD1-531 | GCUACAACUGGGCUGGCGGC | S. aureus | 483 |
| PDCD1-3190 | GACGACUGGCCAGGGCGCCUG | S. aureus | 484 |
| PDCD1-3188 | GACUGGCCAGGGCGCCUG | S. aureus | 485 |
| PDCD1-3216 | GCAUGCCUGGAGCAGCCCCAC | S. aureus | 486 |
| PDCD1-3195 | GGGGGGUUCCAGGGCCUGUCUG | S. aureus | 487 |
| PDCD1-3194 | GGGGGUUCCAGGGCCUGUCUG | S. aureus | 488 |
| PDCD1-94 | GGGGUUCCAGGGCCUGUCUG | S. aureus | 489 |
| PDCD1-557 | GAGAGCCUGCGGGCAGAGCU | S. aureus | 490 |
| PDCD1-3234 | GAGCCUGCGGGCAGAGCU | S. aureus | 491 |
| PDCD1-3196 | GGGGGGGUUCCAGGGCCUGUCUG | S. aureus | 492 |
| PDCD1-3222 | GCAGGGCUGGGGAGAAGGUGG | S. aureus | 493 |
| PDCD1-3220 | GGGCUGGGGAGAAGGUGG | S. aureus | 494 |
| PDCD1-3224 | GAGCAGGGCUGGGGAGAAGGUGG | S. aureus | 495 |
| PDCD1-3773 | GGCCGCCCACGACACCAACCAC | S. aureus | 496 |
| PDCD1-3772 | GCCGCCCACGACACCAACCAC | S. aureus | 497 |
| PDCD1-3770 | GCCCACGACACCAACCAC | S. aureus | 498 |
| PDCD1-3785 | GGUUUGGAACUGGCCGGCUGGC | S. aureus | 499 |
| PDCD1-3784 | GUUUGGAACUGGCCGGCUGGC | S. aureus | 500 |
| PDCD1-108 | GUCUGGGCGGUGCUACAACU | S. pyogenes | 508 |
| PDCD1-105 | GGCGCCCUGGCCAGUCGUCU | S. pyogenes | 509 |
| PDCD1-109 | GGGCGGUGCUACAACUGGGC | S. pyogenes | 510 |
| PDCD1-114 | GGCCAGGAUGGUUCUUAGGU | S. pyogenes | 511 |
| PDCD1-117 | GGAUGGUUCUUAGGUAGGUG | S. pyogenes | 512 |
| PDCD1-2694 | GAAGGCGGCACUCUGGU | S. pyogenes | 513 |
| PDCD1-232 | GCCCUGGCCAGUCGUCU | S. pyogenes | 514 |
| PDCD1-251 | GCCCAGACGACUGGCCA | S. pyogenes | 515 |
| PDCD1-242 | GAUGGUUCUUAGGUAGG | S. pyogenes | 516 |
| PDCD1-2540 | GGCGGAGGUGAGCGGAA | S. pyogenes | 517 |
| PDCD1-2541 | GGAAGGGAAACUGUCCC | S. pyogenes | 518 |
| PDCD1-16 | GCGUGACUUCCACAUGAGCG | S. pyogenes | 519 |
| PDCD1-4 | GCCCUGCUCGUGGUGACCGA | S. pyogenes | 520 |
| PDCD1-52 | GGUGCCGCUGUCAUUGCGCC | S. pyogenes | 521 |
| PDCD1-42 | GCUCUCUUUGAUCUGCGCCU | S. pyogenes | 522 |
| PDCD1-77 | GAAGGUGGCGUUGUCCCCUU | S. pyogenes | 523 |
| PDCD1-15 | GUGUCACACAACUGCCCAAC | S. pyogenes | 524 |
| PDCD1-71 | GCUUGUCCGUCUGGUUGCUG | S. pyogenes | 525 |
| PDCD1-47 | GAUCUGCGCCUUGGGGGCCA | S. pyogenes | 526 |
| PDCD1-53 | GGGCCCUGACCACGCUCAUG | S. pyogenes | 527 |
| PDCD1-57 | GCAGUUGUGUGACACGGAAG | S. pyogenes | 528 |
| PDCD1-21 | GACAGCGGCACCUACCUCUG | S. pyogenes | 529 |
| PDCD1-181 | GUGGAAGUCACGCCCGU | S. pyogenes | 530 |
| PDCD1-133 | GCUCGUGGUGACCGAAG | S. pyogenes | 531 |
| PDCD1-147 | GCCCGGCGCAAUGACAG | S. pyogenes | 532 |
| PDCD1-179 | GCCGCUGUCAUUGCGCC | S. pyogenes | 533 |
| PDCD1-149 | GCGGCACCUACCUCUGU | S. pyogenes | 534 |
| PDCD1-204 | GGUGGCGUUGUCCCCUU | S. pyogenes | 535 |
| PDCD1-200 | GAAGCUCUCCGAUGUGU | S. pyogenes | 536 |
| PDCD1-184 | GUUGUGUGACACGGAAG | S. pyogenes | 537 |
| PDCD1-192 | GCUGGCUGCGGUCCUCG | S. pyogenes | 538 |
| PDCD1-193 | GCUGCGGUCCUCGGGGA | S. pyogenes | 539 |
| PDCD1-385 | GACGUUACCUCGUGCGGCCC | S. pyogenes | 540 |
| PDCD1-266 | GGUGUCGUGGGCGGCCUGCU | S. pyogenes | 541 |
| PDCD1-379 | GCCCACGACACCAACCACCA | S. pyogenes | 542 |
| PDCD1-268 | GCAGCCUGGUGCUGCUAGUC | S. pyogenes | 543 |
| PDCD1-270 | GGUGCUGCUAGUCUGGGUCC | S. pyogenes | 544 |
| PDCD1-382 | GGACCCAGACUAGCAGCACC | S. pyogenes | 545 |
| PDCD1-362 | GGCACUUCUGCCCUUCUCUC | S. pyogenes | 546 |
| PDCD1-267 | GGGCGGCCUGCUGGGCAGCC | S. pyogenes | 547 |

TABLE 7000B

| | | | SEQ |
|---|---|---|---|
| | | Cas9 | ID |
| gRNA Name | Targeting Domain | species | NO: |
| PDCD1-3856 | GGGCAGCCUGGUGCUGCUAGU | S. aureus | 501 |
| PDCD1-720 | GGCAGCCUGGUGCUGCUAGU | S. aureus | 502 |
| PDCD1-3855 | GCAGCCUGGUGCUGCUAGU | S. aureus | 503 |
| PDCD1-369 | GGCUGGCCUGGGUGAGGGGC | S. aureus | 504 |
| PDCD1-3786 | GGGUUUGGAACUGGCCGGCUGGC | S. aureus | 505 |
| PDCD1-3859 | GCUGGGCAGCCUGGUGCUGCUAGU | S. aureus | 506 |
| PDCD1-3781 | GGCCGGCUGGCCUGGGUGAGGGGC | S. aureus | 507 |
| PDCD1-381 | GCAGCACCAGGCUGCCCAGC | S. pyogenes | 548 |
| PDCD1-368 | GCUGGCCUGGGUGAGGGGCU | S. pyogenes | 549 |
| PDCD1-472 | GUUACCUCGUGCGGCCC | S. pyogenes | 550 |
| PDCD1-316 | GUCGUGGGCGGCCUGCU | S. pyogenes | 551 |
| PDCD1-462 | GUUUGGAACUGGCCGGC | S. pyogenes | 552 |
| PDCD1-322 | GGCCGUCAUCUGCUCCC | S. pyogenes | 553 |
| PDCD1-320 | GCUGCUAGUCUGGGUCC | S. pyogenes | 554 |
| PDCD1-318 | GCCUGGUGCUGCUAGUC | S. pyogenes | 555 |

TABLE 7000C

| | | | SEQ |
|---|---|---|---|
| | | | ID |
| Primer name | Sequence | Exon | NO: |
| GWED259 | CACTGCCTCTGTCACTCTCG | PD1_exon_1_5′ | 556 |
| GWED260 | AGGGACTGAGAGTGAAAGGT | PD1_exon_1_3′ | 557 |
| JFPR04 | CAGGATGCCCAAGGGTCAG | PD1_exon_2_5′ | 558 |
| JFPR004R | GGAGCTCCTGATCCTGTGC | PD1_exon_2_3′ | 559 |
| GWED257 | AATGGTGACCGGCATCTCTG | PD1_exon_3_5′ | 560 |
| JFPR005 | CTGCACAGGATCAGGAGCTC | PD1_exon_3_5′ | 561 |
| JFPR005R | AGAATGTGAGTCCTGCAGGC | PD1_exon_3_3′ | 562 |

### Example 9: Enzymatic Synthesis and Delivery of gRNAs to Primary T Cells

Delivery of Cas9 mRNA and gRNA as RNA Molecules to T Cells

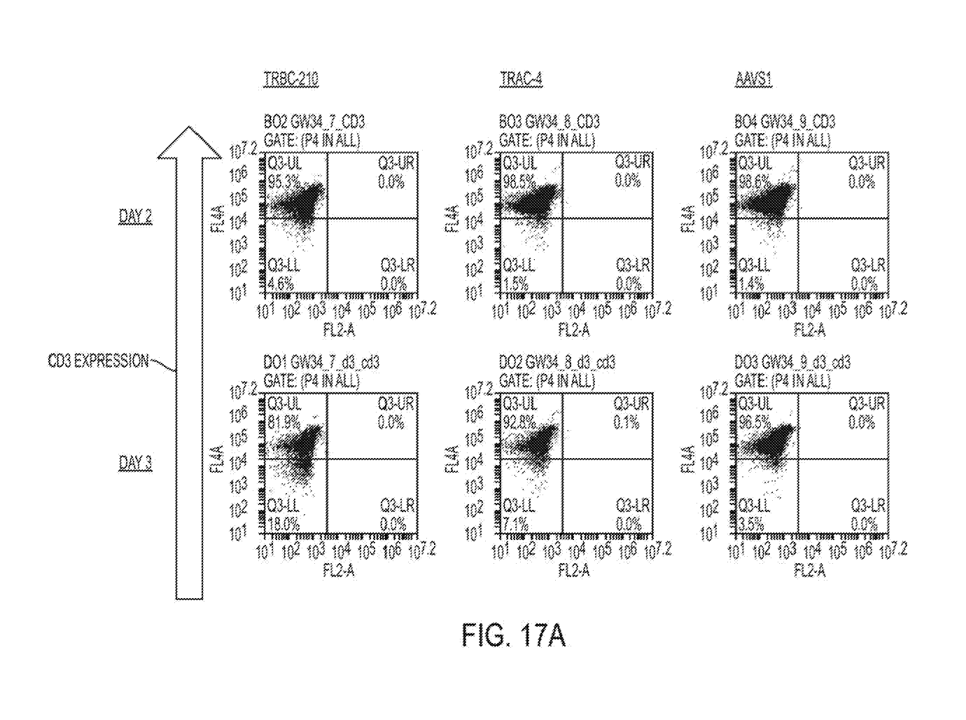

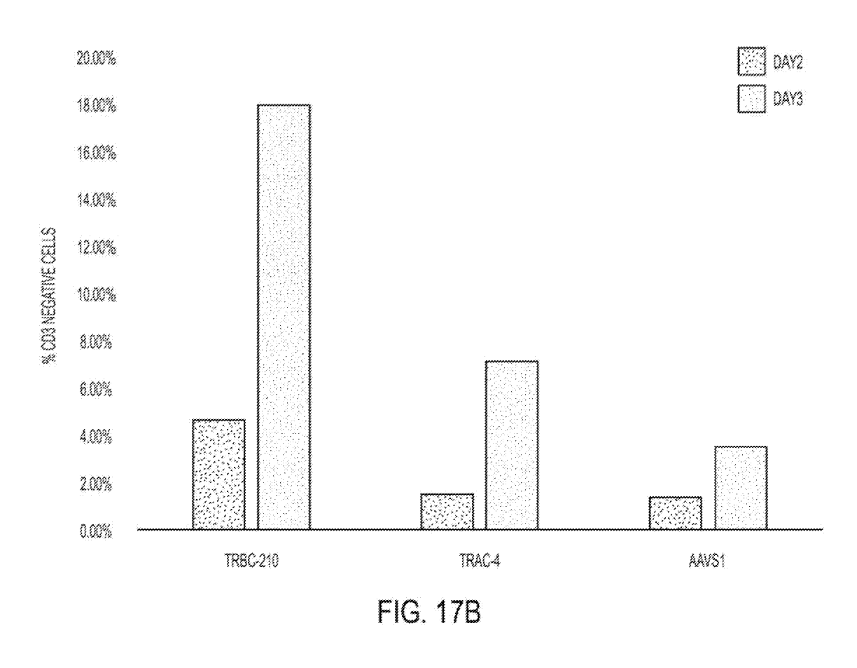

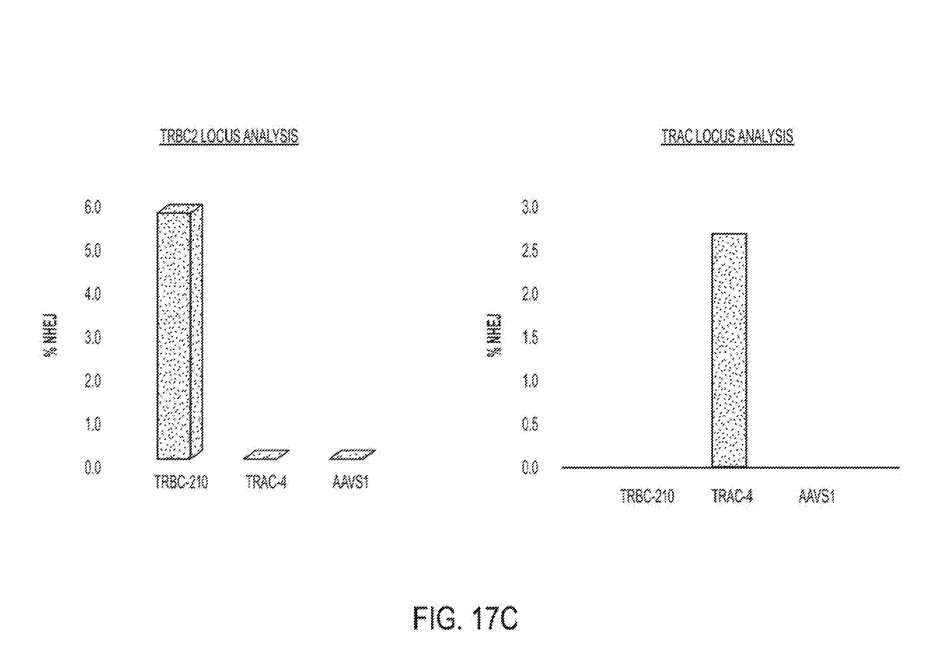

To demonstrate Cas9-mediated cutting in primary CD4+ T cells, S. pyogenes Cas9 and a gRNA designed against the TCR beta chain (TRBC-210 (GCGCUGACGAUCUGGGUGAC) (SEQ ID NO:413)) or the TCR alpha chain (TRAC-4 (GCUGGUACACGGCAGGGUCA) (SEQ ID NO:453)) were delivered as RNA molecules to T cells via electroporation. In this embodiment, both the Cas9 and gRNA were in vitro transcribed using a T7 polymerase. A 5′ ARCA cap was added to both RNA species simultaneous to transcription while a polyA tail was added after transcription to the 3′ end of the RNA species by an E. coli polyA polymerase. To generate CD4+ T cells modified at the TRBC1 and TRBC2 loci, 10 ug of Cas9 mRNA and 10 ug of TRBC-210 (GCGCUGACGAUCUGGGUGAC) (SEQ ID NO:413) gRNA were introduced to the cells by electroporation. In the same experiment, we also targeted the TRAC gene by introducing 10 ug of Cas9 mRNA with 10 ug of TRAC-4 (GCUGGUACACGGCAGGGUCA) (SEQ ID NO:453) gRNA. A gRNA targeting the AAVS1 (GUCCCCUCCACCCCACAGUG) (SEQ ID NO:51201) genomic site was used as an experimental control. Prior to electroporation, the T cells were cultured in RPMI 1640 supplemented with 10% FBS and recombinant IL-2. The cells were activated using CD3/CD28 beads and expanded for at least 3 days. Subsequent to introduction of the mRNA to the activated T cells, CD3 expression on the cells was monitored at 24, 48 and 72 hours post electroporation by flow cytometry using a fluorescein (APC) conjugated antibody specific for CD3. At 72 hours, a population of CD3 negative cells was observed (FIG. 17A and FIG. 17B). To confirm that the generation of CD3 negative cells was a result of genome editing at the TRBC loci, genomic DNA was harvested and a T7E1 assay was performed. Indeed, the data confirm the presence of DNA modifications at the TRBC2 locus and TRAC locus (FIG. 17C).

Delivery of Cas9/gRNA RNP to T Cells

To demonstrate Cas9-mediated cutting in Jurkat T cells, S. aureus Cas9 and a gRNA designed against the TCR alpha chain (TRAC-233 (GUGAAUAGGCAGACAGACUUGUCA) (SEQ ID NO:474)) were delivered as a ribonucleic acid protein complex (RNP) by electroporation. In this embodiment, the Cas9 was expressed in E. coli and purified. Specifically, the HJ29 plasmid encoding Cas9 was transformed into Rosetta™ 2 (DE3) chemically competent cells (EMD Millipore #71400-4) and plated onto LB plates with appropriate antibiotics for selection and incubated at 37° C. overnight. A 10 mL starter culture of Brain Heart Infusion Broth (Teknova #B9993) with appropriate antibiotics was inoculated with 4 colonies and grown at 37° C. with shaking at 220 rpms. After growing overnight, the starter culture was added to 1 L of Terrific Broth Complete (Teknova #T7060) with appropriate antibiotics plus supplements and grown at 37° C. with shaking at 220 rpms. The temperature was gradually reduced to 18° C. and expression of the gene was induced by addition of IPTG to 0.5 mM when the OD600 was greater than 2.0. The induction was allowed to continue overnight followed by harvesting the cells by centrifugation and resuspension in TG300 (50 mM Tris pH8.0, 300 mM NaCl, 20% glycerol, 1 mM TCEP, protease inhibitor tablets (Thermo Scientific #88266)) and stored at −80° C.

The cells were lysed by thawing the frozen suspension, followed by two passes through a LM10 Microfluidizer® set to 18000 psi. The extract was clarified via centrifugation and the soluble extract was captured via batch incubation with Ni-NTA Agarose resin (Qiagen #30230) at 4° C. The slurry was poured into a gravity flow column, washed with TG300+30 mM Imidazole and then eluted the protein of interest with TG300+300 mM Imidazole. The Ni eluent was diluted with an equal volume of HG100 (50 mM Hepes pH7.5, 100 mM NaCl, 10% glycerol, 0.5 mM TCEP) and loaded onto a HiTrap SP HP column (GE Healthcare Life Sciences #17-1152-01) and eluted with a 30 column volume gradient from HG100 to HG1000 (50 mM Hepes pH7.5, 1000 mM NaCl, 10% glycerol, 0.5 mM TCEP). Appropriate fractions were pooled after assaying with an SDS-PAGE gel and concentrated for loading onto a SRT10 SEC300 column (Sepax #225300-21230) equilibrated in HG150 (10 mM Hepes pH7.5, 150 mM NaCl, 20% glycerol, 1 mM TCEP). Fractions were assayed by SDS-PAGE and appropriately pooled, concentrated to at least 5 mg/ml.

The gRNA was generated by in vitro transcription using a T7 polymerase. A 5′ ARCA cap was added to the RNA simultaneous to transcription while a polyA tail was added after transcription to the 3′ end of the RNA species by an E. coli polyA polymerase. Prior to introduction into the cells, the purified Cas9 and gRNA were mixed and allowed to form complexes for 10 minutes. The RNP solution was subsequently introduced into Jurkat T cells by electroporation. Prior to and after electroporation, the cells were cultured in RPMI1640 media supplemented with 10% FBS. CD3 expression on the cells was monitored at 24, 48 and 72 hours post electroporation by flow cytometry using a fluorescein conjugated antibody specific for CD3. At 48 and 72 hours, a population of CD3 negative cells was observed (FIG. 18A and FIG. 18B). To confirm that the generation of CD3 negative cells was a result of genome editing at the TRAC locus, genomic DNA was harvested and a T7E1 assay was performed. Indeed, the data confirm the presence of DNA modifications at the TRAC locus (FIG. 18C).

### Example 10: Assessing Effect of gRNA Modifications on T Cell Viability

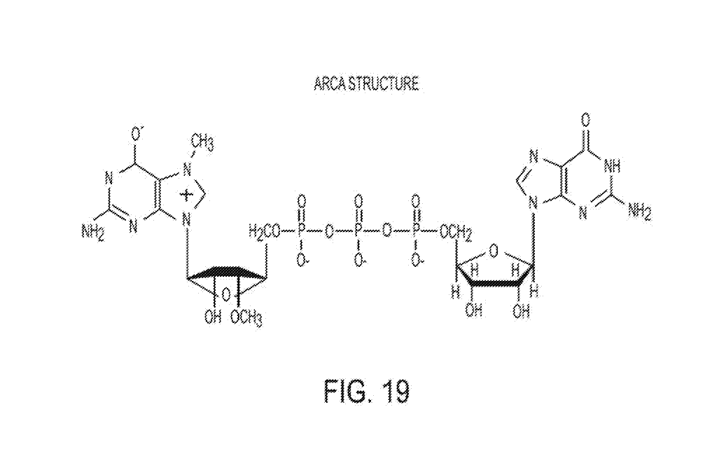

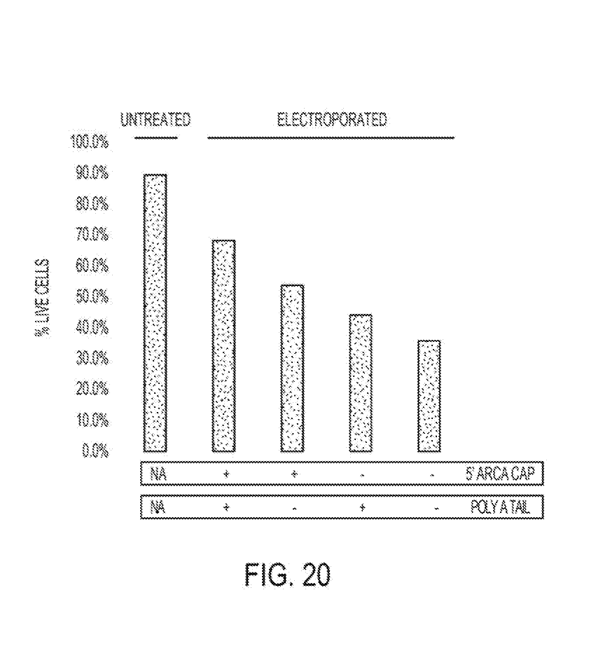

In order to assess how gRNA modifications affect T cell viability, S. pyogenes Cas9 mRNA was delivered to Jurkat T cells in combination with an AAVS1 gRNA (GUCCCCUCCACCCCACAGUG) (SEQ ID NO:387) with or without modification. Specifically, 4 different combinations of modification were analyzed, (1) gRNA with a 5′ Anti-Reverse Cap Analog (ARCA) cap (see FIG. 19) and polyA tail, (2) gRNA with a 5′ ARCA cap only, (3) gRNA with a polyA tail only, (4) gRNA without any modification. In order to generate all of four of the aforemention versions of modified gRNAs, a DNA template comprising a T7 promoter, AAVS1 gRNA target sequence (GUCCCCUCCACCCCACAGUG) (SEQ ID NO:387), and S. pyogenes TRACR sequence. For all gRNAs, T7 polymerase was used to generate the gRNA in the presence of 7.5 mM UTP, 7.5 mM GTP, 7.5 mM CTP and 7.5 mM ATP. In order to modify the gRNAs with a 5′ ARCA cap, 6.0 mM of ARCA analog was added to the NTP pool. Consequently, only 1.5 mM of GTP was added while the rest of the NTP pool remained at the same concentration: 7.5 mM UTP, 7.5 mM CTP and 7.5 mM ATP. In order to add a polyA tail to the gRNAs a recombinant polyA polymerase that was purified from E. coli was used to add a series of As to the end of the transcribed gRNA after the in vitro polymerase reaction was terminated. Termination was achieved by eliminating the DNA template with DNase I. The polyA tail reaction was performed for approximately 40 minutes. Regardless of the gRNA modification, all gRNA preparations were purified by phenol:choroform extraction followed by isopropanol precipitation. Once the gRNAs were generated, Jurkat T cells were electroporated with S. pyogenes Cas9 mRNA (modified with a 5′ ARCA cap and polyA tail) and one of the 4 different modified AAVS1-specific gRNAs. Following electroporation cell viability was determined by performing an Annexin-V and propidium iodide double stain. The fraction of live cells, which do not stain for Annexin-V and PI was determined by flow cytometry. The results are quantified in FIG. 20. Based on the fraction of live cells, it is concluded that gRNAs that have been modified with both a 5′ ARCA cap and polyA tail are the least toxic to Jurkat T cells when introduced by electroporation.

### Example 11: Delivery of Cas9/gRNA RNP to Naïve T Cells

To demonstrate Cas9-mediated cutting in naïve T cells, S. aureus Cas9 and a gRNA designed against the TCR alpha chain with targeting domain GUGAAUAGGCAGACAGACUUGUCA (SEQ ID NO:474) were delivered as a ribonucleic acid protein complex (RNP) by electroporation. In this embodiment, the Cas9 was expressed in E. coli and purified. Specifically, the HJ29 plasmid encoding Cas9 was transformed into Rosetta™ 2 (DE3) chemically competent cells (EMD Millipore #71400-4) and plated onto LB plates with appropriate antibiotics for selection and incubated at 37° C. overnight. A 10 mL starter culture of Brain Heart Infusion Broth (Teknova #B9993) with appropriate antibiotics was inoculated with 4 colonies and grown at 37° C. with shaking at 220 rpms. After growing overnight, the starter culture was added to 1 L of Terrific Broth Complete (Teknova #T7060) with appropriate antibiotics plus supplements and grown at 37° C. with shaking at 220 rpms. The temperature was gradually reduced to 18° C. and expression of the gene was induced by addition of IPTG to 0.5 mM when the OD600 was greater than 2.0. The induction was allowed to continue overnight followed by harvesting the cells by centrifugation and resuspension in TG300 (50 mM Tris pH8.0, 300 mM NaCl, 20% glycerol, 1 mM TCEP, protease inhibitor tablets (Thermo Scientific #88266)) and stored at −80° C.

The cells were lysed by thawing the frozen suspension, followed by two passes through a LM10 Microfluidizer® set to 18000 psi. The extract was clarified via centrifugation and the soluble extract was captured via batch incubation with Ni-NTA Agarose resin (Qiagen #30230) at 4° C. The slurry was poured into a gravity flow column, washed with TG300+30 mM Imidazole and then eluted the protein of interest with TG300+300 mM Imidazole. The Ni eluent was diluted with an equal volume of HG100 (50 mM Hepes pH7.5, 100 mM NaCl, 10% glycerol, 0.5 mM TCEP) and loaded onto a HiTrap SP HP column (GE Healthcare Life Sciences #17-1152-01) and eluted with a 30 column volume gradient from HG100 to HG1000 (50 mM Hepes pH7.5, 1000 mM NaCl, 10% glycerol, 0.5 mM TCEP). Appropriate fractions were pooled after assaying with an SDS-PAGE gel and concentrated for loading onto a SRT10 SEC300 column (Sepax #225300-21230) equilibrated in HG150 (10 mM Hepes pH7.5, 150 mM NaCl, 20% glycerol, 1 mM TCEP). Fractions were assayed by SDS-PAGE and appropriately pooled, concentrated to at least 5 mg/ml.

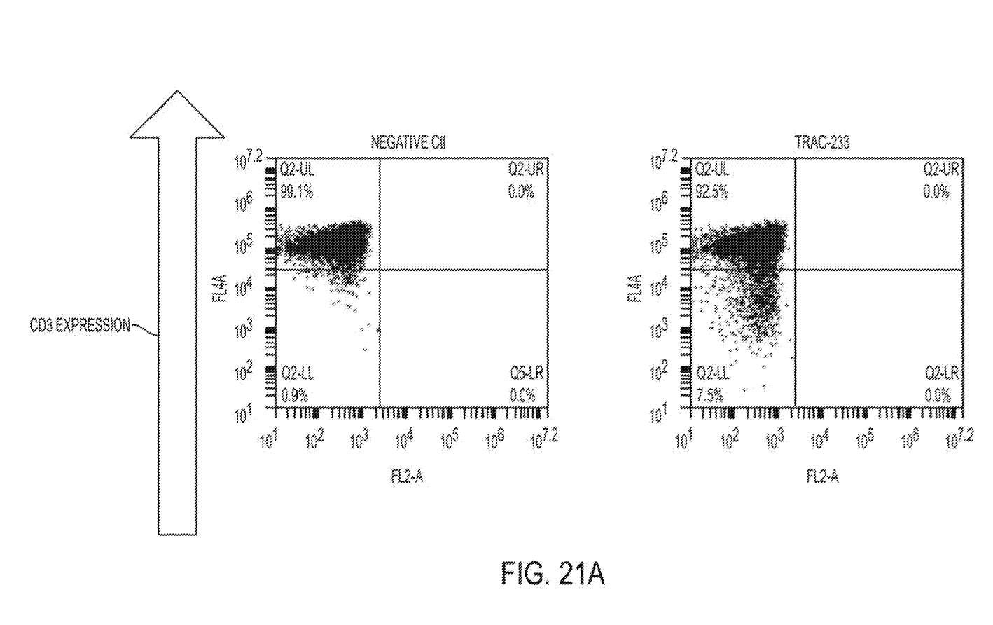

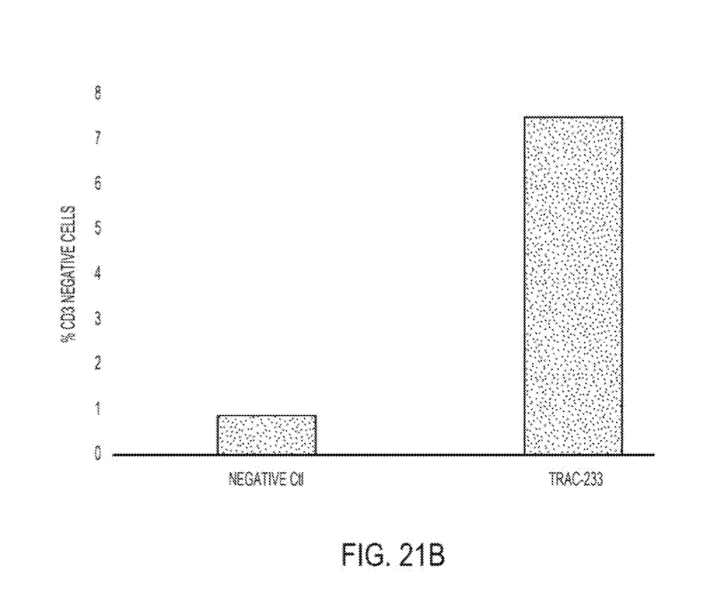

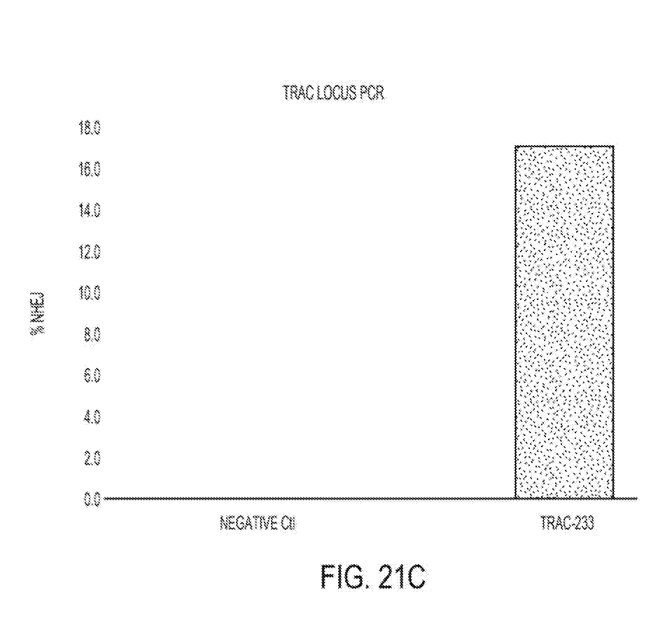

The gRNA with targeting domain GUGAAUAGGCAGACAGACUUGUCA (SEQ ID NO:474) was generated by in vitro transcription using a T7 polymerase. A 5′ ARCA cap was added to the RNA simultaneous to transcription while a polyA tail was added after transcription to the 3′ end of the RNA species by an E. coli polyA polymerase. In this embodiment, the T cells were isolated from fresh cord blood by a Ficoll gradient followed by positive selection using CD3 magnetic beads. The cells were subsequently cultured in RPMI1640 media supplemented with 10% FBS, IL-7 (5 ng/ml) and IL-15 (5 ng/ml). 24 hours after isolation, the cells were electroporated with a RNP solution that was generated by incubating the purified Cas9 and gRNA for 10 minutes at room temperature. CD3 expression on the cells was monitored at 96 hours post electroporation by flow cytometry using an APC-conjugated antibody specific for CD3. A population of CD3 negative cells was observed in cells that in which a functional RNP complex was provided versus a negative control that received the gRNA and a non-functional Cas9 (FIG. 21A and FIG. 21B). To confirm that the generation of CD3 negative cells was a result of genome editing at the TRAC locus, genomic DNA was harvested and a T7E1 assay was performed. Indeed, the data confirm the presence of DNA modifications at the TRAC locus (FIG. 21C).

### Example 12: Targeting of the PDCD1 Locus by Delivery of Cas9 mRNA and gRNA as RNA Molecules or as Cas9/gRNA RNP to Jurkat T Cells

Delivery of Cas9 mRNA and gRNA as RNA Molecules to Jurkat T Cells

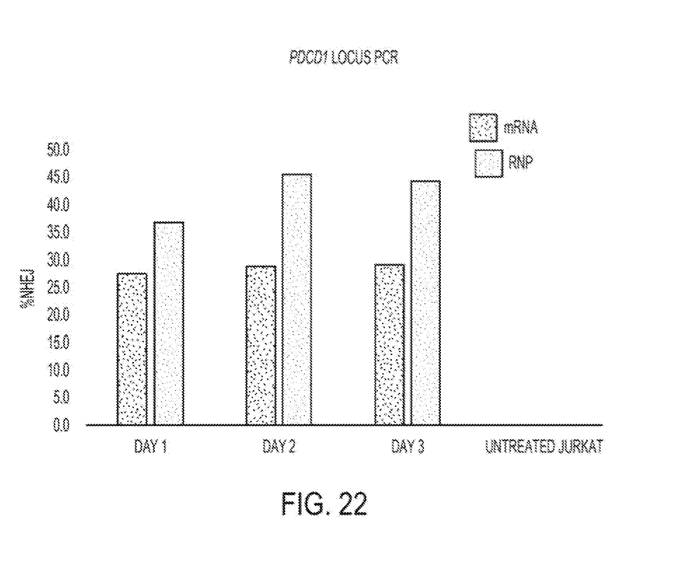

To demonstrate Cas9-mediated cutting at the PDCD1 locus in Jurkat T cells, S. pyogenes Cas9 and a gRNA with a targeting domain GUCUGGGCGGUGCUACAACU (SEQ ID NO:508) designed against the PDCD1 locus were delivered as RNA molecules to T cells via electroporation. In this embodiment, both the Cas9 and gRNA were in vitro transcribed using a T7 polymerase. A 5′ ARCA cap was added to both RNA species simultaneous to transcription while a polyA tail was added after transcription to the 3′ end of the RNA species by an E. coli polyA polymerase. To generate Jurkat T cells modified at the PDCD1 locus, 10 ug of Cas9 mRNA and 10 ug of gRNA with a targeting domain GUCUGGGCGGUGCUACAACU (SEQ ID NO:508) were introduced to the cells by electroporation. Prior to electroporation, the T cells were cultured in RPMI 1640 supplemented with 10% FBS. At 24, 48 and 72 hours, genomic DNA was isolated and a T7E1 assay was performed at the PDCD1 locus. Indeed, the data confirm the presence of DNA modifications at the PDCD1 locus (FIG. 22).

Delivery of Cas9/gRNA RNP to Jurkat T Cells

To demonstrate Cas9-mediated cutting at the PDCD1 locus in Jurkat T cells, S. pyogenes Cas9 and a gRNA designed against the PDCD1 locus with the targeting domain GUCUGGGCGGUGCUACAACU (SEQ ID NO:508) were delivered as a ribonucleic acid protein complex (RNP) by electroporation. In this embodiment, the Cas9 was expressed in E. coli and purified. Specifically, the HJ29 plasmid encoding Cas9 was transformed into Rosetta™ 2 (DE3) chemically competent cells (EMD Millipore #71400-4) and plated onto LB plates with appropriate antibiotics for selection and incubated at 37° C. overnight. A 10 mL starter culture of Brain Heart Infusion Broth (Teknova #B9993) with appropriate antibiotics was inoculated with 4 colonies and grown at 37° C. with shaking at 220 rpms. After growing overnight, the starter culture was added to 1 L of Terrific Broth Complete (Teknova #T7060) with appropriate antibiotics plus supplements and grown at 37° C. with shaking at 220 rpms. The temperature was gradually reduced to 18° C. and expression of the gene was induced by addition of IPTG to 0.5 mM when the OD600 was greater than 2.0. The induction was allowed to continue overnight followed by harvesting the cells by centrifugation and resuspension in TG300 (50 mM Tris pH8.0, 300 mM NaCl, 20% glycerol, 1 mM TCEP, protease inhibitor tablets (Thermo Scientific #88266)) and stored at −80° C.

The cells were lysed by thawing the frozen suspension, followed by two passes through a LM10 Microfluidizer® set to 18000 psi. The extract was clarified via centrifugation and the soluble extract was captured via batch incubation with Ni-NTA Agarose resin (Qiagen #30230) at 4° C. The slurry was poured into a gravity flow column, washed with TG300+30 mM Imidazole and then eluted the protein of interest with TG300+300 mM Imidazole. The Ni eluent was diluted with an equal volume of HG100 (50 mM Hepes pH7.5, 100 mM NaCl, 10% glycerol, 0.5 mM TCEP) and loaded onto a HiTrap SP HP column (GE Healthcare Life Sciences #17-1152-01) and eluted with a 30 column volume gradient from HG100 to HG1000 (50 mM Hepes pH7.5, 1000 mM NaCl, 10% glycerol, 0.5 mM TCEP). Appropriate fractions were pooled after assaying with an SDS-PAGE gel and concentrated for loading onto a SRT10 SEC300 column (Sepax #225300-21230) equilibrated in HG150 (10 mM Hepes pH7.5, 150 mM NaCl, 20% glycerol, 1 mM TCEP). Fractions were assayed by SDS-PAGE and appropriately pooled, concentrated to at least 5 mg/ml.

The gRNA with the targeting domain GUCUGGGCGGUGCUACAACU (SEQ ID NO:508) was generated by in vitro transcription using a T7 polymerase. A 5′ ARCA cap was added to the RNA simultaneous to transcription while a polyA tail was added after transcription to the 3′ end of the RNA species by an E. coli polyA polymerase. Prior to introduction into the cells, the purified Cas9 and gRNA were mixed and allowed to form complexes for 10 minutes. The RNP solution was subsequently introduced into Jurkat T cells by electroporation. Prior to and after electroporation, the cells were cultured in RPMI1640 media supplemented with 10% FBS. At 24, 48 and 72 hours, genomic DNA was isolated and a T7E1 assay was performed at the PDCD1 locus. Indeed, the data confirm the presence of DNA modifications at the PDCD1 locus (FIG. 22).

### Example 13: Purification of S. pyogenes Cas9 Protein

Cas9 was expressed in E. coli and purified. Specifically, the HJ29 plasmid encoding Cas9 was transformed into Rosetta™ 2 (DE3) chemically competent cells (EMD Millipore #71400-4) and plated onto LB plates with appropriate antibiotics for selection and incubated at 37° C. overnight. A 10 mL starter culture of Brain Heart Infusion Broth (Teknova #B9993) with appropriate antibiotics was inoculated with 4 colonies and grown at 37° C. with shaking at 220 rpms. After growing overnight, the starter culture was added to 1 L of Terrific Broth Complete (Teknova #T7060) with appropriate antibiotics plus supplements and grown at 37° C. with shaking at 220 rpms. The temperature was gradually reduced to 18° C. and expression of the gene was induced by addition of IPTG to 0.5 mM when the OD600 was greater than 2.0. The induction was allowed to continue overnight followed by harvesting the cells by centrifugation and resuspension in TG300 (50 mM Tris pH8.0, 300 mM NaCl, 20% glycerol, 1 mM TCEP, protease inhibitor tablets (Thermo Scientific #88266)) and stored at −80° C.

The cells were lysed by thawing the frozen suspension, followed by two passes through a LM10 Microfluidizer® set to 18000 psi. The extract was clarified via centrifugation and the soluble extract was captured via batch incubation with Ni-NTA Agarose resin (Qiagen #30230) at 4° C. The slurry was poured into a gravity flow column, washed with TG300+30 mM Imidazole and then eluted the protein of interest with TG300+300 mM Imidazole. The Ni eluent was diluted with an equal volume of HG100 (50 mM Hepes pH7.5, 100 mM NaCl, 10% glycerol, 0.5 mM TCEP) and loaded onto a HiTrap SP HP column (GE Healthcare Life Sciences #17-1152-01) and eluted with a 30 column volume gradient from HG100 to HG1000 (50 mM Hepes pH7.5, 1000 mM NaCl, 10% glycerol, 0.5 mM TCEP). Appropriate fractions were pooled after assaying with an SDS-PAGE gel and concentrated for loading onto a SRT10 SEC300 column (Sepax #225300-21230) equilibrated in HG150 (10 mM Hepes pH7.5, 150 mM NaCl, 20% glycerol, 1 mM TCEP). Fractions were assayed by SDS-PAGE and appropriately pooled, concentrated to at least 5 mg/ml. Aliquots were stored at −80° C.

### Example 14: In Vitro Transcription of gRNA

A DNA template encoding a modified T7 promoter, gRNA target sequence, and chimeric S. pyogenes gRNA scaffold was assembled by PCR. The 5′ sense primer used for PCR consisted of the modified T7 promoter, gRNA targeting sequence (which was modified for each gRNA based on the desired target site), and sequence from the 5′ end of the S. pyogenes gRNA tracr sequence (GTTTTAGAGCTAGAAATA (SEQ ID NO:51205)). The 3′ anti-sense primer (AAAAGCACCGACTCGGTGCCACTTTTTCAAGTTGATA (SEQ ID NO:51206)) was the reverse complement to the 3′ end of the S. pyogenes gRNA tracr sequence. The DNA template for the PCR reactions was a plasmid containing the S. pyogenes gRNA tracr sequence. The amplified PCR product which was used as a DNA template for in vitro transcription of a target specific gRNA therefore encoded the following: modified T7 promoter-gRNA target sequence-S. pyogenes chimeric gRNA scaffold (i.e., modified T7 promoter followed by gRNA).

Given that the T7 RNA polymerase requires a G to initiate transcription, the T7 promoter typically has two Gs at its 3′ end to ensure transcription of the entire RNA sequence downstream of the promoter. The consequence, however, is that the transcript that is produced may contain at least one if not both of the Gs from the promoter sequence, which may alter the gRNA specificity or the interaction between the gRNA and the Cas9 protein. To address this concern in cases where the gRNA target sequence starts with a G, the two GGs were removed from the following T7 promoter sequence TAATACGACTCACTATAGG (SEQ ID NO:51202) in the gRNA PCR template by using a 5′ sense primer that included the following modified T7 promoter sequence: TAATACGACTCACTATA (SEQ ID NO:51203). For gRNA target sequences that don't start with a G, the T7 promoter sequence encoded in the gRNA PCR template was modified such that only one of the Gs at the 3′ end of the T7 promoter was removed by using a 5′ sense primer that included the following modified T7 promoter sequence: TAATACGACTCACTATAG (SEQ ID NO:51204). gRNAs were generated by in vitro transcription of the DNA templates with the Message Machine™ T7 Ultra Transcription Kit (Ambion). In Example 10, an ARCA cap was added to the 5′ end of the gRNA in the in vitro transcriptions process followed by treatment with E-PAP which adds a poly A tail to the end of the gRNA sequence thus, all gRNAs used in Example 10 were modified at the 5′ end with ARCA cap and at the 3′ end with a polyA tail. For all experiments described in Examples 11-13, gRNAs were in vitro transcribed from a gRNA PCR template that encodes a modified T7 promoter, gRNA, and a polyA tail (20A) 3′ to the gRNA. An ARCA cap was added to the 5′ end of the gRNA in the in vitro transcriptions process, thus, all gRNAs in Examples 11-13 were modified at the 5′ end with ARCA cap and at the 3′ end with a polyA tail).

Modified T7 promoter sequences are not limited to the sequences described herein. For example, T7 promoter sequences (and modifications thereof) can be at least any of the sequences referred to in “Promoters/Catalog/T7” of the Registry of Standard Biological Parts (located at the following http://address: parts.igem.org/Promoters/Catalog/T7). It is to be understood that the present disclosure encompasses methods where a gRNA of the invention is prepared by in vitro transcription from a DNA template that includes a modified T7 promoter as described herein where one or more of the 3′ terminal Gs have been removed (e.g., where the sequence TAATACGACTCACTATAG (SEQ ID NO:51204) is located immediately upstream of a target sequence that lacks a G at it's 5′ end or the sequence TAATACGACTCACTATA (SEQ ID NO:51203) is located immediately upstream of a target sequence that has a G at it's 5′ end). Other variations on these modified T7 promoters will be recognized by those skilled in the art based on other T7 promoter sequences including at least any of the sequences referred to in “Promoters/Catalog/T7” of the Registry of Standard Biological Parts (located at the following http://address: parts.igem.org/Promoters/Catalog/T7 and incorporated herein by reference in its entirety).

### Example 15: Identification of PDCD1 Targeting gRNA Pairs

In order to assess whether S. pyogenes nickases could be used to generate a high percentage of PDCD1 negative T cells, both the D10A and N863A nickases were purified from E. coli in accordance with the method of Example 8. Using a software tool, PDCD1 gRNAs were identified and mapped to the locus of PDCD1. gRNA pairs were chosen for further assessment based on two major criteria: 1) the PAM sequence of the two gRNAs are outward facing; and 2) the distance between the predicted cut sites (4 bp away from the PAM) is greater than 30 bp and less than 90 bp. The chosen gRNAs were generated using a T7-based in vitro transcription reaction as described in Example 9. Each gRNA was complexed with either the D10A nickase or the N863A nickase. After verification that the complexing was complete using DSF (see methods in Section IV herein), the two appropriate RNPs corresponding to the listed pairs were combined at a 1:1 ratio and electroporated into the cells at a dose of 1 ug total RNP/100,000 cells. The RNP was electroporated into 250,000 activated CD4 T cells in a 96-well format (in duplicate) and subsequently cultured in T cell media containing IL-2, IL-7 and IL-15. After 3 days of culture, the cells were activated with PMA/IO for 24 hours and PDCD1 expression was assessed by flow cytometry using a PE-conjugated anti-human-PDCD1 antibody. The percentage of PDCD1 negative cells is plotted in FIG. 31. Delivery of several D10A nickase pairs resulted in >90% PDCD1 negative cells while the same gRNAs pairs when complexed with the N863A nickase gave rise to lower but detectable levels of PDCD1 knockout. A single nickase RNP resulted in negligible loss of PDCD1 expression while a single gRNA complexed with wild-type S. pyogenes resulted in high levels of knockout as expected. Tables 8000A and 8000B provide details for the targeting domains that were used for each of the gRNA pairs.

TABLE 8000A

| | ID | | Distance | |
|---|---|---|---|---|
| | Left | Right | between nicks | |
| Pair | gRNA | gRNA | (bp) | Nickase |
| 1 | 102 | 49 | 31 | D10A |
| 2 | 102 | 51 | 36 | D10A |
| 3 | 102 | 52 | 59 | D10A |
| 4 | 102 | 53 | 65 | D10A |
| 5 | 102 | 97 | 30 | D10A |
| 6 | 102 | 103 | 43 | D10A |
| 7 | 98 | 52 | 44 | D10A |
| 8 | 98 | 53 | 49 | D10A |
| 9 | 98 | 103 | 28 | D10A |
| 10 | 99 | 52 | 49 | D10A |
| 11 | 99 | 53 | 54 | D10A |
| 12 | 99 | 103 | 33 | D10A |
| 13 | 102 | 49 | 31 | N863A |
| 14 | 102 | 51 | 36 | N863A |
| 15 | 102 | 52 | 59 | N863A |
| 16 | 102 | 53 | 65 | N863A |
| 17 | 102 | 97 | 30 | N863A |
| 18 | 102 | 103 | 43 | N863A |
| 19 | 98 | 52 | 44 | N863A |
| 20 | 98 | 53 | 49 | N863A |
| 21 | 98 | 103 | 28 | N863A |
| 22 | 99 | 52 | 49 | N863A |
| 23 | 99 | 53 | 54 | N863A |
| 24 | 99 | 103 | 33 | N863A |

TABLE 8000B

| | | | | SEQ |
|---|---|---|---|---|
| | | | Cas9 | ID |
| ID | gRNA Name | Targeting Domain | species | NO: |
| 49 | PDCD1-108 | GUCUGGGCGGUGCUACAACU | S. pyogenes | 508 |
| 51 | PDCD1-109 | GGGCGGUGCUACAACUGGGC | S. pyogenes | 510 |
| 52 | PDCD1-114 | GGCCAGGAUGGUUCUUAGGU | S. pyogenes | 511 |
| 53 | PDCD1-117 | GGAUGGUUCUUAGGUAGGUG | S. pyogenes | 512 |
| 55 | PDCD1-232 | GCCCUGGCCAGUCGUCU | S. pyogenes | 514 |
| 97 | PDCD1-107 | CGUCUGGGCGGUGCUACAAC | S. pyogenes | 576 |
| 98 | PDCD1-122 | UGUAGCACCGCCCAGACGAC | S. pyogenes | 579 |
| 99 | PDCD1-124 | ACCGCCCAGACGACUGGCCA | S. pyogenes | 581 |
| 102 | PDCD1-126 | CGACUGGCCAGGGCGCCUGU | S. pyogenes | 582 |
| 103 | PDCD1-111 | CUACAACUGGGCUGGCGGCC | S. pyogenes | 766 |
| 104 | PDCD1-121 | CACCUACCUAAGAACCAUCC | S. pyogenes | 723 |

### Example 16: Assessment of In Vivo Activity of PDCD1-Deleted, CAR-Expressing T Cells in Nalm-6 Disseminated Tumor Model

A disseminated tumor xenograft mouse model was generated by injecting NOD/Scid/gc−/− (NSG) mice with Nalm-6 tumor line cells overexpressing PD-L1. Specifically, on day zero (0), mice were injected intravenously (i.v.) with 5×10⁵ cells of a Nalm6 human B cell precursor leukemia cell line overexpressing PD-L1 and transfected with green fluorescence protein and firefly luciferase (Nalm6-PD-L1-ffluc-GFP). Tumor engraftment was allowed to occur for 4 days and verified using bioluminescence imaging. On day 4, mice in each of eight (eight) study groups received no treatment or a single intravenous (i.v.) injection of engineered cells (generated essentially as described in Example 4), at one of various doses/types, as follows: (1) no cells administered (tumor alone), (2) 1×10⁶AAVS1-deleted T cells transduced with a mock control vector; (3) 5×10⁵ AAVS1-deleted cells expressing an anti-CD19 CAR+ T cells; (4) 1×10⁶ AAVS1-deleted anti-CD19 CAR+ T cells; (5) 1×10⁶PDCD1-deleted T cells transduced with a mock control vector; (6) 5×10⁵ PDCD1-deleted anti-CD19 CAR+ T cells; (7) 1×10⁶PDCD1-deleted anti-CD19 CAR+ T cells; and (8) 1×10⁶ anti-CD19 CAR+ T cells that had been subjected to a mock electroporation control.

A. Anti-Tumor Activity

Following treatment, tumor growth over time was monitored by bioluminescence imaging and the average radiance (p/s/cm²/sr) was measured approximately every 5-7 days through day 28. For bioluminescence imaging, mice received intraperitoneal (i.p.) injections of luciferin substrate (CaliperLife Sciences, Hopkinton, Mass.) resuspended in PBS (15 μg/g body weight). As shown in FIG. 32, tumors in control mice (tumor alone and those treated with mock control-transduced AAVS1-deleted T cells or mock control-transduced PDCD1-deleted T cells) continued to grow over the course of the study. By comparison, mice having been administered engineered T cells expressing anti-CD19 CARs, including the PDCD1-deleted anti-CD19 CAR+ T cells, at various doses, exhibited a reduction in the amount of average radiance at all post-treatment time points tested. The results indicated that the PDCD1 deleted CAR+ T cells were able to suppress tumor growth in a mouse cancer model and that PDCD1 deletion did not impair the in vivo anti-tumor function of the CAR+ T cells.

B. Expansion and Persistence of PDCD1 Knockout Cells In Vivo

Bone marrow samples were obtained from mice of a first satellite group and control groups) were analyzed to assess in vivo expansion and persistence of the administered PDCD1-deleted cells. Absolute CD4+ and CD8+ T cell counts (FIGS. 33A and 33B), and absolute PD1+ T cell counts (FIGS. 34A, 34B, and 34C), were determined by flow cytometry.

Results for numbers of circulating CD4+ or CD8+ cells in the bone marrow at day 9 are shown in FIGS. 33A and 33B, respectively. The results indicated that PDCD1-deleted CAR+ T cells expanded and persisted in the mouse model following administration, at similar rates as compared to CAR+ control (AAVS1-deleted and mock electroporated) cells. Counts of PD1+ T cells (CD3+, CD4+ and CD8+) observed in the bone marrow at day 9 are shown in FIGS. 34A, 34B and 34C, respectively. The results were consistent with a conclusion that PDCD1 deletion of PDCD1 was maintained in CAR+ cells after administration to animals with tumors expressing the CAR-targeted antigen and in vivo expansion, and that PD-1-knockout CAR+ T cells persisted in vivo following administration.

### Example 17: Assessment of In Vivo Activity of PDCD1-Deleted, CAR-Expressing T Cells in A549 Subcutaneous Tumor Model

A subcutaneous tumor xenograft mouse model was generated by injecting NOD/Scid/gc−/− (NSG) mice with A549 lung adenocarcinoma cells engineered to express high levels of human CD19. In this study, the overexpression of human CD19 in these cells in the xenograft model permitted assessment of CD19-specific PDCD1 CAR+ T cells in the context of a solid tumor. Additionally, based on separate observations that A549 lung adenocarcinoma cells can upregulate PD-L1 in response to interferon gamma stimulation, the model permitted evaluation of CD19-specific PDCD1 CAR+ T cells in a tumor environment that may express PD-L1 in response to IFN-gamma. On day zero (0), A549-huCD19^{hi} cells were subcutaneously implanted into immune-deficient NSG mice.

In vitro studies demonstrate that upon interaction with the CD19 target antigen, the T cell negative regulatory molecule, PD-1 is upregulated on CAR T cells, including anti-CD19 CAR-expressing cells. Interaction of PD-1 on CART cells with PD-L1 on CD19-expressing tumor cells could limit the activity of the CAR T cells. After tumor engraftment, the mice in three (3) different treatment groups received a single intravenous (i.v.) injection of various 4×10⁶ anti-CD19 CAR-expressing T cell populations generated as described in Example 4: (1) AAVS1-deletedanti-CD19 CAR+ T cells; (2) PDCD1-deleted anti-CD19 CAR+ T cells; and (3) anti-CD19 CAR+ cells not subjected to deletion or electroporation. Mice not injected with any engineered T cells (tumor alone) were assessed as negative controls. Tumor volume was measured on days 11, 14, 19, 23 and 26 post CAR-T cell administration. The results are shown in FIG. 35. As shown, administration of each of the CAR+ cell compositions was observed to reduce tumor growth in this CD19-overexpressing lung adenocarcinoma model as compared to that observed in untreated controls.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

What is claimed:

1. A composition, comprising (a) an engineered immune cell comprising a recombinant receptor that specifically binds to an antigen; and (b) an agent capable of inducing a genetic disruption of a PDCD1 gene encoding a PD-1 polypeptide, wherein said agent is capable of inducing said genetic disruption in, and/or preventing or reducing PD-1 expression in, at least 70%, at least 75%, at least 80%, or at least or greater than 90% of the cells in the composition and/or at least 70%, at least 75%, at least 80%, or at least or greater than 90% of the cells in the composition that express the recombinant receptor.

2. A composition, comprising (a) an engineered immune cell comprising a nucleic acid encoding a recombinant receptor that specifically binds to an antigen; and (b) an agent capable of inducing a genetic disruption of a PDCD1 gene encoding a PD-1 polypeptide, wherein said agent is capable of inducing said genetic disruption in, and/or preventing or reducing PD-1 expression in, at least 70%, at least 75%, at least 80%, or at least or greater than 90% of the cells in the composition and/or at least 70%, at least 75%, at least 80%, or at least or greater than 90%, of the cells in the composition that express the recombinant receptor.

3. The composition of claim 1 or claim 2, wherein the engineered immune cell expresses the recombinant receptor on its surface.

4. A composition, comprising a cell population containing an engineered immune cell that comprises (a) a recombinant receptor that specifically binds to an antigen; and (b) a genetic disruption of a PDCD1 gene encoding a PD-1 polypeptide, said genetic disruption preventing or reducing the expression of said PD-1 polypeptide, wherein:
at least about 70%, at least about 75%, or at least about 80% or at least or greater than about 90% of the cells in the composition contain the genetic disruption; do not express the endogenous PD-1 polypeptide; do not contain a contiguous PDCD1 gene, do not contain a PDCD1 gene, and/or do not contain a functional PDCD1 gene; and/or do not express a PD-1 polypeptide; and/or
at least about 70%, at least about 75%, or at least about 80% or at least or greater than about 90% of the cells in the composition that express the recombinant receptor contain the genetic disruption, do not express the endogenous PD-1 polypeptide, and/or do not express a PD-1 polypeptide.

5. A composition, comprising a cell population containing an engineered immune cell that comprises (a) a recombinant receptor that specifically binds to an antigen, wherein the engineered immune cell is capable of inducing cytotoxicity, proliferating and/or secreting a cytokine upon binding of the recombinant receptor to said antigen; and (b) a genetic disruption of a PDCD1 gene encoding a PD-1 polypeptide, said genetic disruption capable of preventing or reducing the expression of said PD-1 polypeptide, optionally wherein said prevention or reduction is in at least at or about or greater than at or about 70%, 75%, 80%, 85%, or 90% of the cells in the composition and/or of the cells in the composition that express the recombinant receptor.

6. A composition comprising a cell population containing a population of engineered immune cells, each comprising (a) a recombinant receptor that specifically binds to an antigen; and (b) a genetic disruption of a PDCD1 gene encoding a PD-1 polypeptide, wherein said genetic disruption is capable of preventing or reducing the expression of said PD-1 polypeptide, wherein:
the engineered immune cells, on average, exhibit expression and/or surface expression of the receptor at a level that is the same, about the same or substantially the same, as compared to the average expression and/or surface expression level, respectively, of said recombinant receptor in other cells in the composition that comprise the recombinant receptor and do not comprise the genetic disruption, or
the engineered immune cells do not express the PD-1 polypeptide and on average, exhibit expression and/or surface expression of the receptor at a level is the same, about the same, or substantially the same as compared to the average expression and/or surface level, respectively, in cells of the composition that comprise the recombinant receptor and that express the PD-1 polypeptide.

7. The composition of any of claims 1-4 and 6, wherein the recombinant receptor is capable, upon incubation with the antigen, a cell expressing the antigen, and/or an antigen-receptor activating substance, of specifically binding to the antigen, of activating or stimulating the engineered T cell, of inducing cytotoxicity, or of inducing proliferation, survival, and/or cytokine secretion by the immune cell, optionally as measured in an in vitro assay, optionally in an in vitro assay, which optionally comprises incubation for 12, 24, 36, 48, or 60 hours, optionally in the presence of one or more cytokines.

8. The composition of any of claims 1-4, 6 and 7, wherein the engineered immune cell is capable, upon incubation with the antigen, a cell expressing the antigen, and/or an antigen-receptor activating substance, of specifically binding to the antigen, of inducing cytotoxicity, proliferating, surviving, and/or secreting a cytokine, optionally as measured in an in vitro assay, which optionally comprises incubation for 12, 24, 36, 48, or 60 hours, optionally in the presence of one or more cytokines and optionally does or does not comprise exposing the immune cell to a PD-L1-expressing cell.

9. The composition of claim 7 or claim 8, wherein:
the level or degree or extent or duration of the binding, cytotoxicity, proliferation, survival, or cytokine secretion is the same, about the same or substantially the same as compared to that detected or observed for an immune cell comprising the recombinant receptor but not comprising the genetic disruption of a PDCD1 gene, when assessed under the same conditions.

10. The composition of any of claims 6 and 8-9, wherein the binding, cytotoxicity, proliferation, survival, and/or cytokine secretion is as measured, optionally in an in vitro assay, following withdrawal and re-exposure to the antigen, antigen-expressing cell, and/or substance.

11. The composition of any of claims 1-10, wherein the immune cell is a primary cell from a subject.

12. The composition of any of claims 1-11, wherein the immune cell is a human cell.

13. The composition of any of claims 1-12, wherein the immune cell is a white blood cell.

14. The composition of any of claims 1-13, wherein the immune cell is an NK cell or a T cell.

15. The composition of claim 14, wherein the immune cell comprises a plurality of T cells comprising unfractionated T cells, comprises isolated CD8+ cells or is enriched for CD8+ T cells, or comprises isolated CD4+ T cells or is enriched for CD4+ cells, and/or is enriched for a subset thereof selected from the group consisting of naïve cells, effector memory cells, central memory cells, stem central memory cells, effector memory cells, and long-lived effector memory cells.

16. The composition of claim 14 or claim 15, wherein the percentage, of T cells, or T cells expressing the receptor, and comprising the genetic disruption in the composition, that exhibit a non-activated, long-lived memory, or central memory phenotype, is the same or substantially the same as a population of cells the same or substantially the same as the composition but not containing the genetic disruption or but expressing the PD-1 polypeptide.

17. The composition of any of claims 1-16, wherein the percentage of T cells in the composition exhibiting a non-activated, long-lived memory, or central memory phenotype is the same, about the same or substantially the same as compared to the percentage of T cells exhibiting the phenotype in a composition comprising T cells, comprising the recombinant receptor but not comprising the genetic disruption of a PDCD1 gene encoding a PD-1 polypeptide when assessed under the same conditions, which optionally is compared in the absence or presence of contacting or exposing the immune cell to PD-L1.

18. The composition of claim 16 or claim 17, wherein the phenotype is as assessed following incubation of the composition at or about 37° C.±2° C. for at least 12 hours, 24 hours, 48 hours, 96 hours, 6 days, 12 days, 24 days, 36 days, 48 days or 60 days.

19. The composition of claim 18, wherein the incubation is in vitro.

20. The composition of claim 18 or claim 19, wherein at least a portion of the incubation is performed in the presence of a stimulating agent, which at least a portion is optionally for up to 1 hour, 6 hours, 24 hours, or 48 hours of the incubation.

21. The composition of claim 20, wherein the stimulating agent is an agent capable of inducing proliferation of T cells, CD4+ T cells and/or CD8+ T cells.

22. The composition of claim 20 or claim 21, wherein the stimulating agent is or comprises an antibody specific for CD3 an antibody specific for CD28 and/or a cytokine.

23. The composition of any of claims 16-22, wherein the T cell comprising the recombinant receptor comprises one or more phenotypic markers selected from CCR7+, 4-1BB+(CD137+), TIM3+, CD27+, CD62L+, CD127+, CD45RA+, CD45RO−, t-bet^{low}, IL-7Ra+, CD95+, IL-2Rβ+, CXCR3+ or LFA-1+.

24. The composition of any of claims 1-23, wherein the recombinant receptor is a functional non-TCR antigen receptor or a transgenic TCR.

25. The composition of any of claims 1-23, wherein the recombinant receptor is a chimeric antigen receptor (CAR).

26. The composition of claim 25, wherein the CAR comprises an antigen-binding domain that is an antibody or an antibody fragment.

27. The composition of claim 26, wherein the antibody fragment is a single chain fragment.

28. The composition of claim 26 or claim 27, wherein the antibody fragment comprises antibody variable regions joined by a flexible immunoglobulin linker.

29. The composition of any of claims 26-28, wherein the fragment comprises an scFv.

30. The composition of any of claims 1-29, wherein the antigen is associated with a disease or disorder.

31. The composition of claim 30, wherein the disease or disorder is an infectious disease or condition, an autoimmune disease, an inflammatory disease or a tumor or a cancer.

32. The composition of any of claims 1-31, wherein the recombinant receptor specifically binds to a tumor antigen.

33. The composition of any of claims 1-32, wherein the antigen is selected from ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acethycholine receptor, GD2, GD3, HMW-MAA, IL-22R-alpha, IL-13R-alpha2, kdr, kappa light chain, Lewis Y, L1-cell adhesion molecule (CD171), MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1), cyclin A1 (CCNA1), BCMA and interleukin 12.

34. The composition of any of claims 1-33, wherein the recombinant receptor comprises an intracellular signaling domain comprising an ITAM.

35. The composition of claim 34, wherein the intracellular signaling domain comprises an intracellular domain of a CD3-zeta (CD3) chain.

36. The composition of claim 34 or claim 35, wherein the recombinant receptor further comprises a costimulatory signaling region.

37. The composition of claim 36, wherein the costimulatory signaling region comprises a signaling domain of CD28 or 4-1BB.

38. The composition of any of claims 1-3 and 7-37, wherein the agent comprises at least one of (a) a least one gRNA having a targeting domain that is complementary with a target domain of a PDCD1 gene or (b) at least one nucleic acid encoding the at least one gRNA.

39. The composition of any of claims 1-3 and 7-38, wherein the agent comprises at least one complex of a Cas9 molecule and a gRNA having a targeting domain that is complementary with a target domain of a PDCD1 gene.

40. The composition of claim 38 or claim 39, wherein the guide RNA further comprises a first complementarity domain, a second complementarity domain that is complementary to the first complementarity domain, a proximal domain and optionally a tail domain.

41. The composition of claim 40, wherein the first complementarity domain and second complementarity domain are joined by a linking domain.

42. The composition of any of claim 41, wherein the guide RNA comprises a 3′ poly-A tail and a 5′ Anti-Reverse Cap Analog (ARCA) cap.

43. The composition any of claims 39-42, wherein the Cas9 molecule is an enzymatically active Cas9.

44. The composition of any of claims 38-43, wherein the at least one gRNA includes a targeting domain comprising a sequence selected from the group consisting of GUCUGGGCGGUGCUACAACU (SEQ ID NO:508), GCCCUGGCCAGUCGUCU (SEQ ID NO: 514), CGUCUGGGCGGUGCUACAAC (SEQ ID NO:1533), UGUAGCACCGCCCAGACGAC (SEQ ID NO:579), CGACUGGCCAGGGCGCCUGU (SEQ ID NO:582) and CACCUACCUAAGAACCAUCC (SEQ ID NO:723).

45. The composition of any of claims 38-44, wherein the at least one gRNA includes a targeting domain comprising the sequence CGACUGGCCAGGGCGCCUGU (SEQ ID NO:582).

46. The composition of any of claims 38-45, wherein the Cas9 molecule is a nickase and two Cas9 molecule/gRNA molecule complexes are guided by two different gRNA molecules to cleave the target domain with two single stranded breaks on opposing strands of the target domain.

47. The composition of any of claims 39-46, wherein the Cas9 molecule is an S. aureus Cas9 molecule.

48. The composition of any of claims 39-46, wherein the Cas9 molecule is an S. pyogenes Cas9.

49. The composition of any of claims 39-48, wherein the Cas9 molecule lacks an active RuvC domain or an active HNH domain.

50. The composition of any of claims 39-46, 48 and 49, wherein the Cas9 molecule is an S. pyogenes Cas9 molecule comprising a D10A mutation.

51. The composition of any of claims 46-50, wherein the two gRNA molecules comprise targeting domains that are selected from the following pairs of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
(SEQ ID NO: 582)
CGACUGGCCAGGGCGCCUGU
and
(SEQ ID NO: 511)
GGCCAGGAUGGUUCUUAGGU;
(SEQ ID NO: 582)
CGACUGGCCAGGGCGCCUGU
and
(SEQ ID NO: 512)
GGAUGGUUCUUAGGUAGGUG;
(SEQ ID NO: 582)
CGACUGGCCAGGGCGCCUGU
and
(SEQ ID NO: 576)
CGUCUGGGCGGUGCUACAAC;
(SEQ ID NO: 582)
CGACUGGCCAGGGCGCCUGU
and
(SEQ ID NO: 766)
CUACAACUGGGCUGGCGGCC;
(SEQ ID NO: 579)
UGUAGCACCGCCCAGACGAC
and
(SEQ ID NO: 511)
GGCCAGGAUGGUUCUUAGGU;
(SEQ ID NO: 579)
UGUAGCACCGCCCAGACGAC
and
(SEQ ID NO: 512)
GGAUGGUUCUUAGGUAGGUG;
or
(SEQ ID NO: 581)
ACCGCCCAGACGACUGGCCA
and
(SEQ ID NO: 511)
GGCCAGGAUGGUUCUUAGGU.

52. The composition of any of claims 39-46 and 48-51, wherein the Cas9 molecule is an S. pyogenes Cas9 molecule comprising an N863A mutation.

53. The composition of claim 52, wherein the two gRNA molecules comprise targeting domains that are selected from the following pairs of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
or
(SEQ ID NO: 582)
CGACUGGCCAGGGCGCCUGU
and
(SEQ ID NO: 511)
GGCCAGGAUGGUUCUUAGGU.

54. The composition of any of claims 1-53, wherein the genetic disruption comprises creation of a double strand break which is repaired by non-homologous end joining (NHEJ) to effect insertions and deletions (indels) in the PDCD1 gene.

55. The composition of any of claims 1-54, wherein:
at least about 70%, at least about 75%, or at least about 80% of the cells in the composition contain the genetic disruption; do not express the endogenous PD-1 polypeptide; do not contain a contiguous PDCD1 gene, a PDCD1 gene, and/or a functional PDCD1 gene; and/or do not express a PD-1 polypeptide; and/or
at least about 70%, at least about 75%, or at least about 80% of the cells in the composition that express the recombinant receptor contain the genetic disruption, do not express the endogenous PD-1 polypeptide, or do not express a PD-1 polypeptide

56. The composition of claim 4 or claim 55, wherein:
greater than 80%, 81%, 82%, 83%, 84%, 85%, 86%, 87%, 88%, 89%, 90%, 91%, 92%, 93%, 94%, or 95% of the cells in the composition contain the genetic disruption; do not express the endogenous PD-1 polypeptide; do not contain a contiguous PDCD1 gene, a PDCD1 gene, and/or a functional PDCD1 gene; and/or do not express a PD-1 polypeptide; and/or
greater than 80%, 81%, 82%, 83%, 84%, 85%, 86%, 87%, 88%, 89%, 90%, 91%, 92%, 93%, 94%, or 95% of the cells in the composition that express the recombinant receptor contain the genetic disruption, do not express the endogenous PD-1 polypeptide, or do not express a PD-1 polypeptide.

57. The composition of any of claims 1-56, wherein:
at least at or about 90% of the cells in the composition, or cells in the composition that express the recombinant receptor, optionally as assessed by flow cytometry, contain the genetic disruption; do not express the endogenous PD-1 polypeptide; do not contain a contiguous PDCD1 gene, a PDCD1 gene, and/or a functional PDCD1 gene; and/or do not express a PD-1 polypeptide.

58. The composition of any of claims 1-57, wherein both alleles of the gene in the genome are disrupted.

59. The composition of any of claims 1-58, wherein cells in the composition and/or the cells in the composition that express the recombinant receptor are not enriched or selected for cells that contain the genetic disruption; do not express the endogenous PD-1 polypeptide; do not contain a contiguous PDCD1 gene, a PDCD1 gene, and/or a functional PDCD1 gene; and/or do not express a PD-1 polypeptide.

60. The composition of any of claims 1-59, wherein no more than 2, no more than 5 or no more than 10 other genes in each cell in the composition, or each cell in the composition that expresses the recombinant receptor, on average, are disrupted or are disrupted by the agent.

61. The composition of any of claims 1-60, wherein no other genes in each cell in the composition or each cell in the composition that expresses the recombinant receptor are disrupted in the cell or are disrupted by the agent.

62. The composition of any of claims 1-61, further comprising a pharmaceutically acceptable buffer.

63. The composition of any of claims 1-62, wherein, at a point in time following administration of the composition to a subject, optionally having the disease or condition;
cells expressing the recombinant receptor and not expressing PD-1 are detectable in the blood of the subject or blood-derived sample from the subject or in a tissue or biological sample of the subject;
cells containing the genetic disruption are detectable in the blood of the subject or a blood-derived sample from the subject or in a tissue or biological sample of the subject;
cells containing the genetic disruption and expressing the recombinant receptor are detectable in the blood of the subject or a blood-derived sample from the subject or in a tissue or biological sample of the subject.

64. The composition of claim 63, wherein the time-point is at or about 7, 8, 9, 10, 11, 12, 13, or 14 days or 2, 3, 4, 5, 6, 7, 8, 9, 10, 11, or 12 weeks following administration.

65. The composition of claim 63 or 64, wherein said cells detectable in the blood or sample are present at a concentration of at or about or at least at or about 1, 2, 3, 4, 5, 6, 7, 8, 9, 10, or 100 cells/microliter of blood and/or represent at least 10, 20, 25, 30, 35, 40, 45, or 50% or more of T cells in the blood.

66. The composition of any of claims 1-65, wherein, following administration of the composition to a subject:
cells in the composition comprising the genetic disruption expand and/or persist in the subject at a rate, and/or for a time that, is at least as great as, optionally greater than, the expansion and/or persistence of T cells in the composition without the genetic disruption and/or as the expansion and/or persistence of T cells of a reference composition in which T cells express the recombinant receptor but do not comprising the deletion; and/or
cells in the composition comprising the genetic disruption and comprising the recombinant receptor expand and/or persist in the subject at a rate, and/or for a time that, is at least as great as, optionally greater than, the expansion and/or persistence of T cells in the composition without the genetic disruption and/or as the expansion and/or persistence of T cells of a reference composition in which T cells express the recombinant receptor but do not comprising the deletion.

67. The composition of claim 66, wherein the rate or time is at least at or about 1.5-fold or 2-fold or 3-fold greater.

68. A method of producing a genetically engineered immune cell, comprising:
(a) introducing into an immune cell a nucleic acid molecule encoding a recombinant receptor that specifically binds to an antigen; and
(b) introducing into the immune cell an agent capable of inducing a genetic disruption of a PDCD1 gene encoding a PD-1 polypeptide comprising one of (i) at least one gRNA having a targeting domain that is complementary with a target domain of the PDCD1 gene or (ii) at least one nucleic acid encoding the at least one gRNA.

69. A method of producing a genetically engineered immune cell, comprising introducing into an immune cell expressing a recombinant receptor that specifically binds to an antigen an agent capable of inducing a genetic disruption of a PDCD1 gene encoding a PD-1 polypeptide comprising one of (i) at least one gRNA having a targeting domain that is complementary with a target domain of the PDCD1 gene or (ii) at least one nucleic acid encoding the at least one gRNA.

70. The method of claim 68 or claim 69, wherein the agent comprises at least one complex of a Cas9 molecule and a gRNA having a targeting domain that is complementary with a target domain of a PDCD1 gene.

71. The method of any of claims 68-70, wherein the guide RNA further comprises a first complementarity domain, a second complementarity domain that is complementary to the first complementarity domain, a proximal domain and optionally a tail domain.

72. The method of claim 71, wherein the first complementarity domain and second complementarity domain are joined by a linking domain.

73. The method of any of claims 68-72, wherein the guide RNA comprises a 3′ poly-A tail and a 5′ Anti-Reverse Cap Analog (ARCA) cap.

74. The method of any of claims 68-73, wherein introduction comprises contacting the cells with the agent or a portion thereof, in vitro.

75. The method of any of claims 68-74, wherein introduction of the agent comprises electroporation.

76. The method of claim 74 or claim 75, wherein the introduction further comprises incubating the cells, in vitro prior to, during or subsequent to the contacting of the cells with the agent or prior to, during or subsequent to the electroporation.

77. The method of any of claims 68-76, wherein the introduction in (a) comprises transduction and the introduction further comprises incubating the cells, in vitro, prior to, during or subsequent to the transduction.

78. The method of claim 76 or claim 77, wherein at least a portion of the incubation is in the presence of (i) a cytokine selected from the group consisting of IL-2, IL-7, and IL-15, and/or (ii) a stimulating or activating agent or agents, optionally comprising anti-CD3 and/or anti-CD28 antibodies.

79. The method of claim 77 or claim 78, wherein the introduction in (a) comprises:
prior to transduction, incubating the cells with IL-2 at a concentration of 20 U/mL to 200 U/mL, optionally about 100 U/mL; IL-7 at a concentration of 1 ng/mL to 50 ng/mL, optionally about 10 ng/mL and/or IL-15 at a concentration of 0.5 ng/mL to 20 ng/mL, optionally about 5 ng/mL; and
subsequent to transduction, incubating the cells with IL-2 at a concentration of 10 U/mL to 200 U/mL, optionally about 50 U/mL; IL-7 at a concentration of 0.5 ng/mL to 20 ng/mL, optionally about 5 ng/mL and/or IL-15 at a concentration of 0.1 ng/mL to 10 ng/mL, optionally about 0.5 ng/mL.

80. The method of any of claims 76-79, wherein the incubation independently is carried out for up to or approximately 24, 36, 48 hours, 3, 4, 5, 6, 7, 8, 9, 10, 11, 12, 13, 14, 15, 16, 17, 18, 19, 20 or 21 days.

81. The method of any of claims 76-80, wherein the incubation is independently carried out for 24-48 hours or 36-48 hours.

82. The method of any of claims 74-81, wherein the cells are contacted with the agent at a ratio of approximately 1 microgram per 100,000, 200,000, 300,000, 400,000, or 500,000 cells.

83. The method of any of claims 76-82, wherein:
the incubation is at a temperature of 30° C.±2° C. to 39° C.±2° C.; or
the incubation is at a temperature that is at least or about at least 30° C.±2° C., 32° C.±2° C., 34° C.±2° C. or 37° C.±2° C.

84. The method of any of claims 76-83, wherein at least a portion of the incubation is at 30° C.±2° C. and at least a portion of the incubation is at 37° C.±2° C.

85. The method of any of claims 68-84, wherein the method further comprises resting the cells between the introducing in (a) and the introducing in (b).

86. The method of any of claims 70-85, wherein the Cas9 molecule is an enzymatically active Cas9.

87. The method of any of claims 68-86, wherein the at least one gRNA includes a targeting domain comprising a sequence selected from the group consisting of GUCUGGGCGGUGCUACAACU (SEQ ID NO:508), GCCCUGGCCAGUCGUCU (SEQ ID NO: 514), CGUCUGGGCGGUGCUACAAC (SEQ ID NO:1533), UGUAGCACCGCCCAGACGAC (SEQ ID NO:579), CGACUGGCCAGGGCGCCUGU (SEQ ID NO:582) and CACCUACCUAAGAACCAUCC (SEQ ID NO:723).

88. The method of any of claims 68-87, wherein the at least one gRNA includes a targeting domain comprising the sequence CGACUGGCCAGGGCGCCUGU (SEQ ID NO:582).

89. The method of any of claims 68-79, wherein the Cas9 molecule is a nickase and two Cas9 molecule/gRNA molecule complexes are guided by two different gRNA molecules to cleave the target domain with two single stranded breaks on opposing strands of the target domain.

90. The method of any of claims 70-89, wherein the Cas9 molecule is an S. aureus Cas9 molecule.

91. The method of any of claims 70-90, wherein the Cas9 molecule is an S. pyogenes Cas9.

92. The method of any of claims 70-91, wherein the Cas9 molecule lacks an active RuvC domain or an active HNH domain.

93. The method of any of claims 70-89, 91 and 92, wherein the Cas9 molecule is an S. pyogenes Cas9 molecule comprising a D10A mutation.

94. The method of any of claims 89-93, wherein the two gRNA molecules comprise targeting domains that are selected from the following pairs of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
(SEQ ID NO: 582)
CGACUGGCCAGGGCGCCUGU
and
(SEQ ID NO: 511)
GGCCAGGAUGGUUCUUAGGU;
(SEQ ID NO: 582)
CGACUGGCCAGGGCGCCUGU
and
(SEQ ID NO: 512)
GGAUGGUUCUUAGGUAGGUG;
(SEQ ID NO: 582)
CGACUGGCCAGGGCGCCUGU
and
(SEQ ID NO: 576)
CGUCUGGGCGGUGCUACAAC;
(SEQ ID NO: 582)
CGACUGGCCAGGGCGCCUGU
and
(SEQ ID NO: 766)
CUACAACUGGGCUGGCGGCC;
(SEQ ID NO: 579)
UGUAGCACCGCCCAGACGAC
and
(SEQ ID NO: 511)
GGCCAGGAUGGUUCUUAGGU;
(SEQ ID NO: 579)
UGUAGCACCGCCCAGACGAC
and
(SEQ ID NO: 512)
GGAUGGUUCUUAGGUAGGUG;
or
(SEQ ID NO: 581)
ACCGCCCAGACGACUGGCCA
and
(SEQ ID NO: 511)
GGCCAGGAUGGUUCUUAGGU.

95. The method of any of claims 70-89 and 91-94, wherein the Cas9 molecule is an S. pyogenes Cas9 molecule comprising an N863A mutation.

96. The method of claim 94, wherein the two gRNA molecules comprise targeting domains that are selected from the following pairs of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
or
(SEQ ID NO: 582)
CGACUGGCCAGGGCGCCUGU
and
(SEQ ID NO: 511)
GGCCAGGAUGGUUCUUAGGU.

97. The method of any of claims 68-96, wherein the genetic disruption comprises creation of a double strand break which is repaired by non-homologous end joining (NHEJ) to effect insertions and deletions (indels) in the PDCD1 gene.

98. The method of any of claims 68-97, wherein the recombinant receptor is a functional non-TCR antigen receptor or a transgenic TCR.

99. The method of any of claims 68-98, wherein the recombinant receptor is a chimeric antigen receptor (CAR).

100. The method of claim 99, wherein the CAR comprises an antigen-binding domain that is an antibody or an antibody fragment.

101. The method of claim 100, wherein the antibody fragment is a single chain fragment.

102. The method of claim 100 or claim 101, wherein the antibody fragment comprises antibody variable regions joined by a flexible immunoglobulin linker.

103. The method of any of claims 100-102, wherein the fragment comprises an scFv.

104. The method of any of claims 100-103, wherein the antigen is associated with a disease or disorder.

105. The method of claim 104, wherein the disease or disorder is an infectious disease or condition, an autoimmune disease, an inflammatory disease or a tumor or a cancer.

106. The method of any of claims 68-105, wherein the recombinant receptor specifically binds to a tumor antigen.

107. The method of any of claims 68-106, wherein the antigen is selected from ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acethycholine e receptor, GD2, GD3, HMW-MAA, IL-22R-alpha, IL-13R-alpha2, kdr, kappa light chain, Lewis Y, L1-cell adhesion molecule (CD171), MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1), cyclin A1 (CCNA1), BCMA and interleukin 12.

108. The method of any of claims 68-107, wherein the recombinant receptor comprises an intracellular signaling domain comprising an ITAM.

109. The method of claim 108, wherein the intracellular signaling domain comprises an intracellular domain of a CD3-zeta (CD3) chain.

110. The method of claim 108 or claim 109, wherein the recombinant receptor further comprises a costimulatory signaling region.

111. The method of claim 110, wherein the costimulatory signaling region comprises a signaling domain of CD28 or 4-1BB.

112. The method of any of claims 68-111, wherein the nucleic acid encoding the recombinant receptor is a viral vector.

113. The method of claim 112, wherein the viral vector is a retroviral vector.

114. The method of claim 112 or claim 113, wherein the viral vector is a lentiviral vector or a gammaretroviral vector.

115. The method of any of claims 68-114, wherein introduction of the nucleic acid encoding the recombinant vector is by transduction, which optionally is retroviral transduction.

116. The method of any of claims 68-115, wherein the immune cell is a primary cell from a subject.

117. The method of any of claims 68-116, wherein the immune cell is a human cell.

118. The method of any of claims 68-117, wherein the immune cell is a white blood cell.

119. The method of any of claims 68-118, wherein the immune cell is an NK cell or T cell.

120. The method of claim 119, wherein the immune cell is a T cell that is an unfractionated T cell, isolated CD8+ T cell, or isolated CD4+ T cell.

121. The method of any of claims 68-120, that is performed on a plurality of immune cells.

122. The method of any of claims 68-121, wherein subsequent to introducing the agent and introducing the recombinant receptor, cells are not enriched or selected for (a) cells containing the genetic disruption or not expressing the endogenous PD-1 polypeptide, (b) cells expressing the recombinant receptor or both (a) and (b).

123. The method of any of claims 68-122, further comprising enriching or selecting for (a) cells containing the genetic disruption or not expressing the endogenous PD-1 polypeptide, (b) cells expressing the recombinant receptor or for both (a) and (b).

124. The method of any of claims 68-123, further comprising incubating the cells at or at about 37° C.±2° C.

125. The method of claim 124, wherein the incubation is carried out for a time between at or about 1 hour and at or about 96 hours, between at or about 4 hours and at or about 72 hours, between at or about 8 hours and at or about 48 hours, between at or about 12 hours and at or about 36 hours, between at or about 6 hours and at or about 24 hours, between at or about 36 hours and at or about 96 hours, inclusive.

126. The method of claim 125, wherein the incubation or a portion of the incubation is performed in the presence of a stimulating agent.

127. The method of claim 126, wherein the stimulating agent is an agent capable of inducing proliferation of T cells, CD4+ T cells and/or CD8+ T cells.

128. The method of claim 126 or claim 127, wherein the stimulating agent is or comprises an antibody specific for CD3 an antibody specific for CD28 and/or a cytokine.

129. The method of any of claims 68-128, further comprising formulating cells produced by the method in a pharmaceutically acceptable buffer.

130. The method of any of claims 68-129, wherein the method produces a population of cells in which:
at least about 70%, at least about 75%, or at least about 80% of the cells both 1) contain the genetic disruption; do not express the endogenous PD-1 polypeptide; do not contain a contiguous PDCD1 gene, a PDCD1 gene, and/or a functional PDCD1 gene; and/or do not express a PD-1 polypeptide; and 2) express the recombinant receptor; or
at least about 70%, at least about 75%, or at least about 80% of the cells that express the recombinant receptor contain the genetic disruption, do not express the endogenous PD-1 polypeptide, or do not express a PD-1 polypeptide.

131. The method of any of claims 68-130, wherein the method produces a population of cells in which:
greater than 80%, 81%, 82%, 83%, 84%, 85%, 86%, 87%, 88%, 89%, 90%, 91%, 92%, 93%, 94%, or 95% of the cells both 1) contain the genetic disruption; do not express the endogenous PD-1 polypeptide; do not contain a contiguous PDCD1 gene, a PDCD1 gene, and/or a functional PDCD1 gene; and/or do not express a PD-1 polypeptide and 2) express the recombinant receptor; and/or
greater than 80%, 81%, 82%, 83%, 84%, 85%, 86%, 87%, 88%, 89%, 90%, 91%, 92%, 93%, 94%, or 95% of the cells that express the recombinant receptor contain the genetic disruption, do not express the endogenous PD-1 polypeptide, or do not express a PD-1 polypeptide.

132. The method of any of claims 68-131, wherein both alleles of the gene in the genome are disrupted.

133. A genetically engineered immune cell produced by the method of any of claims 68-132.

134. A plurality of genetically engineered immune cells produced by the method of any of claims 68-132.

135. The plurality of genetically engineered immune cells of claim 134, wherein:
at least about 70%, at least about 75%, or at least about 80% of the cells both 1) contain the genetic disruption; do not express the endogenous PD-1 polypeptide; do not contain a contiguous PDCD1 gene, a PDCD1 gene, and/or a functional PDCD1 gene; and/or do not express a PD-1 polypeptide; and 2) express the recombinant receptor; or
at least about 70%, at least about 75%, or at least about 80% of the cells that express the recombinant receptor contain the genetic disruption, do not express the endogenous PD-1 polypeptide, or do not express a PD-1 polypeptide.

136. The plurality of genetically engineered immune cells of claim 134 or claim 135, wherein:
greater than 80%, 81%, 82%, 83%, 84%, 85%, 86%, 87%, 88%, 89%, 90%, 91%, 92%, 93%, 94%, or 95% of the cells both 1) contain the genetic disruption; do not express the endogenous PD-1 polypeptide; do not contain a contiguous PDCD1 gene, a PDCD1 gene, and/or a functional PDCD1 gene; and/or do not express a PD-1 polypeptide and 2) express the recombinant receptor; and/or
greater than 80%, 81%, 82%, 83%, 84%, 85%, 86%, 87%, 88%, 89%, 90%, 91%, 92%, 93%, 94%, or 95% of the cells that express the recombinant receptor contain the genetic disruption, do not express the endogenous PD-1 polypeptide, or do not express a PD-1 polypeptide.

137. A composition comprising the genetically engineered immune cell of claim 133 or the plurality of genetically engineered immune cells of any of claims 134-136, and optionally a pharmaceutically acceptable buffer.

138. A method of treatment, comprising administering the composition of any of claims 1-67 and 137 to a subject having a disease or condition.

139. The method of claim 138, wherein the recombinant receptor specifically binds to an antigen associated with the disease or condition.

140. The method of claim 138 or claim 139, wherein the disease or condition is a cancer, a tumor, an autoimmune disease or disorder, or an infectious disease.

141. The method of claim 140, wherein the cancer or tumor is leukemia, lymphoma, chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, colon cancer, lung cancer, liver cancer, breast cancer, prostate cancer, ovarian cancer, skin cancer, melanoma cancer, bone cancer, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

142. The method of any of claims 139-141, wherein the antigen is selected from the group consisting of orphan tyrosine kinase receptor ROR1, tEGFR, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, MAGE A3, CE7, Wilms Tumor 1 (WT-1), cyclin A1 (CCNA1), BCMA and interleukin 12.

143. The method of any of claims 139-142, wherein the antigen is CD19 or BCMA.

144. The method of any of claims 138-143, wherein the engineered cell administered to the subject has reduced and/or eliminated expression of PD-1 1 day, 2 days, 3 days, 4 days, 5 days, 6 days, 7 days, 10 days, 14 days, one month, two months, or more after administration.

145. The method of any of claims 138-144, wherein the engineered cell administered to the subject persists in the subject for at least 1 day, 2 days, 3 days, 4 days, 5 days, 6 days, 7 days, 10 days, 14 days, one month, two months, or more after administration.

146. The method of claim any of claims 138-145, wherein, at a point in time following administration of the composition:
cells expressing the recombinant receptor and not expressing PD-1 are detectable in the blood of the subject or blood-derived sample from the subject or in a tissue or biological sample of the subject;
cells containing the genetic disruption are detectable in the blood of the subject or a blood-derived sample from the subject or in a tissue or biological sample of the subject;
cells containing the genetic disruption and expressing the recombinant receptor are detectable in the blood of the subject or a blood-derived sample from the subject or in a tissue or biological sample of the subject.

147. The method of any of claims 138-146, wherein the time-point is at or about 7, 8, 9, 10, 11, 12, 13, or 14 days or 2, 3, 4, 5, 6, 7, 8, 9, 10, 11, or 12 weeks following administration.

148. The method of claim 138-147, wherein said cells detectable in the blood or sample are present at a concentration of at or about or at least at or about 1, 2, 3, 4, 5, 6, 7, 8, 9, 10, or 100 cells/microliter of blood and/or represent at least 10, 20, 25, 30, 35, 40, 45, or 50% or more of T cells in the blood.

149. The method of any of claims 138-148, wherein, following administration:
cells in the composition comprising the genetic disruption expand and/or persist in the subject at a rate, and/or for a time that, is at least as great as, optionally greater than, the expansion and/or persistence of T cells in the composition without the genetic disruption and/or as the expansion and/or persistence of T cells of a reference composition in which T cells express the recombinant receptor but do not comprising the deletion; and/or
cells in the composition comprising the genetic disruption and comprising the recombinant receptor expand and/or persist in the subject at a rate, and/or for a time that, is at least as great as, optionally greater than, the expansion and/or persistence of T cells in the composition without the genetic disruption and/or as the expansion and/or persistence of T cells of a reference composition in which T cells express the recombinant receptor but do not comprising the deletion.

150. The method of claim 149, wherein the rate or time is at least at or about 1.5-fold or 2-fold or 3-fold greater.

151. The method of any of claims 140-150, wherein the tumor is a solid tumor.

152. The method of any of claims 140-151, wherein the tumor is not a B cell-derived tumor, is not a leukemia and/or is not a lymphoma.

153. The method of any of claims 140-152, wherein the tumor or cells thereof express or have been observed to express a ligand for PD-1.

154. A pharmaceutical composition comprising an engineered immune cell that comprises (a) a recombinant receptor that specifically binds to an antigen; and (b) a genetic disruption of a PDCD1 gene encoding a PD-1 polypeptide, said genetic disruption preventing or reducing the expression of said PD-1 polypeptide, wherein the engineered cell has a phenotype of reduced and/or eliminated expression of PD-1 prior to administration to a subject and wherein the cells maintain the phenotype for 1 day, 2 days, 3 days, 4 days, 5 days, 6 days, 7 days, 10 days, 14 days, one month, two months, or more after administration to the subject.

155. A composition of any of claims 1-67, 137 and 154 for use in treating a disease or condition in a subject.

156. The composition for use of claim 155, wherein the recombinant receptor specifically binds to an antigen associated with the disease or condition.

157. The composition for use of claim 155 or claim 156, wherein the disease or condition is a cancer, a tumor, an autoimmune disease or disorder, or an infectious disease.

158. The composition for use of any of claims 155-157, wherein the disease or condition is a cancer or tumor, which is a leukemia, lymphoma, chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, colon cancer, lung cancer, liver cancer, breast cancer, prostate cancer, ovarian cancer, skin cancer, melanoma cancer, bone cancer, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

159. The composition for use of any of claims 155-158, wherein the antigen is selected from the group consisting of orphan tyrosine kinase receptor ROR1, tEGFR, Her2, Ll-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, MAGE A3, CE7, Wilms Tumor 1 (WT-1), cyclin A1 (CCNA1), BCMA and interleukin 12.

160. The composition for use of any of claims 155-159, wherein the antigen is CD19 or BCMA.

161. The composition for use of any of claims 155-160, wherein, following administration of the composition to subject,
one or more cells containing the genetic disruption, and optionally containing the recombinant receptor, persists in, and/or is detectable in a tissue or biological sample of, the subject at a time that is at least at or about 1 day, 2 days, 3 days, 4 days, 5 days, 6 days, 7 days, 10 days, 14 days, one month, two months, or more after administration; and/or
at least 50, 60, 70, 80, 85, or 90% of the T cells, or T cells expressing the recombinant receptor, that are detectable in a biological sample or tissue from the subject at a time that is at least at or about 1 day, 2 days, 3 days, 4 days, 5 days, 6 days, 7 days, 10 days, 14 days, one month, two months, or more after administration contain the genetic disruption.

162. A method of altering a T cell comprising contacting the T cell with one or more Cas9 molecule/gRNA molecule complexes, wherein the gRNA molecule(s) in the one or more Cas9 molecule/gRNA molecule complexes comprise a targeting domain which is complementary with a target domain from the PDCD1 gene.

163. A method of altering a T cell comprising contacting the T cell with two Cas9 molecule/gRNA molecule complexes, each complex comprising a gRNA molecule comprising a targeting domain which is complementary with a target domain from the PDCD1 gene.

164. The method of claim 162 or claim 163, wherein the T cell is from a subject suffering from cancer.

165. The method of claim 164, wherein the cancer is selected from the group consisting of: lymphoma, chronic lymphocytic leukemia (CLL), B cell acute lymphocytic leukemia (B-ALL), acute lymphoblastic leukemia, acute myeloid leukemia, non-Hodgkin's lymphoma (NHL), diffuse large cell lymphoma (DLCL), multiple myeloma, renal cell carcinoma (RCC), neuroblastoma, colorectal cancer, breast cancer, ovarian cancer, melanoma, sarcoma, prostate cancer, lung cancer, esophageal cancer, hepatocellular carcinoma, pancreatic cancer, astrocytoma, mesothelioma, head and neck cancer, and medulloblastoma.

166. The method of any of claims 162-165, wherein the T cell is from a subject having cancer or which could otherwise benefit from a mutation at a T cell target position of the PDCD1 gene.

167. The method of any of claims 162-166, wherein the contacting is performed ex vivo.

168. The method of any of claims 162-167, wherein the T cell comprises a recombinant receptor.

169. The method of any of claims 162-168, further comprising contacting the T cell with a nucleic acid encoding a recombinant receptor under conditions to introduce the nucleic acid into the cell.

170. The method of claim 168 or claim 169, wherein the recombinant receptor is a functional non-TCR antigen receptor or a transgenic TCR.

171. The method of any of claims 168-170, wherein the recombinant receptor is a chimeric antigen receptor (CAR).

172. The method of claim 171, wherein the CAR comprises an antigen-binding domain that is an antibody or an antibody fragment.

173. The method of claim 172, wherein the antibody fragment is a single chain fragment.

174. The method of claim 172 or claim 173, wherein the antibody fragment comprises antibody variable regions joined by a flexible immunoglobulin linker.

175. The method of any of claims 172-174, wherein the fragment comprises an scFv.

176. The method of any of claims 172-175, wherein the antigen is associated with a disease or disorder.

177. The method of claim 176, wherein the disease or disorder is an infectious disease or condition, an autoimmune disease, an inflammatory disease or a tumor or a cancer.

178. The method of any of claims 168-177, wherein the recombinant receptor specifically binds to a tumor antigen.

179. The method of any of claims 171-178, wherein the antigen is selected from ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acethycholine e receptor, GD2, GD3, HMW-MAA, IL-22R-alpha, IL-13R-alpha2, kdr, kappa light chain, Lewis Y, L1-cell adhesion molecule (CD171), MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1), cyclin A1 (CCNA1), BCMA and interleukin 12.

180. The method of any of claims 168-179, wherein the recombinant receptor comprises an intracellular signaling domain comprising an ITAM.

181. The method of claim 180, wherein the intracellular signaling domain comprises an intracellular domain of a CD3-zeta (CD3) chain.

182. The method of claim 180 or claim 181, wherein the recombinant receptor further comprises a costimulatory signaling region.

183. The method of claim 182, wherein the costimulatory signaling region comprises a signaling domain of CD28 or 4-1BB.

184. The method of any of claims 164-183, wherein the altered T cell is returned to the subject's body after the step of contacting.

185. The method of any of claims 162-184, wherein the T cell is from a subject suffering from cancer, the contacting is performed ex vivo and the altered T cell is returned to the subject's body after the step of contacting.

186. The method of any of claims 162-185, wherein the one or more Cas9 molecule/gRNA molecule complexes are formed prior to the contacting.

187. The method of any of claims 163-186, wherein the two Cas9 molecule/gRNA molecule complexes are formed prior to the contacting.

188. The method of any of claims 162-187, wherein the gRNA molecule(s) comprise a targeting domain that is the same as, or differs by no more than 3 nucleotides from, a targeting domain from any of SEQ ID NOS: 481-555, 563-1516, 1517-3748, 14657-16670, and 16671-21037

189. The method of claim 188, wherein the gRNA molecule(s) comprise a targeting domain that is selected from SEQ ID NOS: 563-1516.

190. The method of claim 188, wherein the gRNA molecule(s) comprise a targeting domain that is selected from SEQ ID NOS: 1517-3748.

191. The method of claim 188, wherein the gRNA molecule(s) comprise a targeting domain that is selected from SEQ ID NOS: 14657-16670.

192. The method of claim 188, wherein the gRNA molecule(s) comprise a targeting domain that is selected from SEQ ID NOS: 16671-21037.

193. The method of claim 188, wherein the gRNA molecule(s) comprise a targeting domain that is selected from SEQ ID NOS: 481-500 and 508-547.

194. The method of claim 188, wherein the gRNA molecule(s) comprise a targeting domain that is selected from SEQ ID NOS: 501-507 and 548-555.

195. The method of claim 188, wherein the gRNA molecule(s) comprise a targeting domain that is selected from SEQ ID NOS: 508, 514, 576, 579, 582, and 723.

196. The method of claim 188, wherein the gRNA molecule(s) comprise a targeting domain that is selected from SEQ ID NOS: 508, 510, 511, 512, 514, 576, 579, 581, 582, 766, and 723.

197. The method of any of claims 162-196, wherein the gRNA molecule(s) are modified at their 5′ end or comprise a 3′ polyA tail.

198. The method of any of claims 162-196, wherein the gRNA molecule(s) are modified at their 5′ end and comprise a 3′ polyA tail.

199. The method of claim 197 or claim 198, wherein the gRNA molecule(s) lack a 5′ triphosphate group.

200. The method of claim 197 or claim 198, wherein the gRNA molecule(s) include a 5′ cap.

201. The method of claim 200, wherein the 5′ cap comprises a modified guanine nucleotide that is linked to the remainder of the gRNA molecule via a 5′-5′ triphosphate linkage.

202. The method of claim 200, wherein the 5′ cap comprises two optionally modified guanine nucleotides that are linked via an optionally modified 5′-5′ triphosphate linkage.

203. The method of any of claims 197-202, wherein the 3′ polyA tail is comprised of about 10 to about 30 adenine nucleotides.

204. The method of any of claims 197-202, wherein the 3′ polyA tail is comprised of about 20 adenine nucleotides.

205. The method of claim 203 or claim 204, wherein the gRNA molecule(s) including the 3′ polyA tail were prepared by in vitro transcription from a DNA template.

206. The method of claim 205, wherein the 5′ nucleotide of the targeting domain is a guanine nucleotide, the DNA template comprises a T7 promoter sequence located immediately upstream of the sequence that corresponds to the targeting domain, and the 3′ nucleotide of the T7 promoter sequence is not a guanine nucleotide.

207. The method of claim 205, wherein the 5′ nucleotide of the targeting domain is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

208. The method of any of claims 162-207, wherein the one or more Cas9 molecule/gRNA molecule complexes are delivered into the T cell via electroporation.

209. The method of any of claims 163-208, wherein the at least two Cas9 molecule/gRNA molecule complexes are delivered into the T cell via electroporation.

210. The method of any of claims 162-209, wherein the gRNA molecule(s) comprise a targeting domain which is complementary with a target domain from the PDCD1 gene and wherein the gRNA molecule(s) guide the Cas9 molecule to cleave the target domain with an efficiency of cleavage of at least 40%.

211. The method of claim 210, wherein the efficiency of cleavage is determined using a labeled anti-PDCD1 antibody and a flow cytometry assay.

212. The method of any of claims 162-211, wherein the Cas9 molecule is guided by a single gRNA molecule and cleaves the target domain with a single double stranded break.

213. The method of claim 212, wherein the Cas9 molecule is a S. pyogenes Cas9 molecule.

214. The method of any of claims 162-213, wherein the targeting domain is selected from:
(SEQ ID NO: 508)
GUCUGGGCGGUGCUACAACU;
(SEQ ID NO: 514)
GCCCUGGCCAGUCGUCU;
(SEQ ID NO: 576)
CGUCUGGGCGGUGCUACAAC;
(SEQ ID NO: 579)
UGUAGCACCGCCCAGACGAC;
(SEQ ID NO: 582)
CGACUGGCCAGGGCGCCUGU;
or
(SEQ ID NO: 723)
CACCUACCUAAGAACCAUCC.

215. The method of any of claims 162-211, wherein the Cas9 molecule is a nickase and two Cas9 molecule/gRNA molecule complexes are guided by two different gRNA molecules to cleave the target domain with two single stranded breaks on opposing strands of the target domain.

216. The method of claim 215, wherein the Cas9 molecule is a S. pyogenes Cas9 molecule.

217. The method of any of claims 162-216, wherein the S. pyogenes Cas9 molecule has a D10A mutation.

218. The method of any of claims 162-217, wherein the two gRNA molecules comprise targeting domains that are selected from the following pairs of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
(SEQ ID NO: 582)
CGACUGGCCAGGGCGCCUGU
and
(SEQ ID NO: 511)
GGCCAGGAUGGUUCUUAGGU;
(SEQ ID NO: 582)
CGACUGGCCAGGGCGCCUGU
and
(SEQ ID NO: 512)
GGAUGGUUCUUAGGUAGGUG;
(SEQ ID NO: 582)
CGACUGGCCAGGGCGCCUGU
and
(SEQ ID NO: 576)
CGUCUGGGCGGUGCUACAAC;
(SEQ ID NO: 582)
CGACUGGCCAGGGCGCCUGU
and
(SEQ ID NO: 766)
CUACAACUGGGCUGGCGGCC;
(SEQ ID NO: 579)
UGUAGCACCGCCCAGACGAC
and
(SEQ ID NO: 511)
GGCCAGGAUGGUUCUUAGGU;
(SEQ ID NO: 579)
UGUAGCACCGCCCAGACGAC
and
(SEQ ID NO: 512)
GGAUGGUUCUUAGGUAGGUG;
or
(SEQ ID NO: 581)
ACCGCCCAGACGACUGGCCA
and
(SEQ ID NO: 511)
GGCCAGGAUGGUUCUUAGGU.

219. The method of any of claims 162-218, wherein the S. pyogenes Cas9 molecule has a N863A mutation.

220. The method of claim 219, wherein the two gRNA molecules comprise targeting domains that are selected from the following pairs of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
or
(SEQ ID NO: 582)
CGACUGGCCAGGGCGCCUGU
and
(SEQ ID NO: 511)
GGCCAGGAUGGUUCUUAGGU.

221. The method of any of claims 162-220, wherein the gRNA molecule(s) are modular gRNA molecule(s).

222. The method of any of claims 162-220, wherein the gRNA molecule(s) are chimeric gRNA molecule(s).

223. The method of claim 222, wherein the gRNA molecule(s) comprise from 5′ to 3′:
a targeting domain; a first complementarity domain; a linking domain; a second complementarity domain; a proximal domain; and a tail domain.

224. The method of claim 222 or claim 223, wherein the gRNA molecule(s) comprise a linking domain of no more than 25 nucleotides in length and a proximal and tail domain, that taken together, are at least 20 nucleotides in length.

225. The method of any one of claims 210-224, wherein the method is characterized by an efficiency of cleavage of at least 60%.

226. The method of any one of claims 210-224, wherein the method is characterized by an efficiency of cleavage of at least 80%.

227. The method of any one of claims 210-224, wherein the method is characterized by an efficiency of cleavage of at least 90%.

228. The method of any one of claims 210-227, wherein the gRNA molecule is characterized by fewer than 5 off-targets.

229. The method of any one of claims 210-228, wherein the gRNA molecule is characterized by fewer than 2 exonic off-targets.

230. The method of claim 228 or claim 229, wherein off-targets are identified by GUIDE-seq.

231. The method of claim 228 or claim 229, wherein off-targets are identified by Amp-seq.

232. A Cas9 molecule/gRNA molecule complex, wherein the gRNA molecule comprises a targeting domain which is complementary with a target domain from the PDCD1 gene, and the gRNA molecule is modified at its 5′ end and/or comprises a 3′ polyA tail.

233. The Cas9 molecule/gRNA molecule complex of claim 232, wherein the gRNA molecule comprises a targeting domain that is the same as, or differs by no more than 3 nucleotides from, a targeting domain from any of SEQ ID NOS: 481-555, 563-1516, 1517-3748, 14657-16670, and 16671-21037

234. The Cas9 molecule/gRNA molecule complex of claim 232, wherein the gRNA molecule comprises a targeting domain that is selected from SEQ ID NOS: 563-1516.

235. The Cas9 molecule/gRNA molecule complex of claim 232, wherein the gRNA molecule comprises a targeting domain that is selected from SEQ ID NOS: 1517-3748.

236. The Cas9 molecule/gRNA molecule complex of claim 232, wherein the gRNA molecule comprises a targeting domain that is selected from SEQ ID NOS: 14657-16670.

237. The Cas9 molecule/gRNA molecule complex of claim 232, wherein the gRNA molecule comprises a targeting domain that is selected from SEQ ID NOS: 16671-21037.

238. The Cas9 molecule/gRNA molecule complex of claim 232, wherein the gRNA molecule comprises a targeting domain that is selected from SEQ ID NOS: 481-500 and 508-547.

239. The Cas9 molecule/gRNA molecule complex of claim 232, wherein the gRNA molecule comprises a targeting domain that is selected from SEQ ID NOS: 501-507 and 548-555.

240. The Cas9 molecule/gRNA molecule complex of claim 232, wherein the gRNA molecule comprises a targeting domain that is selected from SEQ ID NOS: 508, 514, 576, 579, 582, and 723.

241. The Cas9 molecule/gRNA molecule complex of claim 232, wherein the gRNA molecule comprises a targeting domain that is selected from SEQ ID NOS: 508, 510, 511, 512, 514, 576, 579, 581, 582, 766, and 723.

242. The Cas9 molecule/gRNA molecule complex of any of claims 232-241, wherein the gRNA molecule is modified at its 5′ end.

243. The Cas9 molecule/gRNA molecule complex of claim 242, wherein the gRNA molecule lacks a 5′ triphosphate group.

244. The Cas9 molecule/gRNA molecule complex of claim 242, wherein the gRNA molecule includes a 5′ cap.

245. The Cas9 molecule/gRNA molecule complex of claim 244, wherein the 5′ cap comprises a modified guanine nucleotide that is linked to the remainder of the gRNA molecule via a 5′-5′ triphosphate linkage.

246. The Cas9 molecule/gRNA molecule complex of claim 244, wherein the 5′ cap comprises two optionally modified guanine nucleotides that are linked via an optionally modified 5′-5′ triphosphate linkage.

247. The Cas9 molecule/gRNA molecule complex of any of claims 232-246, wherein the 3′ polyA tail is comprised of about 10 to about 30 adenine nucleotides.

248. The Cas9 molecule/gRNA molecule complex of any of claims 232-246, wherein the 3′ polyA tail is comprised of about 20 adenine nucleotides.

249. The Cas9 molecule/gRNA molecule complex of claim 247 or claim 248, wherein the gRNA molecule including the 3′ polyA tail was prepared by in vitro transcription from a DNA template.

250. The Cas9 molecule/gRNA molecule complex of claim 249, wherein the 5′ nucleotide of the targeting domain is a guanine nucleotide, the DNA template comprises a T7 promoter sequence located immediately upstream of the sequence that corresponds to the targeting domain, and the 3′ nucleotide of the T7 promoter sequence is not a guanine nucleotide.

251. The Cas9 molecule/gRNA molecule complex of claim 249, wherein the 5′ nucleotide of the targeting domain is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

252. The Cas9 molecule/gRNA molecule complex of any of claims 232-251, wherein the Cas9 molecule cleaves a target domain with a double stranded break.

253. The Cas9 molecule/gRNA molecule complex of claim 252, wherein the Cas9 molecule is a S. pyogenes Cas9 molecule.

254. The Cas9 molecule/gRNA molecule complex of any of claims 232-253, wherein the targeting domain is selected from the following group of targeting domains:
(SEQ ID NO: 508)
GUCUGGGCGGUGCUACAACU;
(SEQ ID NO: 514)
GCCCUGGCCAGUCGUCU;
(SEQ ID NO: 576)
CGUCUGGGCGGUGCUACAAC;
(SEQ ID NO: 579)
UGUAGCACCGCCCAGACGAC;
(SEQ ID NO: 582)
CGACUGGCCAGGGCGCCUGU;
or
(SEQ ID NO: 723)
CACCUACCUAAGAACCAUCC.

255. The Cas9 molecule/gRNA molecule complex of any of claims 232-251, wherein the Cas9 molecule cleaves a target domain with a single stranded break.

256. The Cas9 molecule/gRNA molecule complex of claim 255, wherein the Cas9 molecule is a S. pyogenes Cas9 molecule.

257. The Cas9 molecule/gRNA molecule complex of any of claim 232-claim 256, wherein the S. pyogenes Cas9 molecule has a D10A mutation.

258. The Cas9 molecule/gRNA molecule complex of any of claim 232-claim 257, wherein the targeting domain is selected from the following group of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
(SEQ ID NO: 582)
CGACUGGCCAGGGCGCCUGU
and
(SEQ ID NO: 511)
GGCCAGGAUGGUUCUUAGGU;
(SEQ ID NO: 582)
CGACUGGCCAGGGCGCCUGU
and
(SEQ ID NO: 512)
GGAUGGUUCUUAGGUAGGUG;
(SEQ ID NO: 582)
CGACUGGCCAGGGCGCCUGU
and
(SEQ ID NO: 576)
CGUCUGGGCGGUGCUACAAC;
(SEQ ID NO: 582)
CGACUGGCCAGGGCGCCUGU
and
(SEQ ID NO: 766)
CUACAACUGGGCUGGCGGCC;
(SEQ ID NO: 579)
UGUAGCACCGCCCAGACGAC
and
(SEQ ID NO: 511)
GGCCAGGAUGGUUCUUAGGU;
(SEQ ID NO: 579)
UGUAGCACCGCCCAGACGAC
and
(SEQ ID NO: 512)
GGAUGGUUCUUAGGUAGGUG;
or
(SEQ ID NO: 581)
ACCGCCCAGACGACUGGCCA
and
(SEQ ID NO: 511)
GGCCAGGAUGGUUCUUAGGU.

259. The Cas9 molecule/gRNA molecule complex of any of claim 232-256, wherein the S. pyogenes Cas9 molecule has a N863A mutation.

260. The Cas9 molecule/gRNA molecule complex of claim 259, wherein the targeting domain is selected from the following group of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
or
(SEQ ID NO: 582)
CGACUGGCCAGGGCGCCUGU
and
(SEQ ID NO: 511)
GGCCAGGAUGGUUCUUAGGU.

261. The Cas9 molecule/gRNA molecule complex of any of claims 232-260, wherein the gRNA molecule is a modular gRNA molecule.

262. The Cas9 molecule/gRNA molecule complex of any of claims 232-261, wherein the gRNA molecule is a chimeric gRNA molecule.

263. The Cas9 molecule/gRNA molecule complex of claim 262, wherein the gRNA molecule comprises from 5′ to 3′: a targeting domain; a first complementarity domain; a linking domain; a second complementarity domain; a proximal domain; and a tail domain.

264. The Cas9 molecule/gRNA molecule complex of claim 262 or claim 263, wherein the gRNA molecule comprises a linking domain of no more than 25 nucleotides in length and a proximal and tail domain, that taken together, are at least 20 nucleotides in length.

265. A composition comprising at least two Cas9 molecule/gRNA complexes, each complex comprising a gRNA molecule comprising a targeting domain that is complementary to a target domain from a PDCD1 gene.

266. The composition of claim 265, wherein the gRNA molecule comprises a targeting domain that is the same as, or differs by no more than 3 nucleotides from, a targeting domain from any of SEQ ID NOS: 481-555, 563-1516, 1517-3748, 14657-16670, and 16671-21037

267. The composition of claim 265, wherein the gRNA molecule comprises a targeting domain that is selected from SEQ ID NOS: 563-1516.

268. The composition of claim 265, wherein the gRNA molecule comprises a targeting domain that is selected from SEQ ID NOS: 1517-3748.

269. The composition of claim 265, wherein the gRNA molecule comprises a targeting domain that is selected from SEQ ID NOS: 14657-16670.

270. The composition of claim 265, wherein the gRNA molecule comprises a targeting domain that is selected from SEQ ID NOS: 16671-21037.

271. The composition of claim 265, wherein the gRNA molecule comprises a targeting domain that is selected from SEQ ID NOS: 481-500 and 508-547.

272. The composition of claim 265, wherein the gRNA molecule comprises a targeting domain that is selected from SEQ ID NOS: 501-507 and 548-555.

273. The composition of claim 265, wherein the gRNA molecule comprises a targeting domain that is selected from SEQ ID NOS: 508, 514, 576, 579, 582, and 723.

274. The composition of claim 265, wherein the gRNA molecule comprises a targeting domain that is selected from SEQ ID NOS: 508, 510, 511, 512, 514, 576, 579, 581, 582, 766, and 723.

275. The composition of any of claims 265-741, wherein the gRNA molecule is modified at its 5′ end.

276. The composition of claim 275, wherein the gRNA molecule lacks a 5′ triphosphate group.

277. The composition of claim 275, wherein the gRNA molecule includes a 5′ cap.

278. The composition of claim 277, wherein the 5′ cap comprises a modified guanine nucleotide that is linked to the remainder of the gRNA molecule via a 5′-5′ triphosphate linkage.

279. The composition of claim 277, wherein the 5′ cap comprises two optionally modified guanine nucleotides that are linked via an optionally modified 5′-5′ triphosphate linkage.

280. The composition of any of claims 265-279, wherein the 3′ polyA tail is comprised of about 10 to about 30 adenine nucleotides.

281. The composition of any of claims 265-279, wherein the 3′ polyA tail is comprised of about 20 adenine nucleotides.

282. The composition of claim 280 or claim 281, wherein the gRNA molecule including the 3′ polyA tail was prepared by in vitro transcription from a DNA template.

283. The composition of claim 282, wherein the 5′ nucleotide of the targeting domain is a guanine nucleotide, the DNA template comprises a T7 promoter sequence located immediately upstream of the sequence that corresponds to the targeting domain, and the 3′ nucleotide of the T7 promoter sequence is not a guanine nucleotide.

284. The composition of claim 282, wherein the 5′ nucleotide of the targeting domain is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

285. The composition of any of claims 265-284, wherein the Cas9 molecule cleaves a target domain with a double stranded break.

286. The composition of claim 285, wherein the Cas9 molecule is a S. pyogenes Cas9 molecule.

287. The composition of any of claims 265-286, wherein the targeting domain is selected from the following group of targeting domains:
(SEQ ID NO: 508)
GUCUGGGCGGUGCUACAACU;
(SEQ ID NO: 514)
GCCCUGGCCAGUCGUCU;
(SEQ ID NO: 576)
CGUCUGGGCGGUGCUACAAC;
(SEQ ID NO: 579)
UGUAGCACCGCCCAGACGAC;
(SEQ ID NO: 582)
CGACUGGCCAGGGCGCCUGU;
or
(SEQ ID NO: 723)
CACCUACCUAAGAACCAUCC.

288. The composition of any of claims 265-287, wherein the Cas9 molecule cleaves a target domain with a single stranded break.

289. The composition of claim 288, wherein the Cas9 molecule is a S. pyogenes Cas9 molecule.

290. The composition of any of claims 265-289, wherein the S. pyogenes Cas9 molecule has a D10A mutation.

291. The composition of any of claims 265-290, wherein the targeting domain is selected from the following group of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
(SEQ ID NO: 582)
CGACUGGCCAGGGCGCCUGU
and
(SEQ ID NO: 511)
GGCCAGGAUGGUUCUUAGGU;
(SEQ ID NO: 582)
CGACUGGCCAGGGCGCCUGU
and
(SEQ ID NO: 512)
GGAUGGUUCUUAGGUAGGUG;
(SEQ ID NO: 582)
CGACUGGCCAGGGCGCCUGU
and
(SEQ ID NO: 576)
CGUCUGGGCGGUGCUACAAC;
(SEQ ID NO: 582)
CGACUGGCCAGGGCGCCUGU
and
(SEQ ID NO: 766)
CUACAACUGGGCUGGCGGCC;
(SEQ ID NO: 579)
UGUAGCACCGCCCAGACGAC
and
(SEQ ID NO: 511)
GGCCAGGAUGGUUCUUAGGU;
(SEQ ID NO: 579)
UGUAGCACCGCCCAGACGAC
and
(SEQ ID NO: 512)
GGAUGGUUCUUAGGUAGGUG;
or
(SEQ ID NO: 581)
ACCGCCCAGACGACUGGCCA
and
(SEQ ID NO: 511)
GGCCAGGAUGGUUCUUAGGU.

292. The composition of any of claim 265-291, wherein the S. pyogenes Cas9 molecule has a N863A mutation.

293. The composition of claim 292, wherein the targeting domain is selected from the following group of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
or
(SEQ ID NO: 582)
CGACUGGCCAGGGCGCCUGU
and
(SEQ ID NO: 511)
GGCCAGGAUGGUUCUUAGGU.

294. The composition of any of claims 265-293, wherein the gRNA molecule is a modular gRNA molecule.

295. The composition of any of claims 265-294, wherein the gRNA molecule is a chimeric gRNA molecule.

296. The composition of claim 295, wherein the gRNA molecule comprises from 5′ to 3′: a targeting domain; a first complementarity domain; a linking domain; a second complementarity domain; a proximal domain; and a tail domain.

297. The composition of claim 295 or claim 296, wherein the gRNA molecule comprises a linking domain of no more than 25 nucleotides in length and a proximal and tail domain, that taken together, are at least 20 nucleotides in length.

298. A gRNA molecule that comprises a targeting domain which is complementary with a target domain from the PDCD1 gene, wherein the gRNA molecule is modified at its 5′ end and/or comprises a 3′ polyA tail.

299. The gRNA molecule of claim 298, wherein the gRNA molecule comprises a targeting domain that is the same as, or differs by no more than 3 nucleotides from, a targeting domain from any of SEQ ID NOS: 481-555, 563-1516, 1517-3748, 14657-16670, and 16671-21037

300. The gRNA molecule of claim 298, wherein the gRNA molecule comprises a targeting domain that is selected from SEQ ID NOS: 563-1516.

301. The gRNA molecule of claim 298, wherein the gRNA molecule comprises a targeting domain that is selected from SEQ ID NOS: 1517-3748.

302. The gRNA molecule of claim 298, wherein the gRNA molecule comprises a targeting domain that is selected from SEQ ID NOS: 14657-16670.

303. The gRNA molecule of claim 298, wherein the gRNA molecule comprises a targeting domain that is selected from SEQ ID NOS: 16671-21037.

304. The gRNA molecule of claim 298, wherein the gRNA molecule comprises a targeting domain that is selected from SEQ ID NOS: 481-500 and 508-547.

305. The gRNA molecule of claim 298, wherein the gRNA molecule comprises a targeting domain that is selected from SEQ ID NOS: 501-507 and 548-555.

306. The gRNA molecule of claim 298, wherein the gRNA molecule comprises a targeting domain that is selected from SEQ ID NOS: 508, 514, 576, 579, 582, and 723.

307. The gRNA molecule of claim 298, wherein the gRNA molecule comprises a targeting domain that is selected from SEQ ID NOS: 508, 510, 511, 512, 514, 576, 579, 581, 582, 766, and 723.

308. The gRNA molecule of any of claims claim 298-94, wherein the gRNA molecule is modified at its 5′ end.

309. The gRNA molecule of claim 308, wherein the gRNA molecule lacks a 5′ triphosphate group.

310. The gRNA molecule of claim 308, wherein the gRNA molecule includes a 5′ cap.

311. The gRNA molecule of claim 310, wherein the 5′ cap comprises a modified guanine nucleotide that is linked to the remainder of the gRNA molecule via a 5′-5′ triphosphate linkage.

312. The gRNA molecule of claim 310, wherein the 5′ cap comprises two optionally modified guanine nucleotides that are linked via an optionally modified 5′-5′ triphosphate linkage.

313. The gRNA molecule of any of claims 298-312, wherein the gRNA molecule comprises a 3′ polyA tail which is comprised of about 10 to about 30 adenine nucleotides.

314. The gRNA molecule of any of claims 298-312, wherein the gRNA molecule comprises a 3′ polyA tail which is comprised of about 20 adenine nucleotides.

315. The gRNA molecule of claim 313 or 314, wherein the gRNA molecule including the 3′ polyA tail was prepared by in vitro transcription from a DNA template.

316. The gRNA molecule of claim 315, wherein the 5′ nucleotide of the targeting domain is a guanine nucleotide, the DNA template comprises a T7 promoter sequence located immediately upstream of the sequence that corresponds to the targeting domain, and the 3′ nucleotide of the T7 promoter sequence is not a guanine nucleotide.

316. The gRNA molecule of claim 315, wherein the 5′ nucleotide of the targeting domain is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

317. The gRNA molecule of any of claims 298-316, wherein the gRNA molecule is a S. pyogenes gRNA molecule.

318. The gRNA molecule of any of claims 298-317, wherein the targeting domain is selected from the following group of targeting domains:
(SEQ ID NO: 508)
GUCUGGGCGGUGCUACAACU;
(SEQ ID NO: 514)
GCCCUGGCCAGUCGUCU;
(SEQ ID NO: 576)
CGUCUGGGCGGUGCUACAAC;
(SEQ ID NO: 579)
UGUAGCACCGCCCAGACGAC;
(SEQ ID NO: 582)
CGACUGGCCAGGGCGCCUGU;
or
(SEQ ID NO: 723)
CACCUACCUAAGAACCAUCC.

319. The gRNA molecule of claim 318, wherein the targeting domain is selected from the following group of targeting domains:
(SEQ ID NO: 514)
GCCCUGGCCAGUCGUCU;
(SEQ ID NO: 723)
CACCUACCUAAGAACCAUCC.

320. The gRNA molecule of claim 318, wherein the targeting domain is selected from the following group of targeting domains:
(SEQ ID NO: 510)
GGGCGGUGCUACAACUGGGC;
(SEQ ID NO: 511)
GGCCAGGAUGGUUCUUAGGU;
(SEQ ID NO: 512)
GGAUGGUUCUUAGGUAGGUG;
(SEQ ID NO: 581)
ACCGCCCAGACGACUGGCCA
and
(SEQ ID NO: 766)
CUACAACUGGGCUGGCGGCC;

321. The gRNA molecule of claim 318, wherein the targeting domain is selected from the following group of targeting domains:
(SEQ ID NO: 511)
GGCCAGGAUGGUUCUUAGGU;
(SEQ ID NO: 512)
GGAUGGUUCUUAGGUAGGUG;
(SEQ ID NO: 766)
CUACAACUGGGCUGGCGGCC;

322. The gRNA molecule of any of claims 298-321, wherein the gRNA molecule is a modular gRNA molecule.

323. The gRNA molecule of any of claims 298-322, wherein the gRNA molecule is a chimeric gRNA molecule.

324. The gRNA molecule of claim 323, wherein the gRNA molecule comprises from 5′ to 3′: a targeting domain; a first complementarity domain; a linking domain; a second complementarity domain; a proximal domain; and a tail domain.

325. The gRNA molecule of claim 323 or claim 324, wherein the gRNA molecule comprises a linking domain of no more than 25 nucleotides in length and a proximal and tail domain, that taken together, are at least 20 nucleotides in length.

326. A method of making a cell for implantation, comprising contacting the cell with one or more Cas9 molecule/gRNA molecule complexes, wherein the gRNA molecule(s) in the one or more Cas9 molecule/gRNA molecule complexes comprise a targeting domain which is complementary with a target domain from the PDCD1 gene.

327. The method of claim 326, wherein the gRNA molecule(s) comprise a targeting domain which is complementary with a target domain from the PDCD1 gene and wherein the gRNA molecule(s) guide the Cas9 molecule to cleave the target domain with an efficiency of cleavage of at least 40%.

328. The method of claim 327, wherein the efficiency of cleavage is determined using a labeled anti-PDCD1 antibody and a flow cytometry assay

329. The method of any of claims 326-328, wherein the gRNA molecule(s) are modified at their 5′ end or comprise a 3′ polyA tail.

330. The method of any of claims 326-328, wherein the gRNA molecule(s) are modified at their 5′ end and comprise a 3′ polyA tail.

331. The method of claim 329 or claim 330, wherein the gRNA molecule(s) lack a 5′ triphosphate group.

332. The method of claim 329 or claim 330, wherein the gRNA molecule(s) include a 5′ cap.

333. The method of claim 332, wherein the 5′ cap comprises a modified guanine nucleotide that is linked to the remainder of the gRNA molecule via a 5′-5′ triphosphate linkage.

334. The method of claim 332, wherein the 5′ cap comprises two optionally modified guanine nucleotides that are linked via an optionally modified 5′-5′ triphosphate linkage.

335. The method of any of claims 329-334, wherein the 3′ polyA tail is comprised of about 10 to about 30 adenine nucleotides.

336. The method of any of claims 329-334, wherein the 3′ polyA tail is comprised of about 20 adenine nucleotides.

337. The method of claim 335 or claim 336, wherein the gRNA molecule(s) including the 3′ polyA tail were prepared by in vitro transcription from a DNA template.

338. The method of claim 337, wherein the 5′ nucleotide of the targeting domain is a guanine nucleotide, the DNA template comprises a T7 promoter sequence located immediately upstream of the sequence that corresponds to the targeting domain, and the 3′ nucleotide of the T7 promoter sequence is not a guanine nucleotide.

339. The method of claim 337, wherein the 5′ nucleotide of the targeting domain is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

340. The method of any of claims 326-339, wherein the one or more Cas9 molecule/gRNA molecule complexes are delivered into the cell via electroporation.

341. The method of any of claims 326-340, wherein the Cas9 molecule is guided by a single gRNA molecule and cleaves the target domain with a single double stranded break.

342. The method of claim 341, wherein the Cas9 molecule is a S. pyogenes Cas9 molecule.

343. The method of any of claims 326-342, wherein the single gRNA molecule comprises a targeting domain selected from the following targeting domains:
(SEQ ID NO: 508)
GUCUGGGCGGUGCUACAACU;
(SEQ ID NO: 514)
GCCCUGGCCAGUCGUCU;
(SEQ ID NO: 576)
CGUCUGGGCGGUGCUACAAC;
(SEQ ID NO: 579)
UGUAGCACCGCCCAGACGAC;
(SEQ ID NO: 582)
CGACUGGCCAGGGCGCCUGU;
or
(SEQ ID NO: 723)
CACCUACCUAAGAACCAUCC.

344. The method of any of claims 326-343, wherein the Cas9 molecule is a nickase and two Cas9 molecule/gRNA molecule complexes are guided by two different gRNA molecules to cleave the target domain with two single stranded breaks on opposing strands of the target domain.

345. The method of any of claims 326-344, wherein the Cas9 molecule is a S. pyogenes Cas9 molecule having a D10A mutation.

346. The method of any of claims 326-345, wherein the two gRNA molecules comprise targeting domains that are selected from the following pairs of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
(SEQ ID NO: 582)
CGACUGGCCAGGGCGCCUGU
and
(SEQ ID NO: 511)
GGCCAGGAUGGUUCUUAGGU;
(SEQ ID NO: 582)
CGACUGGCCAGGGCGCCUGU
and
(SEQ ID NO: 512)
GGAUGGUUCUUAGGUAGGUG;
(SEQ ID NO: 582)
CGACUGGCCAGGGCGCCUGU
and
(SEQ ID NO: 576)
CGUCUGGGCGGUGCUACAAC;
(SEQ ID NO: 582)
CGACUGGCCAGGGCGCCUGU
and
(SEQ ID NO: 766)
CUACAACUGGGCUGGCGGCC;
(SEQ ID NO: 579)
UGUAGCACCGCCCAGACGAC
and
(SEQ ID NO: 511)
GGCCAGGAUGGUUCUUAGGU;
(SEQ ID NO: 579)
UGUAGCACCGCCCAGACGAC
and
(SEQ ID NO: 512)
GGAUGGUUCUUAGGUAGGUG;
or
(SEQ ID NO: 581)
ACCGCCCAGACGACUGGCCA
and
(SEQ ID NO: 511)
GGCCAGGAUGGUUCUUAGGU.

347. The method of any of claims 326-346, wherein the S. pyogenes Cas9 molecule has a N863A mutation.

348. The method of claim 347, wherein the two gRNA molecules comprise targeting domains that are selected from the following pairs of targeting domains:
(SEQ ID NO: 582)
CGACUGGCCAGGGCGCCUGU
and
(SEQ ID NO: 508)
GUCUGGGCGGUGCUACAACU;
(SEQ ID NO: 582)
CGACUGGCCAGGGCGCCUGU
and
(SEQ ID NO: 510)
GGGCGGUGCUACAACUGGGC;
or
(SEQ ID NO: 582)
CGACUGGCCAGGGCGCCUGU
and
(SEQ ID NO: 511)
GGCCAGGAUGGUUCUUAGGU.

349. The method of any of claims 326-348, wherein the gRNA molecule(s) are modular gRNA molecule(s).

350. The method of any of claims 326-349, wherein the gRNA molecule(s) are chimeric gRNA molecule(s).

351. The method of claim 350, wherein the gRNA molecule(s) comprise from 5′ to 3′: a targeting domain; a first complementarity domain; a linking domain; a second complementarity domain; a proximal domain; and a tail domain.

352. The method of claim 350 or claim 351, wherein the gRNA molecule(s) comprise a linking domain of no more than 25 nucleotides in length and a proximal and tail domain, that taken together, are at least 20 nucleotides in length.

353. The method of any of claims 326-352, wherein the gRNA molecule(s) guide the Cas9 molecule to cleave the target domain with an efficiency of cleavage of at least 60%.

354. The method of any of claims 326-352, wherein the gRNA molecule(s) guide the Cas9 molecule to cleave the target domain with an efficiency of cleavage of at least 80%.

355. The method of any of claims 326-352, wherein the gRNA molecule(s) guide the Cas9 molecule to cleave the target domain with an efficiency of cleavage of at least 90%.

356. The method of any of claims 326-355, wherein the one or more Cas9 molecule/gRNA molecule complexes produce fewer than 5 off-targets.

357. The method of any of claims 326-356, wherein the one or more Cas9 molecule/gRNA molecule complexes produce fewer than 2 exonic off-targets.

358. The method of claim 356 or claim 357, wherein off-targets are identified by GUIDE-seq.

359. The method of claim 356 or claim 357, wherein off-targets are identified by Amp-seq.

360. The method of any of claims 326-359, wherein the contacting is performed ex vivo.

361. The method of any of claims 326-360, wherein the cell is an immune cell.

362. The method of claim 361, wherein the cell is a lymphocyte or antigen presenting cell.

363. The method of claim 362, wherein the cell is a T cell, B cell or antigen presenting cell.

364. The method of any of claims 326-363, wherein the cell is a T cell.

365. The method of any of claims 326-364, wherein the cell comprises a recombinant receptor.

366. The method of any of claims 326-365, further comprising contacting the cell with a nucleic acid encoding a recombinant receptor under conditions to introduce the nucleic acid into the cell.

367. The method of claim 365 or claim 366, wherein the recombinant receptor is a functional non-TCR antigen receptor or a transgenic TCR.

368. The method of any of claims 365-367, wherein the recombinant receptor is a chimeric antigen receptor (CAR).

369. The method of claim 368, wherein the CAR comprises an antigen-binding domain that is an antibody or an antibody fragment.

370. The method of claim 369, wherein the antibody fragment is a single chain fragment.

371. The method of claim 369 or claim 370, wherein the antibody fragment comprises antibody variable regions joined by a flexible immunoglobulin linker.

372. The method of any of claims 369-371, wherein the fragment comprises an scFv.

373. The method of any of claims 369-372, wherein the antigen is associated with a disease or disorder.

374. The method of claim 373, wherein the disease or disorder is an infectious disease or condition, an autoimmune disease, an inflammatory disease or a tumor or a cancer.

375. The method of any of claims 365-374, wherein the recombinant receptor specifically binds to a tumor antigen.

376. The method of any of claims 369-375, wherein the antigen is selected from ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acethycholine e receptor, GD2, GD3, HMW-MAA, IL-22R-alpha, IL-13R-alpha2, kdr, kappa light chain, Lewis Y, L1-cell adhesion molecule (CD171), MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1), cyclin A1 (CCNA1), BCMA andinterleukin 12.

377. The method of any of claims 365-376, wherein the recombinant receptor comprises an intracellular signaling domain comprising an ITAM.

378. The method of claim 377, wherein the intracellular signaling domain comprises an intracellular domain of a CD3-zeta (CD3) chain.

379. The method of claim 377 or 378, wherein the recombinant receptor further comprises a costimulatory signaling region.

380. The method of claim 379, wherein the costimulatory signaling region comprises a signaling domain of CD28 or 4-1BB.

381. A T cell made by the method of any of claims 162-231 and 326-380.

382. A T cell comprising the Cas9 molecule/gRNA molecule complex of any of claims 232-264.

383. A T cell comprising the composition of any of claims 265-297.

384. A method of treating a subject, comprising administering to the subject the T cell of any of claims 381-383.

385. The Cas9 molecule/gRNA molecule complex of any of claims 232-264, the composition of any of claims 265-297, or the T cell of any of claims 381-383, for use in therapy.

386. Use of the Cas9 molecule/gRNA molecule complex of any of claims 232-264 or the composition of any of claims 265-297 in the manufacture of a medicament for treating cancer.
